(19) United States
(12) Patent Application Publication
(10) Pub. No.: US 2008/0312255 A1
(43) Pub. Date: Dec. 18, 2008

(54) PIPERDINE COMPOUNDS AND USES THEREOF-911

(75) Inventors: Gregory Basarab, Waltham, MA (US); Pamela Hill, Waltham, MA (US); Fei Zhou, Waltham, MA (US)

Correspondence Address: ASTRAZENECA R&D BOSTON, 35 GATEHOUSE DRIVE, WALTHAM, MA, 02451-1215, US

(73) Assignee: ASTRAZENECA AB, Sodertalje (SE)

(21) Appl. No.: 12/136,884

(22) Filed: Jun. 11, 2008

Related U.S. Application Data
(60) Provisional application No. 60/943,440, filed on Jun. 12, 2007.

(51) Int. Cl. A61K 31/497; A61K 31/506; C07D 417/14; A61P 31/04; A61K 31/4545

(52) U.S. Cl. 514/255.05; 544/333; 514/256; 546/193; 514/318; 544/405

(57) ABSTRACT

Compounds of formula (I) and their pharmaceutically acceptable salts are described. Processes for their preparation, pharmaceutical compositions containing them, their use as medicaments and their use in the treatment of bacterial infections are also described.

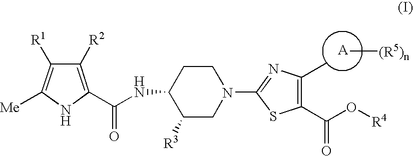

### RELATED APPLICATIONS

This application claims the benefit of U.S. provisional application No. 60/943,440, filed on Jun. 12, 2007, the entire teachings of which are incorporated herein by reference.

The present invention relates to compounds which demonstrate antibacterial activity, processes for their preparation, pharmaceutical compositions containing them as the active ingredient, to their use as medicaments and to their use in the manufacture of medicaments for use in the treatment of bacterial infections in warm-blooded animals such as humans. In particular this invention relates to compounds useful for the treatment of bacterial infections in warm-blooded animals such as humans, more particularly to the use of these compounds in the manufacture of medicaments for use in the treatment of bacterial infections in warm-blooded animals such as humans.

The international microbiological community continues to express serious concern that the evolution of antibiotic resistance could result in strains against which currently available antibacterial agents will be ineffective. In general, bacterial pathogens may be classified as either Gram-positive or Gram-negative pathogens. Antibiotic compounds with effective activity against both Gram-positive and Gram-negative pathogens are generally regarded as having a broad spectrum of activity. The compounds of the present invention are regarded as effective against both Gram-positive and certain Gram-negative pathogens.

Gram-positive pathogens, for example Staphylococci, Enterococci, Streptococci and mycobacteria, are particularly important because of the development of resistant strains which are both difficult to treat and difficult to eradicate from the hospital environment once established. Examples of such strains are methicillin resistant staphylococcus aureus (MRSA), methicillin resistant coagulase negative staphylococci (MRCNS), penicillin resistant Streptococcus pneumoniae and multiple resistant Enterococcus faecium.

The preferred clinically effective antibiotic for treatment of last resort of such resistant Gram-positive pathogens is vancomycin. Vancomycin is a glycopeptide and is associated with various toxicities, including nephrotoxicity. Furthermore, and most importantly, antibacterial resistance to vancomycin and other glycopeptides is also appearing. This resistance is increasing at a steady rate rendering these agents less and less effective in the treatment of Gram-positive pathogens. There is also now increasing resistance appearing towards agents such as β-lactams, quinolones and macrolides used for the treatment of upper respiratory tract infections, also caused by certain Gram negative strains including H. influenzae and M.catarrhalis.

Consequently, in order to overcome the threat of widespread multi-drug resistant organisms, there is an on-going need to develop new antibiotics, particularly those with either a novel mechanism of action and/or containing new pharmacophoric groups.

Deoxyribonucleic acid (DNA) gyrase is a member of the type II family of topoisomerases that control the topological state of DNA in cells (Champoux, J. J.; 2001. Ann. Rev. Biochem. 70: 369-413). Type II topoisomerases use the free energy from adenosine triphosphate (ATP) hydrolysis to alter the topology of DNA by introducing transient double-stranded breaks in the DNA, catalyzing strand passage through the break and resealing the DNA. DNA gyrase is an essential and conserved enzyme in bacteria and is unique among topoisomerases in its ability to introduce negative supercoils into DNA. The enzyme consists of two subunits, encoded by gyrA and gyrB, forming an A₂B₂ tetrameric complex. The A subunit of gyrase (GyrA) is involved in DNA breakage and resealing and contains a conserved tyrosine residue that forms the transient covalent link to DNA during strand passage. The B subunit (GyrB) catalyzes the hydrolysis of ATP and interacts with the A subunit to translate the free energy from hydrolysis to the conformational change in the enzyme that enables strand-passage and DNA resealing.

Another conserved and essential type II topoisomerase in bacteria, called topoisomerase IV, is primarily responsible for separating the linked closed circular bacterial chromosomes produced in replication. This enzyme is closely related to DNA gyrase and has a similar tetrameric structure formed from subunits homologous to Gyr A and to Gyr B. The overall sequence identity between gyrase and topoisomerase IV in different bacterial species is high. Therefore, compounds that target bacterial type II topoisomerases have the potential to inhibit two targets in cells, DNA gyrase and topoisomerase IV; as is the case for existing quinolone antibacterials (Maxwell, A. 1997, Trends Microbiol. 5: 102-109).

DNA gyrase is a well-validated target of antibacterials, including the quinolones and the coumarins. The quinolones (e.g. ciprofloxacin) are broad-spectrum antibacterials that inhibit the DNA breakage and reunion activity of the enzyme and trap the GyrA subunit covalently complexed with DNA (Drlica, K., and X. Zhao, 1997, Microbiol. Molec. Biol. Rev. 61: 377-392). Members of this class of antibacterials also inhibit topoisomerase IV and as a result, the primary target of these compounds varies among species. Although the quinolones are successful antibacterials, resistance generated by mutations in the target (DNA gyrase and topoisomerase IV) is becoming an increasing problem in several organisms, including S. aureus and Streptococcus pneumoniae (Hooper, D. C., 2002, The Lancet Infectious Diseases 2: 530-538). In addition, quinolones, as a chemical class, suffer from toxic side effects, including arthropathy that prevents their use in children (Lipsky, B. A. and Baker, C. A., 1999, Clin. Infect. Dis. 28: 352-364). Furthermore, the potential for cardiotoxicity, as predicted by prolongation of the QT_{c} interval, has been cited as a toxicity concern for quinolones.

There are several known natural product inhibitors of DNA gyrase that compete with ATP for binding the GyrB subunit (Maxwell, A. and Lawson, D. M. 2003, Curr. Topics in Med. Chem. 3: 283-303). The coumarins are natural products isolated from Streptomyces spp., examples of which are novobiocin, chlorobiocin and coumermycin Al. Although these compounds are potent inhibitors of DNA gyrase, their therapeutic utility is limited due to toxicity in eukaryotes and poor penetration in Gram-negative bacteria (Maxwell, A. 1997, Trends Microbiol. 5: 102-109). Another natural product class of compounds that targets the GyrB subunit is the cyclothialidines, which are isolated from Streptomyces filipensis (Watanabe, J. et al 1994, J. Antibiot. 47: 32-36). Despite potent activity against DNA gyrase, cyclothialidine is a poor antibacterial agent showing activity only against some eubacterial species (Nakada, N, 1993, Antimicrob. Agents Chemother. 37: 2656-2661).

Synthetic inhibitors that target the B subunit of DNA gyrase and topoisomeraseIV are known in the art. For example, coumarin-containing compounds are described in patent application number WO 99/35155, 5,6-bicyclic heteroaromatic compounds are described in patent application WO 02/060879, and pyrazole compounds are described in patent application WO 01/52845 (U.S. Pat. No. 6,608,087). AstraZeneca has also published certain applications describing anti-bacterial compounds: WO2005/026149, WO2006/087544, WO2006/087548, WO2006/087543, WO2006/092599 and WO2006/092608.

We have discovered a new class of compounds which are useful for inhibiting DNA gyrase and/or topoisomerase IV.

According to the present invention there is provided a compound of formula (I):

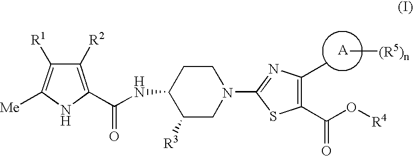

R¹ is chloro or cyano;

R² is hydrogen, chloro or cyano;

R³ is fluoro, methyl, methoxy, ethoxy, propoxy, allyloxy and benzyloxy;

R⁴ is hydrogen or C₁₋₄alkyl;

Ring A is carbocyclyl or heterocyclyl; wherein if said heterocyclyl contains an —NH— moiety that nitrogen may be optionally substituted by a group selected from R⁶; and wherein if said heterocyclyl contains an —N═ moiety that nitrogen may form a quaternary compound with a methyl group;

R⁵ is a substituent on carbon and is selected from halo, nitro, cyano, hydroxy, amino, carboxy, carbamoyl, mercapto, sulphamoyl, sulfo, formyl, ureido, hydroxyiminomethyl, N-hydroxyformamido, hydrazinocarbonyl, N-hydroxyethanimidoyl, amino(hydroxyimino)methyl, C₁₋₄alkyl, C₂₋₄alkenyl, C₂₋₄alkynyl, C₁₋₄alkoxy, C₁₋₄alkanoyl, C₁₋₄alkanoyloxy, N—(C₁₋₄alkyl)amino, N,N—(C₁₋₄alkyl)₂amino, C₁₋₄alkanoylamino, N—(C₁₋₄alkyl)carbamoyl, N,N—(C₁₋₄alkyl)₂carbamoyl, N—(C₁₋₄alkoxy)carbamoyl, N′—(C₁₋₄alkyl)ureido, N′,N′—(C₁₋₄alkyl)₂ureido, N—(C₁₋₄alkyl)-N—(C₁₋₄alkoxy)carbamoyl, C₁₋₄alkylS(O)ₐ wherein a is 0 to 2, C₁₋₄alkoxycarbonyl, C₁₋₄alkoxycarbonylamino, N—(C₁₋₄alkyl)sulphamoyl, N,N—(C₁₋₄alkyl)₂sulphamoyl, C₁₋₄alkylsulphonylamino, C₁₋₄alkylsulphonylaminocarbonyl, N′—(C₁₋₄alkyl)hydrazinocarbonyl, N′,N′—(C₁₋₄alkyl)₂hydrazinocarbonyl, carbocyclyl-R⁷— or heterocyclyl-R⁸—; wherein R⁵ may be optionally substituted on carbon by one or more R⁹; and wherein if said heterocyclyl contains an —NH— moiety that nitrogen may be optionally substituted by a group selected from R¹⁰;

n is 0, 1, 2,or 3;

R⁹ is selected from halo, nitro, cyano, hydroxy, amino, carboxy, carbamoyl, mercapto, sulphamoyl, C₁₋₄alkyl, C₂₋₄alkenyl, C₂₋₄alkynyl, C₁₋₄alkoxy, C₁₋₄alkanoyl, C₁₋₄alkanoyloxy, N—(C₁₋₄alkyl)amino, N,N—(C₁₋₄alkyl)₂amino, C₁₋₄alkanoylamino, N—(C₁₋₄alkyl)carbamoyl, N,N—(C₁₋₄alkyl)₂carbamoyl, C₁₋₄alkylS(O)a wherein a is 0 to 2, C₁₋₄alkoxycarbonyl, N—(C₁₋₄alkyl)sulphamoyl, N,N—(C₁₋₄alkyl)₂sulphamoyl, C₁₋₄alkylsulphonylamino, C₁₋₄alkoxycarbonylamino, carbocyclyl-R¹¹— or heterocyclyl-R¹²—; wherein R⁹ may be optionally substituted on carbon by one or more R¹³; and wherein if said heterocyclyl contains an —NH— moiety that nitrogen may be optionally substituted by a group selected from R¹⁴;

R⁶, R¹⁰ and R¹⁴ are independently selected from C₁₋₄alkyl, C₁₋₄alkanoyl, C₁₋₄alkylsulphonyl, C₁₋₄alkoxycarbonyl, carbamoyl, N—(C₁₋₄alkyl)carbamoyl, N,N—(C₁₋₄alkyl)carbamoyl, benzyl, benzyloxycarbonyl, benzoyl and phenylsulphonyl; wherein R⁶, R¹⁰ and R¹⁴ may be independently optionally substituted on carbon by a group selected from R²⁰;

R⁷, R⁸, R¹¹ and R¹² are independently selected from a direct bond, —O—, —N(R¹⁵)—, —C(O)—, —N(R¹⁶)C(O)—, —C(O)N(R¹⁷)—, —S(O)ₚ—, —SO₂N(R¹⁸)— or —N(R¹⁹)SO₂—; wherein R¹⁵, R¹⁶, R¹⁷, R¹⁸ and R¹⁹ are independently selected from hydrogen or C₁₋₄alkyl and p is 0-2;

R¹³ and R²⁰ are independently selected from halo, nitro, cyano, hydroxy, trifluoromethoxy, trifluoromethyl, amino, carboxy, carbamoyl, mercapto, sulphamoyl, methyl, ethyl, ethenyl, ethynyl, methoxy, ethoxy, 2-trimethylsilylethoxy, acetyl, acetoxy, methylamino, ethylamino, dimethylamino, diethylamino, N-methyl-N-ethylamino, acetylamino, N-methylcarbamoyl, N-ethylcarbamoyl, N,N-dimethylcarbamoyl, N,N-diethylcarbamoyl, N-methyl-N-ethylcarbamoyl, methylthio, ethylthio, methylsulphinyl, ethylsulphinyl, mesyl, ethylsulphonyl, methoxycarbonyl, ethoxycarbonyl, N-methylsulphamoyl, N-ethylsulphamoyl, N,N-dimethylsulphamoyl, N,N-diethylsulphamoyl or N-methyl-N-ethylsulphamoyl;

or a pharmaceutically acceptable salt thereof.

In an alternative embodiment, the present invention there is provided a compound of formula (Ia):

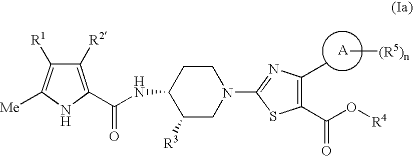

wherein:

R¹ is chloro or cyano;

R²′ is hydrogen, chloro, cyano or methyl;

R³ is fluoro, methyl, methoxy, ethoxy, propoxy, allyloxy and benzyloxy;

R⁴ is hydrogen or C₁₋₄alkyl;

Ring A is carbocyclyl or heterocyclyl; wherein if said heterocyclyl contains an —NH— moiety that nitrogen may be optionally substituted by a group selected from R⁶; and wherein if said heterocyclyl contains an —N═ moiety that nitrogen may form a quaternary compound with a methyl group;

R⁵ is a substituent on carbon and is selected from halo, nitro, cyano, hydroxy, amino, carboxy, carbamoyl, mercapto, sulphamoyl, sulfo, formyl, ureido, hydroxyiminomethyl, N-hydroxyformamido, hydrazinocarbonyl, N-hydroxyethanimidoyl, amino(hydroxyimino)methyl, C₁₋₄alkyl, C₂₋₄alkenyl, C₂₋₄alkynyl, C₁₋₄alkoxy, C₁₋₄alkanoyl, C₁₋₄alkanoyloxy, N—(C₁₋₄alkyl)amino, N,N—(C₁₋₄alkyl)₂amino, C₁₋₄alkanoylamino, N—(C₁₋₄alkyl)carbamoyl, N,N—(C₁₋₄alkyl)₂carbamoyl, N—(C₁₋₄alkoxy)carbamoyl, N′—(C₁₋₄alkyl)ureido, N′,N′—(C₁₋₄alkyl)₂ureido, N—(C₁₋₄alkyl)-N—(C₁₋₄alkoxy)carbamoyl, C₁₋₄alkylS(O)₃ wherein a is 0 to 2, C₁₋₄alkoxycarbonyl, C₁₋₄alkoxycarbonylamino, N—(C₁₋₄alkyl)sulphamoyl, N,N—(C₁₋₄alkyl)₂sulphamoyl, C₁₋₄alkylsulphonylamino, C₁₋₄alkylsulphonylaminocarbonyl, N′—(C₁₋₄alkyl)hydrazinocarbonyl, N′,N′—(C₁₋₄alkyl)₂hydrazinocarbonyl, carbocyclyl-R⁷— or heterocyclyl-R⁸—; wherein R⁵ may be optionally substituted on carbon by one or more R⁹; and wherein if said heterocyclyl contains an —NH— moiety that nitrogen may be optionally substituted by a group selected from R¹⁰;

n is 0, 1, 2, or 3;

R⁹ is selected from halo, nitro, cyano, hydroxy, amino, carboxy, carbamoyl, mercapto, sulphamoyl, C₁₋₄alkyl, C₂₋₄alkenyl, C₂₋₄alkynyl, C₁₋₄alkoxy, C₁₋₄alkanoyl, C₁₋₄alkanoyloxy, N—(C₁₋₄alkyl)amino, N,N—(C₁₋₄alkyl)₂amino, C₁₋₄alkanoylamino, N—(C₁₋₄alkyl)carbamoyl, N,N—(C₁₋₄alkyl)₂carbamoyl, C₁₋₄alkylS(O)₃ wherein a is 0 to 2, C₁₋₄alkoxycarbonyl, N—(C₁₋₄alkyl)sulphamoyl, N,N—(C₁₋₄alkyl)₂sulphamoyl, C₁₋₄alkylsulphonylamino, C₁₋₄alkoxycarbonylamino, carbocyclyl-R¹¹— or heterocyclyl-R¹²—; wherein R⁹ may be optionally substituted on carbon by one or more R¹³; and wherein if said heterocyclyl contains an —NH— moiety that nitrogen may be optionally substituted by a group selected from R¹⁴;

R⁶, R¹⁰ and R¹⁴ are independently selected from C₁₋₄alkyl, C₁₋₄alkanoyl, C₁₋₄alkylsulphonyl, C₁₋₄alkoxycarbonyl, carbamoyl, N—(C₁₋₄alkyl)carbamoyl, N,N—(C₁₋₄alkyl)carbamoyl, benzyl, benzyloxycarbonyl, benzoyl and phenylsulphonyl; wherein R⁶, R¹⁰ and R¹⁴ may be independently optionally substituted on carbon by a group selected from R²⁰;

R⁷, R⁸, R¹¹ and R¹² are independently selected from a direct bond,—O—, —N(R¹⁵)—, —C(O)—, —N(R¹⁶)C(O)—, —C(O)N(R¹⁷)—, —S(O)ₚ—, —SO₂N(R¹⁸)— or —N(R¹⁹)SO₂—; wherein R¹⁵, R¹⁶, R¹⁷, R¹⁸ and R¹⁹ are independently selected from hydrogen or C₁₋₄alkyl and p is 0-2;

R¹³ and R²⁰ are independently selected from halo, nitro, cyano, hydroxy, trifluoromethoxy, trifluoromethyl, amino, carboxy, carbamoyl, mercapto, sulphamoyl, methyl, ethyl, ethenyl, ethynyl, methoxy, ethoxy, 2-trimethylsilylethoxy, acetyl, acetoxy, methylamino, ethylamino, dimethylamino, diethylamino, N-methyl-N-ethylamino, acetylamino, N-methylcarbamoyl, N-ethylcarbamoyl, N,N-dimethylcarbamoyl, N,N-diethylcarbamoyl, N-methyl-N-ethylcarbamoyl, methylthio, ethylthio, methylsulphinyl, ethylsulphinyl, mesyl, ethylsulphonyl, methoxycarbonyl, ethoxycarbonyl, N-methylsulphamoyl, N-ethylsulphamoyl, N,N-dimethylsulphamoyl, N,N-diethylsulphamoyl or N-methyl-N-ethylsulphamoyl;

or a pharmaceutically acceptable salt thereof.

In this specification the term alkyl includes both straight and branched chain alkyl groups. For example, “C₁₋₄alkyl” includes methyl, ethyl, propyl, isopropyl and t-butyl. However references to individual alkyl groups such as propyl are specific for the straight chain version only. An analogous convention applies to other generic terms.

Where optional substituents are chosen from one or more groups it is to be understood that this definition includes all substituents being chosen from one of the specified groups or the substituents being chosen from two or more of the specified groups.

A “heterocyclyl” is a saturated, partially saturated or unsaturated, mono or bicyclic ring containing 4-12 atoms of which at least one atom is chosen from nitrogen, sulphur or oxygen, which may, unless otherwise specified, be carbon or nitrogen linked, wherein a —CH₂— group can optionally be replaced by a —C(O)— and a ring sulphur atom may be optionally oxidised to form the S-oxide(s). In one aspect of the invention a “heterocyclyl” is a saturated, partially saturated or unsaturated, monocyclic ring containing 5 or 6 atoms of which at least one atom is chosen from nitrogen, sulphur or oxygen, it may, unless otherwise specified, be carbon or nitrogen linked, a —CH₂— group can optionally be replaced by a —C(O)— and a ring sulphur atom may be optionally oxidised to form the S-oxides. In a further aspect of the invention a “heterocyclyl” is an unsaturated, carbon-linked, monocyclic ring containing 5 or 6 atoms of which at least one atom is chosen from nitrogen, sulphur or oxygen. Examples and suitable values of the term “heterocyclyl” are morpholino, piperidyl, pyridyl, pyranyl, pyrrolyl, pyrazolyl, isothiazolyl, indolyl, quinolyl, thienyl, 1,3-benzodioxolyl, thiadiazolyl, piperazinyl, thiazolidinyl, pyrrolidinyl, thiomorpholino, pyrrolinyl, homopiperazinyl, 3,5-dioxapiperidinyl, tetrahydropyranyl, imidazolyl, pyrimidyl, pyrazinyl, pyridazinyl, isoxazolyl, N-methylpyrrolyl, 4-pyridone, 1-isoquinolone, 2-pyrrolidone, 4-thiazolidone, pyridine-N-oxide and quinoline-N-oxide. Further examples and suitable values of the term “heterocyclyl” are imidazolyl, 1,2,4-oxadiazolyl, 1,3,4-oxadiazolyl, pyrazolyl, 1,2,4-triazolyl, pyridyl, benzothiazolyl, isoxazolyl, pyrazinyl, pyrimidinyl and thiazolyl.

A “carbocyclyl” is a saturated, partially saturated or unsaturated, mono or bicyclic carbon ring that contains 3-12 atoms; wherein a —CH₂— group can optionally be replaced by a —C(O)—. Particularly “carbocyclyl” is a monocyclic ring containing 5 or 6 atoms or a bicyclic ring containing 9 or 10 atoms. Suitable values for “carbocyclyl” include cyclopropyl, cyclobutyl, 1-oxocyclopentyl, cyclopentyl, cyclopentenyl, cyclohexyl, cyclohexenyl, phenyl, naphthyl, tetralinyl, indanyl or 1-oxoindanyl. A particular example of “carbocyclyl” is phenyl.

An example of “C₁₋₄alkanoyloxy” is acetoxy. Examples of “C₁₋₄alkoxycarbonyl” are methoxycarbonyl, ethoxycarbonyl, n- and t-butoxycarbonyl. Examples of “C₁₋₄alkoxycarbonylamino” are methoxycarbonylamino, ethoxycarbonylamino, n- and t-butoxycarbonylamino. Examples of “C₁₋₄alkoxy” are methoxy, ethoxy and propoxy. Examples of “C₁₋₄alkanoylamino” are formamido, acetamido and propionylamino. Examples of “C₁₋₄alkylS(O)₃ wherein a is 0 to 2” are methylthio, ethylthio, methylsulphinyl, ethylsulphinyl, mesyl and ethylsulphonyl. Examples of “C₁₋₄alkanoyl” are propionyl and acetyl. Examples of “N—(C₁₋₄alkyl)amino” are methylamino and ethylamino. Examples of “N,N—(C₁₋₄alkyl)₂amino” are di-N-methylamino, di-(N-ethyl)amino and N-ethyl-N-methylamino. Examples of “C₂₋₄alkenyl” are vinyl, allyl and 1-propenyl. Examples of “C₂₋₄alkynyl” are ethynyl, 1-propynyl and 2-propynyl. Examples of “N—(C₁₋₄alkyl)sulphamoyl” are N-(methyl)sulphamoyl and N-(ethyl)sulphamoyl. Examples of “N,N—(C₁₋₄alkyl)₂sulphamoyl” are N,N-(dimethyl)sulphamoyl and N-(methyl)-N-(ethyl)sulphamoyl. Examples of “N—(C₁₋₄alkyl)carbamoyl” are methylaminocarbonyl and ethylaminocarbonyl. Examples of “N,N—(C₁₋₄alkyl)₂carbamoyl” are dimethylaminocarbonyl and methylethylaminocarbonyl. Examples of “N—(C₁₋₄alkoxy)carbamoyl” are methoxyaminocarbonyl and isopropoxyaminocarbonyl. Examples of “N—(C₁₋₄alkyl)-N—(C₁₋₄alkoxy)carbamoyl” are N-methyl-N-methoxyaminocarbonyl and N-methyl-N-ethoxyaminocarbonyl. Examples of “N′—(C₁₋₄alkyl)ureido” are N′-methylureido and N′-isopropylureido. Examples of “N′,N′—(C₁₋₄alkyl)₂ureido” are N′N′-dimethylureido and N′-methyl-N′-isopropylureido. Examples of “N′—(C₁₋₄alkyl)hydrazinocarbonyl” are N′-methylhydrazinocarbonyl and N′-isopropylhydrazinocarbonyl. Examples of “N′,N′—(C₁₋₄alkyl)₂hydrazinocarbonyl” are N′N′-dimethylhydrazinocarbonyl and N′-methyl-N′-isopropylhydrazinocarbonyl. Examples of “C₁₋₄alkylsulphonylamino” are methylsulphonylamino, isopropylsulphonylamino and t-butylsulphonylamino. Examples of “C₁₋₄alkylsulphonylaminocarbonyl” are methylsulphonylaminocarbonyl, isopropylsulphonylaminocarbonyl and t-butylsulphonylaminocarbonyl. Examples of “C₁₋₄alkylsulphonyl” are methylsulphonyl, isopropylsulphonyl and t-butylsulphonyl.

A compound of formula (I) or (Ia) may form stable acid or basic salts, and in such cases administration of a compound as a salt may be appropriate, and pharmaceutically acceptable salts may be made by conventional methods such as those described following.

Suitable pharmaceutically-acceptable salts include acid addition salts such as methanesulfonate, tosylate, α-glycerophosphate. fumarate, hydrochloride, citrate, maleate, tartrate and hydrobromide. Also suitable are salts formed with phosphoric and sulfuric acid. In another aspect suitable salts are base salts such as an alkali metal salt for example sodium, an alkaline earth metal salt for example calcium or magnesium, an organic amine salt for example triethylamine, morpholine, N-methylpiperidine, N-ethylpiperidine, procaine, dibenzylamine, N,N-dibenzylethylamine, tris-(2-hydroxyethyl)amine, N-methyl d-glucamine and amino acids such as lysine. There may be more than one cation or anion depending on the number of charged functions and the valency of the cations or anions. In one aspect of the invention the pharmaceutically-acceptable salt is the sodium salt.

However, to facilitate isolation of the salt during preparation, salts which are less soluble in the chosen solvent may be utilised whether pharmaceutically-acceptable or not.

Within the present invention it is to be understood that a compound of the formula (I) or (Ia) or a salt thereof may exhibit the phenomenon of tautomerism and that the formulae drawings within this specification can represent only one of the possible tautomeric forms. It is to be understood that the invention encompasses any tautomeric form which inhibits DNA gyrase and/or topoisomerase IV and is not to be limited merely to any one tautomeric form utilised within the formulae drawings. The formulae drawings within this specification can represent only one of the possible tautomeric forms and it is to be understood that the specification encompasses all possible tautomeric forms of the compounds drawn not just those forms which it has been possible to show graphically herein. The same applies to compound names.

It will be appreciated by those skilled in the art that in addition to the two asymmetric carbons drawn in formula (I) or (Ia), compounds of formula (I) or (Ia) may contain additional asymmetrically substituted carbon(s) and sulphur atom(s), and accordingly may exist in, and be isolated in, as far as those additional asymmetrically substituted carbon(s) and sulphur atom(s) are concerned, optically-active and racemic forms at those positions. It is to be understood that the present invention encompasses any racemic, optically-active, polymorphic or stereoisomeric form, or mixtures thereof, at any additional asymmetrically substituted carbon(s) and sulphur atom(s), which possesses properties useful in the inhibition of DNA gyrase and/or topoisomerase IV.

Optically-active forms may be prepared by procedures known in the art for example, by resolution of the racemic form by recrystallization techniques, by synthesis from optically-active starting materials, by chiral synthesis, by enzymatic resolution, by biotransformation, or by chromatographic separation using a chiral stationary phase.

Some compounds may exhibit polymorphism. It is to be understood that the present invention encompasses any polymorphic form, or mixtures thereof, which form possesses properties useful in the inhibition of DNA gyrase and/or topoisomerase IV It is also to be understood that certain compounds of the formula (I) or (Ia) and salts thereof can exist in solvated as well as unsolvated forms such as, for example, hydrated forms. It is to be understood that the invention encompasses all such solvated forms which inhibit DNA gyrase and/or topoisomerase IV.

There follow particular and suitable values for certain substituents and groups referred to in this specification. These values may be used where appropriate with any of the definitions and embodiments disclosed hereinbefore, or hereinafter. For the avoidance of doubt each stated species represents a particular and independent aspect of this invention.

R¹ is chloro.

R¹ is cyano;

R² is hydrogen.

R² is chloro

R² is cyano.

R² is hydrogen or chloro.

R^{2′} is hydrogen.

R^{2′} is chloro

R^{2′} is cyano.

R^{2′} is hydrogen or chloro.

R³ is fluoro.

R³ is methyl.

R³ is methoxy.

R³ is ethoxy.

R³ is propoxy.

R³ is allyloxy.

R³ is benzyloxy.

R³ is fluoro or methoxy.

R⁴ is hydrogen.

R⁴ is C₁₋₄alkyl.

R⁴ is hydrogen, methyl or ethyl.

R⁴ is methyl or ethyl.

R⁴ is methyl.

R⁴ is ethyl.

Ring A is carbocyclyl.

Ring A is heterocyclyl; wherein if said heterocyclyl contains an —NH— moiety that nitrogen may be optionally substituted by a group selected from R⁶; and wherein if said heterocyclyl contains an —N═ moiety that nitrogen may form a quaternary compound with a methyl group; wherein:

R⁶ is C₁₋₄alkyl; wherein R⁶ may be optionally substituted on carbon by a group selected from R²⁰; and

R²⁰ is selected from methoxy or 2-trimethylsilylethoxy.

Ring A is heterocyclyl; wherein if said heterocyclyl contains an —NH— moiety that nitrogen may be optionally substituted by a group selected from R⁶; and wherein if said heterocyclyl contains an —N═ moiety that nitrogen may form a quaternary compound with a methyl group; wherein:

R⁶ is C₁₋₄alkyl; wherein R⁶ may be optionally substituted on carbon by a group selected from R²⁰; and

R²⁰ is selected from methoxy or ethoxy.

Ring A is pyridyl, 2H-pyrazolyl, isoxazolyl, imidazolyl, pyrazinyl, thiazolyl, pyrimidinyl, 1,2,4-oxadiazolyl, benzothiazolyl, 1,2,4-triazolyl or 1,3,4-oxadiazolyl wherein said imidazolyl or 1,2,4-triazolyl may be optionally substituted on nitrogen by a group selected from R⁶; and wherein if said imidazolyl may form a quaternary compound on an —N═ moiety with a methyl group; wherein

R⁶ is methyl or ethyl; wherein R⁶ may be optionally substituted on carbon by a group selected from R²⁰; and

R²⁰ is selected from methoxy or 2-trimethylsilylethoxy.

Ring A is pyridyl, 2H-pyrazolyl, isoxazolyl, imidazolyl, pyrazinyl, thiazolyl, pyrimidinyl, 1,2,4-oxadiazolyl, benzothiazolyl, 1,2,4-triazolyl or 1,3,4-oxadiazolyl wherein said imidazolyl or 1,2,4-triazolyl may be optionally substituted on nitrogen by a group selected from R⁶; and wherein if said imidazolyl may form a quaternary compound on an —N═ moiety with a methyl group; wherein

R⁶ is methyl or ethyl; wherein R⁶ may be optionally substituted on carbon by a group selected from R²⁰; and

R²⁰ is selected from methoxy or ethoxy.

Ring A is 1-(2-methoxyethyl)imidazol-2-yl, 1-(2-trimethylsilylethoxymethyl)imidazol-2-yl, 1-(methoxymethyl)imidazol-2-yl, 1,2,4-oxadiazol-5-yl, 1,3,4-oxadiazol-2-yl, 1,3-dimethylimidazol-2-yl, 1H-imidazol-2-yl, 1-methylimidazol-4-yl, 2H-pyrazol-3-yl, 2-methyl-1,2,4-triazol-3-yl, 2-pyridyl, benzothiazol-2-yl, isoxazol-5-yl, pyrazin-2-yl, pyrimidin-2-yl, pyrimidin-4-yl, thiazol-2-yl or thiazol-4-yl.

Ring A is 1-(2-methoxyethyl)imidazol-2-yl.

Ring A is 1-(2-trimethylsilylethoxymethyl)imidazol-2-yl.

Ring A is 1-(methoxymethyl)imidazol-2-yl.

Ring A is 1,2,4-oxadiazol-5-yl.

Ring A is 1,3,4-oxadiazol-2-yl.

Ring A is 1,3-dimethylimidazol-2-yl.

Ring A is 1H-imidazol-2-yl.

Ring A is 1-methylimidazol-4-yl.

Ring A is 2H-pyrazol-3-yl.

Ring A is 2-methyl-1,2,4-triazol-3-yl.

Ring A is 2-pyridyl.

Ring A is benzothiazol-2-yl.

Ring A is isoxazol-5-yl.

Ring A is pyrazin-2-yl.

Ring A is pyrimidin-2-yl.

Ring A is pyrimidin-4-yl.

Ring A is thiazol-2-yl.

Ring A is thiazol-4-yl.

R⁵ is a substituent on carbon and is selected from amino, C₁₋₄alkyl, C₁₋₄alkoxy, N—(C₁₋₄alkyl)amino or N,N—(C₁₋₄alkyl)₂amino.

R⁵ is a substituent on carbon and is selected from amino, methyl, methoxy, methylamio or dimethylamino.

n is 0-2.

n is 0.

n is 1

n is 2.

Ring A, R⁵ and n together form 1H-imidazol-2-yl, 2,6-bis(dimethylamino)pyrimidin-4-yl, 2-pyridyl, 2H-pyrazol-3-yl, pyrimidin-4-yl, isoxazol-5-yl, 1-methylimidazol-4-yl, pyrazin-2-yl, 2-aminothiazol-4-yl, 2-dimethylaminothiazol-4-yl, 2-methylaminothiazol-4-yl, 1,3-dimethylimidazol-2-yl, 1,4,5-trimethylimidazol-2-yl, 4,6-dimethoxypyrimidin-2-yl, 3-methyl-1,2,4-oxadiazol-5-yl, benzothiazol-2-yl, 4-methoxypyrimidin-2-yl, pyrimidin-2-yl, 1-methylimidazol-2-yl, 1-(2-methoxyethyl)imidazol-2-yl, 1-(methoxymethyl)imidazol-2-yl, 1-(2-trimethylsilylethoxymethyl)imidazol-2-yl, thiazol-2-yl, 2-methyl-1,2,4-triazol-3-yl, 5-methyl-1,3,4-oxadiazol-2-yl or 1,3,4-oxadiazol-2-yl.

Ring A, R⁵ and n together form R⁵ and n together form 1H-imidazol-2-yl.

Ring A, R⁵ and n together form 2,6-bis(dimethylamino)pyrimidin-4-yl.

Ring A, R⁵ and n together form 2-pyridyl.

Ring A, R⁵ and n together form 2H-pyrazol-3-yl.

Ring A, R⁵ and n together form pyrimidin-4-yl.

Ring A, R⁵ and n together form isoxazol-5-yl.

Ring A, R⁵ and n together form 1-methylimidazol-4-yl.

Ring A, R⁵ and n together form pyrazin-2-yl.

Ring A, R⁵ and n together form 2-aminothiazol-4-yl.

Ring A, R⁵ and n together form 2-dimethylaminothiazol-4-yl.

Ring A, R⁵ and n together form 2-methylaminothiazol-4-yl.

Ring A, R⁵ and n together form 1,3-dimethylimidazol-2-yl.

Ring A, R⁵ and n together form 1,4,5-trimethylimidazol-2-yl.

Ring A, R⁵ and n together form 4,6-dimethoxypyrimidin-2-yl.

Ring A, R⁵ and n together form 3-methyl-1,2,4-oxadiazol-5-yl.

Ring A, R⁵ and n together form benzothiazol-2-yl.

Ring A, R⁵ and n together form 4-methoxypyrimidin-2-yl.

Ring A, R⁵ and n together form pyrimidin-2-yl.

Ring A, R⁵ and n together form 1-methylimidazol-2-yl.

Ring A, R⁵ and n together form 1-(2-methoxyethyl)imidazol-2-yl.

Ring A, R⁵ and n together form 1-(methoxymethyl)imidazol-2-yl.

Ring A, R⁵ and n together form 1-(2-trimethylsilylethoxymethyl)imidazol-2-yl.

Ring A, R⁵ and n together form thiazol-2-yl.

Ring A, R⁵ and n together form 2-methyl-1,2,4-triazol-3-yl.

Ring A, R⁵ and n together form 5-methyl-1,3,4-oxadiazol-2-yl.

Ring A, R⁵ and n together form 1,3,4-oxadiazol-2-yl.

Therefore in a further aspect of the invention there is provided a compound of formula (I) (as depicted above) wherein:

R¹ is chloro or cyano;

R² is hydrogen or chloro;

R³ is fluoro or methoxy;

R⁴ is hydrogen or C₁₋₄alkyl;

Ring A is pyridyl, 2H-pyrazolyl, isoxazolyl, imidazolyl, pyrazinyl, thiazolyl, pyrimidinyl, 1,2,4-oxadiazolyl, benzothiazolyl, 1,2,4-triazolyl or 1,3,4-oxadiazolyl wherein said imidazolyl or 1,2,4-triazolyl may be optionally substituted on nitrogen by a group selected from R⁶; and wherein if said imidazolyl may form a quaternary compound on an —N═ moiety with a methyl group;

R⁶ is methyl or ethyl; wherein R⁶ may be optionally substituted on carbon by a group selected from R²⁰;

R²⁰ is selected from methoxy or 2-trimethylsilylethoxy;

R⁵ is a substituent on carbon and is selected from amino, C₁₋₄alkyl, C₁₋₄alkoxy, N—(C₁₋₄alkyl)amino or N,N—(C₁₋₄alkyl)₂amino; and

n is 0-2;

or a pharmaceutically acceptable salt thereof.

Therefore in a further aspect of the invention there is provided a compound of formula (I) (as depicted above) wherein:

R¹ is chloro or cyano;

R² is hydrogen or chloro;

R³ is fluoro or methoxy;

R⁴ is hydrogen or C₁₋₄alkyl;

Ring A is pyridyl, 2H-pyrazolyl, isoxazolyl, imidazolyl, pyrazinyl, thiazolyl, pyrimidinyl, 1,2,4-oxadiazolyl, benzothiazolyl, 1,2,4-triazolyl or 1,3,4-oxadiazolyl wherein said imidazolyl or 1,2,4-triazolyl may be optionally substituted on nitrogen by a group selected from R⁶; and wherein if said imidazolyl may form a quaternary compound on an —N═ moiety with a methyl group;

R⁶ is methyl or ethyl; wherein R⁶ may be optionally substituted on carbon by a group selected from R²⁰;

R²⁰ is selected from methoxy or ethoxy;

R⁵ is a substituent on carbon and is selected from amino, C₁₋₄alkyl, C₁₋₄alkoxy, N—(C₁₋₄alkyl)amino or N,N—(C₁₋₄alkyl)₂amino; and

n is 0-2;

or a pharmaceutically acceptable salt thereof.

Therefore in a further aspect of the invention there is provided a compound of formula (I) (as depicted above) wherein:

R¹ is chloro or cyano;

R² is hydrogen or chloro;

R³ is fluoro or methoxy;

R⁴ is hydrogen, methyl or ethyl;

Ring A is 1-(2-methoxyethyl)imidazol-2-yl, 1-(2-trimethylsilylethoxymethyl)imidazol-2-yl, 1-(methoxymethyl)imidazol-2-yl, 1,2,4-oxadiazol-5-yl, 1,3,4-oxadiazol-2-yl, 1,3-dimethylimidazol-2-yl, 1H-imidazol-2-yl, 1-methylimidazol-4-yl, 2H-pyrazol-3-yl, 2-methyl-1,2,4-triazol-3-yl, 2-pyridyl, benzothiazol-2-yl, isoxazol-5-yl, pyrazin-2-yl, pyrimidin-2-yl, pyrimidin-4-yl, thiazol-2-yl or thiazol-4-yl;

R⁵ is a substituent on carbon and is selected from amino, methyl, methoxy, methylamio or dimethylamino;

n is 0-2;

or a pharmaceutically acceptable salt thereof.

Therefore in a further aspect of the invention there is provided a compound of formula (I) (as depicted above) wherein:

R¹ and R² are chloro;

R³ is methoxy;

R⁴ is hydrogen;

Ring A is 2-methyl-1,2,4-triazol-3-yl or pyrimidin-2-yl;

n is 0;

or a pharmaceutically acceptable salt thereof.

Therefore in a further aspect of the invention there is provided a compound of formula (Ia) (as depicted above) wherein:

R¹ is chloro or cyano;

R^{2′} is hydrogen or chloro;

R³ is fluoro or methoxy;

R⁴ is hydrogen or C₁₋₄alkyl;

Ring A is pyridyl, 2H-pyrazolyl, isoxazolyl, imidazolyl, pyrazinyl, thiazolyl, pyrimidinyl, 1,2,4-oxadiazolyl, benzothiazolyl, 1,2,4-triazolyl or 1,3,4-oxadiazolyl wherein said imidazolyl or 1,2,4-triazolyl may be optionally substituted on nitrogen by a group selected from R⁶; and wherein if said imidazolyl may form a quaternary compound on an —N═ moiety with a methyl group;

R⁶ is methyl or ethyl; wherein R⁶ may be optionally substituted on carbon by a group selected from R²⁰;

R²⁰ is selected from methoxy or 2-trimethylsilylethoxy;

R⁵ is a substituent on carbon and is selected from amino, C₁₋₄alkyl, C₁₋₄alkoxy, N—(C₁₋₄alkyl)amino or N,N—(C₁₋₄alkyl)₂amino; and

n is 0-2;

or a pharmaceutically acceptable salt thereof.

Therefore in a further aspect of the invention there is provided a compound of formula (Ia) (as depicted above) wherein:

R¹ is chloro or cyano;

R^{2′} is hydrogen or chloro;

R³ is fluoro or methoxy;

R⁴ is hydrogen or C₁₋₄alkyl;

Ring A is pyridyl, 2H-pyrazolyl, isoxazolyl, imidazolyl, pyrazinyl, thiazolyl, pyrimidinyl, 1,2,4-oxadiazolyl, benzothiazolyl, 1,2,4-triazolyl or 1,3,4-oxadiazolyl wherein said imidazolyl or 1,2,4-triazolyl may be optionally substituted on nitrogen by a group selected from R⁶; and wherein if said imidazolyl may form a quaternary compound on an —N═ moiety with a methyl group;

R⁶ is methyl or ethyl; wherein R⁶ may be optionally substituted on carbon by a group selected from R²⁰;

R²⁰ is selected from methoxy or ethoxy;

R⁵ is a substituent on carbon and is selected from amino, C₁₋₄alkyl, C₁₋₄alkoxy, N—(C₁₋₄alkyl)amino or N,N—(C₁₋₄alkyl)₂amino; and

n is 0-2;

or a pharmaceutically acceptable salt thereof.

Therefore in a further aspect of the invention there is provided a compound of formula (Ia) (as depicted above) wherein:

R¹ is chloro or cyano;

R^{2′} is hydrogen or chloro;

R³ is fluoro or methoxy;

R⁴ is hydrogen, methyl or ethyl;

Ring A is 1-(2-methoxyethyl)imidazol-2-yl, 1-(2-trimethylsilylethoxymethyl)imidazol-2-yl, 1-(methoxymethyl)imidazol-2-yl, 1,2,4-oxadiazol-5-yl, 1,3,4-oxadiazol-2-yl, 1,3-dimethylimidazol-2-yl, 1H-imidazol-2-yl, 1-methylimidazol-4-yl, 2H-pyrazol-3-yl, 2-methyl-1,2,4-triazol-3-yl, 2-pyridyl, benzothiazol-2-yl, isoxazol-5-yl, pyrazin-2-yl, pyrimidin-2-yl, pyrimidin-4-yl, thiazol-2-yl or thiazol-4-yl;

R⁵ is a substituent on carbon and is selected from amino, methyl, methoxy, methylamio or dimethylamino;

n is 0-2;

or a pharmaceutically acceptable salt thereof.

Therefore in a further aspect of the invention there is provided a compound of formula (Ia) (as depicted above) wherein:

R¹ and R^{2′} are chloro;

R³ is methoxy;

R⁴ is hydrogen;

Ring A is 2-methyl-1,2,4-triazol-3-yl or pyrimidin-2-yl;

n is 0;

or a pharmaceutically acceptable salt thereof.

Particular compounds of the invention are the compounds of the Examples, each of which provides a further independent aspect of the invention. In further aspects, the present invention also comprises any two or more compounds of the Examples.

In one embodiment of the invention are provided compounds of formula (I) or (Ia), in an alternative embodiment are provided pharmaceutically-acceptable salts of compounds of formula (I) or (Ia).

In a further aspect the present invention provides a process for preparing a compound of formula (I) or (Ia), or a pharmaceutically-acceptable salt thereof.

Thus, the present invention also provides that the compounds of the formula (I) or (Ia) and pharmaceutically-acceptable salts thereof, can be prepared by a process as follows (wherein the variables are as defined above unless otherwise stated):
Process a) reacting a compound of formula (II) or (II′):

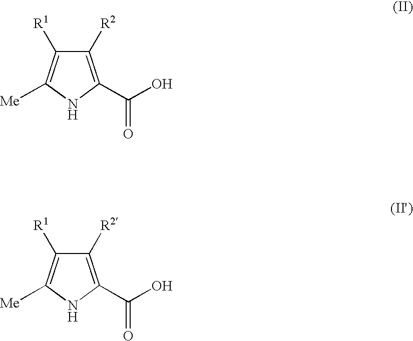

or an activated acid derivative thereof, with a compound of formula (III):

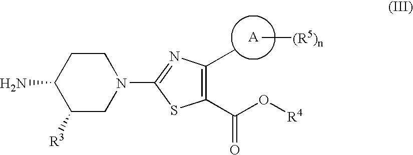

or
Process b) reacting a compound of formula (IV) or (IV′):

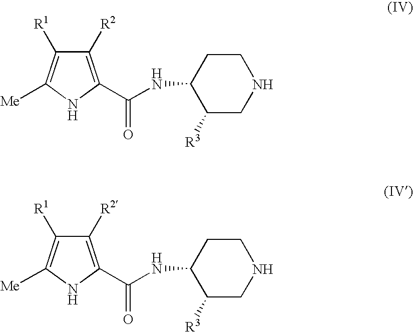

with a compound of formula (V):

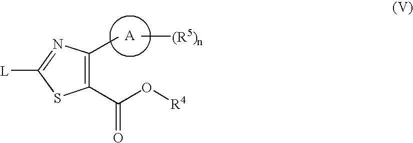

wherein L is a displaceable group; or
Process c) for compounds of formula (I) or (Ia) wherein R⁴ is C₁₋₄alkyl; reacting a compound of formula (I) or (Ia) which is a compound of formula (VI) or (VI′):

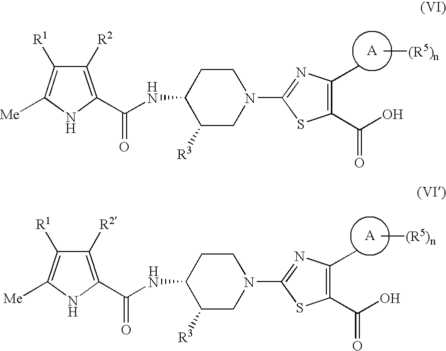

with a compound of formula (VII):

R^{4a}—OH (VI)
wherein R^{4a} is C₁₋₄alkyl;
or
Process d) for compounds of formula (I) or (Ia) wherein R⁴ is hydrogen; deprotecting a compound of formula (VIII) or (VIII′):

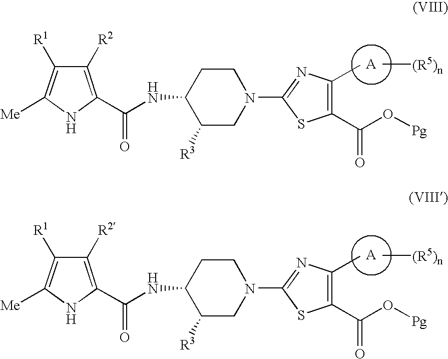

wherein Pg is a carboxylic acid protecting group;
and thereafter if necessary:
i) converting a compound of the formula (I) or (Ia) into another compound of the formula (I)
or (Ia);
ii) removing any protecting groups;
iii) forming a pharmaceutically acceptable salt; and/or
iv) chirally purifying the compound of formula (I) or (Ia).

L is a displaceable group. Suitable values for L include halo, for example chloro and bromo, pentafluorophenoxy and 2,5-oxopyrrolidin-1-yloxy.

Pg is a carboxylic acid protecting group. Suitable values for Pg are defined herein below.

Specific reaction conditions for the above reaction are as follows.
Process a) Compounds of formula (II) or (II′) and (III) may be coupled together in the presence of a suitable coupling reagent. Standard peptide coupling reagents known in the art can be employed as suitable coupling reagents, or for example carbonyldiimidazole and dicyclohexyl-carbodiimide, optionally in the presence of a catalyst such as dimethylaminopyridine or 4-pyrrolidinopyridine, optionally in the presence of a base for example triethylamine, pyridine, or 2,6-di-alkyl-pyridines such as 2,6-lutidine or 2,6-di-tert-butylpyridine. Suitable solvents include dimethylacetamide, dichloromethane, benzene, tetrahydrofuran and dimethylformamide. The coupling reaction may conveniently be performed at a temperature in the range of −40 to 40° C.

Suitable activated acid derivatives include acid halides, for example acid chlorides, and active esters, for example pentafluorophenyl esters. The reaction of these types of compounds with amines is well known in the art, for example they may be reacted in the presence of a base, such as those described above, and in a suitable solvent, such as those described above. The reaction may conveniently be performed at a temperature in the range of −40 to 40° C.

Compounds of formula (III) may be prepared according to Scheme 1:

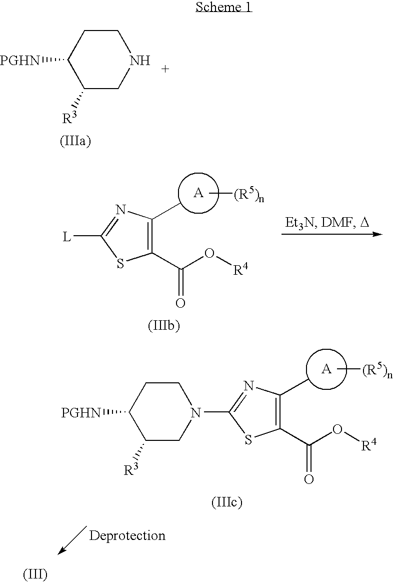

wherein PG is a nitrogen protecting group such as those defined herein below; and L is a displaceable group such as those defined herein above.

Compounds of formula (II) or (II′) are commercially available compounds, or they are known in the literature, or they are prepared by standard processes known in the art.
Process b) Compounds of formula (IV) or (IV′) and (V) in a suitable solvent such as dimethylformamide or N-methylpyrrolindine and optionally in the presence of a base such as triethylamine or diisopropylamine are heated together at a temperature range between 50 to 100° C.

Compounds of formula (IV) may be prepared according to Scheme 2:

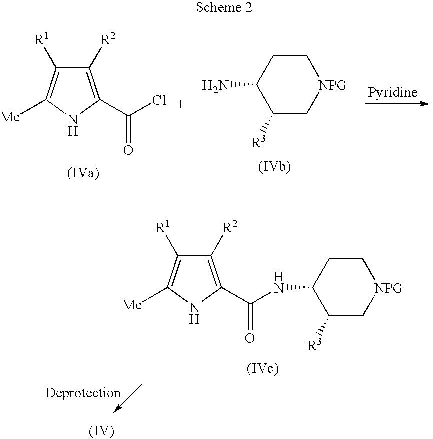

wherein PG is a nitrogen protecting group such as those defined herein below. Compounds of formula (IV′) can be prepared by an analogous procedure.

Compounds of formula (V) may be prepared according to Scheme 3:

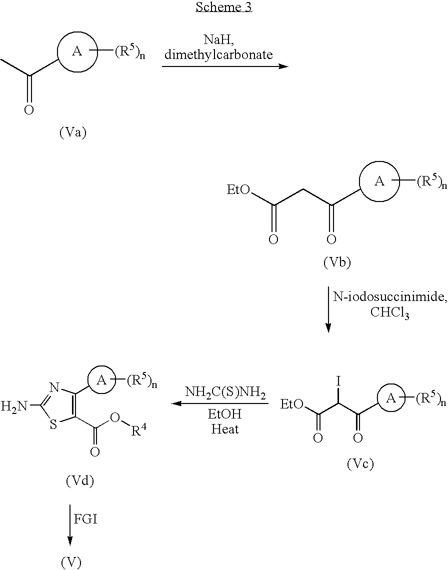

Wherein FGI is functional group interconversion of the NH₂ group to the required “L”.
Process c) Compounds of formula (VI) or (VI′) and (VII) in a suitable solvent such as methanol, ethanol, or tetrahydrofuran in the presence of a base such as sodium hydroxide, lithium hydroxide, or barium hydroxide are reacted at a temperature range of 25 to 100° C.

Compounds of formula (VI) or (VI′) may be prepared by a suitable modification of the reactions described herein to make a compound of formula (I) or (Ia) wherein R⁴ is hydrogen.

Compounds of formula (VII) are commercially available compounds, or they are known in the literature, or they are prepared by standard processes known in the art.
Process d) Suitable deprotection conditions are described hereinbelow.

Compounds of formula (VIII) or (VIII′) may be prepared by a suitable modification of the reactions described herein to make a compound of formula (I) or (Ia).

The formation of a pharmaceutically-acceptable salt is within the skill of an ordinary organic chemist using standard techniques.

It will be appreciated that certain of the various ring substituents in the compounds of the present invention may be introduced by standard aromatic substitution reactions or generated by conventional functional group modifications either prior to or immediately following the processes mentioned above, and as such are included in the process aspect of the invention. The reagents used to introduce such ring substituents are either commercially available or are made by processes known in the art.

Introduction of substituents into a ring may convert one compound of the formula (I) or (Ia) into another compound of the formula (I) or (Ia). Such reactions and modifications include, for example, introduction of a substituent by means of an aromatic substitution reaction, reduction of substituents, alkylation of substituents, oxidation of substituents, esterification of substituents, amidation of substituents, formation of heteroaryl rings. The reagents and reaction conditions for such procedures are well known in the chemical art. Particular examples of aromatic substitution reactions include the introduction of alkoxides, diazotization reactions followed by introduction of thiol group, alcohol group, halogen group. Examples of modifications include; oxidation of alkylthio to alkylsulphinyl or alkylsulphonyl.

The skilled organic chemist will be able to use and adapt the information contained and referenced within the above references, and accompanying Examples therein and also the Examples herein, to obtain necessary starting materials, and products. If not commercially available, the necessary starting materials for the procedures such as those described above may be made by procedures which are selected from standard organic chemical techniques, techniques which are analogous to the synthesis of known, structurally similar compounds, or techniques which are analogous to the above described procedure or the procedures described in the examples. It is noted that many of the starting materials for synthetic methods as described above are commercially available and/or widely reported in the scientific literature, or could be made from commercially available compounds using adaptations of processes reported in the scientific literature. The reader is further referred to Advanced Organic Chemistry, 4^{th} Edition, by Jerry March, published by John Wiley & Sons 1992, for general guidance on reaction conditions and reagents.

It will also be appreciated that in some of the reactions mentioned herein it may be necessary/desirable to protect any sensitive groups in compounds. The instances where protection is necessary or desirable are known to those skilled in the art, as are suitable methods for such protection. Conventional protecting groups may be used in accordance with standard practice (for illustration see T. W. Greene, Protective Groups in Organic Synthesis, John Wiley and Sons, 1991).

Examples of a suitable protecting group for a hydroxy group is, for example, an acyl group, for example an alkanoyl group such as acetyl, an aroyl group, for example benzoyl, a silyl group such as trimethylsilyl or an arylmethyl group, for example benzyl. The deprotection conditions for the above protecting groups will necessarily vary with the choice of protecting group. Thus, for example, an acyl group such as an alkanoyl or an aroyl group may be removed, for example, by hydrolysis with a suitable base such as an alkali metal hydroxide, for example lithium or sodium hydroxide. Alternatively a silyl group such as trimethylsilyl may be removed, for example, by fluoride or by aqueous acid; or an arylmethyl group such as a benzyl group may be removed, for example, by hydrogenation in the presence of a catalyst such as palladium-on-carbon.

A suitable protecting group for an amino group is, for example, an acyl group, for example an alkanoyl group such as acetyl, an alkoxycarbonyl group, for example a methoxycarbonyl, ethoxycarbonyl or t-butoxycarbonyl group, an arylmethoxycarbonyl group, for example benzyloxycarbonyl, or an aroyl group, for example benzoyl. The deprotection conditions for the above protecting groups necessarily vary with the choice of protecting group. Thus, for example, an acyl group such as an alkanoyl or alkoxycarbonyl group or an aroyl group may be removed for example, by hydrolysis with a suitable base such as an alkali metal hydroxide, for example lithium or sodium hydroxide. Alternatively an acyl group such as a t-butoxycarbonyl group may be removed, for example, by treatment with a suitable acid as hydrochloric, sulphuric or phosphoric acid or trifluoroacetic acid and an arylmethoxycarbonyl group such as a benzyloxycarbonyl group may be removed, for example, by hydrogenation over a catalyst such as palladium-on-carbon, or by treatment with a Lewis acid for example boron tris(trifluoroacetate). A suitable alternative protecting group for a primary amino group is, for example, a phthaloyl group which may be removed by treatment with an alkylamine, for example dimethylaminopropylamine or 2-hydroxyethylamine, or with hydrazine.

A suitable protecting group for a carboxy group is, for example, an esterifying group, for example a methyl or an ethyl group which may be removed, for example, by hydrolysis with a base such as sodium hydroxide, or for example a t-butyl group which may be removed, for example, by treatment with an acid, for example an organic acid such as trifluoroacetic acid, or for example a benzyl group which may be removed, for example, by hydrogenation over a catalyst such as palladium-on-carbon, or for example, an allyl group which may be removed, for example, by use of a palladium catalyst such as palladium acetate.

The protecting groups may be removed at any convenient stage in the synthesis using conventional techniques well known in the chemical art, or they may be removed during a later reaction step or work-up.

Optically active forms of a compound of the invention may be obtained by carrying out one of the above procedures using an optically active starting material (formed, for example, by asymmetric induction of a suitable reaction step), or by resolution of a racemic form of the compound or intermediate using a standard procedure, or by chromatographic separation of diastereoisomers (when produced). Enzymatic techniques may also be useful for the preparation of optically active compounds and/or intermediates.

Similarly, when a pure regioisomer of a compound of the invention is required, it may be obtained by carrying out one of the above procedures using a pure regioisomer as a starting material, or by resolution of a mixture of the regioisomers or intermediates using a standard procedure.

### Enzyme Potency Testing Methods

E. coli GyrB ATPase Inhibition Activity: Compounds can be tested for inhibition of E. coli GyrB ATPase activity using an ammonium molybdate/malachite green-based phosphate detection assay (Lanzetta, P. A., L. J. Alvarez, P. S. Reinach, and O. A. Candia, 1979, 100: 95-97). Assays can be performed in multiwell plates in 30 μl reactions containing: 50 mM Hepes buffer pH 7.5, 75 mM ammonium acetate, 8.0 mM magnesium chloride, 0.5 mM ethylenediaminetetraacetic acid, 5% glycerol, 1 mM 1,4-Dithio-DL-threitol, 200 nM bovine serum albumin, 1.6 μg/ml sheared salmon sperm DNA, 400 pM E. coli GyrA, 400 pM E. coli GyrB, 250 μM ATP, and compound in dimethylsulfoxide. Reactions can be quenched with 30 μl of ammonium molybdate/malachite green detection reagent containing 1.2 mM malachite green hydrochloride, 8.5 mM ammonium molybdate tetrahydrate, and 1 M hydrochloric acid. Plates can be read in an absorbance plate reader at 650 nm and percent inhibition values are calculated using dimethylsulfoxide (2%)-containing reactions as 0% inhibition and EDTA-containing (2.4 μM) reactions as 100% inhibition controls. An IC₅₀ measurement of compound potency for each compound can be determined from reactions performed in the presence of 10 different compound concentrations.

E. coli Topoisomerase IV ATPase Inhibition Activity: Compounds can be tested for inhibition of E. coli topoisomerase IV ATPase activity as described above for E. coli GyrB except the 30 μl reactions contained the following: 20 mM TRIS buffer pH 8, 50 mM ammonium acetate, 8 mM magnesium chloride, 5% glycerol, 5 mM 1,4-Dithio-DL-threitol, 0.005% Brij-35, 5 μg/ml sheared salmon sperm DNA, 500 pM E. coli ParC, 500 pM E. coli ParE, 160 μM ATP, and compound in dimethylsulfoxide. An IC₅₀ measurement of compound potency for each compound can be determined from reactions performed in the presence of 10 different compound concentrations.

Compounds of the invention were tested in an assay substantially similar to the assay described above for measuring the inhibition of E. coli GyrB ATPase and E. coli Topoisomerase IV ATPase. In general, the compounds of the invention have IC₅₀ values of <200 μg/ml in one or both assays.

S. aureus GyrB ATPase Inhibition Activity: Compounds may be tested for inhibition of S. aureus GyrB ATPase activity using an ammonium molybdate/malachite green-based phosphate detection assay (Lanzetta, P. A., L. J. Alvarez, P. S. Reinach, and O. A. Candia, 1979, 100: 95-97). Assays can be performed in multiwell plates in 30 μl reactions containing: 50 mM Hepes buffer pH 7.5, 75 mM ammonium acetate, 8.0 mM magnesium chloride, 0.5 mM ethylenediaminetetraacetic acid, 5% glycerol, 1.0 mM 1,4-Dithio-DL-threitol, 200 nM bovine serum albumin, 1.0 μg/ml sheared salmon sperm DNA, 250 pM E. coli GyrA, 250 pM S. aureus GyrB, 250 μM ATP, and compound in dimethylsulfoxide. Reactions can be quenched with 30 pl of ammonium molybdate/malachite green detection reagent containing 1.2 mM malachite green hydrochloride, 8.5 mM ammonium molybdate tetrahydrate, and 1 M hydrochloric acid. Plates can be read in an absorbance plate reader at 650 nm and percent inhibition values can be calculated using dimethylsulfoxide (2%)-containing reactions as 0% inhibition and EDTA-containing (2.4 μM) reactions as 100% inhibition controls. An IC₅₀ measurement of compound potency for each compound can be determined from reactions performed in the presence of 10 different compound concentrations.

Compounds of the invention were tested in an assay substantially similar to the assay described above for measuring the inhibition of S. aureus GyrB ATPase. Percent inhibition of S. aureus GyrB ATPase at a compound concentration of 1 μM (unless otherwise noted) is disclosed in the following table:

| Compound # | % Inhibition |
|---|---|
| 1 | 99.2 |
| 2 | 101.0 |
| 3 | 98.6 |
| 4 | 97.7 |
| 5 | 99.4 |
| 6 | 99.9 |
| 7 | 97.1 |
| 8 | 105.9 |
| 9 | 102.1 |
| 10 | 101.3 |
| 11 | 92.0 |
| 12 | 104.9 |
| 13 | 100.7 |
| 14 | 98.3 |
| 15 | 100.7 |
| 16 | 100.5 |
| 17 | 97.7 |
| 18 | 100.6 |
| 19 | 101.9 |
| 20 | 98.6 |
| 21 | 99.1 |
| 22 | 101.0 |
| 23 | 100.4 |
| 24 | 65.9 |
| 25 | 98.3 |
| 26 | 97.4 |
| 27 | 99.9 |
| 28 | 87.1 |
| 29 | 101.0 |
| 30 | 101.8 |
| 31 | 99.3 |
| 32 | 99.9 |
| 33 | 101.3 |
| 34 | 99.6 |
| 35 | 96.6 |
| 36 | 99.6 |
| 37 | 101.0 |
| 38 | 97.3 |
| 39 | 100.7 |
| 40 | 100.7 |
| 41 | 103.7 |
| 42 | 99.9 |
| 43 | 102.0 |
| 44 | 99.6 |
| 45 | 100.3 |
| 46 | 101.5 |
| 47 | 100.6 |
| 48 | 100.2 |
| 49 | 112.5 |
| 50 | 97.0 |
| 51 | No data available |
| 52 | 101.1 |
| 53 | 100.4 |
| 54 | 97.8 |
| 55 | 97.9 |
| 56 | 93.9 |
| 57 | No data available |
| 58 | 99.5 |
| 59 | 100.0 |
| 60 | 103.6 |
| 61 | 94.7 |
| 62 | 99.9 |
| 63 | 99.8 |
| 64 | 96.2 |
| 65 | 98.3 |
| 66 | 100.6 |
| 67 | 75.2 |
| 68 | 97.0 |
| 69 | 100.1 |
| 70 | 99.5 |
| 71 | 101.1 |
| 72 | 101.2 |
| 73 | 99.9 |
| 74 | 100.5 |
| 75 | 93.1 |
| 76 | 99.2 |
| 77 | 100.4 |
| 78 | 100.8 |
| 79 | 100.7 |
| 80 | 99.7 |
| 81 | 100.5 |
| 82 | 102.9 |
| 83 | 102.0 |
| 84 | 102.7 |
| 85 | 102.1 |
| 86 | 99.9 |
| 87 | 71.0 |
| 88 | No data available |
| 89 | No data available |
| 90 | 100.7 |
| 91 | 96.5 |
| 92 | 100.3 |
| 93 | 99.3 |
| 94 | 106.2 |
| 95 | No data available |
| 96 | 90.2 |
| 97 | 91.5 |
| 98 | 84.4 |
| 99 | 101.9 |
| 100 | 111.7 |
| 101 | 93.1 |
| 102 | 81.4 |
| 103 | 85.7 |
| 104 | 86.4 |
| 105 | 103.3 |
| 106 | 100.9 |
| 107 | No data available |
| 108 | 86.6 |
| 109 | 101.0 |
| 110 | No data available |
| 111 | 104.9 |
| 112 | 81.1 |
| 113 | 92.9 |
| 114 | 82.9 |
| 115 | 87.5 |
| 116 | 101.1 |
| 117 | 108.5 |
| 118 | 92.1 |
| 119 | 79.4 |
| 120 | 103.5 |
| 121 | 103.2 |
| 122 | 102.9 |
| 123 | 103.5 |
| 124 | 103.7 |
| 125 | 103.4 |
| 126 | No data available |
| 127 | No data available |
| 128 | No data available |
| 129 | No data available |
| 130 | No data available |
| 131 | No data available |
| 132 | No data available |
| 133 | No data available |
| 134 | No data available |
| 135 | No data available |
| 136 | No data available |
| 137 | No data available |
| 138 | No data available |
| 139 | No data available |
| 140 | No data available |
| 141 | 116.0 |
| 142 | No data available |
| 143 | 98.8 |
| 144 | 113.9 |
| 145 | 112.6 |
| 146 | 115.9 |
| 147 | No data available |
| 148 | 99.9 |
| 149 | 103.3 |
| 150 | 101.6 |
| 151 | 114.8 |
| 152 | No data available |
| 153 | 98.6 |
| 154 | 112.0 |
| 155 | 109.6 |
| 156 | No data available |
| 157 | No data available |
| 158 | No data available |
| 159 | 31.0 |
| 160 | 4.5 |
| 161 | 14.0* |
| 162 | No data available |
| 163 | No data available |
| 164 | No data available |
| 165 | No data available |
| 166 | No data available |
| 167 | No data available |
| 168 | No data available |
| 169 | No data available |
| 170 | No data available |
| 171 | No data available |
| 172 | No data available |
| 173 | No data available |

*Note: Compound was tested at a concentration of 5 μM.

### Bacterial Susceptibility Testing Methods

Compounds may be tested for antimicrobial activity by susceptibility testing in liquid media. Compounds may be dissolved in dimethylsulfoxide and tested in 10 doubling dilutions in the susceptibility assays. The organisms used in the assay may be grown overnight on suitable agar media and then suspended in a liquid medium appropriate for the growth of the organism. The suspension can be a 0.5 McFarland and a further 1 in 10 dilution can be made into the same liquid medium to prepare the final organism suspension in 100 μL. Plates can be incubated under appropriate conditions at 37° C. for 24 hrs prior to reading. The Minimum Inhibitory Concentration (MIC) may be determined as the lowest drug concentration able to reduce growth by 80% or more.

In an assay comparable to the above, Example 11 had an MIC of 0.06 μg/ml against Streptococcus pneumoniae.

According to a further feature of the invention there is provided a compound of the formula (I) or (Ia), or a pharmaceutically-acceptable salt thereof for use in a method of treatment of the human or animal body by therapy.

We have found that compounds of the present invention inhibit bacterial DNA gyrase and/or topoisomerase IV and are therefore of interest for their antibacterial effects. In one aspect of the invention the compounds of the invention inhibit bacterial DNA gyrase and are therefore of interest for their antibacterial effects. In one aspect of the invention, the compounds of the invention inhibit topoisomerase IV and are therefore of interest for their antibacterial effects. In one aspect of the invention the compounds of the invention inhibit both DNA gyrase and topoisomerase IV and are therefore of interest for their antibacterial effects.

It is expected that the compounds of the present invention will be useful in treating bacterial infections. In one aspect of the invention “infection” or “bacterial infection” refers to a gynecological infection. In one aspect of the invention “infection” or “bacterial infection” refers to a respiratory tract infection (RTI). In one aspect of the invention “infection” or “bacterial infection” refers to a sexually transmitted disease. In one aspect of the invention “infection” or “bacterial infection” refers to a urinary tract infection. In one aspect of the invention “infection” or “bacterial infection” refers to acute exacerbation of chronic bronchitis (ACEB). In one aspect of the invention “infection” or “bacterial infection” refers to acute otitis media. In one aspect of the invention “infection” or “bacterial infection” refers to acute sinusitis. In one aspect of the invention “infection” or “bacterial infection” refers to an infection caused by drug resistant bacteria. In one aspect of the invention “infection” or “bacterial infection” refers to catheter-related sepsis. In one aspect of the invention “infection” or “bacterial infection” refers to chancroid. In one aspect of the invention “infection” or “bacterial infection” refers to chlamydia. In one aspect of the invention “infection” or “bacterial infection” refers to community-acquired pneumoniae (CAP). In one aspect of the invention “infection” or “bacterial infection” refers to complicated skin and skin structure infection. In one aspect of the invention “infection” or “bacterial infection” refers to uncomplicated skin and skin structure infection. In one aspect of the invention “infection” or “bacterial infection” refers to endocarditis. In one aspect of the invention “infection” or “bacterial infection” refers to febrile neutropenia. In one aspect of the invention “infection” or “bacterial infection” refers to gonococcal cervicitis. In one aspect of the invention “infection” or “bacterial infection” refers to gonococcal urethritis. In one aspect of the invention “infection” or “bacterial infection” refers to hospital-acquired pneumonia (HAP). In one aspect of the invention “infection” or “bacterial infection” refers to osteomyelitis. In one aspect of the invention “infection” or “bacterial infection” refers to sepsis. In one aspect of the invention “infection” or “bacterial infection” refers to syphilis.

In one aspect of the invention an “infection” or “bacterial infection” refers to an infection caused by Acinetobacter baumanii. In one aspect of the invention an “infection” or “bacterial infection” refers to an infection caused by Acinetobacter haemolyticus. In one aspect of the invention an “infection” or “bacterial infection” refers to an infection caused by Acinetobacter junii. In one aspect of the invention an “infection” or “bacterial infection” refers to an infection caused by Acinetobacter johnsonii. In one aspect of the invention an “infection” or “bacterial infection” refers to an infection caused by Acinetobacter lwoffl. In one aspect of the invention an “infection” or “bacterial infection” refers to an infection caused by Bacteroides bivius. In one aspect of the invention an “infection” or “bacterial infection” refers to an infection caused by Bacteroides fragilis. In one aspect of the invention an “infection” or “bacterial infection” refers to an infection caused by Burkholderia cepacia. In one aspect of the invention an “infection” or “bacterial infection” refers to an infection caused by Campylobacter jejuni. In one aspect of the invention an “infection” or “bacterial infection” refers to an infection caused by Chlamydia pneumoniae. In one aspect of the invention an “infection” or “bacterial infection” refers to an infection caused by Chlamydia urealyticus. In one aspect of the invention an “infection” or “bacterial infection” refers to an infection caused by Chlamydophila pneumoniae. In one aspect of the invention an “infection” or “bacterial infection” refers to an infection caused by Clostridium dfficili. In one aspect of the invention an “infection” or “bacterial infection” refers to an infection caused by Enterobacter aerogenes. In one aspect of the invention an “infection” or “bacterial infection” refers to an infection caused by Enterobacter cloacae. In one aspect of the invention an “infection” or “bacterial infection” refers to an infection caused by Enterococcus faecalis. In one aspect of the invention an “infection” or “bacterial infection” refers to an infection caused by Enterococcus faecium. In one aspect of the invention an “infection” or “bacterial infection” refers to an infection caused by Escherichia coli. In one aspect of the invention an “infection” or “bacterial infection” refers to an infection caused by Gardnerella vaginalis. In one aspect of the invention an “infection” or “bacterial infection” refers to an infection caused by Haemophilus parainfluenzae. In one aspect of the invention an “infection” or “bacterial infection” refers to an infection caused by Haemophilus influenzae. In one aspect of the invention an “infection” or “bacterial infection” refers to an infection caused by Helicobacter pylori. In one aspect of the invention an “infection” or “bacterial infection” refers to an infection caused by Klebsiella pneumoniae. In one aspect of the invention an “infection” or “bacterial infection” refers to an infection caused by Legionella pneumophila. In one aspect of the invention an “infection” or “bacterial infection” refers to an infection caused by Methicillin-resistant Staphylococcus aureus. In one aspect of the invention an “infection” or “bacterial infection” refers to an infection caused by Methicillin-susceptible Staphylococcus aureus. In one aspect of the invention an “infection” or “bacterial infection” refers to an infection caused by Moraxella catarrhalis. In one aspect of the invention an “infection” or “bacterial infection” refers to an infection caused by Morganella morganii. In one aspect of the invention an “infection” or “bacterial infection” refers to an infection caused by Mycoplasma pneumoniae. In one aspect of the invention an “infection” or “bacterial infection” refers to an infection caused by Neisseria gonorrhoeae. In one aspect of the invention an “infection” or “bacterial infection” refers to an infection caused by Penicillin-resistant Streptococcus pneumoniae. In one aspect of the invention an “infection” or “bacterial infection” refers to an infection caused by Penicillin-susceptible Streptococcus pneumoniae. In one aspect of the invention an “infection” or “bacterial infection” refers to an infection caused by Peptostreptococcus magnus. In one aspect of the invention an “infection” or “bacterial infection” refers to an infection caused by Peptostreptococcus micros. In one aspect of the invention an “infection” or “bacterial infection” refers to an infection caused by Peptostreptococcus anaerobius. In one aspect of the invention an “infection” or “bacterial infection” refers to an infection caused by Peptostreptococcus asaccharolyticus. In one aspect of the invention an “infection” or “bacterial infection” refers to an infection caused by Peptostreptococcus prevotii. In one aspect of the invention an “infection” or “bacterial infection” refers to an infection caused by Peptostreptococcus tetradius. In one aspect of the invention an “infection” or “bacterial infection” refers to an infection caused by Peptostreptococcus vaginalis. In one aspect of the invention an “infection” or “bacterial infection” refers to an infection caused by Proteus mirabilis. In one aspect of the invention an “infection” or “bacterial infection” refers to an infection caused by Pseudomonas aeruginosa. In one aspect of the invention an “infection” or “bacterial infection” refers to an infection caused by Quinolone-Resistant Staphylococcus aureus. In one aspect of the invention an “infection” or “bacterial infection” refers to an infection caused by Quinolone-Resistant Staphylococcus epidermis. In one aspect of the invention an “infection” or “bacterial infection” refers to an infection caused by Salmonella typhi. In one aspect of the invention an “infection” or “bacterial infection” refers to an infection caused by Salmonella paratyphi. In one aspect of the invention an “infection” or “bacterial infection” refers to an infection caused by Salmonella enteritidis. In one aspect of the invention an “infection” or “bacterial infection” refers to an infection caused by Salmonella typhimurium. In one aspect of the invention an “infection” or “bacterial infection” refers to an infection caused by Serratia marcescens. In one aspect of the invention an “infection” or “bacterial infection” refers to an infection caused by Staphylococcus aureus. In one aspect of the invention an “infection” or “bacterial infection” refers to an infection caused by Staphylococcus epidermidis. In one aspect of the invention an “infection” or “bacterial infection” refers to an infection caused by Staphylococcus saprophyticus. In one aspect of the invention an “infection” or “bacterial infection” refers to an infection caused by Streptoccocus agalactiae. In one aspect of the invention an “infection” or “bacterial infection” refers to an infection caused by Streptococcus agalactiae. In one aspect of the invention an “infection” or “bacterial infection” refers to an infection caused by Streptococcus pneumoniae. In one aspect of the invention an “infection” or “bacterial infection” refers to an infection caused by Streptococcus pyogenes. In one aspect of the invention an “infection” or “bacterial infection” refers to an infection caused by Stenotrophomonas maltophilia. In one aspect of the invention an “infection” or “bacterial infection” refers to an infection caused by Ureaplasma urealyticum. In one aspect of the invention an “infection” or “bacterial infection” refers to an infection caused by Vancomycin-Resistant Enterococcus faecium. In one aspect of the invention an “infection” or “bacterial infection” refers to an infection caused by Vancomycin-Resistant Enterococcus faecalis. In one aspect of the invention an “infection” or “bacterial infection” refers to an infection caused by Vancomycin-Resistant Staphylococcus aureus. In one aspect of the invention an “infection” or “bacterial infection” refers to an infection caused by Vancomycin-Resistant Staphylococcus epidermis.

In one aspect of the invention an “infection” or “bacterial infection” refers to an infection caused by Acinetobacter spp. In one aspect of the invention an “infection” or “bacterial infection” refers to an infection caused by Bacteroides spp. In one aspect of the invention an “infection” or “bacterial infection” refers to an infection caused by Burkholderia spp. In one aspect of the invention an “infection” or “bacterial infection” refers to an infection caused by Campylobacter spp. In one aspect of the invention an “infection” or “bacterial infection” refers to an infection caused by Chlamydia spp. In one aspect of the invention an “infection” or “bacterial infection” refers to an infection caused by Chlamydophila spp. In one aspect of the invention an “infection” or “bacterial infection” refers to an infection caused by Clostridium spp. In one aspect of the invention an “infection” or “bacterial infection” refers to an infection caused by Enterobacter spp. In one aspect of the invention an “infection” or “bacterial infection” refers to an infection caused by Enterococcus spp. In one aspect of the invention an “infection” or “bacterial infection” refers to an infection caused by Escherichia spp. In one aspect of the invention an “infection” or “bacterial infection” refers to an infection caused by Gardnerella spp. In one aspect of the invention an “infection” or “bacterial infection” refers to an infection caused by Haemophilus spp. In one aspect of the invention an “infection” or “bacterial infection” refers to an infection caused by Helicobacter spp. In one aspect of the invention an “infection” or “bacterial infection” refers to an infection caused by Klebsiella spp. In one aspect of the invention an “infection” or “bacterial infection” refers to an infection caused by Legionella spp. In one aspect of the invention an “infection” or “bacterial infection” refers to an infection caused by Moraxella spp. In one aspect of the invention an “infection” or “bacterial infection” refers to an infection caused by Morganella spp. In one aspect of the invention an “infection” or “bacterial infection” refers to an infection caused by Mycoplasma spp. In one aspect of the invention an “infection” or “bacterial infection” refers to an infection caused by Neisseria spp. In one aspect of the invention an “infection” or “bacterial infection” refers to an infection caused by Peptostreptococcus spp. In one aspect of the invention an “infection” or “bacterial infection” refers to an infection caused by Proteus spp. In one aspect of the invention an “infection” or “bacterial infection” refers to an infection caused by Pseudomonas spp. In one aspect of the invention an “infection” or “bacterial infection” refers to an infection caused by Salmonella spp. In one aspect of the invention an “infection” or “bacterial infection” refers to an infection caused by Serratia spp. In one aspect of the invention an “infection” or “bacterial infection” refers to an infection caused by Staphylococcus spp. In one aspect of the invention an “infection” or “bacterial infection” refers to an infection caused by Streptoccocus spp. In one aspect of the invention an “infection” or “bacterial infection” refers to an infection caused by Stenotrophomonas spp. In one aspect of the invention an “infection” or “bacterial infection” refers to an infection caused by Ureaplasma spp. In one aspect of the invention an “infection” or “bacterial infection” refers to an infection caused by aerobes. In one aspect of the invention an “infection” or “bacterial infection” refers to an infection caused by obligate anaerobes. In one aspect of the invention an “infection” or “bacterial infection” refers to an infection caused by facultative anaerobes. In one aspect of the invention an “infection” or “bacterial infection” refers to an infection caused by gram-positive bacteria. In one aspect of the invention an “infection” or “bacterial infection” refers to an infection caused by gram-negative bacteria. In one aspect of the invention an “infection” or “bacterial infection” refers to an infection caused by gram-variable bacteria. In one aspect of the invention an “infection” or “bacterial infection” refers to an infection caused by atypical respiratory pathogens.

According to a further feature of the present invention there is provided a method for producing an antibacterial effect in a warm blooded animal, such as man, in need of such treatment, which comprises administering to said animal an effective amount of a compound of the present invention, or a pharmaceutically-acceptable salt thereof.

According to a further feature of the invention there is provided a method for inhibition of bacterial DNA gyrase and/or topoisomerase IV in a warm-blooded animal, such as a human being, in need of such treatment which comprises administering to said animal an effective amount of a compound of formula (I) or (Ia) or a pharmaceutically acceptable salt thereof as defined hereinbefore.

According to a further feature of the invention there is provided a method of treating a bacterial infection in a warm-blooded animal, such as a human being, in need of such treatment which comprises administering to said animal an effective amount of a compound of formula (I) or (Ia) or a pharmaceutically acceptable salt thereof as defined hereinbefore.

According to a further feature of the invention there is provided a method of treating a bacterial infection selected from a gynecological infection, a respiratory tract infection (RTI), a sexually transmitted disease, a urinary tract infection, acute exacerbation of chronic bronchitis (ACEB), acute otitis media, acute sinusitis, an infection caused by drug resistant bacteria, catheter-related sepsis, chancroid, chlamydia, community-acquired pneumoniae (CAP), complicated skin and skin structure infection, uncomplicated skin and skin structure infection, endocarditis, febrile neutropenia, gonococcal cervicitis, gonococcal urethritis, hospital-acquired pneumonia (HAP), osteomyelitis, sepsis and/or syphilis in a warm-blooded animal, such as a human being, in need of such treatment which comprises administering to said animal an effective amount of a compound of formula (I) or (Ia) or a pharmaceutically acceptable salt thereof as defined hereinbefore.

A further feature of the present invention is a compound of formula (I) or (Ia) and pharmaceutically acceptable salts thereof for use as a medicament. Suitably the medicament is an antibacterial agent.

According to a further aspect of the invention there is provided the use of a compound of formula (I) or (Ia), or a pharmaceutically acceptable salt thereof in the manufacture of a medicament for the production of an anti-bacterial effect in a warm-blooded animal such as a human being.

According to a further aspect of the invention there is provided the use of a compound of formula (I) or (Ia), or a pharmaceutically acceptable salt thereof in the manufacture of a medicament for the inhibition of bacterial DNA gyrase and/or topoisomerase IV in a warm-blooded animal such as a human being.

Thus according to a further aspect of the invention there is provided the use of a compound of formula (I) or (Ia), or a pharmaceutically acceptable salt thereof in the manufacture of a medicament for the treatment of a bacterial infection in a warm-blooded animal such as a human being.

Thus according to a further aspect of the invention there is provided the use of a compound of formula (I) or (Ia), or a pharmaceutically acceptable salt thereof in the manufacture of a medicament for the treatment of a bacterial infection selected from a gynecological infection, a respiratory tract infection (RTI), a sexually transmitted disease, a urinary tract infection, acute exacerbation of chronic bronchitis (ACEB), acute otitis media, acute sinusitis, an infection caused by drug resistant bacteria, catheter-related sepsis, chancroid, chlamydia, community-acquired pneumoniae (CAP), complicated skin and skin structure infection, uncomplicated skin and skin structure infection, endocarditis, febrile neutropenia, gonococcal cervicitis, gonococcal urethritis, hospital-acquired pneumonia (HAP), osteomyelitis, sepsis and/or syphilis in a warm-blooded animal such as a human being.

According to a further aspect of the invention there is provided a compound of formula (I) or (Ia), or a pharmaceutically acceptable salt thereof for use in the production of an anti-bacterial effect in a warm-blooded animal such as a human being.

According to a further aspect of the invention there is provided a compound of formula (I) or (Ia), or a pharmaceutically acceptable salt thereof for use in inhibition of bacterial DNA gyrase and/or topoisomerase IV in a warm-blooded animal such as a human being.

Thus according to a further aspect of the invention there is provided a compound of formula (I) or (Ia), or a pharmaceutically acceptable salt thereof for use in the treatment of a bacterial infection in a warm-blooded animal such as a human being.

Thus according to a further aspect of the invention there is provided a compound of formula (I) or (Ia), or a pharmaceutically acceptable salt thereof for use in the treatment of a bacterial infection selected from a gynecological infection, a respiratory tract infection (RTI), a sexually transmitted disease, a urinary tract infection, acute exacerbation of chronic bronchitis (ACEB), acute otitis media, acute sinusitis, an infection caused by drug resistant bacteria, catheter-related sepsis, chancroid, chlamydia, community-acquired pneumoniae (CAP), complicated skin and skin structure infection, uncomplicated skin and skin structure infection, endocarditis, febrile neutropenia, gonococcal cervicitis, gonococcal urethritis, hospital-acquired pneumonia (HAP), osteomyelitis, sepsis and/or syphilis in a warm-blooded animal such as a human being.

In order to use a compound of the formula (I) or (Ia) or a pharmaceutically-acceptable salt thereof, for the therapeutic (including prophylactic) treatment of mammals including humans, in particular in treating infection, it is normally formulated in accordance with standard pharmaceutical practice as a pharmaceutical composition.

Therefore in another aspect the present invention provides a pharmaceutical composition which comprises a compound of the formula (I) or (Ia) or a pharmaceutically-acceptable salt thereof, and a pharmaceutically-acceptable diluent or carrier.

According to a further aspect of the invention there is provided a pharmaceutical composition which comprises a compound of formula (I) or (Ia) as defined hereinbefore or a pharmaceutically acceptable salt thereof, in association with a pharmaceutically acceptable excipient or carrier for use in producing an anti-bacterial effect in a warm-blooded animal, such as a human being.

According to a further aspect of the invention there is provided a pharmaceutical composition which comprises a compound of formula (I) or (Ia) as defined hereinbefore or a pharmaceutically acceptable salt thereof, in association with a pharmaceutically acceptable excipient or carrier for use in inhibition of bacterial DNA gyrase and/or topoisomerase IV in a warm-blooded animal, such as a human being.

According to a further aspect of the invention there is provided a pharmaceutical composition which comprises a compound of formula (I) or (Ia) as defined hereinbefore or a pharmaceutically acceptable salt thereof, in association with a pharmaceutically acceptable excipient or carrier for use in the treatment of a bacterial infection in a warm-blooded animal, such as a human being.

According to a further aspect of the invention there is provided a pharmaceutical composition which comprises a compound of formula (I) or (Ia) as defined hereinbefore or a pharmaceutically acceptable salt thereof, in association with a pharmaceutically acceptable excipient or carrier for use in the treatment of a gynecological infection, a respiratory tract infection (RTI), a sexually transmitted disease, a urinary tract infection, acute exacerbation of chronic bronchitis (ACEB), acute otitis media, acute sinusitis, an infection caused by drug resistant bacteria, catheter-related sepsis, chancroid, chlamydia, community-acquired pneumoniae (CAP), complicated skin and skin structure infection, uncomplicated skin and skin structure infection, endocarditis, febrile neutropenia, gonococcal cervicitis, gonococcal urethritis, hospital-acquired pneumonia (HAP), osteomyelitis, sepsis and/or syphilis in a warm-blooded animal, such as a human being.

The compositions of the invention may be in a form suitable for oral use (for example as tablets, lozenges, hard or soft capsules, aqueous or oily suspensions, emulsions, dispersible powders or granules, syrups or elixirs), for topical use (for example as creams, ointments, gels, or aqueous or oily solutions or suspensions), for administration by inhalation (for example as a finely divided powder or a liquid aerosol), for administration by insufflation (for example as a finely divided powder) or for parenteral administration (for example as a sterile aqueous or oily solution for intravenous, subcutaneous, intramuscular or intramuscular dosing or as a suppository for rectal dosing).

The compositions of the invention may be obtained by conventional procedures using conventional pharmaceutical excipients, well known in the art. Thus, compositions intended for oral use may contain, for example, one or more colouring, sweetening, flavouring and/or preservative agents.

Suitable pharmaceutically acceptable excipients for a tablet formulation include, for example, inert diluents such as lactose, sodium carbonate, calcium phosphate or calcium carbonate, granulating and disintegrating agents such as corn starch or algenic acid; binding agents such as starch; lubricating agents such as magnesium stearate, stearic acid or talc; preservative agents such as ethyl or propyl p-hydroxybenzoate, and anti-oxidants, such as ascorbic acid. Tablet formulations may be uncoated or coated either to modify their disintegration and the subsequent absorption of the active ingredient within the gastrointestinal tract, or to improve their stability and/or appearance, in either case, using conventional coating agents and procedures well known in the art.

Compositions for oral use may be in the form of hard gelatin capsules in which the active ingredient is mixed with an inert solid diluent, for example, calcium carbonate, calcium phosphate or kaolin, or as soft gelatin capsules in which the active ingredient is mixed with water or an oil such as peanut oil, liquid paraffin, or olive oil.

Aqueous suspensions generally contain the active ingredient in finely powdered form together with one or more suspending agents, such as sodium carboxymethylcellulose, methylcellulose, hydroxypropylmethylcellulose, sodium alginate, polyvinyl-pyrrolidone, gum tragacanth and gum acacia; dispersing or wetting agents such as lecithin or condensation products of an alkylene oxide with fatty acids (for example polyoxethylene stearate), or condensation products of ethylene oxide with long chain aliphatic alcohols, for example heptadecaethyleneoxycetanol, or condensation products of ethylene oxide with partial esters derived from fatty acids and a hexitol such as polyoxyethylene sorbitol monooleate, or condensation products of ethylene oxide with long chain aliphatic alcohols, for example heptadecaethyleneoxycetanol, or condensation products of ethylene oxide with partial esters derived from fatty acids and a hexitol such as polyoxyethylene sorbitol monooleate, or condensation products of ethylene oxide with partial esters derived from fatty acids and hexitol anhydrides, for example polyethylene sorbitan monooleate. The aqueous suspensions may also contain one or more preservatives (such as ethyl or propyl p-hydroxybenzoate, anti-oxidants (such as ascorbic acid), colouring agents, flavouring agents, and/or sweetening agents (such as sucrose, saccharine or aspartame).

Oily suspensions may be formulated by suspending the active ingredient in a vegetable oil (such as arachis oil, olive oil, sesame oil or coconut oil) or in a mineral oil (such as liquid paraffin). The oily suspensions may also contain a thickening agent such as beeswax, hard paraffin or cetyl alcohol. Sweetening agents such as those set out above, and flavouring agents may be added to provide a palatable oral preparation. These compositions may be preserved by the addition of an anti-oxidant such as ascorbic acid.

Dispersible powders and granules suitable for preparation of an aqueous suspension by the addition of water generally contain the active ingredient together with a dispersing or wetting agent, suspending agent and one or more preservatives. Suitable dispersing or wetting agents and suspending agents are exemplified by those already mentioned above. Additional excipients such as sweetening, flavouring and colouring agents, may also be present.

The pharmaceutical compositions of the invention may also be in the form of oil-in-water emulsions. The oily phase may be a vegetable oil, such as olive oil or arachis oil, or a mineral oil, such as for example liquid paraffin or a mixture of any of these. Suitable emulsifying agents may be, for example, naturally-occurring gums such as gum acacia or gum tragacanth, naturally-occurring phosphatides such as soya bean, lecithin, an esters or partial esters derived from fatty acids and hexitol anhydrides (for example sorbitan monooleate) and condensation products of the said partial esters with ethylene oxide such as polyoxyethylene sorbitan monooleate. The emulsions may also contain sweetening, flavouring and preservative agents.

Syrups and elixirs may be formulated with sweetening agents such as glycerol, propylene glycol, sorbitol, aspartame or sucrose, and may also contain a demulcent, preservative, flavouring and/or colouring agent.

The pharmaceutical compositions may also be in the form of a sterile injectable aqueous or oily suspension, which may be formulated according to known procedures using one or more of the appropriate dispersing or wetting agents and suspending agents, which have been mentioned above. A sterile injectable preparation may also be a sterile injectable solution or suspension in a non-toxic parenterally-acceptable diluent or solvent, for example a solution in 1,3-butanediol.

Compositions for administration by inhalation may be in the form of a conventional pressurised aerosol arranged to dispense the active ingredient either as an aerosol containing finely divided solid or liquid droplets. Conventional aerosol propellants such as volatile fluorinated hydrocarbons or hydrocarbons may be used and the aerosol device is conveniently arranged to dispense a metered quantity of active ingredient.

For further information on formulation the reader is referred to Chapter 25.2 in Volume 5 of Comprehensive Medicinal Chemistry (Corwin Hansch; Chairman of Editorial Board), Pergamon Press 1990.

The amount of active ingredient that is combined with one or more excipients to produce a single dosage form will necessarily vary depending upon the host treated and the particular route of administration. For example, a formulation intended for oral administration to humans will generally contain, for example, from 0.5 mg to 2 g of active agent compounded with an appropriate and convenient amount of excipients which may vary from about 5 to about 98 percent by weight of the total composition. Dosage unit forms will generally contain about 1 mg to about 500 mg of an active ingredient. For further information on Routes of Administration and Dosage Regimes the reader is referred to Chapter 25.3 in Volume 5 of Comprehensive Medicinal Chemistry (Corwin Hansch; Chairman of Editorial Board), Pergamon Press 1990.

As stated above the size of the dose required for the therapeutic or prophylactic treatment of a particular disease state will necessarily be varied depending on the host treated, the route of administration and the severity of the illness being treated. In one aspect of the invention a daily dose in the range of 1-50 mg/kg is employed. However the daily dose will necessarily be varied depending upon the host treated, the particular route of administration, and the severity of the illness being treated. Accordingly the optimum dosage may be determined by the practitioner who is treating any particular patient.

In addition to its use in therapeutic medicine, compounds of formula (I) or (Ia) and their pharmaceutically acceptable salts are also useful as pharmacological tools in the development and standardisation of in-vitro and in-vivo test systems for the evaluation of the effects of inhibitors of DNA gyrase and/or topoisomerase IV in laboratory animals such as cats, dogs, rabbits, monkeys, rats and mice, as part of the search for new therapeutic agents.

In the above other, pharmaceutical composition, process, method, use and medicament manufacture features, the alternative and particular embodiments of the compounds of the invention described herein also apply.

### Combinations

The compounds of the invention described herein may be applied as a sole therapy or may involve, in addition to a compound of the invention, one or more other substances and/or treatments. Such conjoint treatment may be achieved by way of the simultaneous, sequential or separate administration of the individual components of the treatment. Where the administration is sequential or separate, the delay in administering the second component should not be such as to lose the beneficial effect of the combination. Suitable classes and substances may be selected from one or more of the following:
i) other antibacterial agents for example macrolides e.g. erythromycin, azithromycin or clarithromycin; quinolones e.g. ciprofloxacin or levofloxacin; β-lactams e.g. penicillins e.g. amoxicillin or piperacillin; cephalosporins e.g. ceftriaxone or ceftazidime; carbapenems, e.g. meropenem or imipenem etc; aminoglycosides e.g. gentamicin or tobramycin; or oxazolidinones; and/or
ii) anti-infective agents for example, an antifungal triazole e.g. or amphotericin; and/or
iii) biological protein therapeutics for example antibodies, cytokines, bactericidal/permeability-increasing protein (BPI) products; and/or
iv) efflux pump inhibitors.

Therefore, in a further aspect of the invention there is provided a compound of the formula (I) or (Ia), or a pharmaceutically acceptable salt thereof and a chemotherapeutic agent selected from:
i) one or more additional antibacterial agents; and/or
ii) one or more anti-infective agents; and/or
iii) biological protein therapeutics for example antibodies, cytokines, bactericidal/permeability-increasing protein (BPI) products; and/or
iv) one or more efflux pump inhibitors.

### EXAMPLES

The invention is now illustrated but not limited by the following Examples in which unless otherwise stated :
(i) evaporations were carried out by rotary evaporation in-vacuo and work-up procedures were carried out after removal of residual solids by filtration;
(ii) operations were generally carried out at ambient temperature, that is typically in the range 18-26° C. and without exclusion of air unless otherwise stated, or unless the skilled person would otherwise work under an inert atmosphere;
(iii) column chromatography (by the flash procedure) was used to purify compounds and was performed on Merck Kieselgel silica (Art. 9385) unless otherwise stated;
(iv) yields are given for illustration only and are not necessarily the maximum attainable;
(v) the structure of the end-products of the invention were generally confirmed by NMR and mass spectral techniques; proton magnetic resonance spectra is quoted and was generally determined in DMSO-d₆ unless otherwise stated using a Bruker DRX-300 spectrometer operating at a field strength of 300 MHz. Chemical shifts are reported in parts per million downfield from tetramethysilane as an internal standard (δ scale) and peak multiplicities are shown thus: s, singlet; d, doublet; AB or dd, doublet of doublets; dt, doublet of triplets; dm, doublet of multiplets; t, triplet, m, multiplet; br, broad;
(vi) fast-atom bombardment (FAB) mass spectral data were generally obtained using a Platform spectrometer (supplied by Micromass) run in electrospray and, where appropriate, either positive ion data or negative ion data were collected or using Agilent 1100 series LC/MSD equipped with Sedex 75ELSD, run in atmospheric pressure chemical ionisation mode and, where appropriate, either positive ion data or negative ion data were collected; mass spectra were run with an electron energy of 70 electron volts in the chemical ionization (CI) mode using a direct exposure probe; where indicated ionization was effected by electron impact (EI), fast atom bombardment (FAB) or electrospray (ES); values for m/z are given; generally, only ions which indicate the parent mass are reported;
(vii) each intermediate was generally purified to the standard required for the subsequent stage and was characterised in sufficient detail to confirm that the assigned structure was correct; purity was assessed by high pressure liquid chromatography, thin layer chromatography, or NMR and identity was determined by infra-red spectroscopy (IR), mass spectroscopy or NMR spectroscopy as appropriate;
(vii) the following abbreviations may be used:

DMF is N,N-dimethylformamide;

SM is starting material;

DMSO is dimethylsulfoxide;

CDCl₃ is deuterated chloroform;

MS is mass spectroscopy;

EtOAc is ethyl acetate;

THF is tetrahydrofuran;

MeOH is methanol;

TFA is trifluoroacetic acid;

EtOH is ethanol;

DCM is dichloromethane;

HATU is N-[(dimethylamino)-1H,2,3-triazolo[4,5-b-]pyridin-1-ylmethylene]-N-methylmethanaminium hexafluorophosphate N-oxide;

DIEA is diisopropyl ethyl amine; and
(viii) temperatures are quoted as ° C.

### Example 1

### 2-((3S,4R)-4-{[(3,4-Dichloro-5-methyl-1H-pyrrol-2-yl)carbonyl]amino}-3-methoxypiperidin-1-yl)-4-pyrimidin-2-yl-1,3-thiazole-5-carboxylic acid

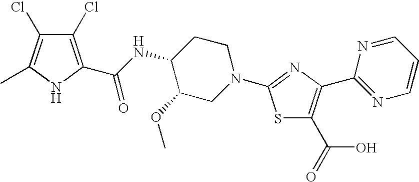

To a suspension of ethyl 2-((3S,4R)-4-{[(3,4-dichloro-5-methyl-1H-pyrrol-2-yl)carbonyl]amino}-3-methoxypiperidin-1-yl)-4-pyrimidin-2-yl-1,3-thiazole-5-carboxylate (Example 43; 0.04 g, 0.074 mmol) in MeOH was added lithium hydroxide (0.11 ml, 0.22 mmol, 2N). The reaction was heated in the microwave for 30 mins at 100° C. The crude reaction was diluted with water and acidified with 1N HCl. The resulting precipitate was filtered, washed with water, and dried (0.021 g). MS (ES) (M+H)⁺: 511 for C₂₀H₂₀Cl₂N₆O₄S; NMR: 1.75 (m, 2H), 2.17 (s, 3H), 3.38 (s, 3H), 4.04 (s, 1H), 4.26 (m, 2H), 7.18 (d, 1H), 7.63 (d, 1H), 8.98 (s, 1H, 12.15 (s, 1H.

### Examples 2-42

The following Examples were prepared by the procedure described in Example 1 from the starting materials (SM) indicated.

| Ex | Compound | Data | SM |
|---|---|---|---|
| 2 | 2-((3S,4R)-4-{[(3,4-Dichloro-5- | MS (ES) (M + H)⁺: 511 for | Example 81 |
| | methyl-1H-pyrrol-2- | C₂₀H₂₀Cl₂N₆O₄S | |
| | yl)carbonyl]amino}-3- | NMR: 1.72 (m, 2 H), 2.12 (s, 3 H), | |
| | methoxypiperidin-1-yl)-4- | 3.27 (m, 3 H), 3.33 (s, 3 H), 3.42 (s, | |
| | pyrimidin-4-yl-1,3-thiazole-5- | 1 H), 3.53 (m, 1 H), 4.24 (m, 1 H), | |
| | carboxylic acid | 7.09 (d, 1 H), 8.21 (d, 1 H), 9.07 (d, | |
| | 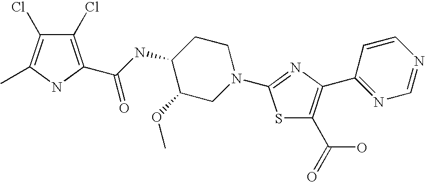 | 1 H), 9.36 (s, 1 H), 12.10 (s, 1 H) | |
| 3 | 2′-((3S,4R)-4-{[(3,4-Dichloro-5- | MS (ES) (M + H)⁺: 516 for | Example 44 |
| | methyl-1H-pyrrol-2- | C₁₉H₁₉Cl₂N₅O₄S₂ | |
| | yl)carbonyl]amino}-3- | NMR: 1.72 (m, 2 H), 2.12 (s, 3 H), | |
| | methoxypiperidin-1-yl)-2,4′-bi-1,3- | 3.32 (m, 1 H), 3.38 (s, 3 H), 3.54 (s, | |
| | thiazole-5′-carboxylic acid | 1 H), 3.86 (m, 1 H), 4.25 (m, 1 H), | |
| | 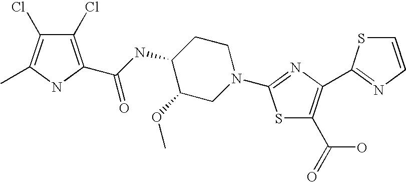 | 7.18 (d, 1 H), 7.80 (d, 2 H), 12.21 (s,1 H) | |
| 4 | 2-((3S,4R)-4-{[(3,4-Dichloro-5- | MS (ES) (M + H)⁺: 499 for | Example 45 |
| | methyl-1H-pyrrol-2- | C₁₉H₁₇Cl₂FN₆O₃S | |
| | yl)carbonyl]amino}-3- | NMR: 1.88 (m, 2 H), 2.20 (s, 3 H), | |
| | fluoropiperidin-1-yl)-4-pyrimidin- | 3.57 (m, 1 H), 4.39 (m, 3 H), 4.91 (d, | |
| | 2-yl-1,3-thiazole-5-carboxylic acid | 1 H), 7.28 (d, 1 H), 7.65 (t, 1 H), 9.00 | |
| | 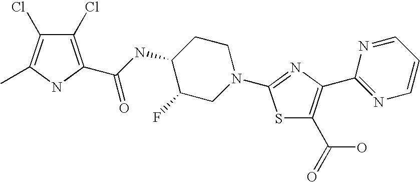 | (d, 2 H), 12.11 (s, 1 H), 15.08 (s, 1 H) | |
| 5 | 2-((3S,4R)-4-{[(3,4-Dichloro-5- | MS (ES) (M + H)⁺: 499 for | Example 46 |
| | methyl-1H-pyrrol-2- | C₁₉H₁₇Cl₂FN₆O₃S | |
| | yl)carbonyl]amino}-3- | NMR: 1.83 (m, 2 H), 2.13 (s, 3 H), | |
| | fluoropiperidin-1-yl)-4-pyrimidin- | 3.53 (m, 2 H), 4.06 (m, 1 H), 4.39 | |
| | 4-yl-1,3-thiazole-5-carboxylic acid | (m, 2 H), 4.85 (d, 1 H), 7.24 (d, 1 H), | |
| | 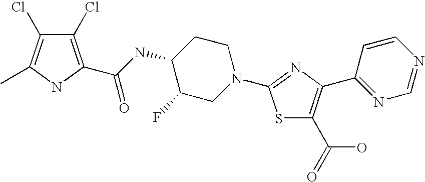 | 8.19 (t, 1 H), 9.07 (d, 1 H), 9.36 (s,1 H), 12.05 (s, 1 H) | |
| 6 | 2′-((3S,4R)-4-{[(3,4-Dichloro-5- | MS (ES) (M + H)⁺: 504 for | Example 47 |
| | methyl-1H-pyrrol-2- | C₁₈H₁₆Cl₂FN₅O₃S₂ | |
| | yl)carbonyl]amino}-3- | NMR: 1.89 (m, 2 H), 2.20 (s, 3 H), | |
| | fluoropiperidin-1-yl)-2,4′-bi-1,3- | 4.09 (m, 2 H), 4.40 (m, 3 H), 4.92 (d, | |
| | thiazole-5′-carboxylic acid | 1 H), 7.32 (d, 1 H), 8.15 (m, 2 H), | |
| | 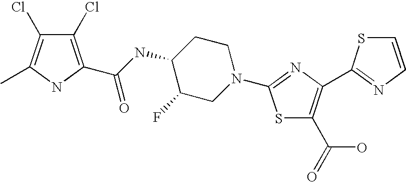 | 12.13 (s, 1 H) | |
| 7 | 2-((3S,4R)-4-{[(3,4-Dichloro-5- | MS (ES) (M + H)⁺: 510 for | Example 48 |
| | methyl-1H-pyrrol-2- | C₂₁H₂₁Cl₂N₅O₄S | |
| | yl)carbonyl]amino}-3- | NMR: 1.80 (m, 2 H), 2.19 (s, 3 H), | |
| | methoxypiperidin-1-yl)-4-pyridin- | 3.40 (s, 5H), 3.59 (s, 1 H), 4.04 (m, | |
| | 2-yl-1,3-thiazole-5-carboxylic acid | 1 H), 4.29 (m, 1 H), 4.46 (m, 1 H), | |
| | 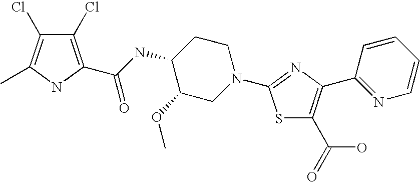 | 7.17 (d, 1 H), 7.71 (t, 1 H), 8.25 (t,1 H), 8.43 (d, 1 H), 8.76 (s, 1 H),12.17 (s, 1 H) | |
| 8 | 2-((3S,4R)-4-{[(3,4-Dichloro-5- | MS (ES) (M + H)⁺: 498 for | Example 49 |
| | methyl-1H-pyrrol-2- | C₂₀H₁₈Cl₂FN₅O₃S | |
| | yl)carbonyl]amino}-3- | NMR: 1.90 (m, 2 H), 2.20 (s, 3 H), | |
| | fluoropiperidin-1-yl)-4-pyridin-2- | 3.40 (s, 1 H), 3.59 (m, 1 H), 4.12 (m, | |
| | yl-1,3-thiazole-5-carboxylic acid | 1 H), 4.45 (m, 2 H), 4.93 (d, 1 H), | |
| | 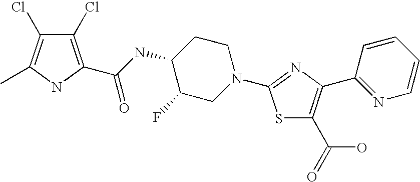 | 7.29 (d, 1 H), 7.72 (t, 1 H), 8.26 (t,1 H), 8.43 (d, 1 H), 8.76 (s, 1 H),12.11 (s, 1 H) | |
| 9 | 4-[2,6- | MS (ES) (M + H)⁺: 597 for | Example 50 |
| | Bis(dimethylamino)pyrimidin-4- | C₂₄H₃₀Cl₂N₈O₄S | |
| | yl]-2-((3S,4R)-4-{[(3,4-dichloro-5- | NMR: 1.70 (m, 2 H), 2.12 (s, 3 H), | |
| | methyl-1H-pyrrol-2- | 3.09 (s, 12 H), 3.28 (s, 3 H), 3.32 (s, | |
| | yl)carbonyl]amino}-3- | 2 H), 3.49 (s, 1 H), 3.79 (m, 1 H), | |
| | methoxypiperidin-1-yl)-1,3- | 4.19 (m, 1 H), 4.37 (m, 1 H), 6.83 (s, | |
| | thiazole-5-carboxylic acid | 1 H), 7.09 (d, 1 H), 12.09 (s, 1 H) | |
| | 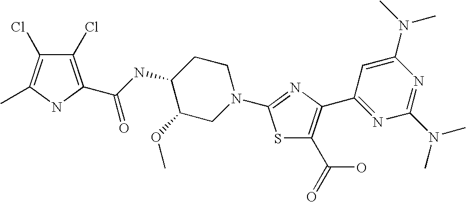 | | |
| 10 | 4-[2,6- | MS (ES) (M + H)⁺: 585 for | Example 51 |
| | Bis(dimethylamino)pyrimidin-4- | C₂₃H₂₇Cl₂FN₈O₃S | |
| | yl]-2-((3S,4R)-4-{[(3,4-dichloro-5- | NMR: 1.88 (m, 2 H), 2.20 (s, 3 H), | |
| | methyl-1H-pyrrol-2- | 3.35 (s, 12 H), 3.51 (m, 1 H), 4.05 | |
| | yl)carbonyl]amino}-3- | (m, 1 H), 4.38 (m, 2 H), 4.89 (d, 1 H), | |
| | fluoropiperidin-1-yl)-1,3-thiazole- | 6.90 (s, 1 H), 7.28 (d, 1 H), 12.12 (s, | |
| | 5-carboxylic acid | 1 H) | |
| | 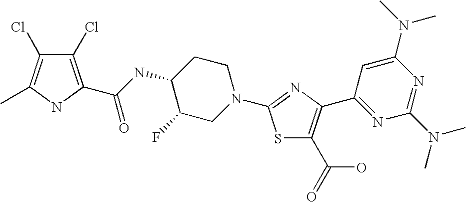 | | |
| 11 | 2-((3S,4R)-4-{[(3,4-Dichloro-5- | MS (ES) (M + H)⁺: 571 for | Example 52 |
| | methyl-1H-pyrrol-2- | C₂₂H₂₄Cl₂N₆O₆S | |
| | yl)carbonyl]amino}-3- | NMR: 1.70 (m, 2 H), 2.12 (s, 3 H), | |
| | methoxypiperidin-1-yl)-4-(4,6- | 3.27 (s, 6 H), 3.49 (m, 1 H), 3.84 (s, | |
| | dimethoxypyrimidin-2-yl)-1,3- | 6 H), 4.22 (m, 2 H), 6.29 (s, 1 H), | |
| | thiazole-5-carboxylic acid | 7.09 (d, 1 H), 12.10 (s, 1 H), 13.80 (s, | |
| | 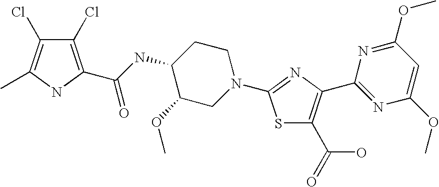 | 1 H) | |
| 12 | 2-((3S,4R)-4-{[(3,4-Dichloro-5- | MS (ES) (M + H)⁺: 511 for | Example 53 |
| | methyl-1H-pyrrol-2- | C₂₀H₂₀Cl₂N₆O₄S | |
| | yl)carbonyl]amino}-3- | NMR: 1.78 (m, 2 H), 2.17 (s, 3 H), | |
| | methoxypiperidin-1-yl)-4-pyrazin- | 3.33 (s, 3 H), 3.39 (s, 3 H), 4.01 (m, | |
| | 2-yl-1,3-thiazole-5-carboxylic acid | 1 H), 4.26 (m, 1 H), 4.27 (m, 1 H), | |
| | 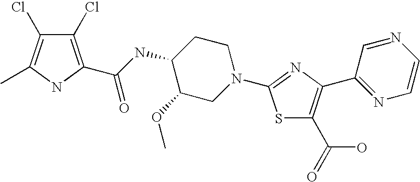 | 7.15 (d, 1 H), 8.77 (s, 1 H), 8.83 (s,1 H), 9.34 (s, 1 H), 12.16 (s, 1 H),15.29 (s, 1 H) | |
| 13 | 4-(1,3-Benzothiazol-2-yl)-2- | MS (ES) (M + H)⁺: 566 for | Example 54 |
| | ((3S,4R)-4-{[(3,4-dichloro-5- | C₂₃H₂₁Cl₂N₅O₄S₂ | |
| | methyl-1H-pyrrol-2- | NMR: 1.80 (m, 2 H), 2.17 (s, 3 H), | |
| | yl)carbonyl]amino}-3- | 3.35 (m, 3 H), 3.42 (s, 3 H), 3.61 (s, | |
| | methoxypiperidin-1-yl)-1,3- | 1 H), 4.29 (m, 1 H), 7.16 (d, 1 H), | |
| | thiazole-5-carboxylic acid | 7.64 (m, 2 H), 8.13 (d, 1 H), 8.27 (d, | |
| | 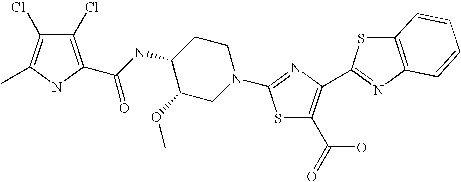 | 1 H), 12.16 (s, 1 H), | |
| 14 | 2-((3S,4R)-4-{[(3,4-Dichloro-5- | MS (ES) (M + H)⁺: 499 for | Example 55 |
| | methyl-1H-pyrrol-2- | C₁₉H₁₇Cl₂FN₆O₃S | |
| | yl)carbonyl]amino}-3- | NMR: 1.89 (m, 2 H), 2.20 (s, 3 H), | |
| | methoxypiperidin-1-yl)-4-pyrazin- | 3.36 (m, 1 H), 3.59 (dd, 1 H), 4.14 | |
| | 2-yl-1,3-thiazole-5-carboxylic acid | (m, 1 H), 4.44 (m, 2 H), 4.92 (d, 1 H), | |
| | 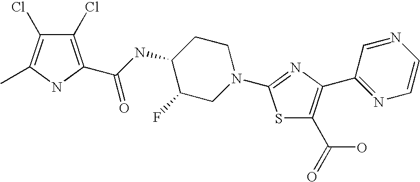 | 7.29 (d, 1 H), 8.78 (s, 1 H), 8.83 (s,1 H), 9.31 (s, 1 H), 12.12 (s, 1 H) | |
| 15 | 4-(1,3-Benzothiazol-2-yl)-2- | MS (ES) (M + H)⁺: 554 for | Example 56 |
| | ((3S,4R)-4-{[(3,4-dichloro-5- | C₂₂H₁₈Cl₂FN₅O₃S₂ | |
| | methyl-1H-pyrrol-2- | NMR: 1.86 (m, 2 H), 2.13 (s, 3 H), | |
| | yl)carbonyl]amino}-3- | 3.28 (m, 1 H), 3.59 (dd, 1 H), 4.08 | |
| | fluoropiperidin-1-yl)-1,3-thiazole- | (m, 1 H), 4.37 (m, 2 H), 4.89 (d, 1 H), | |
| | 5-carboxylic acid | 7.23 (d, 1 H), 7.58 (m, 2 H), 8.08 (d, | |
| | 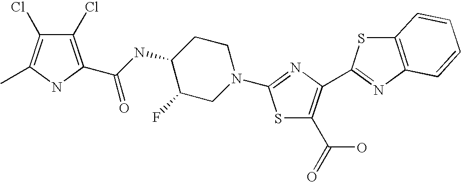 | 2 H), 8.21 (d, 1 H), 8.83 (s, 1 H), 9.31(s, 1 H), 12.05 (s, 1 H), 15.99 (s, 1 H) | |
| 16 | 2-((3S,4R)-4-{[(3,4-Dichloro-5- | MS (ES) (M + H)⁺: 630 for | Example 57 |
| | methyl-1H-pyrrol-2- | C₂₅H₃₄Cl₂N₆O₅SSi | |
| | yl)carbonyl]amino}-3- | NMR: −0.13 (s, 9 H), 0.82 (m, 2 H), | |
| | methoxypiperidin-1-yl)-4-(1-{[2- | 1.84 (m, 2 H), 2.23 (s, 3 H), 3.43 (m, | |
| | (trimethylsilyl)ethoxy]methyl}-1H- | 4 H), 3.61 (m, 2 H), 4.05 (m, 1 H), | |
| | imidazol-2-yl)-1,3-thiazole-5- | 4.30 (m, 2 H), 6.04 (m, 2 H), 7.15 (d, | |
| | carboxylic acid | 1 H), 7.43 (s, 1 H), 7.76 (s, 1 H), | |
| | 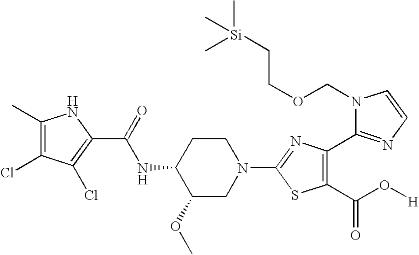 | 12.27 (s, 1 H). | |
| 17 | 2-((3S,4R)-4-{[(3,4-Dichloro-5- | MS (ES) (M + H)⁺: 543 for | Example 58 |
| | methyl-1H-pyrrol-2- | C₂₁H₂₄Cl₂N₆O₅S | |
| | yl)carbonyl]amino}-3- | NMR: 1.83 (m, 2 H), 2.26 (s, 3 H), | |
| | methoxypiperidin-1-yl)-4-[1- | 3.34 (s, 3 H), 3.44 (m, 4 H), 3.68 (m, | |
| | (methoxymethyl)-1H-imidazol-2- | 1 H), 4.02 (m, 1 H), 4.34 (m, 2 H), | |
| | yl]-1,3-thiazole-5-carboxylic acid | 6.01 (s, 2 H), 7.22 (d, 1 H), 7.42 (s, | |
| | 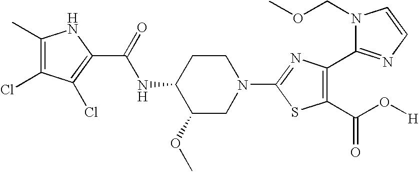 | 1 H), 7.74 (s, 1 H), 12.22 (s, 1 H). | |
| 18 | 2-((3S,4R)-4-{[(3,4-Dichloro-5- | MS (ES) (M + H)⁺: 499 for | Example 86 |
| | methyl-1H-pyrrol-2- | C₁₉H₂₀Cl₂N₆O₄S | |
| | yl)carbonyl]amino}-3- | NMR: 1.64 (m, 2 H), 2.26 (s, 3 H), | |
| | methoxypiperidin-1-yl)-4-(1H- | 3.48 (m, 4 H), 3.66 (m, 1 H), 4.00 | |
| | imidazol-2-yl)-1,3-thiazole-5- | (m, 1 H), 4.32 (m, 2 H), 4.54 (m, | |
| | carboxylic acid | 1 H), 7.25 (d, 1 H), 7.41 (s, 2 H), | |
| | 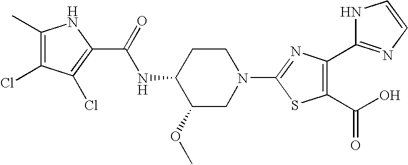 | 12.25 (s, 1 H). | |
| 19 | 2-((3S,4R)-4-{[(3,4-Dichloro-5- | MS (ES) (M + H)⁺: 531 for | Example 59 |
| | methyl-1H-pyrrol-2- | C₂₀H₂₁Cl₂FN₆O₄S | |
| | yl)carbonyl]amino}-3- | NMR: 1.73 (m, 2 H), 2.24 (s, 3 H), | |
| | fluoropiperidin-1-yl)-4-[1- | 3.44 (m, 4 H), 3.71 (s, 3 H), 3.70 (dd, | |
| | (methoxymethyl)-1H-imidazol-2- | 1 H), 4.03 (m, 1 H), 4.44 (m, 2 H), | |
| | yl]-1,3-thiazole-5-carboxylic acid | 5.05 (d, 1 H), 6.02 (s, 2 H), 7.31 (d, | |
| | 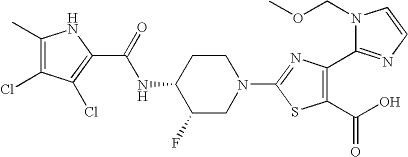 | 1 H), 7.43 (s, 1 H), 7.72 (s, 2 H),12.13 (s, 1 H). | |
| 20 | 2-((3S,4R)-4-{[(3,4-Dichloro-5- | MS (ES) (M + H)⁺: 487 for | Example 87 |
| | methyl-1H-pyrrol-2- | C₁₈H₁₇Cl₂FN₆O₃S | |
| | yl)carbonyl]amino}-3- | NMR: 1.91 (m, 2 H), 2.21 (s, 3 H), | |
| | fluoropiperidin-1-yl)-4-(1H- | 3.15-3.82 (m, 4 H), 4.14 (m, 1 H), | |
| | imidazol-2-yl)-1,3-thiazole-5- | 4.23-4.44 (m, 2 H), 5.02 (d, 1 H), | |
| | carboxylic acid | 7.32 (d, 1 H), 7.45 (s, 2 H), 12.11 (s, | |
| | 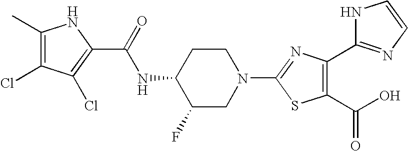 | 1 H). | |
| 21 | 2-((3S,4R)-4-{[(3,4-Dichloro-5- | MS (ES) (M + H)⁺: 513 for | Example 88 |
| | methyl-1H-pyrrol-2- | C₂₀H₂₂Cl₂N₆O₄S | |
| | yl)carbonyl]amino}-3- | NMR: 1.82 (m, 2 H), 2.22 (s, 3 H), | |
| | methoxypiperidin-1-yl)-4-(1- | 3.42 (s, 3 H), 3.63 (m, 1 H), 4.03 (m, | |
| | methyl-1H-imidazol-2-yl)-1,3- | 1 H), 4.12 (s, 3 H), 4.31 (m, 2 H), | |
| | thiazole-5-carboxylic acid | 7.24 (d, 1 H), 7.34 (s, 1 H), 7.53 (s, | |
| | 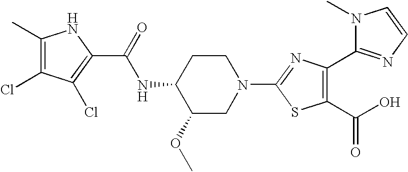 | 1 H), 12.22 (s, 1 H). | |
| 22 | 2-[5-Carboxy-2-((3S,4R)-4-{[(3,4- | MS (ES) (M + H)⁺: 527 for | Example 89 |
| | dichloro-5-methyl-1H-pyrrol-2- | C₂₁H₂₅Cl₂N₆O₄S | |
| | yl)carbonyl]amino}-3- | NMR: 1.81 (m, 2 H), 2.24 (s, 3 H), | |
| | methoxypiperidin-1-yl)-1,3-thiazol- | 3.42 (m, 4 H), 3.62 (m, 1 H), 3.73 (s, | |
| | 4-yl]-1,3-dimethyl-1H-imidazol-3- | 6 H), 4.01 (m, 3 H), 4.34 (m, 2 H), | |
| | ium trifluoroacetate | 7.27 (d, 1 H), 7.95 (s, 2 H), 12.24 (s, | |
| | 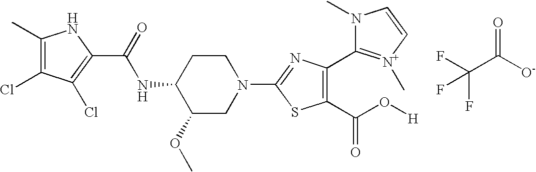 | 1 H). | |
| 23 | 2-((3S,4R)-4-{[(3,4-Dichloro-5- | MS (ES) (M + H)⁺: 499 for | Example 62 |
| | methyl-1H-pyrrol-2- | C₁₉H₂₀Cl₂N₆O₄S | |
| | yl)carbonyl]amino}-3- | NMR: 1.82 (m, 2 H), 2.22 (s, 3 H), | |
| | methoxypiperidin-1-yl)-4-(1H- | 3.34 (m, 4 H), 3.62 (m, 1 H), 4.16 | |
| | pyrazol-5-yl)-1,3-thiazole-5- | (m, 1 H), 4.32 (m, 1 H), 4.54 (m, | |
| | carboxylic acid | 1 H), 7.21 (d, 1 H), 7.70 (s, 1 H), 7.96 | |
| | 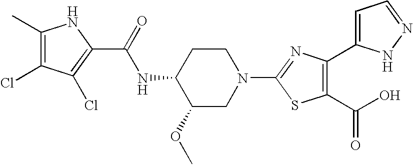 | (s, 1 H), 12.23 (s, 1 H), 13.75 (s, 1 H). | |
| 24 | 2-((3S,4R)-4-{[(3,4-Dichloro-5- | MS (ES) (M + H)⁺: 487 for | Example 63 |
| | methyl-1H-pyrrol-2- | C₁₈H₁₇Cl₂FN₆O₃S | |
| | yl)carbonyl]amino}-3- | NMR: 1.91 (m, 2 H), 2.22 (s, 3 H), | |
| | fluoropiperidin-1-yl)-4-(1H- | 3.64 (dd, 1 H), 3.74 (m, 1 H), 4.35 | |
| | pyrazol-5-yl)-1,3-thiazole-5- | (m, 1 H), 4.43 (m, 1 H), 5.03 (d, 1 H), | |
| | carboxylic acid | 6.85 (s, 1 H), 7.34 (d, 1 H), 8.02 (s, | |
| | 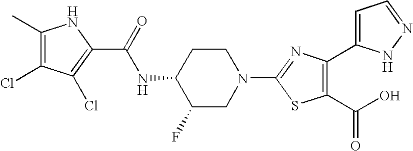 | 1 H), 12.10 (s, 1 H), 13.83 (s, 1 H). | |
| 25 | 2-((3S,4R)-4-{[(3,4-Dichloro-5- | MS (ES) (M + H)⁺: 557 for | Example 64 |
| | methyl-1H-pyrrol-2- | C₂₂H₂₆Cl₂N₆O₅S | |
| | yl)carbonyl]amino}-3- | NMR: 1.84 (m, 2 H), 2.21 (s, 3 H), | |
| | methoxypiperidin-1-yl)-4-[1-(2- | 3.22 (s, 3 H), 3.43 (m, 4 H), 3.62 (m, | |
| | methoxyethyl)-1H-imidazol-2-yl]- | 1 H), 3.74 (m, 2 H), 4.03 (m, 1 H), | |
| | 1,3-thiazole-5-carboxylic acid | 4.35 (m, 2 H), 4.82 (m, 2 H), 7.26 (d, | |
| | 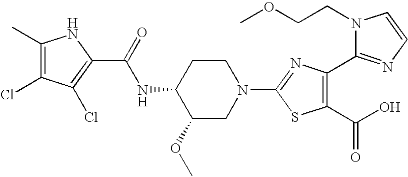 | 1 H), 7.30 (s, 1 H), 7.54 (s, 1 H),12.22 (s, 1 H) | |
| 26 | 2-((3S,4R)-4-{[(3,4-Dichloro-5- | MS (ES) (M + H)⁺: 545 for | Example 65 |
| | methyl- 1H-pyrrol-2- | C₂₁H₂₃Cl₂FN₆O₄S | |
| | yl)carbonyl]amino}-3- | NMR: 1.92 (m, 2 H), 2.24 (s, 3 H), | |
| | fluoropiperidin-1-yl)-4-[1-(2- | 3.22 (s, 3 H), 3.53 (m, 1 H), 3.65 (m, | |
| | methoxyethyl)-1H-imidazol-2-yl]- | 1 H), 3.73 (s, 3 H), 4.04 (m, 1 H), | |
| | 1,3-thiazole-5-carboxylic acid | 4.35 (m, 2 H), 4.82 (m, 2 H), 5.01 (d, | |
| | 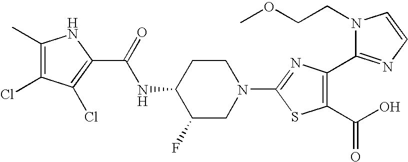 | 1 H), 7.31 (m, 2 H), 7.34 (s, 1 H),7.54 (s, 1 H), 12.10 (s, 1 H). | |
| 27 | 2-((3S,4R)-4-{[(3,4-Dichloro-5- | MS (ES) (M + H)⁺: 514 for | Example 66 |
| | methyl-1H-pyrrol-2- | C₁₉H₂₁Cl₂N₇O₄S | |
| | yl)carbonyl]amino}-3- | NMR: 1.82 (m, 2 H), 2.23 (s, 3 H), | |
| | methoxypiperidin-1-yl)-4-(1- | 3.33-3.42 (m, 4 H), 3.61 (m, 1 H), | |
| | methyl-1H-1,2,4-triazol-5-yl)-1,3- | 4.04 (m, 1 H), 4.14 (s, 3 H), 4.34 (m, | |
| | thiazole-5-carboxylic acid | 2 H), 7.23 (d, 1 H), 8.25 (s, 1 H), | |
| | 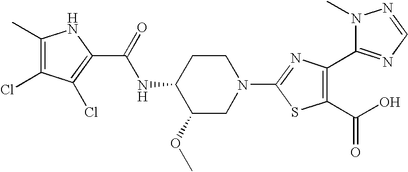 | 12.21 (s, 1 H). 15.45, (s, 1 H). | |
| 28 | 2-((3S,4R)-4-{[(3,4-Dichloro-5- | MS (ES) (M + H)⁺: 502 for | Example 67 |
| | methyl-1H-pyrrol-2- | C₁₈H₁₈Cl₂FN₇O₃S | |
| | yl)carbonyl]amino}-3- | NMR: 1.91 (m, 2 H), 2.22 (s, 3 H), | |
| | fluoropiperidin-1-yl)-4-(1-methyl- | 3.32-3.45 (m, 4 H), 3.61 (dd, 1 H), | |
| | 1H-1,2,4-triazol-5-yl)-1,3-thiazole- | 4.11 (m, 4 H), 4.32-4.51 (m, 2 H), | |
| | 5-carboxylic acid | 5.00 (d, 1 H), 7.35 (d, 1 H), 8.22 (s, | |
| | 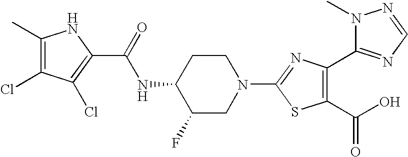 | 1 H), 12.13 (s, 1 H). | |
| 29 | 2-((3S,4R)-4-{[(3-Chloro-4-cyano- | MS (ES) (M + H)⁺: 505 for | Example 80 |
| | 5-methyl-1H-pyrrol-2- | C₂₀H₂₁ClN₈O₄S | |
| | yl)carbonyl]amino}-3- | NMR: 1.82 (m, 2 H), 2.32 (s, 3 H), | |
| | methoxypiperidin-1-yl)-4-(1- | 3.34-3.35 (m, 4 H), 3.61 (m, 1 H), | |
| | methyl-1H-1,2,4-triazol-5-yl)-1,3- | 4.02 (m, 1 H), 4.11 (s, 3 H), 4.33 (m, | |
| | thiazole-5-carboxylic acid | 2 H), 7.35 (d, 1 H), 8.25 (s, 1 H), | |
| | 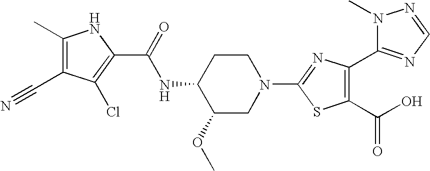 | 12.72 (s, 1 H), 15.42 (s, 1 H). | |
| 30 | 2-((3S,4R)-4-{[(3-Chloro-4-cyano- | MS (ES) (M + H)⁺: 549 for | Example 69 |
| | 5-methyl-1H-pyrrol-2- | C₂₃H₂₆ClN₇O₅S | |
| | yl)carbonyl]amino}-3- | NMR: 1.82 (m, 2 H), 2.33 (s, 3 H), | |
| | methoxypiperidin-1-yl)-4-[1-(2- | 3.22 (s, 3 H), 3.34 (m, 4 H), 3.63 (m, | |
| | methoxyethyl)-1H-imidazol-2-yl]- | 1 H), 3.73 (m, 2 H), 3.95 (m, 1 H), | |
| | 1,3-thiazole-5-carboxylic acid | 4.33 (m, 2 H), 4.82 (m, 2 H), 7.31 | |
| | 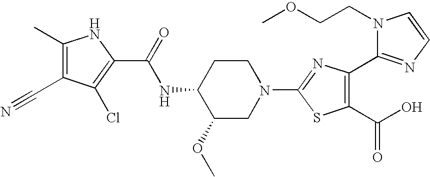 | (m, 2 H), 7.51 (s, 1 H), 12.70 (s, 1 H). | |
| 31 | 2-((3S,4R)-4-{[(4-Cyano-5-methyl- | MS (ES) (M + H)⁺: 471 for | Example 68 |
| | 1H-pyrrol-2-yl)carbonyl]amino}-3- | C₂₀H₂₂N₈O₄S | |
| | methoxypiperidin-1-yl)-4-(1- | NMR: 1.57 (m, 1 H), 1.83 (m, 1 H), | |
| | methyl-1H-1,2,4-triazol-5-yl)-1,3- | 2.34 (s, 3 H), 3.34 (m, 4 H), 3.62 (m, | |
| | thiazole-5-carboxylic acid | 1 H), 4.04 (m, 1 H), 4.09 (s, 3 H), | |
| | 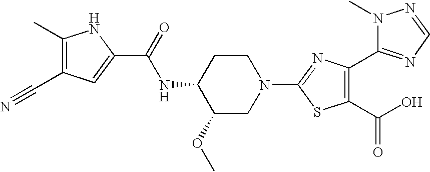 | 4.23-4.34 (m, 2 H), 7.20 (s, 1 H),8.03 (d, 1 H), 8.25 (s, 1 H), 12.29 (s,1 H), 15.43 (s, 1 H). | |
| 32 | 2-((3S,4R)-4-{[(3,4-Dichloro-5- | MS (ES) (M + H)⁺: 541 for | Example 70 |
| | methyl-1H-pyrrol-2- | C₂₂H₂₆Cl₂N₆O₄S | |
| | yl)carbonyl]amino}-3- | NMR: 1.81 (m, 2 H), 2.24 (s, 9 H), | |
| | methoxypiperidin-1-yl)-4-(1,4,5- | 3.34 (m, 4 H), 3.64 (m, 1 H), 3.65 (s, | |
| | trimethyl-1H-imidazol-2-yl)-1,3- | 3 H), 4.01 (m, 1 H), 4.05 (s, 3 H), | |
| | thiazole-5-carboxylic acid | 4.28 (m, 2 H), 7.17 (d, 1 H), 12.23 (s, | |
| | 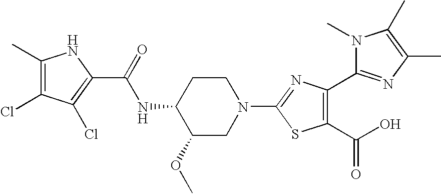 | 1 H). | |
| 33 | 2′-Amino-2-((3S,4R)-4-{[(3,4- | MS (ES) (M + H)⁺: 529 for | Example 71 |
| | dichloro-5-methyl-1H-pyrrol-2- | C₁₉H₂₀Cl₂N₆O₄S₂ | |
| | yl)carbonyl]amino}-3- | NMR: 1.78 (m, 2 H), 2.21 (s, 3 H), | |
| | methoxypiperidin-1-yl)-4,4′-bi-1,3- | 3.33 (m, 4 H), 3.51 (m, 1 H), 3.67 (s, | |
| | thiazole-5-carboxylic acid | 3 H), 4.02 (m, 1 H), 4.28 (m, 2 H), | |
| | 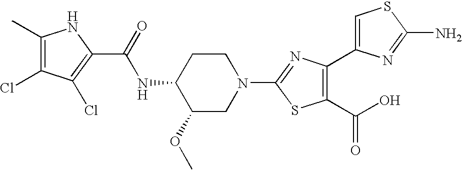 | 7.15 (d, 1 H), 7.25 (s, 2 H), 8.11 (s,2 H), 12.15 (s, 1 H). | |
| 34 | 2-((3S,4R)-4-{[(3,4-Dichloro-5- | MS (ES) (M + H)⁺: 543 for | Example 72 |
| | methyl-1H-pyrrol-2- | C₂₀H₂₂Cl₂N₆O₄S₂ | |
| | yl)carbonyl]amino}-3- | NMR: 1.78 (m, 2 H), 2.17 (s, 3 H), | |
| | methoxypiperidin-1-yl)-2′- | 2.81 (s, 3 H), 3.22-3.44 (m, 4 H), | |
| | (methylamino)-4,4′-bi-1,3-thiazole- | 3.51 (m, 1 H), 3.88 (m, 1 H), 4.22 | |
| | 5-carboxylic acid | (m, 2 H), 7.22 (m, 1 H), 7.51 (m, | |
| | 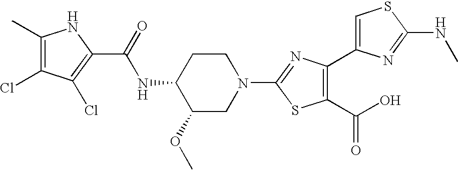 | 1 H). | |
| 35 | 2-((3S,4R)-4-{[(3,4-Dichloro-5- | MS (ES) (M + H)⁺: 560 for | Example 73 |
| | methyl-1H-pyrrol-2- | C₂₁H₂₄Cl₂N₆O₄S₂ | |
| | yl)carbonyl]amino}-3- | NMR: 1.75 (m, 2 H), 2.21 (s, 3 H), | |
| | methoxypiperidin-1-yl)-2′- | 3.16 (s, 6 H), 3.28-3.43 (m, 4 H), | |
| | (dimethylamino)-4,4′-bi-1,3- | 3.55 (m, 1 H), 3.95 (m, 1 H), 4.32 | |
| | thiazole-5-carboxylic acid | (m, 2 H), 7.18 (d, 1 H), 7.43 (s, 1 H), | |
| | 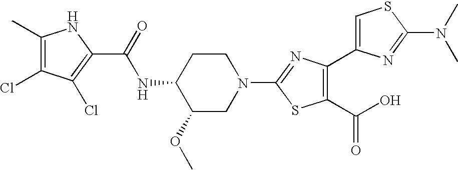 | 12.21 (s, 1 H). | |
| 36 | 2′-Amino-2-((3S,4R)-4-{[(3,4- | MS (ES) (M + H)⁺: 519 for | Example 74 |
| | dichloro-5-methyl-1H-pyrrol-2- | C₁₈H₁₇Cl₂FN₆O₃S₂ | |
| | yl)carbonyl]amino}-3- | NMR: 1.81 (m, 2 H), 2.21 (s, 3 H), | |
| | fluoropiperidin-1-yl)-4,4′-bi-1,3- | 3.15 (d, 1 H), 3.35 (m, 4 H), 3.86 (m, | |
| | thiazole-5-carboxylic acid | 1 H), 4.22 (m, 2 H), 4.91 (d, 1 H), | |
| | 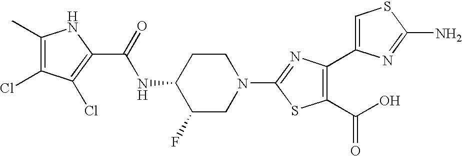 | 7.02 (m, 1 H), 7.64 (m, 2 H). | |
| 37 | 2-((3S,4R)-4-{[(3,4-Dichloro-5- | MS (ES) (M + H)⁺: 533 for | Example 75 |
| | methyl-1H-pyrrol-2- | C₁₉H₁₉Cl₂FN₆O₃S₂ | |
| | yl)carbonyl]amino}-3- | NMR: 1.81 (m, 2 H), 2.22 (s, 3 H), | |
| | fluoropiperidin-1-yl)-2′- | 2.92 (m, 3 H), 3.32 (m, 4 H), 3.88 | |
| | (methylamino)-4,4′-bi-1,3-thiazole- | (m, 1 H), 4.29 (m, 2 H), 4.91 (d, 1 H), | |
| | 5-carboxylic acid | 7.25-7.46 (m, 2 H). | |
| | 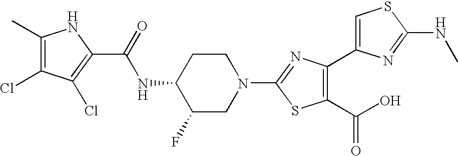 | | |
| 38 | 2-((3S,4R)-4-{[(3,4-Dichloro-5- | MS (ES) (M + H)⁺: 547 for | Example 76 |
| | methyl-1H-pyrrol-2- | C₂₀H₂₁Cl₂FN₆O₃S₂ | |
| | yl)carbonyl]amino}-3- | NMR: 1.91 (m, 2 H), 2.22 (s, 3 H), | |
| | fluoropiperidin-1-yl)-2′- | 3.14 (s, 6 H), 3.34 (m, 4 H), 3.61 (dd, | |
| | (dimethylamino)-4,4′-bi-1,3- | 1 H), 4.08 (m, 1 H), 4.42 (m, 2 H), | |
| | thiazole-5-carboxylic acid | 5.01 (d, 1 H), 7.27 (d, 1 H), 7.39 (s, | |
| | 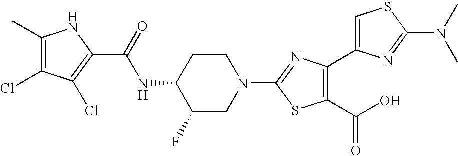 | 1 H), 12.14 (s, 1 H). | |
| 39 | 2-((3S,4R)-4-{[(3,4-Dichloro-5- | MS (ES) (M + H)⁺: 501 for | Example 79 |
| | methyl-1H-pyrrol-2- | C₁₉H₁₉Cl₂FN₆O₃S | |
| | yl)carbonyl]amino}-3- | NMR: 1.91 (m, 2 H), 2.22 (s, 3 H), | |
| | fluoropiperidin-1-yl)-4-(1-methyl- | 3.23 (m, 4 H), 3.58 (m, 1 H), 4.10 | |
| | 1H-imidazol-2-yl)-1,3-thiazole-5- | (m, 4 H), 4.42 (m, 2 H), 5.01 (d, 1 H), | |
| | carboxylic acid | 7.29 (m, 2 H), 7.50 (s, 1 H), 12.14 (s, | |
| | 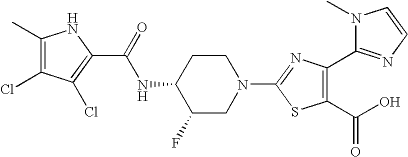 | 1 H). | |
| 40 | 2-((3S,4R)-4-{[(3,4-Dichloro-5- | MS (ES) (M + H)⁺: 513 for | Example 77 |
| | methyl-1H-pyrrol-2- | C₂₀H₂₂Cl₂N₆O₄S | |
| | yl)carbonyl]amino}-3- | NMR: 1.81 (m, 2 H), 2.22 (s, 3 H), | |
| | methoxypiperidin-1-yl)-4-(1- | 3.41 (m, 4 H), 3.62 (m, 1 H), 3.77 (s, | |
| | methyl-1H-imidazol-4-yl)-1,3- | 3 H), 4.02 (m, 1 H), 4.18-4.41 (m, | |
| | thiazole-5-carboxylic acid | 2 H), 7.23 (m, 1 H), 7.81 (s, 1 H), | |
| | 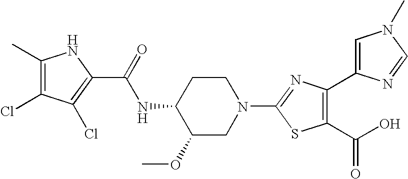 | 8.10 (s, 1 H), 12.27 (s, 1 H). | |
| 41 | 2-((3S,4R)-4-{[(3,4-Dichloro-5- | MS (ES) (M + H)⁺: 502 for | Example 78 |
| | methyl-1H-pyrrol-2- | C₂₁H₂₃Cl₂FN₆O₃S | |
| | yl)carbonyl]amino}-3- | NMR: 1.91 (m, 2 H), 2.22 (s, 3 H), | |
| | fluoropiperidin-1-yl)-4-(1-methyl- | 3.34 (m, 4 H), 3.57 (m, 1 H), 3.81 (s, | |
| | 1H-imidazol-4-yl)-1,3-thiazole-5- | 3 H), 4.04 (m, 1 H), 4.25-4.41 (m, | |
| | carboxylic acid | 2 H), 5.01 (d, 1 H), 7.28 (d, 1 H), | |
| | 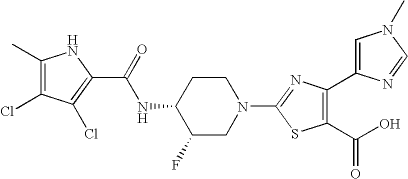 | 7.79(s, 1 H), 8.20 (s, 1 H), 12.13 (s,1 H). | |
| 42 | 2-((3S,4R)-4-{[(3,4-Dichloro-5- | MS (ES) (M + H)⁺: 542 for | Example 82 |
| | methyl-1H-pyrrol-2- | C₂₁H₂₂Cl₂N₆O₅S | |
| | yl)carbonyl]amino}-3- | NMR: 1.78 (m, 2 H), 2.19 (s, 3 H), | |
| | methoxypiperidin-1-yl)-4-(4- | 3.35 (m, 3 H), 3.42 (s, 3 H), 3.57 (m, | |
| | methoxypyrimidin-2-yl)-1,3- | 1 H), 3.99 (s, 3 H), 4.30 (m, 1 H), | |
| | thiazole-5-carboxylic acid | 4.43 (m, 1 H), 7.08 (d, 1 H), 7.18 (d, | |
| | 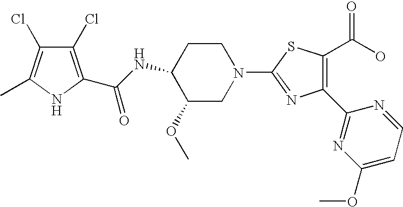 | 1 H), 8.69 (d, 1 H), 12.17 (br, 1 H). | |

### Example 43

### Ethyl 2-((3S,4R)-4-{[(3,4-dichloro-5-methyl-1H-pyrrol-2-yl)carbonyl]amino}-3-methoxypiperidin-1-yl)-4-pyrimidin-2-yl-1,3-thiazole-5-carboxylate

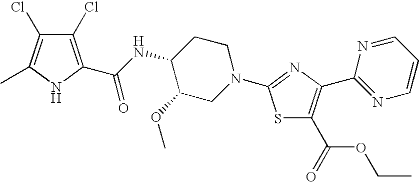

A suspension of 3,4-dichloro-N-[(3S,4R)-3-methoxypiperidin-4-yl]-5-methyl-1H-pyrrole-2-carboxamide hydrochloride (WO2006087543, 0.08 g, 0.23 mmol), ethyl 2-chloro-4-pyrimidin-2-yl-1,3-thiazole-5-carboxylate (Intermediate 1; 0.057 g, 0.21 mmol), and solid sodium bicarbonate (0.056 g, 0.69 mmol) in DMF (3 ml) was heated in the microwave for 30 mins at 100° C. LCMS indicates conversion of starting material to product. The crude reaction mixture was added dropwise to a pH 4 citric acid buffer. The resulting solid was filtered, washed with water and dried (0.057 g). MS (ES) (M+H)⁺: 539 for C₂₂H₂₄Cl₂N₆O₄S; NMR: 0.092 (t, 3H), 1.69 (m, 2H), 2.12 (s, 3H), 3.31 (m, 5H), 3.50 (s, 1H), 3.91 (q, 2H), 4.19 (m, 2H), 7.09 (d, 1H), 7.49 (t, 1H), 8.80 (d, 1H), 12.10 (s, 1H)

### Examples 44-85

The following Examples were prepared by the procedure described in Example 43 from the starting materials (SM) indicated.

| Ex | Compound | Data | SM |
|---|---|---|---|
| 44 | 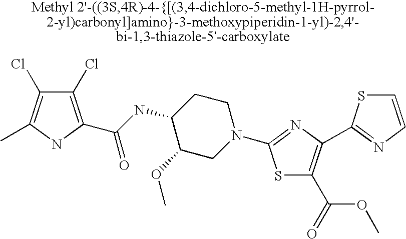 | MS (ES) (M + H)⁺: 530 forC₂₀H₂₁Cl₂N₅O₄S₂NMR: 1.76 (m, 2H), 2.17 (s,3H), 3.32 (m, 3H), 3.38 (s,3H), 3.57 (s, 1H), 3.69 (s,3H), 4.28 (m, 2H), 7.15 (d,1H), 7.90 (s, 1H), 7.94 (s,1H), 12.15 (s, 1H) | 3,4-dichloro-N-[(3S,4R)-3-methoxypiperidin-4-yl]-5-methyl-1H-pyrrole-2-carboxamidehydrochloride andIntermediate 3 |
| 45 | 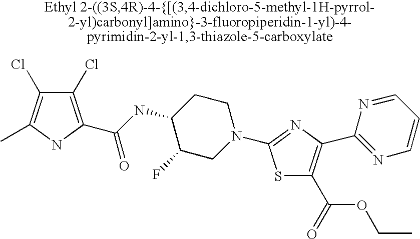 | MS (ES) (M + H)⁺: 527 forC₂₁H₂₁Cl₂FN₆O₃SNMR: 0.98 (t, 3H), 1.86 (m,2H), 2.18 (s, 3H), 3.57 (m,1H), 3.97 (m, 3H), 4.33 (m,2H), 4.88 (d, 1H), 7.29 (d,1H), 7.55 (t, 1H), 8.86 (d,2H), 12.10 (s, 1H) | 3,4-dichloro-N-[(3S,4R)-3-fluoropiperidin-4-yl]-5-methyl-1H-pyrrole-2-carboxamidehydrochloride(WO2006087543)and Intermediate 1 |
| 46 | 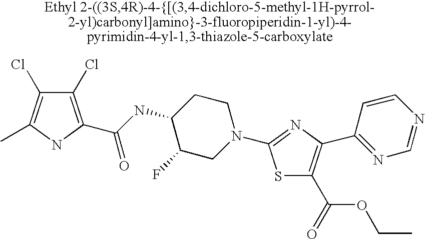 | MS (ES) (M + H)⁺: 527 forC₂₁H₂₁Cl₂FN₆O₃SNMR: 1.03 (t, 3H), 1.81 (m,2H), 2.12 (s, 3H), 3.51 (m,1H), 3.65 (m, 1H), 4.01 (m,2H), 4.28 (m, 2H), 4.83 (d,1H), 7.20 (d, 1H), 7.67 (d,1H), 8.83 (d, 1H), 9.17 (s,1H), 12.04 (s, 1H) | 3,4-dichloro-N-[(3S,4R)-3-fluoropiperidin-4-yl]-5-methyl-1H-pyrrole-2-carboxamidehydrochloride andIntermediate 2 |
| 47 | 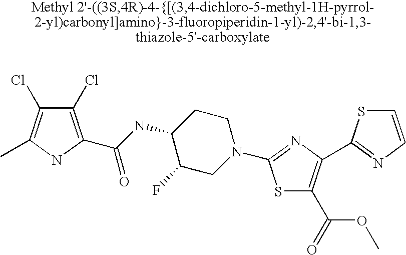 | MS (ES) (M + H)⁺: 518 forC₁₉H₁₈Cl₂FN₅O₃S₂NMR: 1.82 (m, 2H), 2.13 (s,3H), 3.65 (s, 3H), 3.96 (m,1H), 4.29 (m, 2H), 4.85 (d,1H), 7.22 (d, 1H), 7.86 (d,2H), 12.04 (s, 1H) | 3,4-dichloro-N-[(3S,4R)-3-fluoropiperidin-4-yl]-5-methyl-1H-pyrrole-2-carboxamidehydrochloride andIntermediate 3 |
| 48 | 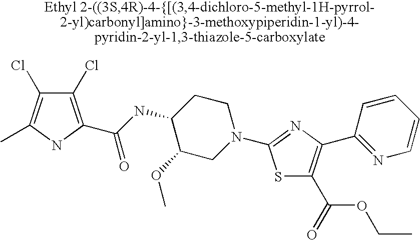 | MS (ES) (M + H)⁺: 538 forC₂₃H₂₅Cl₂N₅O₄SNMR: 1.06 (t, 3H), 1.76 (m,2H), 2.19 (s, 3H), 3.29 (m,2H), 3.39 (s, 4H), 3.56 (m,1H), 4.03 (q, 2H), 4.26 (m,2H), 7.16 (d, 1H), 7.40 (dd,1H), 7.55 (d, 1H), 8.58 (d,1H), 12.17 (s, 1H) | 3,4-dichloro-N-[(3S,4R)-3-methoxypiperidin-4-yl]-5-methyl-1H-pyrrole-2-carboxamidehydrochloride andIntermediate 13 |
| 49 | 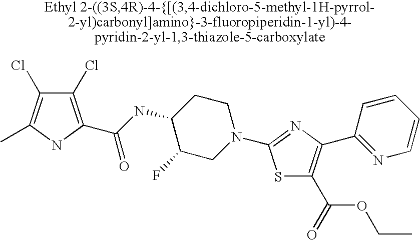 | MS (ES) (M + H)⁺: 526 forC₂₂H₂₂Cl₂FN₅O₃SNMR: 1.07 (t, 3H), 1.87 (m,2H), 2.19 (s, 3H), 3.32 (m,2H), 3.61 (m, 1H), 4.04 (q,2H), 4.35 (m, 2H), 4.89 (d,1H), 7.28 (d, 1H), 7.43 (dd,1H), 7.59 (d, 1H), 7.81 (t,1H), 8.59 (d, 1H), 12.11 (s,1H) | 3,4-dichloro-N-[(3S,4R)-3-fluoropiperidin-4-yl]-5-methyl-1H-pyrrole-2-carboxamidehydrochloride andIntermediate 13 |
| 50 | 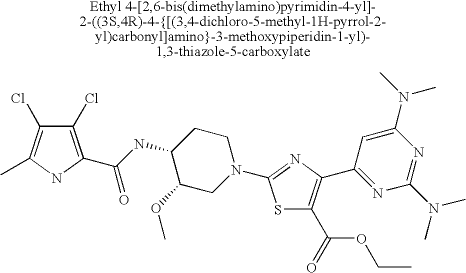 | MS (ES) (M + H)⁺: 625 forC₂₆H₃₄Cl₂N₈O₄SNMR: 1.13 (t, 3H), 1.76 (m,2H), 2.17 (s, 3H), 3.08 (s,12H), 3.32 (m, 3H), 3.95 (m,1H), 4.13 (q, 2H), 4.27 (m,2H), 6.26 (s, 1H), 7.14 (d,1H), 12.16 (s, 1H) | 3,4-dichloro-N-[(3S,4R)-3-methoxypiperidin-4-yl]-5-methyl-1H-pyrrole-2-carboxamidehydrochloride andIntermediate 4 |
| 51 | 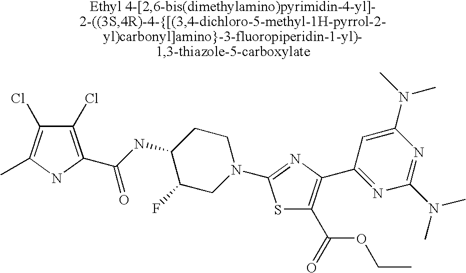 | MS (ES) (M + H)⁺: 613 forC₂₅H₃₁Cl₂FN₈O₃SNMR: 1.07 (t, 3H), 1.79 (m,2H), 2.13 (s, 3H), 3.00 (s,12H, 3.27 (m, 1H), 3.48 (m,1H), 4.03 (q, 2H), 4.19 (m,2H), 4.83 (d, 1H), 6.03 (s,1H), 7.22 (d, 1H), 12.05 (s,1H) | 3,4-dichloro-N-[(3S,4R)-3-fluoropiperidin-4-yl]-5-methyl-1H-pyrrole-2-carboxamidehydrochloride andIntermediate 4 |
| 52 | 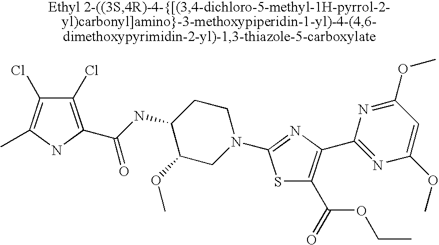 | MS (ES) (M + H)⁺: 599 forC₂₄H₂₈Cl₂N₆O₆SNMR: 0.96 (t, 3H), 1.70 (m,2H), 2.12 (s, 3H), 3.27 (s,6H), 3.79 (m, 6H), 3.95 (q,2H), 4.22 (m, 2H), 6.23 (s,1H), 7.09 (d, 1H), 12.10 (s,1H) | 3,4-dichloro-N-[(3S,4R)-3-methoxypiperidin-4-yl]-5-methyl-1H-pyrrole-2-carboxamidehydrochloride andIntermediate 5 |
| 53 | 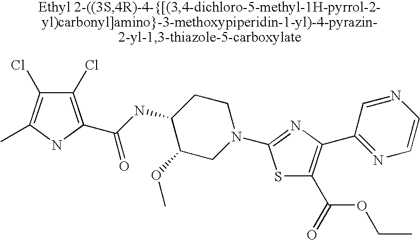 | MS (ES) (M + H)⁺: 539 forC₂₂H₂₄Cl₂N₆O₄SNMR: 1.08 (t, 3H), 1.77 (m,2H), 2.19 (s, 3H), 3.33 (s,3H), 3.39 (s, 3H), 3.57 (s,1H), 4.06 (q, 2H), 4.11 (m,2H), 7.16 (d, 1H), 8.67 (d,2H), 8.84 (s, 1H), 12.16 (s,1H) | 3,4-dichloro-N-[(3S,4R)-3-methoxypiperidin-4-yl]-5-methyl-1H-pyrrole-2-carboxamidehydrochloride andIntermediate 6 |
| 54 | 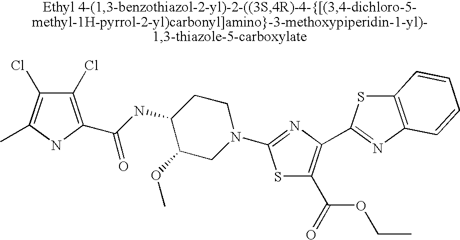 | MS (ES) (M + H)⁺: 594 forC₂₅H₂₅Cl₂N₅O₄S₂NMR: 1.17 (t, 3H), 1.78 (m,2H), 2.17 (s, 3H), 3.32 (s,3H), 3.41 (s, 5H), 4.17 (q,2H), 4.44 (m, 1H), 7.15 (d,1H), 7.57 (m, 2H), 8.10 (d,1H), 8.14 (d, 1H), 12.15 (s,1H) | 3,4-dichloro-N-[(3S,4R)-3-methoxypiperidin-4-yl]-5-methyl-1H-pyrrole-2-carboxamidehydrochloride andIntermediate 7 |
| 55 | 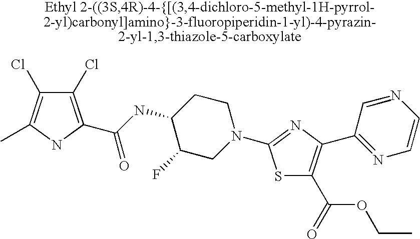 | MS (ES) (M + H)⁺: 527 forC₂₁H₂₁Cl₂FN₆O₃SNMR: 1.17 (t, 3H), 1.78 (m,2H), 2.17 (s, 3H), 3.32 (s,3H), 3.41 (s, 5H), 4.17 (q,2H), 4.44 (m, 1H), 7.15 (d,1H), 7.57 (m, 2H), 8.10 (d,1H), 8.14 (d, 1H), 12.15 (s,1H) | 3,4-dichloro-N-[(3S,4R)-3-fluoropiperidin-4-yl]-5-methyl-1H-pyrrole-2-carboxamidehydrochloride andIntermediate 6 |
| 56 | 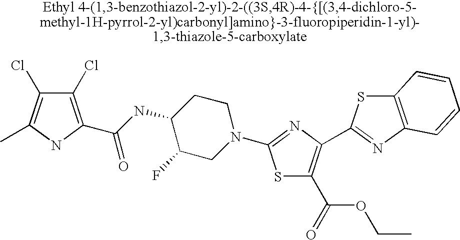 | MS (ES) (M + H)⁺: 582 forC₂₄H₂₂Cl₂FN₅O₃S₂NMR: 1.12 (t, 3H), 1.84 (m,2H), 2.13 (s, 3H), 3.27 (s,3H), 3.55 (dd, 1H), 3.98 (m,1H), 4.14 (q, 2H), 4.27 (m,2H), 4.86 (d, 1H), 7.23 (d,1H), 7.45 (m, 2H), 8.00 (d,1H), 8.09 (d, 1H), 12.04 (s,1H) | 3,4-dichloro-N-[(3S,4R)-3-fluoropiperidin-4-yl]-5-methyl-1H-pyrrole-2-carboxamidehydrochloride andIntermediate 7 |
| 57 | 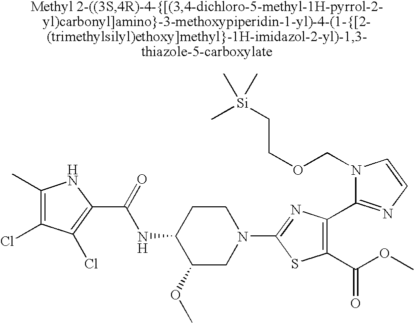 | MS (ES) (M + H)⁺: 643 forC₂₆H₃₆Cl₂N₆O₅SSi | Intermediate 14and 3,4-dichloro-N-[(3S,4R)-3-methoxypiperidin-4-yl]-5-methyl-1H-pyrrole-2-carboxamidehydrochloride |
| 58 | 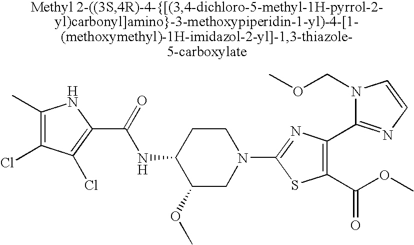 | MS (ES) (M + H)⁺: 557 forC₂₂H₂₆Cl₂N₆O₅SNMR: 1.81 (m, 2H), 2.23 (s,3H), 3.14 (s, 3H), 3.43 (m,4H), 3.58 (m, 1H), 3.65 (s,3H), 4.02 (m, 1H), 4.34 (m,2H), 5.26 (s, 2H), 7.15 (d,1H), 7.39 (s, 2H), 12.22 (s,1H). | Intermediate 15and 3,4-dichloro-N-[(3S,4R)-3-methoxypiperidin-4-yl]-5-methyl-1H-pyrrole-2-carboxamidehydrochloride |
| 59 | 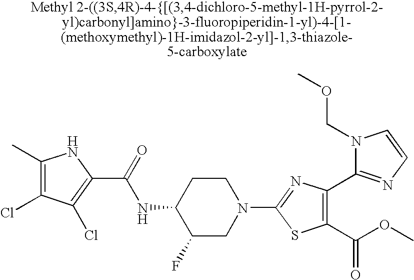 | MS (ES) (M + H)⁺: 545 forC₂₁H₂₃Cl₂FN₆O₄SNMR: 1.91 (m, 2H), 2.22 (s,3H), 3.11 (s, 3H), 3.42 (m,4H, 3.48 (m, 1H), 3.61 (s,3H), 3.66 (m, 1H), 4.02 (m,1H), 4.33-4.46 (m, 1H), 5.04(d, 1H), 5.18 (s, 2H), 7.03(s, 1H), 7.32 (d, 1H), 7.42(s, 2H), 12.18 (s, 1H). | Intermediate 15and 3,4-dichloro-N-[(3S,4R)-3-fluoropiperidin-4-yl]-5-methyl-1H-pyrrole-2-carboxamidehydrochloride |
| 60 | 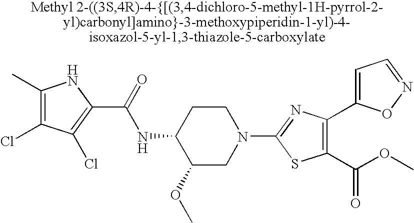 | MS (ES) (M + H)⁺: 514 forC₂₀H₂₁Cl₂N₅O₅SNMR: 1.81 (m, 2H), 2.22 (s,3H), 3.32 (m, 4H), 3.59 (m,1H), 3.77 (s, 3H), 4.03 (m,1H), 4.23 (m, 2H), 7.15 (d,1H), 7.27 (s, 1H), 8.69 (s,1H), 12.20 (s, 1H). | Intermediate 59and 3,4-dichloro-N-[(3S,4R)-3-methoxypiperidin-4-yl]-5-methyl-1H-pyrrole-2-carboxamidehydrochloride |
| 61 | 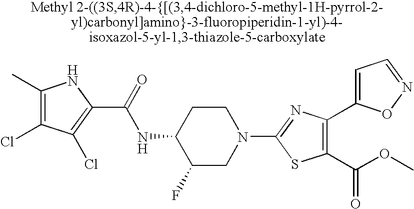 | MS (ES) (M + H)⁺: 503 forC₁₉H₁₈Cl₂FN₅O₄SNMR: 1.91 (m, 2H), 2.21 (s,3H), 3.65 (dd, 1H), 3.85 (s,3H), 4.09 (m, 1H), 4.86 (m,2H), 5.02 (d, 1H), 7.34 (m,2H), 8.74 (s, 1H), 12.18 (s,1H). | Intermediate 59and 3,4-dichloro-N-[(3S,4R)-3-fluoropiperidin-4-yl]-5-methyl-1H-pyrrole-2-carboxamidehydrochloride |
| 62 | 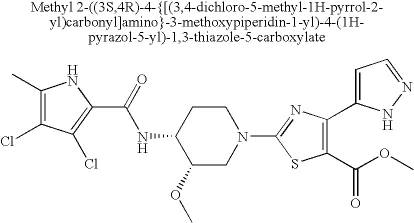 | MS (ES) (M + H)⁺: 513 forC₂₀H₂₂Cl₂N₆O₄SNMR: 1.82 (m, 2H), 2.24 (s,3H), 3.34 (m, 4H), 3.57 (m,1H), 3.81 (s, 3H), 4.13 (m,1H), 4.25 (m, 2H), 7.24 (d,1H), 7.25 (s, 1H), 7.58 (s,1H), 12.21 (s, 1H), 13.34 (s,1H). | Intermediate 60and 3,4-dichloro-N-[(3S,4R)-3-methoxypiperidin-4-yl]-5-methyl-1H-pyrrole-2-carboxamidehydrochloride |
| 63 | 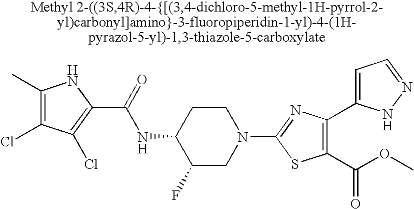 | MS (ES) (M + H)⁺: 501 forC₁₉H₁₉Cl₂FN₆O₃SNMR: 1.91 (m, 2H), 2.22 (s,3H), 3.65 (dd, 1H), 3.84 (s,3H, 4.07 (m, 1H), 4.86 (m,2H), 5.03 (d, 1H), 7.32 (m,2H), 8.71 (s, 1H), 12.19 (s,1H). | Intermediate 60and 3,4-dichloro-N-[(3S,4R)-3-fluoropiperidin-4-yl]-5-methyl-1H-pyrrole-2-carboxamidehydrochloride |
| 64 | 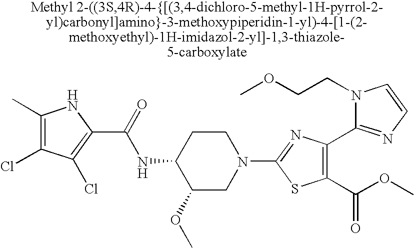 | MS (ES) (M + H)⁺: 571 forC₂₃H₂₈Cl₂N₆O₅SNMR: 1.82 (m, 2H), 2.22 (s,3H), 3.22 (s, 3H), 3.34 (m,4H), 3.58 (m, 1H), 3.69 (m,2H), 4.02 (m, 1H), 4.34 (m,2H), 4.77 (m, 2H), 7.23 (d,1H), 7.31 (s, 1H), 7.52 (s,1H), 12.22 (s, 1H). | Intermediate 8and 3,4-dichloro-N-[(3S,4R)-3-methoxypiperidin-4-yl]-5-methyl-1H-pyrrole-2-carboxamidehydrochloride |
| 65 | 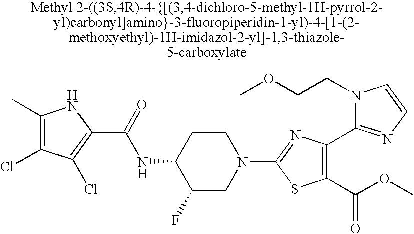 | MS (ES) (M + H)⁺: 559 forC₂₂H₂₅Cl₂FN₆O₄SNMR: 1.91 (m, 2H), 2.22 (s,3H), 3.21 (s, 3H), 3.34-3.46(m, 4H), 3.52 (t, 1H), 3.48-3.86 (m, 5H), 4.02 (m, 3H),4.22-4.52 (m, 2H), 5.01 (d,1H), 6.95 (s, 1H), 7.28 (m,2H), 12.18 (s, 1H). | Intermediate 8and 3,4-dichloro-N-[(3S,4R)-3-fluoropiperidin-4-yl]-5-methyl-1H-pyrrole-2-carboxamidehydrochloride |
| 66 | 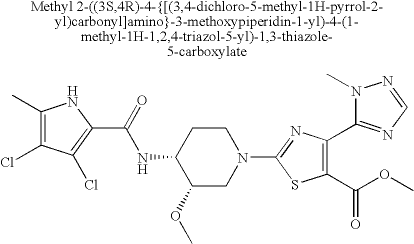 | MS (ES) (M + H)⁺: 528 forC₂₀H₂₃Cl₂N₇O₄SNMR: 1.81 (m, 2H), 2.22 (s,3H), 3.34 (m, 4H), 3.57 (m,1H), 3.71 (s, 6H), 4.01 (m,1H), 4.28 (m, 2H), 7.15 (d,1H), 8.04 (s, 1H), 12.23 (s,1H). | Intermediate 9and 3,4-dichloro-N-[(3S,4R)-3-methoxypiperidin-4-yl]-5-methyl-1H-pyrrole-2-carboxamidehydrochloride |
| 67 | 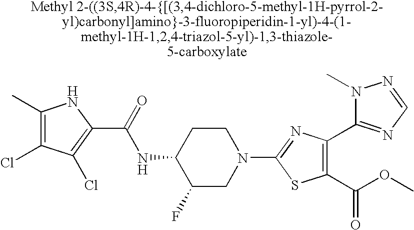 | MS (ES) (M + H)⁺: 516 forC₁₉H₂₀Cl₂FN₇O₃SNMR: 1.91 (m, 2H), 2.22 (s,3H), 3.32 (m, 4H), 3.57 (m,1H), 3.75 (s, 6H), 4.02 (m,1H), 4.43 (m, 2H), 5.02 (d,1H), 7.27 (d, 1H), 8.03 (s,1H), 12.18 (s, 1H). | Intermediate 9and 3,4-dichloro-N-[(3S,4R)-3-fluoropiperidin-4-yl]-5-methyl-1H-pyrrole-2-carboxamidehydrochloride |
| 68 | 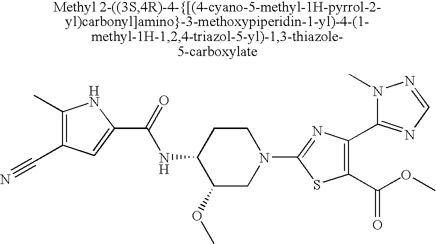 | MS (ES) (M + H)⁺: 485 forC₂₁H₂₄N₈O₄SNMR: 1.64 (m, 1H), 1.87-2.04 (m, 1H), 2.33 (s, 3H),3.33 (m, 4H), 3.58 (m, 1H),3.71 (s, 6H), 4.01 (m, 1H),4.18-4.36 (m, 2H), 7.26 (s,1H), 7.91 (d, 1H), 8.04 (s,1H), 12.33 (s, 1H). | Intermediate 65andIntermediate 9 |
| 69 | 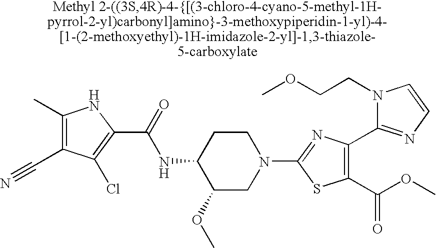 | MS (ES) (M + H)⁺: 562 forC₂₄H₂₈ClN₇O₅SNMR: 1.81 (m, 2H), 2.22 (s,3H), 3.23 (s, 3H), 3.33 (m,4H), 3.54 (m, 2H), 3.59 (m,4H), 3.91 (m, 1H), 4.01 (m,2H), 4.33 (m, 2H), 6.95 (s,1H), 7.33 (s, 1H), 7.35 (d,1H), 12.71 (s, 1H). | Intermediate 8andIntermediate 64 |
| 70 | 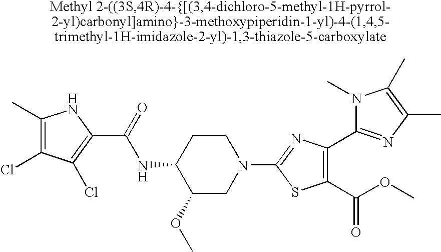 | MS (ES) (M + H)⁺: 555 forC₂₃H₂₈Cl₂N₆O₄SNMR: 1.75 (m, 2H), 2.03 (s,3H), 2.15 (s, 3H), 2.25 (s,3H), 3.35 (m, 4H), 3.56 (m,1H), 3.65 (s, 3H), 4.01 (m,1H), 4.29 (m, 2H), 7.20 (d,1H), 12.23 (s, 1H). | Intermediate 11and 3,4-dichloro-N-[(3S,4R)-3-methoxypiperidin-4-yl]-5-methyl-1H-pyrrole-2-carboxamidehydrochloride |
| 71 | 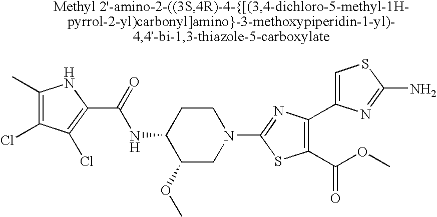 | MS (ES) (M + H)⁺: 545 forC₂₀H₂₂Cl₂N₆O₄S₂NMR: 1.75 (m, 2H), 2.23 (s,3H), 3.41 (m, 4H), 3.57 (m,1H), 3.68 (s, 3H), 4.02 (m,1H), 4.25 (m, 2H), 7.02 (m,2H), 7.15 (m, 2H), 12.24 (s,1H). | Intermediate 55and 3,4-dichloro-N-[(3S,4R)-3-methoxypiperidin-4-yl]-5-methyl-1H-pyrrole-2-carboxamidehydrochloride |
| 72 | 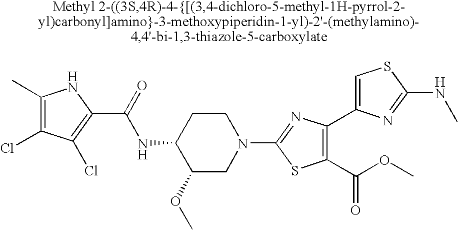 | MS (ES) (M + H)⁺: 559 forC₂₁H₂₄Cl₂N₆O₄S₂NMR: 1.74 (m, 2H), 2.22 (s,3H), 2.75 (d, 3H), 3.34-3.48(m, 4H), 3.55 (m, 1H), 3.65(s, 3H), 3.91 (m, 1H), 4.18(m, 2H), 7.13 (s, 1H), 7.23(d, 1H), 7.53 (m, 1H), 12.27(s, 1H). | Intermediate 56and 3,4-dichloro-N-[(3S,4R)-3-methoxypiperidin-4-yl]-5-methyl-1H-pyrrole-2-carboxamidehydrochloride |
| 73 | 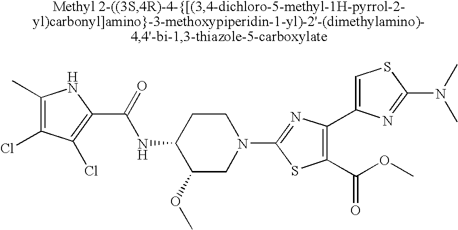 | MS (ES) (M + H)⁺: 573 forC₂₂H₂₆Cl₂N₆O₄S₂NMR: 1.81 (m, 2H), 2.22 (s,3H), 3.08 (s, 6H), 3.27-3.45(m, 4H), 3.55 (m, 1H), 3.71(s, 3H), 3.89 (m, 1H), 4.32(m, 2H), 7.17 (s, 1H), 7.20(d, 1H), 12.24 (s, 1H). | Intermediate 57and 3,4-dichloro-N-[(3S,4R)-3-methoxypiperidin-4-yl]-5-methyl-1H-pyrrole-2-carboxamidehydrochloride |
| 74 | 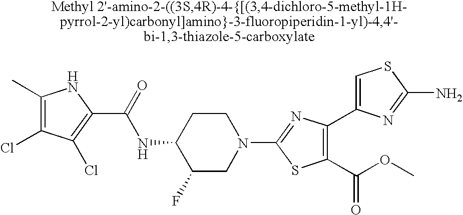 | MS (ES) (M + H)⁺: 533 forC₁₉H₁₉Cl₂FN₆O₃S₂NMR: 1.81 (m, 2H), 2.22 (s,3H), 3.32 (m, 4H), 3.46 (dd,1H), 3.69 (s, 3H), 4.02 (m,1H), 4.41 (m, 2H), 5.01 (d,1H), 7.02 (s, 2H), 7.23 (s,1H), 7.37 (d, 1H), 12.10 (s,1H). | Intermediate 55and 3,4-dichloro-N-[(3S,4R)-3-fluoropiperidin-4-yl]-5-methyl-1H-pyrrole-2-carboxamidehydrochloride |
| 75 | 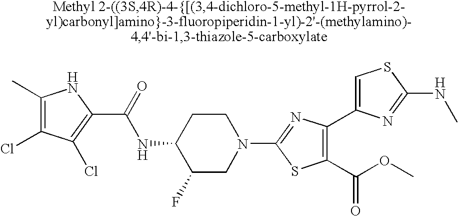 | MS (ES) (M + H)⁺: 547 forC₂₀H₂₁Cl₂FN₆O₃S₂NMR: 1.91 (m, 2H), 2.23 (s,3H), 2.81 (d, 3H), 3.34 (m,4H), 3.55 (dd, 1H), 3.71 (s,3H), 4.06 (m, 1H), 4.31 (m,2H), 5.02 (d, 1H), 7.08 (s,2H), 7.29 (d, 1H), 7.51 (m,1H), 12.19 (s, 1H). | Intermediate 56and 3,4-dichloro-N-[(3S,4R)-3-fluoropiperidin-4-yl]-5-methyl-1H-pyrrole-2-carboxamidehydrochloride |
| 76 | 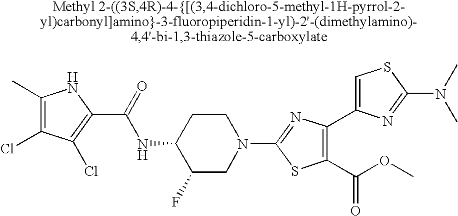 | MS (ES) (M + H)⁺: 561 forC₂₁H₂₃Cl₂FN₆O₃S₂NMR: 1.82 (m, 2H), 2.22 (s,3H), 3.03 (s, 6H), 3.34 (m,4H), 3.57 (dd, 1H), 3.69 (s,3H), 4.05 (m, 1H), 4.31 (m,2H), 5.03 (d, 1H), 7.12 (s,1H), 7.31 (d, 1H), 7.52 (m,1H), 12.10 (s, 1H). | Intermediate 57and 3,4-dichloro-N-[(3S,4R)-3-fluoropiperidin-4-yl]-5-methyl-1H-pyrrole-2-carboxamidehydrochloride |
| 77 | 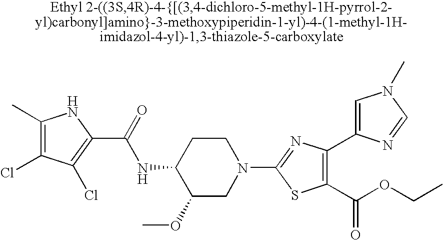 | MS (ES) (M + H)⁺: 541 forC₂₂H₂₆Cl₂N₆O₄SNMR: 1.22 (t, 3H), 1.75 (m,2H), 2.23 (s, 3H), 3.34 (m,4H), 3.48 (m, 1H), 3.71 (s,3H), 4.03 (m, 1H), 4.13 (m,2H), 4.17-4.34 (m, 2H), 7.15(s, 1H), 7.61 (s, 1H), 8.04 (s,1H), 12.20 (s, 1H). | Intermediate 12and 3,4-dichloro-N-[(3S,4R)-3-methoxypiperidin-4-yl]-5-methyl-1H-pyrrole-2-carboxamidehydrochloride |
| 78 | 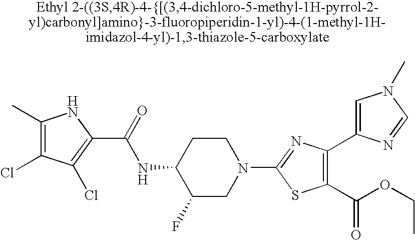 | MS (ES) (M + H)⁺: 529 forC₂₁H₂₃Cl₂FN₆O₃SNMR: 1.23 (t, 3H), 1.85 (m,2H), 2.22 (s, 3H), 3.35 (m,4H), 3.51 (m, 1H), 3.69 (s,3H), 4.03 (m, 1H), 4.15 (m,2H), 4.34 (m, 2H), 5.01 (d,1H), 7.27 (d, 1H), 7.59 (s,1H), 8.03 (s, 1H), 12.13 (s,1H). | Intermediate 12and 3,4-dichloro-N-[(3S,4R)-3-fluoropiperidin-4-yl]-5-methyl-1H-pyrrole-2-carboxamidehydrochloride |
| 79 | 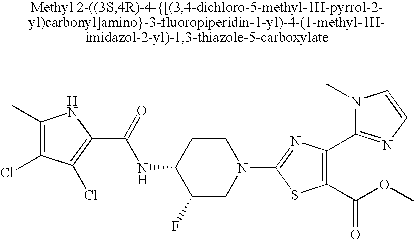 | MS (ES) (M + H)⁺: 515 forC₂₀H₂₁Cl₂FN₆O₃SNMR: 1.91 (m, 2H), 2.22 (s,3H), 3.34 (m, 4H), 3.58 (m,1H), 3.55 (s, 3H), 3.65 (s,3H), 4.10 (m, 1H), 4.28 (m,2H), 5.02 (d, 1H), 7.03 (s,1H), 7.34 (m, 2H), 12.18 (s,1H). | Intermediate 10and 3,4-dichloro-N-[(3S,4R)-3-fluoropiperidin-4-yl]-5-methyl-1H-pyrrole-2-carboxamidehydrochloride |
| 80 | 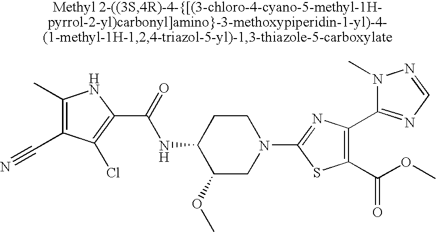 | MS (ES) (M + H)⁺: 519 forC₂₁H₂₃ClN₈O₄SNMR: 1.81 (m, 2H), 2.34 (s,3H), 3.32 (m, 4H), 3.58 (m,1H), 3.65 (s, 2H), 3.70 (s3H), 4.01 (m, 1H), 4.32 (m,2H), 7.31 (d, 1H), 8.04 (s,1H), 12.76 (s, 1H). | Intermediate 64andIntermediate 9 |
| 81 | 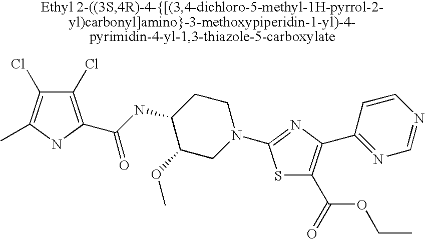 | MS (ES) (M + H)⁺: 539 forC₂₂H₂₄Cl₂N₆O₄SNMR: 1.02 (t, 3H), 1.70 (m,2H), 2.12 (s, 3H), 3.25 (m,2H), 3.31 (s, 3H), 3.50 (s,1H), 4.00 (q, 2H), 4.22 (m,3H), 7.09 (d, 1H), 7.66 (d,1H), 8.83 (d, 1H), 9.17 (s,1H), 12.10 (s, 1H) | 3,4-dichloro-N-[(3S,4R)-3-methoxypiperidin-4-yl]-5-methyl-1H-pyrrole-2-carboxamidehydrochloride andIntermediate 2 |
| 82 | 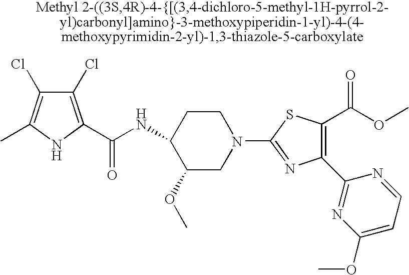 | MS (ES) (M + H)⁺: 556 forC₂₂H₂₄Cl₂N₆O₅S | 3,4-dichloro-N-[(3S,4R)-3-methoxypiperidin-4-yl]-5-methyl-1H-pyrrole-2-carboxamidehydrochloride andIntermediate 70 |
| 83 | 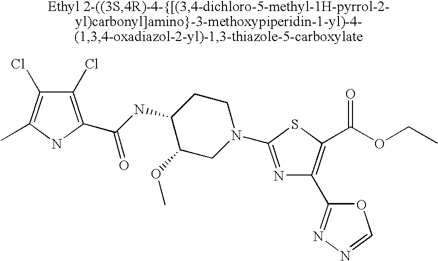 | MS (ES) (M + H)⁺: 529 forC₂₀H₂₂Cl₂N₆O₅S | 3,4-dichloro-N-[(3S,4R)-3-methoxypiperidin-4-yl]-5-methyl-1H-pyrrole-2-carboxamidehydrochloride andIntermediate 75 |
| 84 | 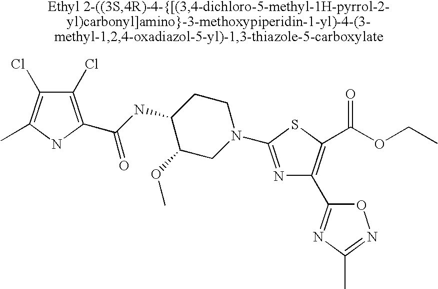 | MS (ES) (M + H)⁺: 544 forC₂₁H₂₄Cl₂N₆O₅SNMR (CDCl₃): 1.28 (t, 3H),2.03 (m, 2H), 2.30 (s, 3H),2.53 (s, 3H), 3.22 (d, 1H),3.30 (m, 1H), 3.48 (s, 3H),3.52 (m, 1H), 4.02 (m, 1H),4.26 (q, 2H), 4.32 (m, 1H),4.56 (m, 1H), 7.23 (d, 1H),9.27 (br, 1H). | 3,4-dichloro-N-[(3S,4R)-3-methoxypiperidin-4-yl]-5-methyl-1H-pyrrole-2-carboxamidehydrochloride andIntermediate 78 |
| 85 | 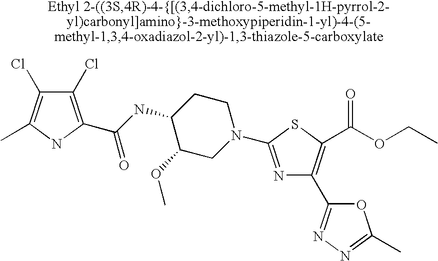 | MS (ES) (M + H)⁺: 544 forC₂₁H₂₄Cl₂N₆O₅SNMR (CDCl₃): 1.29 (t, 3H),2.03 (m, 2H), 2.30 (s, 3H),2.64 (s, 3H), 3.22 (d, 1H),3.30 (m, 1H), 3.49 (s, 3H),3.52 (m, 1H), 4.01 (m, 1H),4.26 (q, 2H), 4.32 (m, 1H),4.58 (m, 1H), 7.25 (d, 1H),9.25 (br, 1H) | 3,4-dichloro-N-[(3S,4R)-3-methoxypiperidin-4-yl]-5-methyl-1H-pyrrole-2-carboxamidehydrochloride andIntermediate 79 |

### Example 86

### Methyl 2-((3S,4R)-4-{[(3,4-dichloro-5-methyl-1H-pyrrol-2-yl)carbonyl]amino}-3-methoxypiperidin-1-yl)-4-(1H-imidazol-2-yl)-1,3-thiazole-5-carboxylate

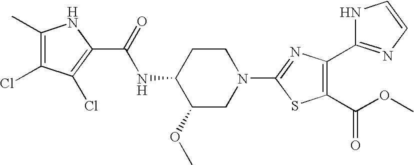

A solution of methyl 2-((3S,4R)-4-{[(3,4-dichloro-5-methyl-1H-pyrrol-2-yl)carbonyl]amino}-3-methoxypiperidin-1-yl)-4-[1-(methoxymethyl)-1H-imidazol-2-yl]-1,3-thiazole-5-carboxylate (Example 58; 173 mg 0.31 mmol) in 3 ml 8:1 acetic acid-water was heated at 120° C. for 2 hours in a microwave reactor. Solvent was removed and the residue was partitioned between NaHCO₃ (aq) and EtOAc. The EtOAc was separated and washed with brine. Drying (MgSO₄) and removal of solvent gave a solid that was chromatographed on silica gel (100% EtOAc followed by gradient elution to 20% MeOH in EtOAc). Product (78 mg) was isolated as a solid. MS (ES) (M+H)⁺: 513 for C₂₂H₂₆Cl₂N₆O₅S; NMR: 1.83 (m, 2H), 2.24 (s, 3H), 3.34 (s, 3H), 3.45 (m, 4H), 3.59 (m, 1H), 3.82 (s, 3H), 4.02 (m, 1H), 4.33 (m, 2H), 7.15 (m, 3H), 7.41 (s, 2H), 12.20 (s, 1H), 12.77 (s, 1H). Example 87

The following Example was synthesized by an analogous method to Example 86 from the starting material (SM) given in the table below.

| Ex | Compound | Data | SM |
|---|---|---|---|
| 87 | 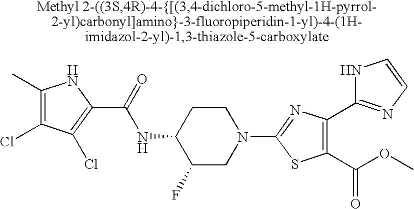 | MS (ES) (M + H)⁺: 501 forC₁₉H₁₉Cl₂FN₆O₃SNMR: 1.91 (m, 2H), 2.22 (s, 3H), 3.41(m, 4H), 3.46-3.73 (m, 2H), 3.81 (s, 3H),4.11 (m, 1H), 4.26-4.53 (m, 2H), 5.01 (d,1H), 7.21 (s, 2H), 7.31 (d, 1H), 12.16 (s,1H), 12.74 (s, 1H). | Example 59 |

### Example 88 and Example 89

### Methyl 2-((3S,4R)-4-{[(3,4-dichloro-5-methyl-1H-pyrrol-2-yl)carbonyl]amino}-3-methoxypiperidin-1-yl)-4-(1-methyl-1H-imidazol-2-yl)-1 3-thiazole-5-carboxylate and 2-[2-((3S,4R)-4-{[(3,4-Dichloro-5-methyl-1H-pyrrol-2-yl)carbonyl]amino}-3-methoxypiperidin-1-yl)-5-(methoxycarbonyl)-1,3-thiazol-4-yl]-1,3-dimethyl-1H-imidazol-3-ium iodide

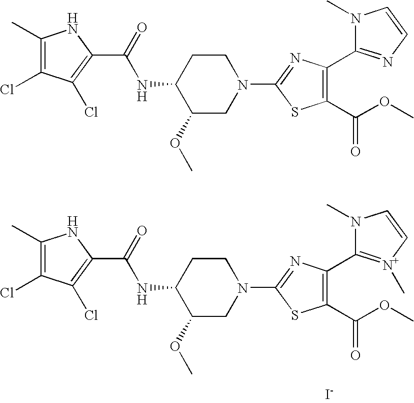

A solution of 280 mg (0.55 mmol) of methyl 2-((3S,4R)-4-{[(3,4-dichloro-5-methyl-1H-pyrrol-2-yl)carbonyl]amino}-3-methoxypiperidin-1-yl)-4-(1H-imidazol-2-yl)-1,3-thiazole-5-carboxylate (Example 86), 46 mg (0.55 mmol) NaHCO₃ and 35 μl (0.56 mmol) methyl iodide in 3 ml CH₃CN was heated at 80° C. for 30 min in a microwave reactor. The mixture was diluted with water, which was saturated with NaCl before being extracted with 4 times with DCM. The extracts were dried (MgSO₄) and concentrated, and the residue was chromatographed on silica gel (100% DCM followed by gradient elution to 15% MeOH in DCM) to afford 2 components. The second eluting component (75 mg), corresponds to the second titled compound, MS (ES) (M+H)⁺: 541 for C₂₂H₂₇Cl₂N₆O₄S; NMR: 1.81 (m, 2H), 2.22 (s, 3H), 3.43 (m, 4H), 3.54 (m, 1H), 3.66 (m, 1H), 3.71 (s, 6H), 4.01 (m, 1H), 4.31 (m, 2H), 7.15 (d, 1H), 7.87 (s, 2H), 12.20 (s, 1H). The first eluting component was chromatographed again (100% EtOAc followed by gradient elution to 20% MeOH in EtOAc) to afford 2 components with the first eluting component (70 mg) corresponding to starting material [MS (ES) (M+H)⁺: 513 for C₂₀H₂₂Cl₂N₆O₅S] and the second eluting component (103 mg) corresponding to the first titled compound, MS (ES) (M+H)⁺: 527 for C₂₁H₂₄Cl₂N₆O₄S; NMR: 1.81 (m, 2H), 2.22 (s, 3H), 3.44 (s, 3H), 3.52 (s, 3H), 3.62 (s, 3H), 4.04 (m, 1H), 4.27 (m, 2H), 7.05 (s, 1H), 7.22 (d, 1H), 7.25 (s, 1H), 12.20 (s, 1H).

### Example 90

### 2-((3S,4R)-4-{[(3,4-Dichloro-5-methyl-1H-pyrrol-2-yl)carbonyl]amino}-3-methoxypiperidin-1-yl)-4-(5-methyl-1,3,4-oxadiazol-2-yl)-1,3-thiazole-5-carboxylic acid

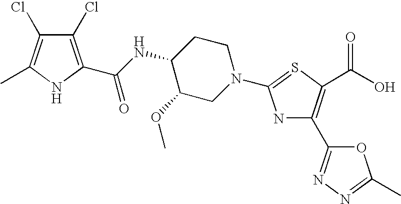

Ethyl 2-((3S,4R)-4-{[(3,4-dichloro-5-methyl-1H-pyrrol-2-yl)carbonyl]amino}-3-methoxypiperidin-1-yl)-4-(5-methyl-1,3,4-oxadiazol-2-yl)-1,3-thiazole-5-carboxylate (Example 85; 140 mg, 0.26 mmol) was dissolved in 1,4-dioxane/2 M NaOH (9 ml:3 ml), the mixture was stirred at room temperature for 3 hrs, LC-MS showed the completion of the reaction. The reaction mixture was washed with ether (10 ml), the aqueous layer was collected and acidified to pH=3 with 2 M HCl solution. The resulting precipitate was collected by filtration and washed with H₂O, dried under high vacuum. The desired product was obtained as white solid (127 mg). MS (ES) (M+H)⁺: 516 for C₁₉H₂₀Cl₂N₆O₅S; NMR: 1.77 (m, 2H), 2.19 (s, 3H), 2.58 (s, 3H), 3.22 (d, 1H), 3.30 (m, 1H), 3.38 (s, 3H), 3.57 (m, 1H), 4.01 (m, 1H), 4.26 (q, 2H), 7.17 (d, 1H), 12.16 (s, 1H), 13.29 (br, 1H).

### Example 91

The following compound was synthesized according to the procedure described for Example 90.

| Ex | Compound | Data | SM |
|---|---|---|---|
| 91 | 2-((3S,4R)-4-{[(3,4-Dichloro-5- | MS (ES) (M + H)⁺: 516 for | Example 84 |
| | methyl-1H-pyrrol-2- | C₁₉H₂₀Cl₂N₆O₅S | |
| | yl)carbonyl]amino}-3- | NMR: 1.77 (m, 2 H), 2.19 (s, | |
| | methoxypiperidin-1-yl)-4-(5-methyl- | 3 H), 2.44 (s, 3 H), 3.30 (m, 1 H), | |
| | 1,3,4-oxadiazol-2-yl)-1,3-thiazole-5- | 3.37 (s, 3 H), 3.40 (m, 1 H), 3.57 | |
| | carboxylic acid | (m, 1 H), 3.97 (m, 1 H), 4.27 (m, | |
| | 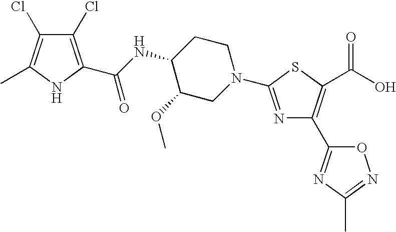 | 2 H), 7.17 (d, 1 H), 12.16 (s, 1 H),13.35 (br, 1 H). | |

### Examples 92-108

The following compounds were synthesized according to the procedure described for Example 43.

| Ex | Compound | Data | SM |
|---|---|---|---|
| 92 | methyl 2-((3S,4R)-4-(3,4-dichloro-5- | MS (ES): 529 (MH⁺) for | 3,4-dichloro- |
| | methyl-1H-pyrrole-2-carboxamido)- | C₂₀H₂₃Cl₂N₇O₄S | N-[(3S,4R)-3- |
| | 3-methoxypiperidin-1-yl)-4-(1- | ¹H-NMR: 1.77 (m, 2 H); 2.19 (s, | methoxypiperidin- |
| | methyl-1H-1,2,3-triazol-4-yl)thiazole- | 3 H); 3.27 (m, 2 H); 3.39 (s, 3 H); | 4-yl]-5- |
| | 5-carboxylate | 3.58 (m, 1 H); 3.72 (s, 3 H); | methyl-1H- |
| | 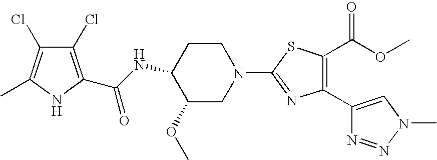 | 4.03 (m, 1 H); 4.12 (s, 3 H); 4.29(m, 2 H); 7.18 (d, 1 H); 8.68 (s,1 H); 12.16 (s, 1 H). | pyrrole-2-carboxamidehydrochlorideandIntermediate81 |
| 93 | methyl 2-((3S,4R)-4-(3,4-dichloro-5- | MS (ES): 554 (MH⁺) for | 3,4-dichloro- |
| | methyl-1H-pyrrole-2-carboxamido)- | C₂₃H₂₆Cl₂N₆O₄S | N-[(3S,4R)-3- |
| | 3-methoxypiperidin-1-yl)-4-(3,5- | ¹H-NMR: 1.77 (m, 2 H); 2.19 (s, | methoxypiperidin- |
| | dimethylpyrazin-2-yl)thiazole-5- | 3 H); 2.31 (s, 3 H); 2.46 (s, 3 H); | 4-yl]-5- |
| | carboxylate | 3.27 (m, 2 H); 3.37 (s, 3 H); | methyl-1H- |
| | 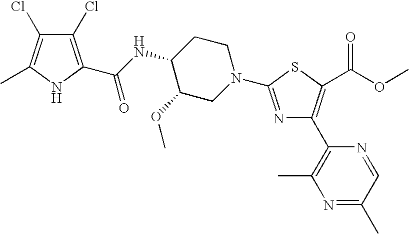 | 3.58 (s, 3 H); 3.58 (m, 1 H);4.03 (m, 1 H); 4.29 (m, 2 H); 7.18(d, 1 H); 8.44 (s, 1 H); 12.16 (s,1 H). | pyrrole-2-carboxamidehydrochlorideandIntermediate82 |
| 94 | methyl 2-((3S,4R)-4-(3,4-dichloro-5- | MS (ES): 555 (MH⁺) for | 3,4-dichloro- |
| | methyl-1H-pyrrole-2-carboxamido)- | C₂₃H₂₅Cl₂N₅O₅S | N-[(3S,4R)-3- |
| | 3-methoxypiperidin-1-yl)-4-(6- | ¹H-NMR: 1.77 (m, 2 H); 2.19 (s, | methoxypiperidin- |
| | methoxypyridin-2-yl)thiazole-5- | 3 H); 3.30 (m, 1 H); 3.40 (s, 3 H); | 4-yl]-5- |
| | carboxylate | 3.40 (m, 1 H); 3.58 (m, 1 H); 3.63 | methyl-1H- |
| | 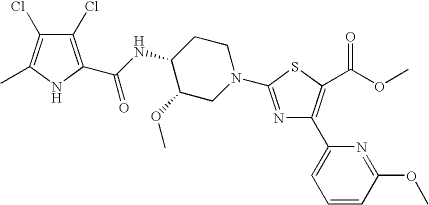 | (s, 3 H); 3.81 (s, 3 H); 3.94 (m,1 H); 4.29 (m, 2 H); 6.83 (d, 1 H);7.17 (d, 1 H); 7.22 (d, 1 H); 7.75(t, 1 H); 12.16 (s, 1 H). | pyrrole-2-carboxamidehydrochlorideandIntermediate83 |
| 95 | ethyl 2-((3S,4R)-4-(3,4-dichloro-5- | MS (ES): 569 (MH⁺) for | 3,4-dichloro- |
| | methyl-1H-pyrrole-2-carboxamido)- | C₂₄H₂₇Cl₂N₅O₅S | N-[(3S,4R)-3- |
| | 3-methoxypiperidin-1-yl)-4-(4- | ¹H-NMR: 1.27 (t, 3 H); 1.97 (m, | methoxypiperidin- |
| | methoxypyridin-2-yl)thiazole-5- | 2 H); 2.27 (s, 3 H); 3.20~3.40 (m, | 4-yl]-5- |
| | carboxylate | 4H); 3.54 (s, 3 H); 3.58 (m, 1 H); | methyl-1H- |
| | 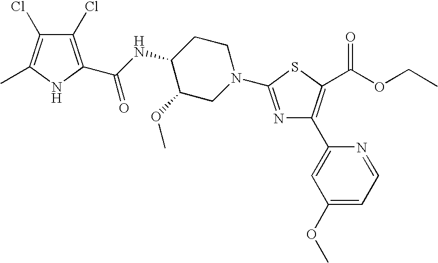 | 4.01 (s, 3 H); 4.25 (q, 2 H); 4.31(m, 1 H); 7.03 (d, 1 H); 7.24 (d,1 H); 7.25 (d, 1 H); 8.81 (s, 1 H);9.35 (s, 1 H). | pyrrole-2-carboxamidehydrochlorideandIntermediate84 |
| 96 | ethyl 2-((3S,4R)-4-(3,4-dichloro-5- | MS (ES): 554 (MH⁺) for | 3,4-dichloro- |
| | methyl-1H-pyrrole-2-carboxamido)- | C₂₃H₂₆Cl₂N₆O₄S | N-[(3S,4R)-3- |
| | 3-methoxypiperidin-1-yl)-4-(5- | ¹H-NMR: 1.10 (t, 3 H); 1.77 (m, | methoxypiperidin- |
| | methylpyrazin-2-yl)thiazole-5- | 2 H); 2.18 (s, 3 H); 2.55 (s, 3 H); | 4-yl]-5- |
| | carboxylate | 3.38 (s, 3 H); 3.39 (m, 3 H); 3.56 | methyl-1H- |
| | 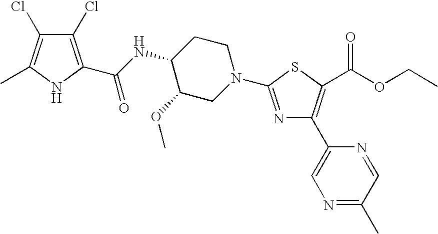 | (m, 1 H); 4.06 (q, 2 H); 4.29 (m,2 H); 7.15 (d, 1 H); 8.58 (s, 1 H);8.69 (s, 1 H); 12.14 (s, 1 H). | pyrrole-2-carboxamidehydrochlorideandIntermediate85 |
| 97 | ethyl 2-((3S,4R)-4-(3,4-dichloro-5- | MS (ES): 557 (MH⁺) for | 3,4-dichloro- |
| | methyl-1H-pyrrole-2-carboxamido)- | C₂₃H₂₄Cl₂FN₅O₄S | N-[(3S,4R)-3- |
| | 3-methoxypiperidin-1-yl)-4-(3- | ¹H-NMR (CDCl₃): 1.19 (t, 3 H); | methoxypiperidin- |
| | fluoropyridin-2-yl)thiazole-5- | 1.90 (m, 1 H); 2.0 (m, 1 H); 2.27 | 4-yl]-5- |
| | carboxylate | (s, 3 H); 3.25 (m, 2 H); 3.46 (s, | methyl-1H- |
| | 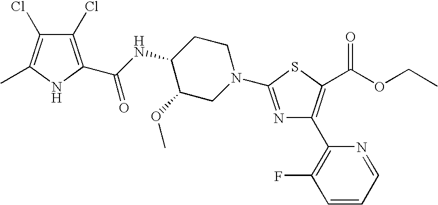 | 3 H); 3.52 (m, 1 H); 4.10 (m, 1 H);4.17 (q, 2 H); 4.30 (m, 1 H); 4.53(m, 1 H); 7.20 (d, 1 H); 7.49 (dd,1 H); 7.61 (t, 1 H); 8.58 (d, 1 H);9.30 (s, 1 H). | pyrrole-2-carboxamidehydrochlorideandIntermediate86 |
| 98 | Ethyl 2-((3S,4R)-4-(3,4-dichloro-5- | MS (ES): 557 (MH⁺) for | 3,4-dichloro- |
| | methyl-1H-pyrrole-2-carboxamido)- | C₂₃H₂₄FCl₂N₅O₄S | N-[(3S,4R)-3- |
| | 3-fluoropiperidin-1-yl)-4-(4- | ¹H-NMR *CDCl₃) 1.77 (m, 2 H); | fluoropiperidin- |
| | methoxypyridin-2-yl)thiazole-5- | 2.08 (m, 3 H); 2.27 (s, 3 H); 3.33 | 4-yl]-5- |
| | carboxylate | (m, 2 H); 3.48 (m, 1 H); 4.14 (s, | methyl-1H- |
| | 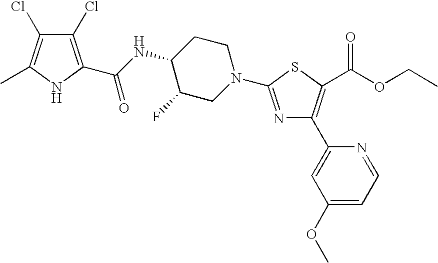 | 3 H); 4.33 (q, 2 H); 4.66 (m, 1 H);4.87 (m, 1 H); 5.05 (m, 1 H); 7.21(d, 1 H); 8.46 (s, 1 H); 8.78 (d,1 H); 9.0 (d, 1 H); 9.42 (s, 1 H). | pyrrole-2-carboxamidehydrochlorideandIntermediate84 |
| 99 | methyl 2-((3S,4R)-4-(3,4-dichloro-5- | MS (ES): 517 (MH⁺) for | 3,4-dichloro- |
| | methyl-1H-pyrrole-2-carboxamido)- | C₁₉H₂₀Cl₂FN₇O₃S | N-[(3S,4R)-3- |
| | 3-fluoropiperidin-1-yl)-4-(1-methyl- | ¹H-NMR: 1.87 (m, 2 H); 2.18 (s, | fluoropiperidin- |
| | 1H-1,2,3-triazol-4-yl)thiazole-5- | 3 H); 3.35 (m, 2 H); 3.72 (s, 3 H); | 4-yl]-5- |
| | carboxylate | 4.10 (s, 3 H); 4.11 (m, 1 H); 4.39 | methyl-1H- |
| | 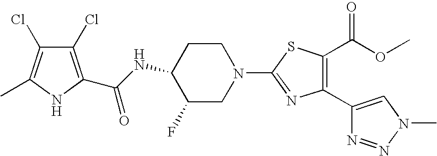 | (m, 2 H); 4.90~5.06 (d, 1 H); 7.27(d, 1 H); 8.68 (s, 1 H); 12.10 (s,1 H). | pyrrole-2-carboxamidehydrochlorideandIntermediate81 |
| 100 | methyl 2-((3S,4R)-4-(3,4-dichloro-5- | MS (ES): 543 (MH⁺) for | 3,4-dichloro- |
| | methyl-1H-pyrrole-2-carboxamido)- | C₂₂H₂₂Cl₂FN₅O₄S | N-[(3S,4R)-3- |
| | 3-fluoropiperidin-1-yl)-4-(6- | ¹H-NMR: 1.88 (m, 2 H); 2.20 (s, | fluoropiperidin- |
| | methoxypyridin-2-yl)thiazole-5- | 3 H); 3.39 (m, 1 H); 3.52 (m, 1 H); | 4-yl]-5- |
| | carboxylate | 3.64 (s, 3 H); 3.82 (s, 3 H); 4.03 | methyl-1H- |
| | 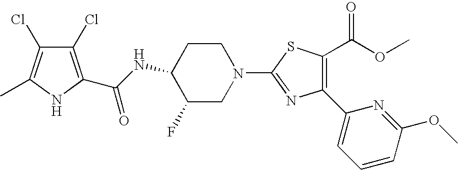 | (m, 1 H); 4.35 (m, 2 H);4.90~5.06 (d, 1 H); 6.85 (d, 1 H);7.22 (d, 1 H); 7.27 (d, 1 H); 7.76(t, 1 H); 12.11 (s, 1 H). | pyrrole-2-carboxamidehydrochlorideandIntermediate83 |
| 101 | methyl 2-((3S,4R)-4-(3,4-dichloro-5- | MS (ES): 542 (MH⁺) for | 3,4-dichloro- |
| | methyl-1H-pyrrole-2-carboxamido)- | C₂₂H₂₃Cl₂FN₆O₃S | N-[(3S,4R)-3- |
| | 3-fluoropiperidin-1-yl)-4-(6- | ¹H-NMR: 1.10 (t, 3 H); 1.88 (m, | fluoropiperidin- |
| | methoxypyridin-2-yl)thiazole-5- | 2 H); 2.20 (s, 3 H); 2.55 (s, 3 H); | 4-yl]-5- |
| | carboxylate | 3.31 (m, 2 H); 3.52~4.04 (dd, | methyl-1H- |
| | 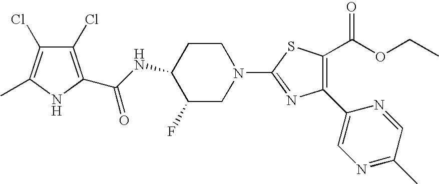 | 1 H); 4.08 (q, 2 H); 4.35 (m, 2 H);4.90~5.06 (d, 1 H); 7.27 (d, 1 H);8.59 (s, 1 H); 8.70 (s, 1 H); 12.09(s, 1 H). | pyrrole-2-carboxamidehydrochlorideandIntermediate85 |
| 102 | ethyl 2-((3S,4R)-4-(3,4-dichloro-5- | MS (ES): 545 (MH⁺) for | 3,4-dichloro- |
| | methyl-1H-pyrrole-2-carboxamido)- | C₂₂H₂₁Cl₂F₂N₅O₃S | N-[(3S,4R)-3- |
| | 3-fluoropiperidin-1-yl)-4-(3- | ¹H-NMR CDCl3: 1.18 (t, 3 H); | fluoropiperidin- |
| | fluoropyridin-2-yl)thiazole-5- | 2.09 (m, 3 H); 2.29 (s, 3 H); 3.31 | 4-yl]-5- |
| | carboxylate | (m, 1 H); 3.50~3.71 (dd, 1 H); | methyl-1H- |
| | 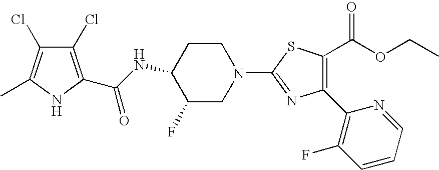 | 4.17 (q, 2 H); 4.31 (m, 1 H); 4.53(m, 1 H); 4.83~4.99 (d, 1 H); 7.02(d, 1 H); 7.41 (dd, 1 H); 7.51 (t,1 H); 8.54 (d, 1 H); 10.01 (s, 1 H). | pyrrole-2-carboxamidehydrochlorideandIntermediate86 |
| 103 | methyl 2-((3S,4R)-4-(4-chloro-3- | MS (ES): 520 (MH⁺) for | Intermediate |
| | cyano-5-methyl-1H-pyrrole-2- | C₂₁H₂₃ClN₈O₄S | 119 and |
| | carboxamido)-3-methoxypiperidin-1- | ¹H-NMR: 1.77 (m, 2 H); 2.21 (s, | Intermediate |
| | yl)-4-(1-methyl-1H-1,2,3-triazol-4- | 3 H); 3.33 (m, 2 H); 3.36 (s, 3 H); | 81 |
| | yl)thiazole-5-carboxylate | 3.58 (m, 1 H); 3.73 (s, 3 H); | |
| | 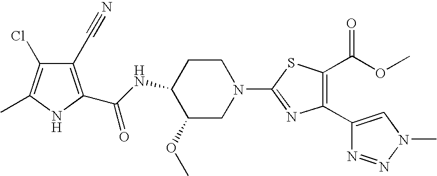 | 4.03 (m, 1 H); 4.12 (s, 3 H); 4.28(m, 2 H); 7.77 (d, 1 H); 8.69 (s,1 H); 12.68 (s, 1 H). | |
| 104 | methyl 2-((3S,4R)-4-(4-chloro-3- | MS (ES) 520 (MH⁺) for | Intermediate |
| | cyano-5-methyl-1H-pyrrole-2- | C₂₁H₂₃ClN₈O₄S | 119 and |
| | carboxamido)-3-methoxypiperidin-1- | ¹H-NMR: 1.75 (m, 2 H); 2.20 (s, | Intermediate |
| | yl)-4-(1-methyl-1H-1,2,4-triazol-5- | 3 H); 3.34 (s, 3 H); 3.37 (m, 2 H); | 9 |
| | yl)thiazole-5-carboxylate | 3.58 (m, 1 H); 3.60 (s, 3 H); 3.72 | |
| | 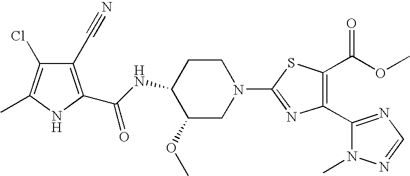 | (s, 3 H); 3.96 (m, 1 H); 4.25 (m,2 H); 7.75 (d, 1 H); 8.01 (s, 1 H);12.67 (s, 1 H). | |
| 105 | ethyl 2-((3S,4R)-4-(4-chloro-3-cyano- | MS (ES): 531 (MH⁺) for | Intermediate |
| | 5-methyl-1H-pyrrole-2- | C₂₃H₂₄ClN₇O₄S | 119 and |
| | carboxamido)-3-methoxypiperidin-1- | ¹H-NMR: 1.08 (t, 3 H); 1.77 (m, | Intermediate 1 |
| | yl)-4-(pyrimidin-2-yl)thiazole-5- | 2 H); 2.21 (s, 3 H); 3.33 (m, 2 H); | |
| | carboxylate | 3.35 (s, 3 H); 3.58 (m, 1 H); 4.10 | |
| | 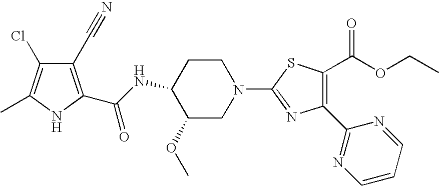 | (q, 2 H); 4.28 (m, 2 H); 7.76 (d,1 H); 8.71 (m, 1 H); 8.67 (m, 1 H);8.84 (m, 1 H); 12.68 (s, 1 H). | |
| 106 | ethyl 2-((3S,4R)-4-(4-chloro-3-cyano- | MS (ES): 548 (MH⁺) for | Intermediate |
| | 5-methyl-1H-pyrrole-2- | C₂₄H₂₄ClFN₆O₄S | 119 and |
| | carboxamido)-3-methoxypiperidin-1- | ¹H-NMR: 1.03 (t, 3 H); 1.75 (m, | Intermediate |
| | yl)-4-(3-fluoropyridin-2-yl)thiazole- | 2 H); 2.21 (s, 3 H); 3.34 (s, 3 H); | 86 |
| | 5-carboxylate | 3.36 (m, 2 H); 3.56 (m, 1 H); 4.02 | |
| | 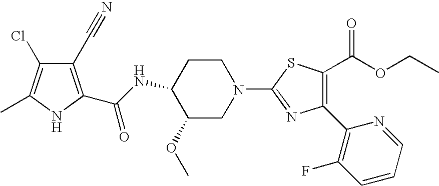 | (m, 3 H); 4.26 (m, 2 H); 7.56 (m,1 H); 7.80 (m, 2 H); 8.46 (d, 1 H);12.68 (s, 1 H). | |
| 107 | ethyl 2-((3S,4R)-4-(4-chloro-3-cyano- | MS (ES): 545 (MH⁺) for | Intermediate |
| | 5-methyl-1H-pyrrole-2- | C₂₄H₂₆ClN₇O₄S | 119 and |
| | carboxamido)-3-methoxypiperidin-1- | | Intermediate |
| | yl)-4-(5-methylpyrazin-2-yl)thiazole- | | 85 |
| | 5-carboxylate | | |
| | 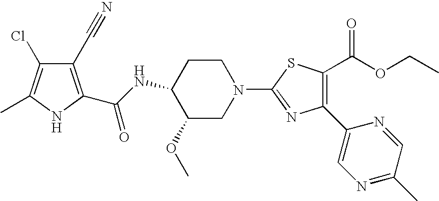 | | |
| 108 | ethyl 2-((3S,4R)-4-(4-chloro-3-cyano- | MS (ES): 533 (MH⁺) for | Intermediate |
| | 5-methyl-1H-pyrrole-2- | C₂₃H₂₆ClN₇O₄S | 119 and |
| | carboxamido)-3-methoxypiperidin-1- | ¹H-NMR: 1.28 (t, 3 H); 1.75 (m, | Intermediate |
| | yl)-4-(1-methyl-1H-imidazol-4- | 2 H); 2.21 (s, 3 H); 3.17 (m, 2 H); | 12 |
| | yl)thiazole-5-carboxylate | 3.33 (m, 2 H); 3.36 (s, 3 H); 3.61 | |
| | 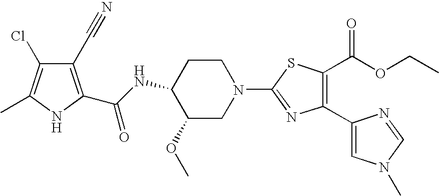 | (m, 1 H); 3.94 (s, 3 H); 4.29 (m,3 H); 7.77 (d, 1 H); 8.55 (s, 1 H);9.17 (s, 1 H); 12.72 (br, 1 H). | |

### Examples 109-125

The following compounds were synthesized according to the procedure described for Example 1.

| Ex | Compound | Data | SM |
|---|---|---|---|
| 109 | 2-((3S,4R)-4-(3,4-dichloro-5-methyl- | MS (ES): 515 (MH⁺) for | Example 92 |
| | 1H-pyrrole-2-carboxamido)-3- | C₁₉H₂₁Cl₂N₇O₄S | |
| | methoxypiperidin-1-yl)-4-(1-methyl- | ¹H-NMR: 1.77 (m, 2 H); 2.19 (s, | |
| | 1H-1,2,3-triazol-4-yl)thiazole-5- | 3 H); 3.27 (m, 2 H); 3.39 (s, 3 H); | |
| | carboxylic acid | 3.58 (m, 1 H); 4.03 (m, 1 H); 4.15 | |
| | 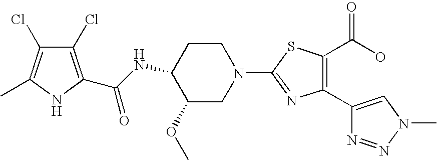 | (s, 3 H); 4.29 (m, 2 H); 7.18 (d,1 H); 8.70 (s, 1 H); 12.17 (s, 1 H);14.15 (s, br, 1 H). | |
| 110 | 2-((3S,4R)-4-(3,4-dichloro-5-methyl- | MS (ES): 540 (MH⁺) for | Example 93 |
| | 1H-pyrrole-2-carboxamido)-3- | C₂₂H₂₄Cl₂N₆O₄S | |
| | methoxypiperidin-1-yl)-4-(3,5- | ¹H-NMR: 1.77 (m, 2 H); 2.19 (s, | |
| | dimethylpyrazin-2-yl)thiazole-5- | 3 H); 2.31 (s, 3 H); 2.46 (s, 3 H); | |
| | carboxylic acid | 3.27 (m, 2 H); 3.37 (s, 3 H); | |
| | 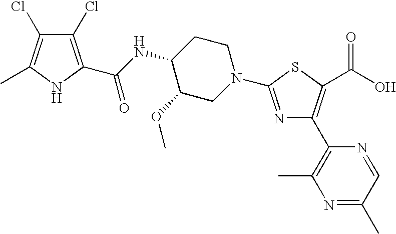 | 3.58 (s, 3 H); 3.58 (m, 1 H);4.03 (m, 1 H); 4.29 (m, 2 H); 7.18(d, 1 H); 8.44 (s, 1 H); 12.16 (s,1 H). | |
| 111 | 2-((3S,4R)-4-(3,4-dichloro-5-methyl- | MS (ES): 541 (MH⁺) for | Example 94 |
| | 1H-pyrrole-2-carboxamido)-3- | C₂₂H₂₃Cl₂N₅O₅S | |
| | methoxypiperidin-1-yl)-4-(6- | ¹H-NMR: 1.77 (m, 2 H); 2.19 (s, | |
| | methoxypyridin-2-yl)thiazole-5- | 3 H); 3.30 (m, 1 H); 3.40 (s, 3 H); | |
| | carboxylic acid | 3.40 (m, 1 H); 3.58 (m, 1 H); 3.99 | |
| | 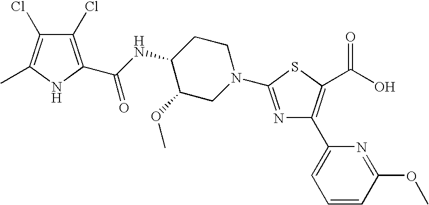 | (s, 3 H); 4.27 (m, 2 H); 4.40 (m,1 H); 7.17 (d, 1 H); 7.18 (d, 1 H);7.97 (d, 1 H); 8.10 (t, 1 H); 12.16(s, 1 H). | |
| 112 | 2-((3S,4R)-4-(3,4-dichloro-5-methyl- | MS (ES): 541 (MH⁺) for | Example 95 |
| | 1H-pyrrole-2-carboxamido)-3- | C₂₂H₂₃Cl₂N₅O₅S | |
| | methoxypiperidin-1-yl)-4-(4- | ¹H-NMR: 1.79 (m, 2 H); 2.19 (s, | |
| | methoxypyridin-2-yl)thiazole-5- | 3 H); 3.32 (m, 2 H); 3.40 (s, 3 H); | |
| | carboxylic acid | 3.58 (m, 1 H); 3.99 (s, 3 H); 4.28 | |
| | 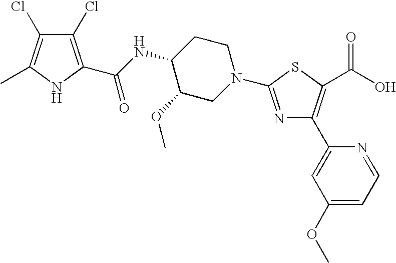 | (m, 2 H); 4.50 (m, 1 H); 7.17 (d,1 H); 7.30 (d, 1 H), 7.89 (s, 1 H),8.62 (d, 1 H); 12.16 (s, 1 H). | |
| 113 | 2-((3S,4R)-4-(3,4-dichloro-5-methyl- | MS (ES): 526 (MH⁺) for | Example 96 |
| | 1H-pyrrole-2-carboxamido)-3- | C₂₁H₂₂Cl₂N₆O₄S | |
| | methoxypiperidin-1-yl)-4-(5- | ¹H-NMR: 1.77 (m, 2 H); 2.19 (s, | |
| | methylpyrazin-2-yl)thiazole-5- | 3 H); 2.61 (s, 3 H); 3.40 (s, 3 H); | |
| | carboxylic acid | 3.38 (m, 2 H); 3.59 (m, 1 H); 4.00 | |
| | 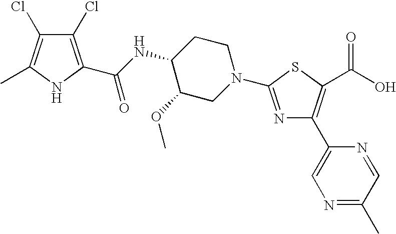 | (m, 1 H); 4.29 (m, 1 H); 4.46 (m,1 H); 7.16 (d, 1 H); 8.68 (s, 1 H);9.29 (s, 1 H); 12.14 (s, 1 H);15.75 (s, 1 H). | |
| 114 | 2-((3S,4R)-4-(3,4-dichloro-5-methyl- | MS (ES): 529 (MH⁺) for | Example 97 |
| | 1H-pyrrole-2-carboxamido)-3- | C₂₂H₂₀Cl₂FN₅O₄S | |
| | methoxypiperidin-1-yl)-4-(3- | ¹H-NMR: 1.77 (m, 2 H); 2.19 (s, | |
| | fluoropyridin-2-yl)thiazole-5- | 3 H); 3.37 (s, 3 H); 3.40 (m, 2 H); | |
| | carboxylic acid | 3.56 (m, 1 H); 3.96 (m, 1 H); 4.26 | |
| | 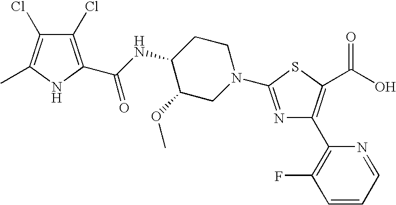 | (m, 2 H); 7.17 (d, 1 H); 7.54 (dd,1 H); 7.79 (t, 1 H); 8.46 (d, 1 H);12.16 (s, 1 H); 12.82 (br, 1 H). | |
| 115 | 2-((3S,4R)-4-(3,4-dichloro-5-methyl- | MS (ES): 529 (MH⁺) for | Example 98 |
| | 1H-pyrrole-2-carboxamido)-3- | C₂₁H₂₀FCl₂N₅O₄S | |
| | fluoropiperidin-1-yl)-4-(3- | ¹H-NMR: 1.77 (m, 2 H); 2.19 (s, | |
| | fluoropyridin-2-yl)thiazole-5- | 3 H); 3.40 (m, 2 H); 3.37 (s, 3 H); | |
| | carboxylic acid | 3.56 (m, 1 H); 3.96 (m, 1 H); 4.26 | |
| | 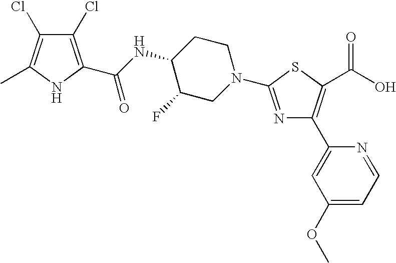 | (m, 2 H); 7.17 (d, 1 H); 7.54 (t,1 H); 7.75 (t, 1 H); 8.46 (s, 1 H);12.16 (s, 1 H); 12.82 (br, 1 H). | |
| 116 | 2-((3S,4R)-4-(3,4-dichloro-5-methyl- | MS (ES): 503 (MH⁺) for | Example 99 |
| | 1H-pyrrole-2-carboxamido)-3- | C₁₁H₁₈Cl₂FN₇O₃S | |
| | fluoropiperidin-1-yl)-4-(1-methyl-1H- | ¹H-NNMR: 1.88 (m, 2 H); 2.19 | |
| | 1,2,3-triazol-4-yl)thiazole-5- | (s, 3 H); 3.27 (m, 2 H); 3.35 (m, | |
| | carboxylic acid | 2 H); 3.53 (m, 1 H); 3.65 (m, 1 H); | |
| | 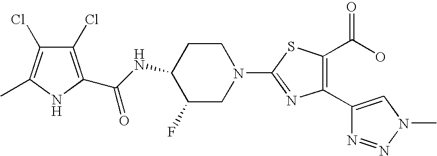 | 4.13 (m, 1 H); 4.39 (m, 2 H);4.90~5.06 (d, 1 H),; 7.29 (d,1 H); 8.70 (s, 1 H); 12.10 (s, 1 H). | |
| 117 | 2-((3S,4R)-4-(3,4-dichloro-5-methyl- | MS (ES): 529 (MH⁺) for | Example 100 |
| | 1H-pyrrole-2-carboxamido)-3- | C₂₁H₂₀Cl₂FN₅O₄S | |
| | fluoropiperidin-1-yl)-4-(6- | ¹H-NMR: 1.88 (m, 2 H); 2.20 (s, | |
| | methoxypyridin-2-yl)thiazole-5- | 3 H); 3.39 (m, 2 H); 3.50~3.71 | |
| | carboxylic acid | (dd, 1 H); 3.97 (s, 3 H); 4.14 (m, | |
| | 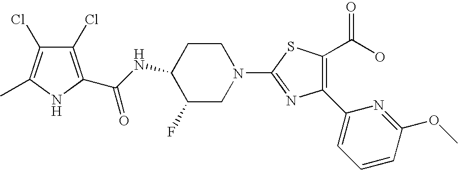 | 1 H); 4.43 (m, 2 H); 4.92~5.08 (d,1 H); 7.16 (d, 1 H); 7.30 (d, 1 H);7.98 (d, 1 H); 8.10 (t, 1 H); 12.12(s, 1 H). | |
| 118 | 2-((3S,4R)-4-(3,4-dichloro-5-methyl- | MS (ES): 514 (MH⁺) for | Example 101 |
| | 1H-pyrrole-2-carboxamido)-3- | C₂₀H₁₉Cl₂FN₆O₃S | |
| | fluoropiperidin-1-yl)-4-(5- | ¹H-NMR: 1.89 (m, 2 H); 2.20 (s, | |
| | methylpyrazin-2-yl)thiazole-5- | 3 H); 2.61 (s, 3 H); 3.41 (m, 1 H); | |
| | carboxylic acid | 3.50~3.71 (dd, 1 H); 4.11 (m, | |
| | 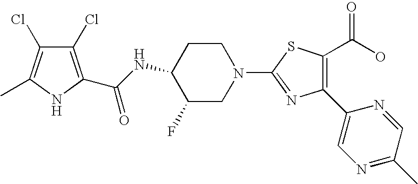 | 1 H); 4.43 (m, 2 H); 4.92~5.08 (d,1 H); 7.27 (d, 1 H); 8.68 (s, 1 H);9.25 (s, 1 H); 12.10 (s, 1 H); 15.64(s, 1 H). | |
| 119 | 2-((3S,4R)-4-(3,4-dichloro-5-methyl- | MS (ES): 517 (MH⁺) for | Example 102 |
| | 1H-pyrrole-2-carboxamido)-3- | C₂₀H₁₇Cl₂F₂N₅O₃S | |
| | fluoropiperidin-1-yl)-4-(3- | ¹H-NMR: 1.87 (m, 2 H); 2.19 (s, | |
| | fluoropyridin-2-yl)thiazole-5- | 3 H); 3.29 (m, 1 H); 3.50~3.71 | |
| | carboxylic acid | | |
| | 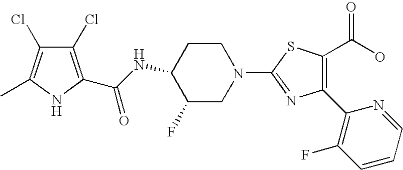 | (dd, 1 H); 4.01 (m, 1 H); 4.32 (m,2 H); 4.89~5.05 (d, 1 H); 7.32 (d,1 H); 7.54 (dd, 1 H); 7.76 (t, 1 H);8.46 (d, 1 H); 12.13 (s, 1 H);12.88 (br, 1 H). | |
| 120 | 2-((3S,4R)-4-(4-chloro-3-cyano-5- | MS (ES): 505 (MH⁺) for | Example 103 |
| | methyl-1H-pyrrole-2-carboxamido)- | C₂₀H₂₁ClN₈O₄S | |
| | 3-methoxypiperidin-1-yl)-4-(1- | ¹H-NMR: 1.74 (m, 2 H); 2.21 (s, | |
| | methyl-1H-1,2,3-triazol-4-yl)thiazole- | 3 H); 3.33 (m, 2 H); 3.50 (s, 3 H); | |
| | 5-carboxylic acid | 3.58 (m, 1 H); 4.01 (m, 1 H); 4.15 | |
| | 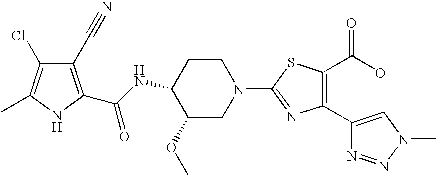 | (s, 3 H); 4.28 (m, 2 H); 7.98 (d,1 H); 8.70 (s, 1 H); 13.03 (s, 1 H);14.20 (br, 1 H). | |
| 121 | 2-((3S,4R)-4-(4-chloro-3-cyano-5- | MS (ES): 506 (MH⁺) for | Example 104 |
| | methyl-1H-pyrrole-2-carboxamido)- | C₂₀H₂₁ClN₈O₄S | |
| | 3-methoxypiperidin-1-yl)-4-(1- | ¹H-NMR DMSO-D6 (DMSO-d₆) | |
| | methyl-1H-1,2,4-triazol-5-yl)thiazole- | δ: 1.77 (m, 2 H); 2.21 (s, 3 H); | |
| | 5-carboxylic acid | 3.33 (m, 2 H); 3.50 (s, 3 H); | |
| | 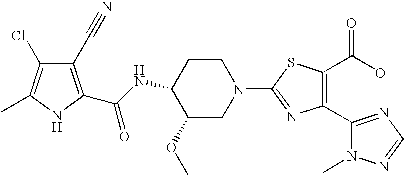 | 3.58 (m, 1 H); 4.01 (m, 1 H); 4.08(s, 3 H); 4.28 (m, 2 H); 8.05 (d,1 H); 8.25 (s, 1 H); 13.16 (s, 1 H);15.47 (br, 1 H). | |
| 122 | 2-((3S,4R)-4-(4-chloro-3-cyano-5- | MS (ES): 503 (MH⁺) for | Example 105 |
| | methyl-1H-pyrrole-2-carboxamido)- | C₂₁H₂₀ClN₇O₄S | |
| | 3-methoxypiperidin-1-yl)-4- | ¹H-NMR: 1.76 (m, 2 H); 2.20 (s, | |
| | (pyrimidin-2-yl)thiazole-5-carboxylic | 3 H); 3.33 (m, 2 H); 3.50 (s, 3 H); | |
| | acid | 3.60 (m, 1 H); 3.99 (m, 1 H); 4.28 | |
| | 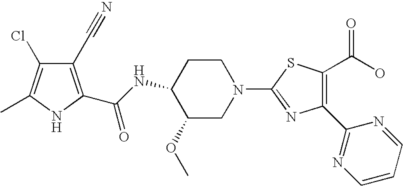 | (m, 2 H); 7.94 (d, 1 H); 8.78 (s,1 H); 8.83 (s, 1 H); 9.35 (s, 1 H);12.30 (s, 1 H); 15.28 (br, 1 H). | |
| 123 | 2-((3S,4R)-4-(4-chloro-3-cyano-5- | MS (ES): 520 (MH⁺) for | Example 106 |
| | methyl-1H-pyrrole-2-carboxamido)- | C₂₂H₂₀FClN₆O₄5 | |
| | 3-methoxypiperidin-1-yl)-4-(3- | ¹H-NMR: 1.75 (m, 2 H); 2.21 (s, | |
| | fluoropyridin-2-yl)thiazole-5- | 3 H); 3.33 (m, 2 H); 3.36 (s, 3 H); | |
| | carboxylic acid | 3.58 (m, 1 H); 3.98 (m, 1 H); 4.26 | |
| | 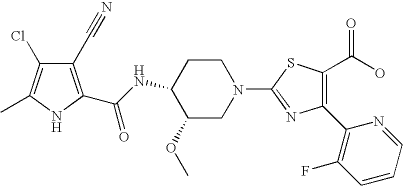 | (m, 2 H); 7.54 (m, 1 H); 7.78 (m,2 H); 8.46 (d, 1 H); 12.76 (br,1 H). | |
| 124 | 2-((3S,4R)-4-(4-chloro-3-cyano-5- | MS (ES): 517 (MH⁺) for | Example 107 |
| | methyl-1H-pyrrole-2-carboxamido)- | C₂₂H₂₂ClN₇O₄S | |
| | 3-methoxypiperidin-1-yl)-4-(5- | ¹H-NMR: 1.75 (m, 2 H); 2.21 (s, | |
| | methylpyrazin-2-yl)thiazole-5- | 3 H); 2.60 (s, 3 H); 3.33 (m, 2 H); | |
| | carboxylic acid | 3.35 (s, 3 H); 3.50 (m, 1 H); 3.98 | |
| | 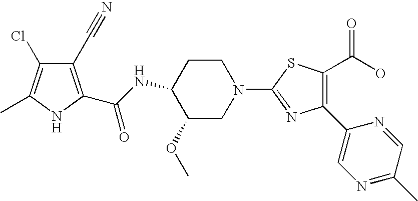 | (m, 1 H); 4.28 (m, 2 H); 7.85 (d,1 H); 8.62 (s, 1 H); 9.29 (s, 1 H);12.84 (s, 1 H); 15.77 (br, 1 H). | |
| 125 | 2-((3S,4R)-4-(4-chloro-3-cyano-5- | MS (ES): 505 (MH⁺) for | Example 108 |
| | methyl-1H-pyrrole-2-carboxamido)- | C₂₁H₂₂ClN₇O₄S | |
| | 3-methoxypiperidin-1-yl)-4-(1- | ¹H-NMR: 1.75 (m, 2 H); 2.21 (s, | |
| | methyl-1H-imidazol-4-yl)thiazole-5- | 3 H); 3.33 (m, 2 H); 3.36 (s, 3 H); | |
| | carboxylic acid | 3.58 (m, 1 H); 3.82 (s, 3 H); 3.98 | |
| | 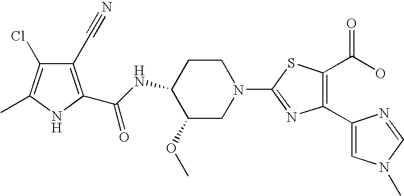 | (m, 1 H); 4.26 (m, 2 H); 7.82 (d,1 H); 7.93 (s, 1 H); 8.37 (s, 1 H);12.76 (br, 1 H). | |

### Examples 126-140

The following compounds were synthesized according to the procedure described for Example 43.

| Ex | Compound | Data | SM |
|---|---|---|---|
| 126 | Methyl 2-[(3S,4R)-3-(benzyloxy)-4- | MS (ES): 604. (M + H). | Intermediate |
| | {[(3,4-dichloro-5-methyl-1H-pyrrol- | ¹H NMR: 1.84 (m, 2 H), 2.18 (s, | 103 and |
| | 2-yl)carbonyl]amino}piperidin-1-yl]- | 3 H), 3.34 (m, 3 H), 3.72 (s, 6 H), | Intermediate 9 |
| | 4-(1-methyl-1H-1,2,4-triazol-5-yl)- | 4.38 (br s, 1 H), 4.53 (d, 1 H), | |
| | 1,3-thiazole-5-carboxylate | 4.76 (d, 1 H), 7.13 (d, 1 H), 7.32 | |
| | 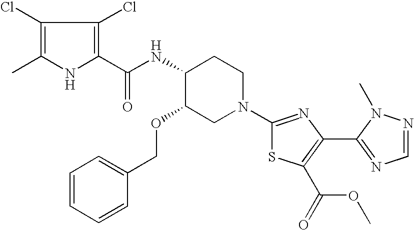 | (br s, 5 H), 8.02 (s, 1 H), 12.18 (s,1 H). | |
| 127 | Methyl 2-[(3S,4R)-3-(benzyloxy)-4- | ¹H NMR: 1.82 (br s, 2 H), 2.17 | Intermediate |
| | {[(3,4-dichloro-5-methyl-1H-pyrrol- | (s, 3 H), 3.13 (s, 3 H), 3.31 (m, | 103 and |
| | 2-yl)carbonyl]amino}piperidin-1-yl]- | 1 H), 3.46 (t, 3 H), 3.61 (s, 3 H), | Intermediate 8 |
| | 4-[1-(2-methoxyethyl)-1H-imidazol- | 3.82 (s, 1 H), 3.97 (t, 3 H), 4.32 | |
| | 2-yl]-1,3-thiazole-5-carboxylate | (m, 2 H), 4.48 (d, 1 H), 4.72 (d, | |
| | 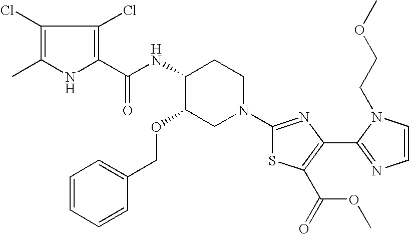 | 1 H), 6.94 (s, 2 H), 7.06 (d, 1 H),7.28 (s, 5 H), 12.09 (s, 2 H). | |
| 128 | Ethyl 2-[(3S,4R)-3-(benzyloxy)-4- | MS (ES): 632 (M + H); | Intermediate |
| | {[(3,4-dichloro-5-methyl-1H-pyrrol- | ¹H NMR: 1.15 (t, 3 H), 1.87 (m, | 103 and |
| | 2-yl)carbonyl]amino}piperidin-1-yl]- | 2 H), 2.19 (s, 3 H), 3.47 (m, 2 H), | Intermediate |
| | 4-(3-fluoropyridin-2-yl)-1,3-thiazole- | 3.87 (s, 1 H), 4.08 (q, 2 H), 4.38 | 86 |
| | 5-carboxylate | (m, 2 H), 4.49 (d, 1 H), 4.74 (d, | |
| | 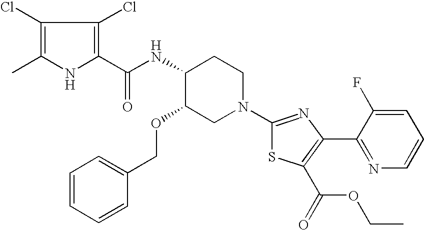 | 1 H), 7.15 (d, 1 H), 7.26 (s, 5 H),7.64 (d, 1 H), 7.78 (m, 1 H), 8.48(s, 1 H), 12.18 (s, 1 H). | |
| 129 | Ethyl 2-[(3S,4R)-3-(benzyloxy)-4- | MS (ES): 615 (M + H); ¹H NMR: | Intermediate |
| | {[(3,4-dichloro-5-methyl-1H-pyrrol- | 1.15 (t, 3 H), 1.86 (m, 2 H), 2.21 (s, | 103 and |
| | 2-yl)carbonyl]amino}piperidin-1-yl]- | 3 H), 3.45 (d, 2 H), 3.84 (s, 1 H), 4.13 | Intermediate 6 |
| | 4-(pyrazin-2-yl)-1,3-thiazole-5- | (q, 2 H), 4.36 (m, 2 H), 4.55 (d, 1 H), | |
| | carboxylate | 4.79 (d, 1 H), 7.16 (d, 1 H), 7.25 (s, | |
| | 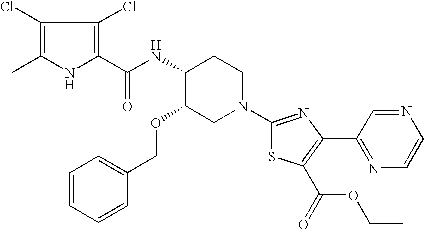 | 5 H), 8.65 (d, 1 H), 8.72 (d, 1 H),8.75 (s, 1 H), 12.16 (s, 1 H). | |
| 130 | Ethyl 2-[(3S,4R)-3-(benzyloxy)-4- | ¹H NMR: 1.12 (t, 3 H), 1.85 (d, | Intermediate |
| | {[(3,4-dichloro-5-methyl-1H-pyrrol- | 2 H), 2.18 (s, 3 H), 3.34 (d, 1 H), | 103 and |
| | 2-yl)carbonyl]amino}piperidin-1-yl]- | 3.84 (s, 1 H), 4.14 (q, 2 H), 4.36 | Intermediate |
| | 4-(1H-tetrazol-5-yl)-1,3-thiazole-5- | (d, 1 H), 4.51 (d, 1 H), 4.73 (d, | 112 |
| | carboxylate | 1 H), 7.08 (d, 1 H), 7.20-7.38 (m, | |
| | 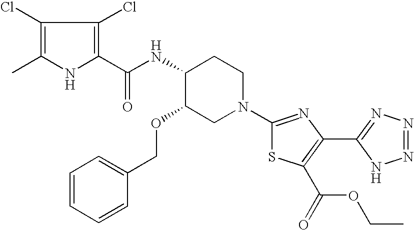 | 5 H), 12.09 (s, 1 H). | |
| 131 | Methyl 2-[(3S,4R)-3-(benzyloxy)-4- | ¹H NMR: 1.68 (d, 2 H), 2.19 (s, | Intermediate |
| | {[(4-chloro-5-methyl-1H-pyrrol-2- | 3 H), 3.72 (m, 6 H), 3.21 (s, 2 H), | 104 and |
| | yl)carbonyl]amino}piperidin-1-yl]-4- | 4.26 (m, 2 H), 4.51 (d, 1 H), 4.68 | Intermediate 9 |
| | (1-methyl-1H-1,2,4-triazol-5-yl)-1,3- | (d, 1 H), 6.92 (s, 1 H), 7.21 (d, | |
| | thiazole-5-carboxylate | 3 H), 7.31 (d, 2 H), 7.74 (d, 1 H), | |
| | 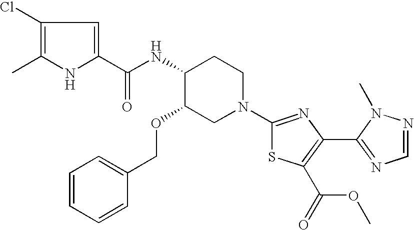 | 8.12 (s, 1 H), 11.62 (s, 2 H). | |
| 132 | Ethyl 2-[(3S,4R)-3-(benzyloxy)-4- | ¹H NMR: 1.12 (t, 3 H), 1.85 (d, | Intermediate |
| | {[(4-chloro-5-methyl-1H-pyrrol-2- | 2 H), 2.18 (s, 3 H), 3.34 (d, 1 H), | 104 and |
| | yl)carbonyl]amino}piperidin-1-yl]-4- | 3.84 (s, 1 H), 4.14 (q, 2 H), 4.36 | Intermediate |
| | (1H-tetrazol-5-yl)-1,3-thiazole-5- | (d, 1 H), 4.51 (d, 1 H), 4.73 (d, | 112 |
| | carboxylate | 1 H), 6.88 (s, 1 H), 7.20-7.38 (m, | |
| | 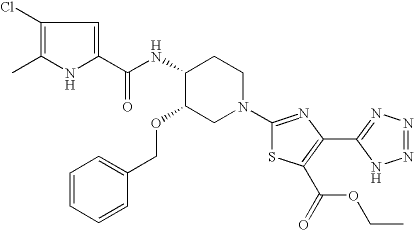 | 5 H), 7.72 (d, 1 H), 12.09 (s, 1 H).MS (ES): 571 (M + H). | |
| 133 | Methyl 2-[(3S,4R)-3-(benzyloxy)-4- | ¹H NMR: 1.82 (br s, 2 H), 2.17 | Intermediate |
| | {[(4-chloro-5-methyl-1H-pyrrol-2- | (s, 3 H), 3.13 (s, 3 H), 3.31 (m, | 104 and |
| | yl)carbonyl]amino}piperidin-1-yl]-4- | 1 H), 3.46 (t, 3 H), 3.61 (s, 3 H), | Intermediate 8 |
| | [1-(2-methoxyethyl)-1H-imidazol-2- | 3.82 (s, 1 H), 3.97 (t, 3 H), 4.32 | |
| | yl]-1,3-thiazole-5-carboxylate | (m, 2 H), 4.48 (d, 1 H), 4.72 (d, | |
| | 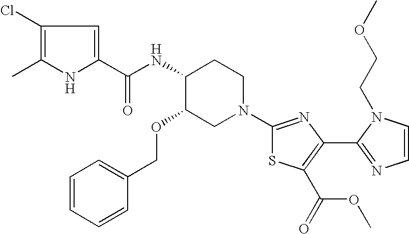 | 1 H), 6.94 (s, 2 H), 6.95 (s, 1 H),7.25 (m, 2 H), 7.28 (m, 3 H), 7.71(d, 1 H), 11.60 (s, 1 H). | |
| 134 | Ethyl 2-[(3S,4R)-3-(benzyloxy)-4- | ¹H NMR: 1.05 (t, 3 H), 1.33 (m, | Intermediate |
| | {[(4-chloro-5-methyl-1H-pyrrol-2- | 1 H), 1.69 (m, 1 H), 2.19 (s, 3 H), | 104 and |
| | yl)carbonyl]amino}piperidin-1-yl]-4- | 2.71 (s, 1 H), 3.45 (m, 2 H), 3.80 | Intermediate |
| | (3-fluoropyridin-2-yl)-1,3-thiazole-5- | (s, 1 H), 4.06 (q, 2 H), 4.27 (d, | 86 |
| | carboxylate | 2 H), 4.51 (d, 1 H), 6.85 (s, 1 H), | |
| | 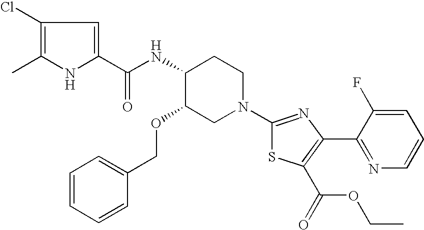 | 7.27 (d, 5 H), 7.53 (m, 1 H), 7.72(d, 1 H), 7.81 (t, 1 H), 8.51 (s,1 H), 11.61 (s, 1 H). | |
| 135 | Ethyl 2-[(3S,4R)-3-(benzyloxy)-4- | ¹H NMR: 1.14 (t, 3 H), 1.64 (m, | Intermediate |
| | {[(4-chloro-5-methyl-1H-pyrrol-2- | 1 H), 1.95 (m, 1 H), 2.19 (s, 3 H), | 104 and |
| | yl)carbonyl]amino}piperidin-1-yl]-4- | 2.71 (s, 1 H), 3.42 (d, 2 H), 3.80 | Intermediate 6 |
| | (pyrazin-2-yl)-1,3-thiazole-5- | (s, 1 H), 4.15 (q, 2 H), 4.35 (m, | |
| | carboxylate | 3 H), 4.48 (d, 1 H), 4.56 (d, 1 H), | |
| | 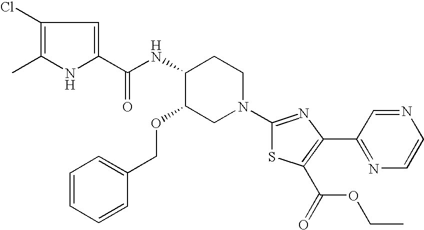 | 6.85 (s, 1 H), 7.24 (d, 5 H), 7.76(d, 1 H), 8.75 (t, 3 H), 11.60 (s,1 H). | |
| 136 | Methyl 2-[(3S,4R)-3-(benzyloxy)-4- | ¹H NMR: 1.86 (m, 2 H), 2.21 (s, | Intermediate |
| | {[(4-chloro-3-cyano-5-methyl-1H- | 3 H), 3.58 (m, 4 H), 3.72 (s, 6 H), | 105 and |
| | pyrrol-2-yl)carbonyl]amino}piperidin-1- | 3.85 (s, 1 H), 4.38 (br s, 1 H), | Intermediate 9 |
| | yl]-4-(1-methyl-1H-1,2,4-triazol-5-yl)- | 4.59 (d, 1 H), 4.67 (d, 1 H), 7.19 | |
| | 1,3-thiazole-5-carboxylate | (s, 3 H), 7.28 (s, 2 H), 7.74 (d, | |
| | 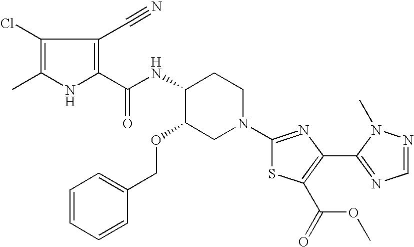 | 1 H), 8.01 (s, 1 H), 12.58 (s, 1 H). | |
| 137 | Ethyl2-[(3S,4R)-3-(benzyloxy)-4- | ¹H NMR: 1.12 (t, 3 H), 1.85 (d, | Intermediate |
| | {[(4-chloro-3-cyano-5-methyl-1H- | 2 H), 2.18 (s, 3 H), 3.34 (d, 1 H), | 105 and |
| | pyrrol-2- | 3.84 (s, 1 H), 4.14 (q, 2 H), 4.36 | Intermediate |
| | yl)carbonyl]amino}piperidin-1-yl]-4- | (d, 1 H), 4.51 (d, 1 H), 4.73 (d, | 112 |
| | (1H-tetrazol-5-yl)-1,3-thiazole-5- | 1 H), 7.20-7.38 (m, 5 H), 7.72 (d, | |
| | carboxylate | 1 H), 12.09 (s, 1 H). | |
| | 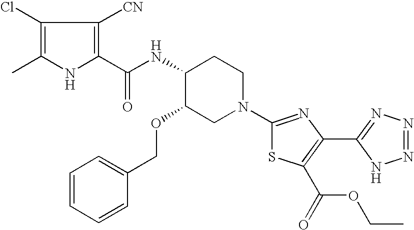 | | |
| 138 | Methyl 2-[(3S,4R)-3-(benzyloxy)-4- | ¹H NMR: 1.82 (br s, 2 H), 2.17 | Intermediate |
| | {[(4-chloro-3-cyano-5-methyl-1H- | (s, 3 H), 3.13 (s, 3 H), 3.31 (m, | 105 and |
| | pyrrol-2-yl)carbonyl]amino}piperidin- | 1 H), 3.46 (t, 3 H), 3.61 (s, 3 H), | Intermediate 8 |
| | 1-yl]-4-[1-(2-methoxyethyl)-1H- | 3.82 (s, 1 H), 3.97 (t, 3 H), 4.32 | |
| | imidazol-2-yl]-1,3-thiazole-5- | (m, 2 H), 4.48 (d, 1 H), 4.72 (d, | |
| | carboxylate | 1 H), 6.94 (s, 2 H), 7.18 (s, 2 H), | |
| | 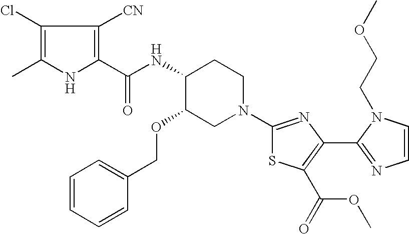 | 7.25 (d, 3 H), 7.63 (d, 1 H), 12.60(s, 1 H). | |
| 139 | Ethyl 2-[(3S,4R)-3-(benzyloxy)-4- | ¹H NMR: 1.05 (t, 3 H), 1.78 (m, | Intermediate |
| | {[(4-chloro-3-cyano-5-methyl-1H- | 1 H), 1.91 (m, 1 H), 2.23 (s, 3 H), | 105 and |
| | pyrrol-2- | 3.43 (m, 2 H), 3.83 (s, 1 H), 4.08 | Intermediate |
| | yl)carbonyl]amino}piperidin-1-yl]-4- | (q, 2 H), 4.23 (d, 2 H), 4.45 (d, | 86 |
| | (3-fluoropyridin-2-yl)-1,3-thiazole-5- | 1 H), 4.63 (d, 1 H), 7.19 (s, 3 H), | |
| | carboxylate | 7.21 (s, 2 H), 7.52 (t, 1 H), 7.64 | |
| | 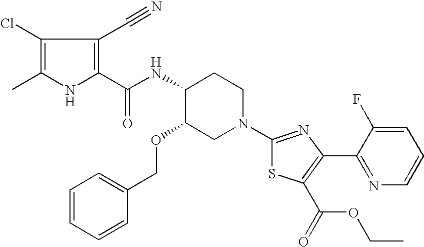 | (m, 1 H), 7.82 (t, 1 H), 8.52 (s,1 H), 12.60 (s, 1 H). | |
| 140 | Ethyl 2-[(3S,4R)-3-(benzyloxy)-4- | ¹H N NMR: 1.09 (t, 3 H), 1.79 (d, | Intermediate |
| | {[(4-chloro-3-cyano-5-methyl-1H- | 1 H), 2.00 (d, 1 H), 2.23 (s, 3 H), | 105 and |
| | pyrrol-2- | 3.43 (d, 2 H), 3.84 (br s, 1 H), | Intermediate 6 |
| | yl)carbonyl]amino}piperidin-1-yl]-4- | 4.05 (q, 2 H), 4.26 (m, 2 H), 4.53 | |
| | (pyrazin-2-yl)-1,3-thiazole-5- | (d, 1 H), 4.74 (d, 1 H), 7.18 (s, | |
| | carboxylate | 3 H), 7.21 (s, 2 H), 7.65 (br s, | |
| | 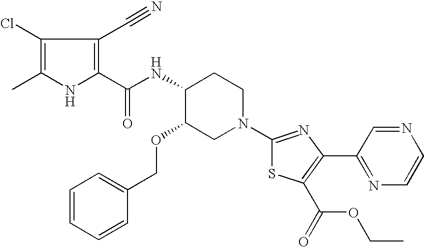 | 1 H), 8.79 (m, 3 H), 12.61 (s, 1 H). | |

### Examples 141-155

The following compounds were synthesized according to the procedure described for Example 1.

| Ex | Compound | Data | SM |
|---|---|---|---|
| 141 | 2-[(3S,4R)-3-(benzyloxy)-4-{[(3,4- | ¹H NMR: 1.79 (m, 1 H), 2.05 (m, | Example 126 |
| | dichloro-5-methyl-1H-pyrrol-2- | 1 H), 2.21 (s, 3 H), 3.55 (m, 1 H), | |
| | yl)carbonyl]amino}piperidin-1-yl]-4- | 3.81 (s, 1 H), 4.13 (s, 3 H), 4.38 | |
| | (1-methyl-1H-1,2,4-triazol-5-yl)-1,3- | (br s, 1 H), 4.58 (d, 1 H), 4.72 (d, | |
| | thiazole-5-carboxylic acid | 1 H), 7.18 (s, 3 H), 7.29 (s, 3 H), | |
| | 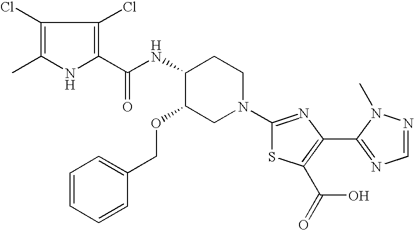 | 7.29 (s, 3 H), 7.97 (m, 1 H), 8.31(s, 1 H), 12.96 (br s, 1 H). | |
| 142 | 2-[(3S,4R)-3-(benzyloxy)-4-{[(3,4- | ¹H NMR: δ 1.85 (d, 2 H), 2.18 (s, | Example 130 |
| | dichloro-5-methyl-1H-pyrrol-2- | 3 H), 3.34 (d, 1 H), 3.84 (s, 1 H), | |
| | yl)carbonyl]amino}piperidin-1-yl]-4- | 4.36 (d, 1 H), 4.51 (d, 1 H), 4.73 | |
| | (1H-tetrazol-5-yl)-1 ,3-thiazole-5- | (d, 1 H), 7.08 (d, 1 H), 7.20-7.38 | |
| | carboxylic acid | (m, 5 H), 12.09 (s, 1 H). | |
| | 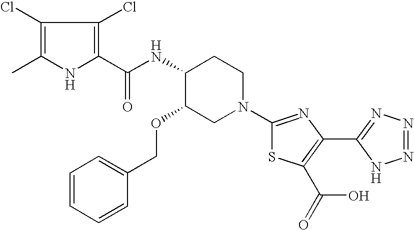 | | |
| 143 | 2-[(3S,4R)-3-(benzyloxy)-4-{[(3,4- | ¹H NMR: 1.82 (br s, 2 H), 2.17 | Example 127 |
| | dichloro-5-methyl-1H-pyrrol-2- | (s, 3 H), 3.31 (m, 1 H), 3.46 (t, | |
| | yl)carbonyl]amino}piperidin-1-yl]-4- | 3 H), 3.61 (s, 3 H), 3.82 (s, 1 H), | |
| | [1-(2-methoxyethyl)-1H-imidazol-2- | 3.97 (t, 3 H), 4.32 (m, 2 H), 4.48 | |
| | yl]-1,3-thiazole-5-carboxylic acid | (d, 1 H), 4.72 (d, 1 H), 6.94 (s, | |
| | 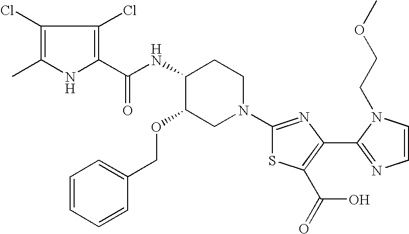 | 2 H), 7.06 (d, 1 H), 7.28 (s, 5 H),12.09 (s, 2 H). | |
| 144 | 2-[(3S,4R)-3-(benzyloxy)-4-{[(3,4- | ¹H NMR: 1.81 (m, 2 H), 2.19 (s, | Example 128 |
| | dichloro-5-methyl-1H-pyrrol-2- | 3 H), 3.42 (m, 2 H), 3.81 (s, 1 H), | |
| | yl)carbonyl]amino}piperidin-1-yl]-4- | 3.95 (m, 1 H), 4.38 (m, 2 H), 4.47 | |
| | (3-fluoropyridin-2-yl)-1,3-thiazole-5- | (d, 1 H), 4.76 (d, 1 H), 7.13 (d, | |
| | carboxylic acid | 1 H), 7.26 (s, 5 H), 7.59 (d, 1 H), | |
| | 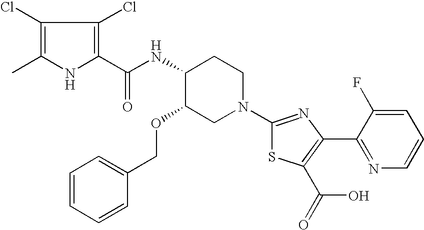 | 7.85 (m, 1 H), 8.49 (s, 1 H), 12.12(s, 1 H), 12.84 (brs, 1 H). | |
| 145 | 2-[(3S,4R)-3-(benzyloxy)-4-{[(3,4- | ¹H NMR: 1.82 (m, 2 H), 2.18 s, | Example 129 |
| | dichloro-5-methyl-1H-pyrrol-2- | 3 H), 3.43 (m, 4 H), 3.82 (s, 1 H), | |
| | yl)carbonyl]amino}piperidin-1-yl]-4- | 4.38 (br s, 1 H), 4.51 (d, 1 H), | |
| | (pyrazin-2-yl)-1,3-thiazole-5- | 4.76 (d, 1 H), 7.23 (s, 5 H), 7.13 | |
| | carboxylic acid | (m, 1 H), 8.83 (d, 2 H), 9.24 (s, | |
| | 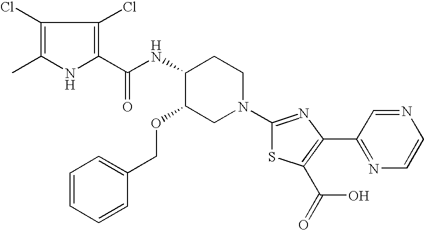 | 1 H), 12.01 (s, 1 H). | |
| 146 | 2-[(3S,4R)-3-(benzyloxy)-4-{[(4- | ¹H NMR: 1.72 (d, 1 H), 2.10 (d, | Example 131 |
| | chloro-5-methyl-1H-pyrrol-2- | 1 H), 2.22 (s, 3 H), 3.45 (d, 2 H), | |
| | yl)carbonyl]amino}piperidin-1-yl]-4- | 3.84 (s, 1 H), 4.16 (s, 3 H), 4.31 | |
| | (1-methyl-1H-1,2,4-triazol-5-yl)-1,3- | (br s, 2 H), 4.57 (d, 1 H), 4.65 (d, | |
| | thiazole-5-carboxylic acid | 1 H), 6.95 (s, 1 H), 7.14 (d, 3 H), | |
| | 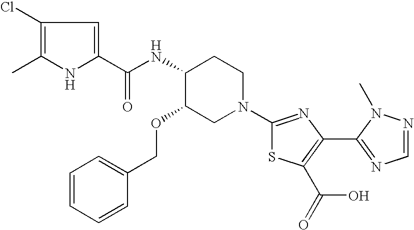 | 7.28 (d, 2 H), 7.76 (d, 1 H), 8.28(s, 1 H), 11.65 (s, 1 H), 15.81 (s,1 H). | |
| 147 | 2-[(3S,4R)-3-(benzyloxy)-4-{[(4- | ¹H NMR: 1.85 (d, 2 H), 2.18 (s, | Example 132 |
| | chloro-5-methyl-1H-pyrrol-2- | 3 H), 3.34 (d, 1 H), 3.84 (s, 1 H), | |
| | yl)carbonyl]amino}piperidin-1-yl]-4- | 4.36 (d, 1 H), 4.51 (d, 1 H), 4.73 | |
| | (1H-tetrazol-5-yl)-1,3-thiazole-5- | (d, 1 H), 6.88 (s, 1 H), 7.20-7.38 | |
| | carboxylic acid | (m, 5 H), 7.72 (d, 1 H), 12.09 (s, | |
| | 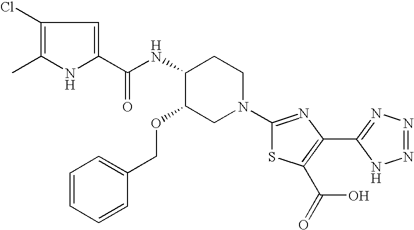 | 1 H). | |
| 148 | 2-[(3S,4R)-3-(benzyloxy)-4-{[(4- | ¹H NMR: 1.82 (br s, 2 H), 2.17 | Example 133 |
| | chloro-5-methyl-1H-pyrrol-2- | (s, 3 H), 3.31 (m, 1 H), 3.46 (t, | |
| | yl)carbonyl]amino}piperidin-1-yl]-4- | 3 H), 3.61 (s, 3 H), 3.82 (s, 1 H), | |
| | [1-(2-methoxyethyl)-1H-imidazol-2- | 3.97 (t, 3 H), 4.32 (m, 2 H), 4.48 | |
| | yl]-1,3-thiazole-5-carboxylic acid | (d, 1 H), 4.72 (d, 1 H), 6.94 (s, | |
| | 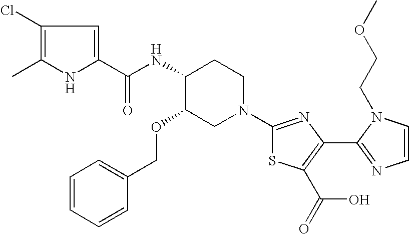 | 2 H), 6.95 (s, 1 H), 7.25 (m, 2 H),7.28 (m, 3 H), 7.71 (d, 1 H) 11.60(s, 1 H). | |
| 149 | 2-[(3S,4R)-3-(benzyloxy)-4-{[(4- | ¹H NMR: 1.67 (d, 2 H), 2.18 (s, | Example 134 |
| | chloro-5-methyl-1H-pyrrol-2- | 3 H), 3.45 (m, 3 H), 3.79 (s, 1 H), | |
| | yl)carbonyl]amino}piperidin-1-yl]-4- | 4.02 (m, 1 H), 4.38 (m, 2 H), 4.51 | |
| | (3-fluoropyridin-2-yl)-1,3-thiazole-5- | (d, 1 H), 4.71 (d, 1 H), 6.91 (s, | |
| | carboxylic acid | 1 H), 7.31 (m, 5 H), 7.59 (m, 1 H), | |
| | 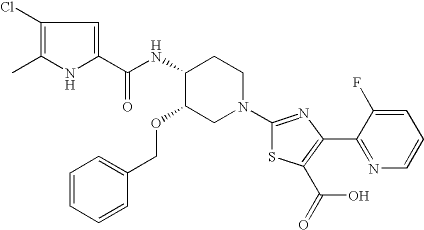 | 7.81 (m, 3 H), 8.51 (s, 1 H), 11.61(s, 1 H), 12.82 (m, 1 H). | |
| 150 | 2-[(3S,4R)-3-(benzyloxy)-4-{[(4- | ¹H NMR: 1.72 (d, 2 H), 2.21 (s, | Example 135 |
| | chloro-5-methyl-1H-pyrrol-2- | 3 H), 3.45 (m, 3 H), 3.84 (s, 1 H), | |
| | yl)carbonyl]amino}piperidin-1-yl]-4- | 4.02 (br s, 1 H), 4.31 (m, 1 H), | |
| | (pyrazin-2-yl)-1,3-thiazole-5- | 4.55 (d, 1 H), 4.75 (d, 1 H), 6.93 | |
| | carboxylic acid | (s, 1 H), 7.21 (d, 5 H), 7.72 (d, | |
| | 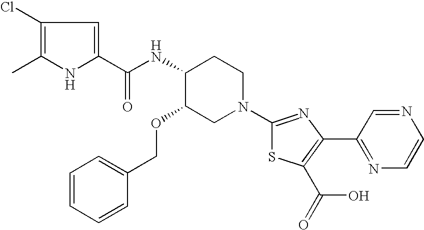 | 1 H), 8.81 (d, 2 H), 9.41 (s, 1 H),11.61 (s, 1 H), 15.39 (s, 1 H). | |
| 151 | 2-[(3S,4R)-3-(benzyloxy)-4-{[(4- | ¹H NMR: 1.86 (m, 2 H), 2.21 (s, | Example 136 |
| | chloro-3-cyano-5-methyl-1H-pyrrol- | 3 H), 3.52 (m, 4 H), 3.79 (s, 3 H), | |
| | 2-yl)carbonyl]amino}piperidin-1-yl]- | 3.94 (m, 1 H), 4.39 (br s, 1 H), | |
| | 4-(1-methyl-1H-1,2,4-triazol-5-yl)- | 4.47 (d, 1 H), 4.78 (d, 1 H), 7.19 | |
| | 1,3-thiazole-5-carboxylic acid | (d, 1 H), 7.27 (d, 5 H), 7.95 (s, | |
| | 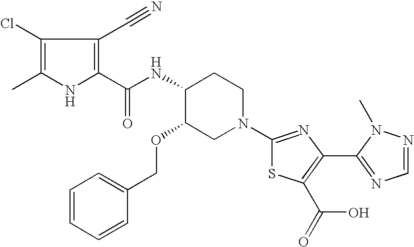 | 1 H). | |
| 152 | 2-[(3S,4R)-3-(benzyloxy)-4-{[(4- | ¹H NMR: 1.85 (d, 2 H), 2.18 (s, | Example 137 |
| | chloro-3-cyano-5-methyl-1H-pyrrol- | 3 H), 3.34 (d, 1 H), 3.84 (s, 1 H), | |
| | 2-yl)carbonyl]amino}piperidin-1-yl]- | 4.36 (d, 1 H), 4.51 (d, 1 H), 4.73 | |
| | 4-(1H-tetrazol-5-yl)-1,3-thiazole-5- | (d, 1 H), 7.20-7.38 (m, 5 H), 7.72 | |
| | carboxylic acid | (d, 1 H), 12.09 (s, 1 H). | |
| | 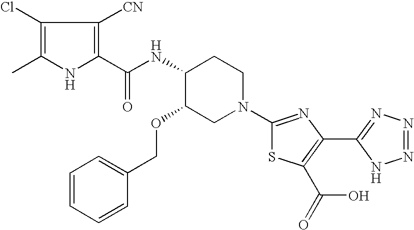 | | |
| 153 | 2-[(3S,4R)-3-(benzyloxy)-4- | ¹H NMR: 1.82 (br s, 2 H), 2.17 | Example 138 |
| | {[(4chloro-3-cyano-5-methyl-1H- | (s, 3 H), 3.31 (m, 1 H), 3.46 (t, | |
| | pyrrol-2-yl)carbonyl]amino}piperidin- | 3 H), 3.61 (s, 3 H), 3.82 (s, 1 H), | |
| | 1-yl]-4-[1-(2-methoxyethyl)-1H- | 3.97 (t, 3 H), 4.32 (m, 2 H), 4.48 | |
| | imidazol-2-yl[-1,3-thiazole-5- | (d, 1 H), 4.72 (d, 1 H), 6.94 (s, | |
| | carboxylic acid | 2 H), 7.18 (s, 2 H), 7.25 (d, 3 H), | |
| | 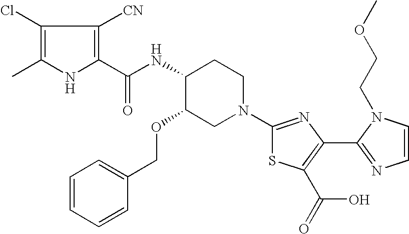 | 7.63 (d, 1 H), 12.60 (s, 1 H). | |
| 154 | 2-[(3S,4R)-3-(benzyloxy)-4-{[(4- | ¹H NMR: 1.72 (m, 1 H), 1.92 (m, | Example 139 |
| | chloro-3-cyano-5-methyl-1H-pyrrol- | 1 H), 2.24 (s, 3 H), 3.45 (m, 2 H), | |
| | 2-yl)carbonyl]amino}piperidin-1-yl]- | 3.81 (s, 1 H), 4.03 (br s, 1 H), | |
| | 4-(3-fluoropyridin-2-yl)-1,3-thiazole- | 4.32 (m, 2 H), 4.54 (d, 1 H), 4.63 | |
| | 5-carboxylic acid | (d, 1 H), 7.18 (d, 3 H), 7.23 (d, | |
| | 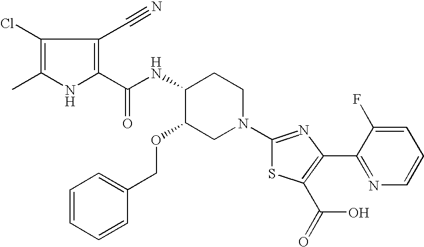 | 2 H), 7.52 (m, 1 H), 7.87 (m, 2 H),8.49 (d, 1 H), 12.73 (s, 1 H). | |
| 155 | 2-[(3S,4R)-3-(benzyloxy)-4-{[(4- | ¹H NMR: 1.84 (d, 1 H), 2.03 (m, | Example 140 |
| | chloro-3-cyano-5-methyl-1H-pyrrol- | 1 H), 2.21 (s, 3 H), 3.45 (m, 2 H), | |
| | 2-yl)carbonyl]amino}piperidin-1-yl]- | 3.84 (s, 1 H), 4.12 (m, 1 H), 4.32 | |
| | 4-(pyrazin-2-yl)-1,3-thiazole-5- | (m, 2 H), 4.56 (d, 1 H), 4.65 (d, | |
| | carboxylic acid | 1 H), 7.15 (s, 3 H), 7.25 (s, 2 H), | |
| | 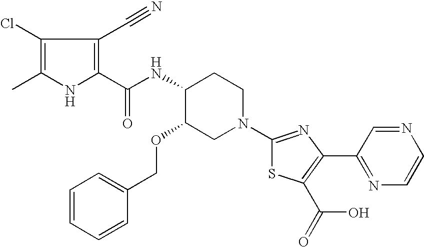 | 7.94 (d, 1 H), 8.87 (d, 2 H), 9.25(s, 1 H), 12.97 (s, 1 H). | |

### Examples 156-158

The following compounds were synthesized according to the procedure described for Example 43.

| Ex | Compound | Data | SM |
|---|---|---|---|
| 156 | Methyl 2-[(3S,4R)-4-{[(3,4-dichloro [symbol] | ¹H NMR (400 MHz, DMSO-d₆): | Intermediate |
| | methyl-1H-pyrrol-2-yl)carbonyl]am [symbol] | 0.81 (t, 3 H), 1.47 (m, 2 H), 1.79 (br | 115 and |
| | 3-propoxypiperidin-1-yl]-4-(1-meth [symbol] | s, 2 H), 2.18 (s, 3 H), 3.44 (m, 3 H), | Intermediate 9 |
| | 1,2,4-triazol-5-yl)-1,3-thiazole-5- | 3.58 (q, 2 H), 3.65 (s, 3 H), 3.71 (s, | |
| | carboxylate | 3 H), 3.91 (br s, 1 H), 4.29 (m, 2 H), | |
| | 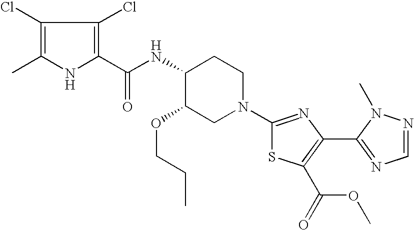 | 7.10 (d, 1 H), 8.00 (s, 1 H), 12.04 (s,1H). | |
| 157 | Ethyl 2-[(3S,4R)-4-{[(3,4- | ¹H NMR (400 MHz, DMSO-d₆): | Intermediate |
| | dichloro-5-methyl-1H-pyrrol-2- | 0.80 (t, 3 H), 1.04 (t, 3 H), 1.45 (m, | 115 and |
| | yl)carbonyl]amino}-3- | 2 H), 1.78 (br s, 2 H), 2.18 (s, 3 H), | Intermediate |
| | propoxypiperidin-1-yl]-4-(3- | 3.37 (m, 2 H), 3.60 (t, 2 H), 3.64 (br | 86 |
| | fluoropyridin-2-yl)-1,3-thiazole-5- | s, 1 H), 4.05 (q, 2 H), 4.29 (m, 2 H), | |
| | carboxylate | 7.09 (d, 1 H), 7.56 (m, 1 H), 7.80 (t, | |
| | 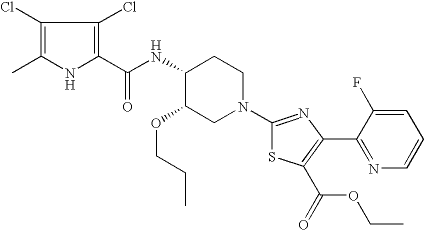 | 1 H), 8.45 (s, 1 H), 12.14 (s, 1 H). | |
| 158 | Ethyl 2-[(3S,4R)-4-{[(3,4- | ¹H NMR (400 MHz, DMSO-d₆): | Intermediate |
| | dichloro-5-methyl-1H-pyrrol-2- | 0.80 (t, 3 H), 1.04 (t, 3 H), 1.45 (m, | 115 and |
| | yl)carbonyl]amino}-3- | 2 H), 1.78 (br s, 2 H), 2.18 (s, 3 H), | Intermediate 6 |
| | propoxypiperidin-1-yl]-4-(pyrazin- | 3.39 (m, 5 H), 4.10 (q, 2 H), 4.25 (br | |
| | 2-yl)-1,3-thiazole-5-carboxylate | s, 2 H), 7.11 (d, 1 H), 8.69 (d, 2 H), | |
| | 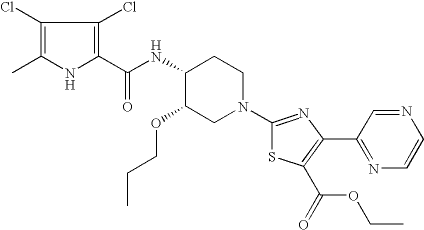 | 8.81 (s, 1 H), 12.14 (s, 1 H). | |

[symbol]

indicates data missing or illegible when filed

### Examples 159-161

The following compounds were synthesized according to the procedure described for Example 1.

| Ex | Compound | Data | SM |
|---|---|---|---|
| 159 | 2-[(3S,4R)-4-{[(3,4-dichloro-5- | ¹H NMR (400 MHz, DMSO-d₆): | Example 156 |
| | methyl-1H-pyrrol-2- | 0.81 (t, 3 H), 1.47 (m, 2 H), 1.79 | |
| | yl)carbonyl]amino}-3- | (br s, 2 H), 2.18 (s, 3 H), 3.44 (m, | |
| | propoxypiperidin-1-yl]-4-(1-methyl- | 3 H), 3.58 (q, 2 H), 3.65 (s, 3 H), | |
| | 1H-1,2,4-triazol-5-yl)-1,3-thiazole-5- | 3.91 (br s, 1 H), 4.29 (m, 2 H), | |
| | carboxylic acid | 7.10 (d, 1 H), 8.00 (s, 1 H), 12.04 | |
| | 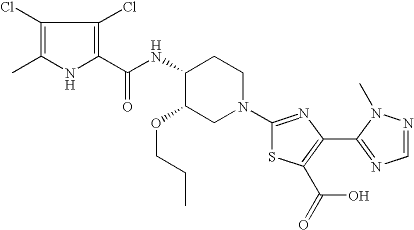 | (s, 1 H). | |
| 160 | 2-[(3S,4R)-4-{[(3,4-dichloro-5- | ¹H NMR (400 MHz, DMSO-d₆): | Example 157 |
| | methyl-1H-pyrrol-2- | 1.04 (t, 3 H), 1.45 (m, 2 H), 1.78 | |
| | yl)carbonyl]amino}-3- | (br s, 2 H), 2.18 (s, 3 H), 3.37 (m, | |
| | propoxypiperidin-1-yl]-4-(3- | 2 H), 3.60 (t, 2 H), 3.64 (br s, 1 H), | |
| | fluoropyridin-2-yl)-1,3-thiazole-5- | 4.29 (m, 2 H), 7.09 (d, 1 H), 7.56 | |
| | carboxylic acid | (m, 1 H), 7.80 (t, 1 H), 8.45 (s, | |
| | 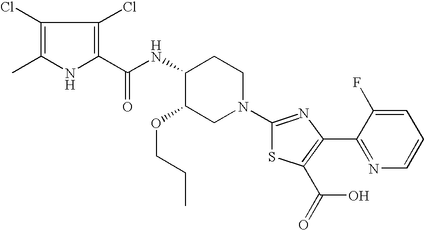 | 1 H), 12.14 (s, 1 H). | |
| 161 | 2-[(3S,4R)-4-{[(3,4-dichloro-5- | ¹H NMR (400 MHz, DMSO-d₆): | Example 158 |
| | methyl-1H-pyrrol-2- | 1.04 (t, 3 H), 1.45 (m, 2 H), 1.78 | |
| | yl)carbonyl]amino}-3- | (br s, 2 H), 2.18 (s, 3 H), 3.39 (m, | |
| | propoxypiperidin-1-yl]-4-(pyrazin-2- | 5 H), 4.25 (br s, 2 H), 7.11 (d, | |
| | yl)-1,3-thiazole-5-carboxylic acid | 1 H), 8.69 (d, 2 H), 8.81 (s, 1 H), | |
| | 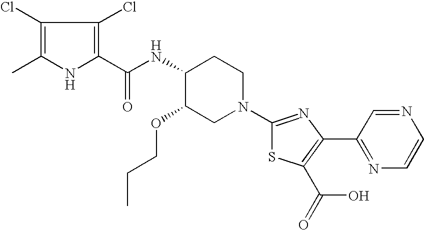 | 12.14 (s, 1 H). | |

### Example 162

### 2-{(3S,4R)-4-[(4-chloro-3-cyano-5-methyl-1H-pyrrole-2-carbonyl)-amino]-3-fluoropiperidin-1-yl}-4-pyrazin-2-yl-thiazole-5-carboxylic acid ethyl ester

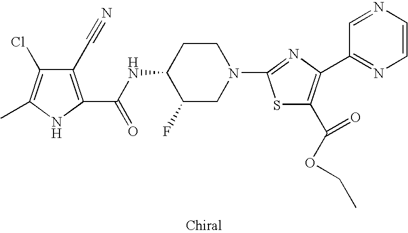

In a 10 mL round-bottomed flask, were taken 4-chloro-3-cyano-N-((3S,4R)-3-fluoropiperidin-4-yl)-5-methyl-1H-pyrrole-2-carboxamide hydrochloride (WO 2006/087543, 107 mg, 0.33 mmol) and ethyl 2-chloro-4-(pyrazin-2-yl)thiazole-5-carboxylate (Intermediate 116, 90 mg, 0.33 mmol) in 2 mL of DMF and stirred at RT. To this diisopropylethylamine (0.174 mL, 1 mmol) was added, and the resulting reaction mixture was stirred at 60° C. for 4 hrs. The reaction mixture was monitored by LCMS and LCMS profile showed completion of reaction after 4 hr. The reaction mixture was evaporated in vacuo and ice cool water was added. The mixture was sonicated and the solid precipitate was filtered and dried under high vacuum to afford the title compound as solid (175 mg).

MS (ES⁺): 518 for C₂₂H₂₅ClN₇O₃S

### Examples 163-167

The following compounds were prepared in a manner analogous to Example 162 from starting materials listed and corresponding chloro thiazoles. The resulting crude material was taken for next step without further purification.

| | | m/z | |
|---|---|---|---|
| Ex | Compound | (ES⁺) | SM |
| 163 | 2-{(3S,4R)-4-[(4-Chloro-3,5-dimethyl-1H- | 507 | Intermediate 6 and |
| | pyrrole-2-carbonyl)-amino]-3-fluoro-piperidin-1- | | Intermediate 117 |
| | yl}-4-pyrazin-2-yl-thiazole-5-carboxylic acid ethyl | | |
| | ester | | |
| | 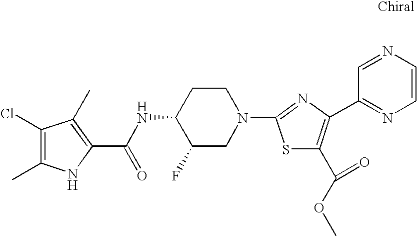 | | |
| 164 | 2-{(3S,4R)-4-[(4-chloro-3,5-dimethyl-1H- | 507 | Intermediate 2 and |
| | pyrrole-2carbonyl)-amino]-3-fluoro-piperidin-1- | | Intermediate 117 |
| | yl}-4-pyrimidin-4-yl-thiazole-5-carboxylic acid | | |
| | ethyl ester | | |
| | 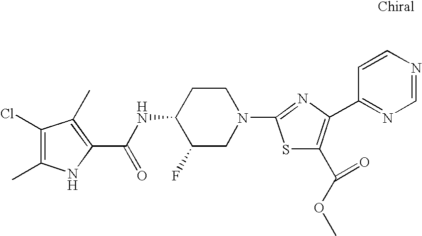 | | |
| 165 | 2-{(3S,4R)-4-[(4-Chloro-3-cyano-5-methyl-1H- | 531 | Intermediate 2 and |
| | pyrrole-2-carbonyl)-amino]- | | Intermediate 119 |
| | 3-methoxy-piperidin-1-yl}-4-pyrimidin-4-yl- | | |
| | thiazole-5-carboxylic acid | | |
| | ethyl ester | | |
| | 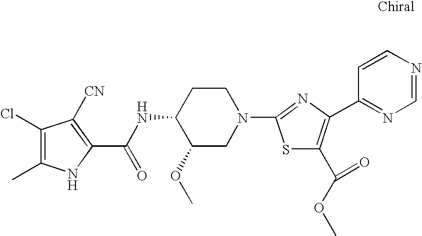 | | |
| 166 | 2-{(3S,4R)-4-[(4-Chloro-3-cyano-5-methyl-1H- | 533 | Intermediate 60 and |
| | pyrrole-2-carbonyl)amino]- | | Intermediate 119 |
| | 3-methoxy-piperidin-1-yl}-4-(1-methyl-1H- | | |
| | pyrazol-3-yl)-thiazole-5-carboxylic acid ethyl ester | | |
| | 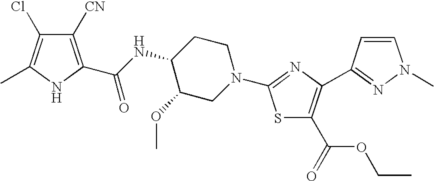 | | |
| 167 | 2-{(3S,4R)-4-[(3,4-Dichloro-5-methyl-1H- | 542 | Intermediate 60 and |
| | pyrrole-2-carbonyl)-amino]-3-methoxy-piperidin- | | 3,4-Dichloro-5- |
| | 1-yl}-4-(1-methyl- | | methyl-1H-pyrrole-2- |
| | 1H-pyrazol-3-yl)-thiazole-5-carboxylic acid ethyl | | carboxylic acid ((3S, |
| | ester | | 4R)-3-methoxy- |
| | 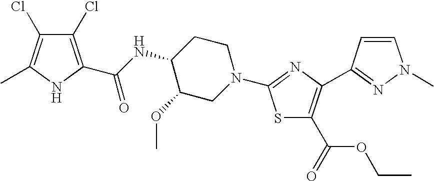 | | piperidin-4-yl)-amide(WO 2006087543) |

### Example 168

### 2-{(3S,4R)-4-[(4-chloro-3-cyano-5-methyl-1H-pyrrole-2-carbonyl)-amino]-3-piperidin-1-yl}-4-pyrazin-2-yl-thiazole-5-carboxylic acid

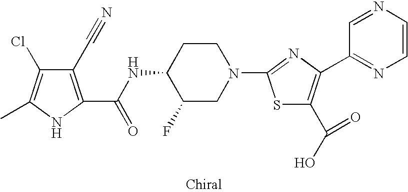

A solution of lithium hydroxide monohydrate (131 mg, 5.48 mmol) in water (1 mL) was added to a stirred solution of Ethyl 2-((3S,4R)-4-(4-chloro-3-cyano-5-methyl-1H-pyrrole-2-carboxamido)-3-fluoropiperidin-1-yl)-4-(pyrazin-2-yl) thiazole-5-carboxylate (Example 162, 142 mg, 0.27 mmol) in THF (8 mL) and MeOH (2 mL) and the resultant solution was stirred at 60° C. overnight. The reaction mixture was concentrated and dissolved in water (5 mL). The solution was acidified with 6N hydrochloric acid, and the precipitate that formed was collected, washed with water and dried under high vacuum (82 mg, 61 1%).

MS (ES⁺): 490 for C₂₀H₁₇ClN₇O₃S

H¹NMR [DMSO-d⁶] δ: 1.85-1.95(m, 2H); 2.20(s,3H); 3.50-3.60 (m,2H); 4.15 (d, 1H; 4.25-4.43 (m, 2H); 4.90-5.10 (d, 1H; 8.30(d,1H); 8.90(d,2H);9.30(s,1H); 13.0(bs,1H); 15.15(bs,1H);

### Examples 169-173

The following compounds were synthesized by an analogous method to Example 168 from the starting material (SM) given in the table below.

| | | | ¹HNMR | |
|---|---|---|---|---|
| | | | (300 MHz) | |
| Ex | Compound | M/Z | (δ) | SM |
| 169 | 2-[(3S,4R)-4-{[(4-chloro-3,5-dimethyl-1H- | 479 | H¹NMR [DMSO- | Example |
| | pyrrol-2-yl)carbonyl]amino}-3-fluoropiperidin- | | d⁶] δ: 1.80-1.95 (m, | 163 |
| | 1-yl]-4-pyrazin-2-yl-1,3-thiazole-5-carboxylic | | 2 H); 2.17 (s, 3 H); | |
| | acid | | 2.21 (s, 3 H); 3.35-3.42 | |
| | 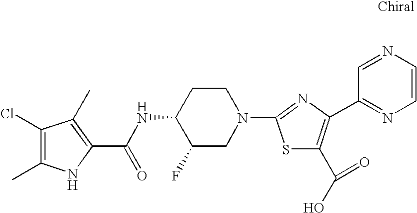 | | (m, 1 H); 3.50-3.70(m, 2 H); 4.15 (d,1 H); 4.25-4.47 (m,2 H); 4.90-5.05 (d,1 H); 7.45 (d, 1 H);8.85 (d, 2 H);9.30 (s, 1 H);11.35 (bs, 1 H);15.10 (bs, 1 H); | |
| 170 | 2-[(3S,4R)-4-{[(4-chloro-3,5-dimethyl-1H- | 479 | H¹NMR [DMSO- | Example |
| | pyrrol-2-yl)carbonyl]amino}-3-fluoropiperidin- | | d⁶] δ: 1.79-1.91 (m, | 164 |
| | 1-yl]-4-pyrimidin-4-yl-1,3-thiazole-5-carboxylic | | 2 H); 2.15 (d, 6 H); | |
| | acid | | 3.35-3.42 (m, 1 H); | |
| | 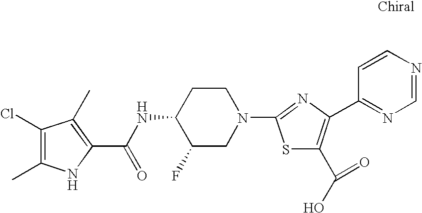 | | 3.50-3.70(m, 2 H); 4.15 (d,1 H); 4.25-4.47 (m,2 H); 4.85-5.05 (d,1 H); 7.40 (d, 1 H);8.25 (d, 2 H);9.10 (d, 2 H);9.40 (s, 1 H);11.30 (bs, 1 H); | |
| 171 | 2-[(3S,4R)-4-{[(4-chloro-3-cyano-5-methyl-1H- | 502 | H¹NMR [DMSO- | Example |
| | pyrrol-2-yl)carbonyl]amino}-3- | | d⁶] δ: 1.75-1.90 (m, | 165 |
| | methoxypiperidin-1-yl]-4-pyrimidin-4-yl-1,3- | | 2 H); 2.20 (s, 3 H); | |
| | thiazole-5-carboxylic acid | | 3.25 (s, 3 H); 3.40-3.50 | |
| | 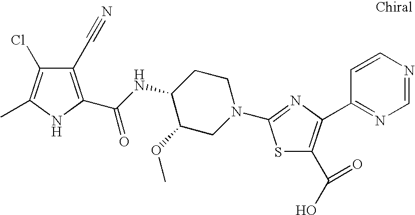 | | (m, 2 H); 3.60(s, 1 H); 4.05 (bs,1 H); 4.25-4.47 (m,2 H); 7.75 (d, 1 H);8.30 (d, 2 H); 9.15 (d,1 H); 9.35 (s, 1 H)12.65 (bs, 1 H); | |
| 172 | 2-[(3S,4R)-4-{[(4-chloro-3-cyano-5-methyl-1H- | 504 | H¹NMR [DMSO- | Example |
| | pyrrol-2-yl)carbonyl]amino}-3- | | d⁶] δ: 1.75-1.90 (m, | 166 |
| | methoxypiperidin-1-yl]-4-(1-methyl-1H- | | 2 H); 2.20 (s, 3 H); | |
| | pyrazol-3-yl)-1,3-thiazole-5-carboxylic acid | | 3.35-3.50 (m, | |
| | 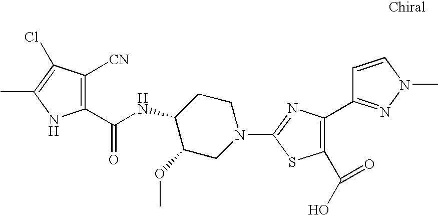 | | 5 H); 3.60 (s, 1 H);3.90-4.0 (m, 4 H);4.25-4.45 (m, 2 H);6.85 (s, 1 H); 7.82(d,1 H); 7.95 (s, 1 H);12.75 (bs, 1 H); | |
| 173 | 2-[(3S,4R)-4-{[(3,4-dichloro-5-methyl-1H- | 513 | H¹NMR [DMSO- | Example |
| | pyrrol-2-yl)carbonyl]amino}-3- | | d⁶] δ: 1.75-1.85 (m, | 167 |
| | methoxypiperidin-1-yl]-4-(1-methyl-1H- | | 2 H); 2.20 (s, 3 H); | |
| | pyrazol-3-yl)-1,3-thiazole-5-carboxylic acid | | 3.25 (s, 3 H); 3.30-3.40 | |
| | 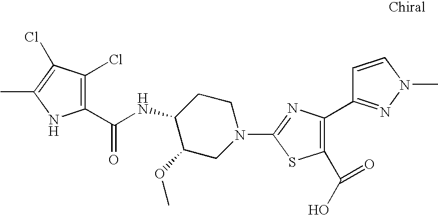 | | 3.40 (m, 2 H); 3.60(s, 1 H); 3.95-4.0 (m,4 H); 4.25-4.45 (m,2 H); 6.80 (s, 1 H);7.15 (d, 1 H); 7.95 (s,1 H); 12.10 (bs, 1 H);15.0 (bs, 1 H); | |

### Preparation of Starting Materials

### Intermediate 1

### Ethyl 2-chloro-4-pyrimidin-2-yl-1,3-thiazole-5-carboxylate

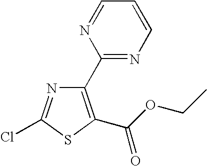

Ethyl 2-amino-4-pyrimidin-2-yl-1,3-thiazole-5-carboxylate (Intermediate 23; 0.55 g, 2.2 mmol) was suspended in glacial acetic acid (20 ml) and concentrated HCl (30 ml). The solution was cooled to 0° C. and to this a solution of sodium nitrite in water (15 ml) was added dropwise. After stirring at 0° C. for 10 mins, the reaction was slowly warmed to room temperature and stirred for 1 hour. Once complete by LCMS, a solution of urea (0.25 g) in water (10 ml) was added dropwise. After stirring at room temperature for 30 mins, solvent was removed under reduced pressure. The residue was partitioned with sat. NaHCO₃ (aq) and EtOAc. Extraction with EtOAc (×3), drying with MgSO₄ and concentrating yielded an orange oil which was used without purification (0.20 g). MS (ES) (M+H)⁺: 270 for C₁₀H₈ClN₃O₂S

### Intermediates 2-12

The following Intermediates were prepared by the procedure described in Intermediate 1 from the starting materials (SM) indicated.

| Int | Compound | Data | SM |
|---|---|---|---|
| 2 | Ethyl 2-chloro-4-pyrimidin-4- | MS (ES) (M + H)⁺: 271 for | Intermediate 24 |
| | yl-1,3-thiazole-5-carboxylate | C₁₀H₈ClN₃O₂S | |
| | 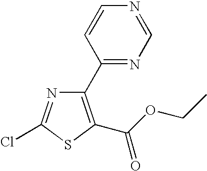 | NMR: 1.19 (t, 3 H), 4.24 (q, 2 H),7.92 (d, 2 H), 8.98 (d, 2 H), 9.29 (s,1 H) | |
| 3 | Methyl 2′-chloro-2,4′-bi-1,3- | MS (ES) (M + H)⁺: 261 for | Intermediate 25 |
| | thiazole-5′-carboxylate | C₈H₅ClN₂O₂S₂ | |
| | 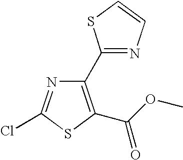 | NMR: 3.84 (s, 3 H), 8.02 (m, 2 H) | |
| 4 | Ethyl 4-[2,6- | MS (ES) (M + H)⁺: 356 for | Intermediate 26 |
| | bis(dimethylamino)pyrimidin-4- | C₁₄H₁₈ClN₅O₂S | |
| | yl]-2-chloro-1,3-thiazole-5- | NMR: 1.21 (t, 3 H), 3.08 (s, 3 H), | |
| | carboxylate | 3.10 (s, 3 H), 4.25 (q 2 H), 6.45 (s, | |
| | 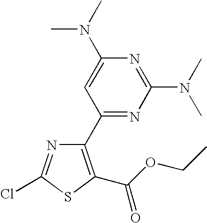 | 1 H) | |
| 5 | Ethyl 2-chloro-4-(4,6- | MS (ES) (M + H)⁺: 330 for | Intermediate 27 |
| | dimethoxypyrimidin-2-yl)-1,3- | C₁₂H₁₂ClN₃O₄S | |
| | thiazole-5-carboxylate | NMR: 1.14 (t, 3 H), 3.89 (s, 6 H), | |
| | 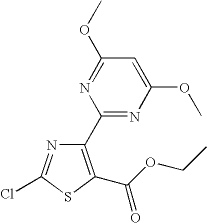 | 4.21 (q, 2 H), 6.37 (s, 1 H) | |
| 6 | Ethyl 2-chloro-4-pyrazin-2-yl- | MS (ES) (M + H)⁺: 270 for | Intermediate 28 |
| | 1,3-thiazole-5-carboxylate | C₁₀H₈ClN₃O₂S | |
| | 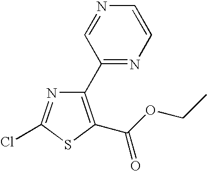 | NMR: 1.16 (t, 3 H), 4.22 (q, 2 H),8.76 (m, 2 H), 9.03 (s, 1 H) | |
| 7 | Ethyl 4-(1,3-benzothiazol-2-yl)- | MS (ES) (M + H)⁺: 325 for | Intermediate 29 |
| | 2-chloro-1,3-thiazole-5- | C₁₃H₉ClN₂O₂S₂ | |
| | carboxylate | NMR: 1.27 (t, 3 H), 4.34 (q, 2 H), | |
| | 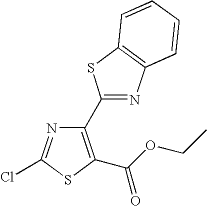 | 7.59 (m, 2 H), 8.08 (d, 1 H), 8.22(d, 1 H), | |
| 8 | Methyl 2-chloro-4-[1-(2- | MS (ES) (M + H)⁺: 302 for | Intermediate 19 |
| | methoxyethyl)-1H-imidazol-2- | C₁₁H₁₂ClN₃O₃S | |
| | yl]-1,3-thiazole-5-carboxylate | NMR: 3.34 (s, 3 H), 3.62 (m, 2 H), | |
| | 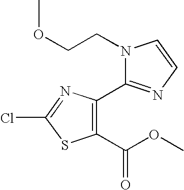 | 3.81 (s, 3 H), 4.22 (m, 2 H), 7.24 (s,2 H). | |
| 9 | Methyl 2-chloro-4-(1-methyl- | MS (ES) (M + H)⁺: 259 for | Intermediate 20 |
| | 1H-1,2,4-triazol-5-yl)-1,3- | C₈H₇ClN₄O₂S | |
| | thiazole-5-carboxylate | NMR: 3.92 (s, 6 H), 8.04 (s, 1 H). | |
| | 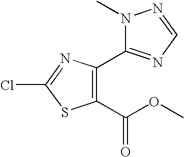 | | |
| 10 | Methyl 2-chloro-4-(1-methyl- | MS (ES) (M + H)⁺: 258 for | Intermediate 16 |
| | 1H-imidazol-2-yl)-1,3-thiazole- | C₉H₈ClN₃O₂S | |
| | 5-carboxylate | NMR: 3.73 (s, 3 H), 3.81 (s, 3 H), | |
| | 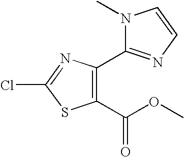 | 7.03 (s, 1 H), 7.21 (s, 1 H). | |
| 11 | Methyl 2-chloro-4-(1,4,5- | MS (ES) (M + H)⁺: 286 for | Intermediate 21 |
| | trimethyl-1H-imidazol-2-yl)- | C₁₁H₁₂ClN₃O₂S | |
| | 1,3-thiazole-5-carboxylate | NMR: 2.03 (s, 3 H), 2.13 (s, 3 H), | |
| | 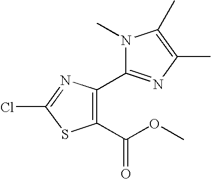 | 3.34 (s, 3 H), 3.61 (s, 3 H), 7.90 (s,2 H). | |
| 12 | Ethyl 2-chloro-4-(1-methyl-1H- | MS (ES) (M + H)⁺: 272 for | Intermediate 22 |
| | imidazol-4-yl)-1,3-thiazole-5- | C₁₀H₁₀ClN₃O₂S | |
| | carboxylate | NMR: 1.42 (t, 3 H), 3.81 (s, 3 H), | |
| | 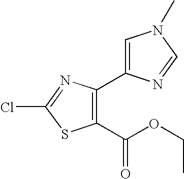 | 4.35 (q, 2 H), 7.50 (s, 1 H), 8.21 (s,1 H). | |

### Intermediate 13

### Ethyl 2-chloro-4-pyridin-2-yl-1,3-thiazole-5-carboxylate

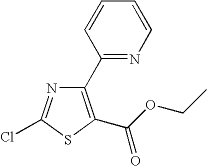

To a solution of ethyl 2-oxo-4-pyridin-2-yl-2,3-dihydro-1,3-thiazole-5-carboxylate (0.42 g, 1.7 mmol) in neat phosphorus oxychloride (5 ml) was added pyridine and the reaction was heated to reflux. After 3 hrs, LCMS indicates complete conversion. The reaction mixture was concentrated to remove phosphorus oxychloride. The residue was diluted with water and extracted three times with EtOAc, dried with MgSO₄ and concentrated to a brown oil (0.35 g).

MS (ES) (M+H)⁺: 269 for C₁₁H₉ClN₂O₂S; NMR: 1.16 (t, 3H), 4.21 (q, 2H), 7.52 (dd, 1H), 7.82 (d, 1H), 7.96 (t 1H), 8.65 (d, 1H)

### Intermediate 14

### Methyl 2-chloro-4-(1-{[2-(trimethylsilyl)ethoxylmethyl}-1H-imidazol-2-yl)-1,3-thiazole-5-carboxylate

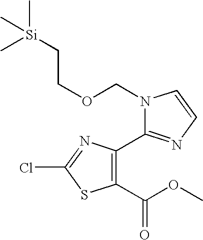

t-Butylnitrite (0.81 ml, 6.25 mmol) was added dropwise to a mixture of 1.5 g (4.2 mmol) methyl 2-amino-4-(1-{[2-(trimethylsilyl)ethoxy]methyl}-1H-imidazol-2-yl)-1,3-thiazole-5-carboxylate (Intermediate 17) and 900 mg CuCl₂ in 30 ml CH₃CN at room temperature. After stirring 1 hour, the mixture was quenched with NaHSO₃ (aq) and diluted with water before being extracted twice with EtOAc. The EtOAc extracts were washed with brine, dried (MgSO₄) and concentrated to afford an oil that was chromatographed on silica gel (100% DCM followed by gradient elution to 90% EtOAc in DCM) to give 800 mg of a solid: MS (ES) (M+H)⁺: 374 for C₁₄H₂₀N₃O₃SSi; NMR: −0.13 (s, 9H), 0.75 (m, 2H), 3.32 (m, 4H), 3.87 (s, 3H), 5.32 (s, 2H), 6.04 (s, 2H).

### Intermediate 15

The following Intermediate was synthesized by an analogous method to Intermediate 14 from the starting materials (SM) given in the table below.

| Int | Compound | Data | SM |
|---|---|---|---|
| 15 | Methyl 2-chloro-4-[1-(methoxymethyl)-1H- | MS (ES) (M + H)⁺: | Intermediate 18 |
| | imidazol-2-yl]-1,3-thiazole-5-carboxylate | 269 for | |
| | 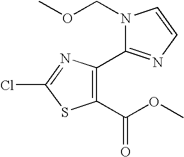 | C₁₀H₁₀ClN₃O₃SNMR: 3.22 (s, 3 H),3.78 (s, 3 H), 5.35 (s,2 H), 7.32 (s, 2 H). | |

### Intermediate 16

### Methyl 2-amino-4-(1-methyl-1H-imidazol-2-yl)-1,3-thiazole-5-carboxylate

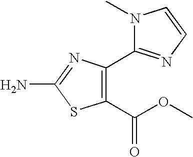

N-Iodosuccinimide (9.3 g, 41 mmol) was added to a mixture of 7.52 g (41 mmol) methyl 3-(1-methyl-1H-imidazol-2-yl)-3-oxopropanoate (Intermediate 48) and 7.5 g Amberlyst-15 resin in 400 ml EtOAc followed by stirring for 1 hour at room temperature. The resin was filtered off and rinsed with EtOAc. Solvent was removed from the filtrate and the residue was taken up in diethyl ether. Insoluble material was filtered off and rinsed with additional ether. Solvent was removed from the filtrate and the residue was dissolved in 200 ml MeOH before added 4.7 g (62 mmol) thiourea. The mixture was heated at reflux for 1 hour. Solvent was removed and the residue was taken up in aqueous Na₂CO₃. Insoluble material was collected by filtration and rinsed well with water. The solids were dried in vacuo affording 4.51 g of product: MS (ES) (M+H)⁺: 239 for C₉H₁₀N₄O₂S; NMR: 3.48 (s, 3H), 3.57 (s, 3H), 6.90 (s, 1H), 7.12 (s, 1H), 7.98 (s, 2H).

### Intermediates 17-22

The following Intermediates were synthesized by an analogous method to Intermediate 16 from the starting materials (SM) given in the table below.

| Int | Compound | Data | SM |
|---|---|---|---|
| 17 | Methyl 2-amino-4-(1-{[2- | MS (ES) (M + H)⁺: 355 | Intermediate 45 |
| | (trimethylsilyl)ethoxy]methyl}-1H- | for C₁₄H₂₂N₄O₃SSi | |
| | imidazol-2-yl)-1,3-thiazole-5-carboxylate | NMR: −0.14 (s, 9 H), | |
| | 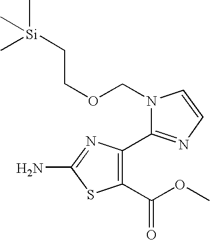 | 0.67 (m, 2 H), 3.34 (m,2 H), 3.62 (s, 3 H), 5.28(s, 2 H), 7.03 (s, 1 H),7.35 (s, 1 H), 7.95 (s,2 H). | |
| 18 | Methyl 2-amino-4-[1-(methoxymethyl)-1H- | MS (ES) (M + H)⁺: 269 | Intermediate 46 |
| | imidazol-2-yl]-1,3-thiazole-5-carboxylate | for C₁₀H₁₂N₄O₃S | |
| | 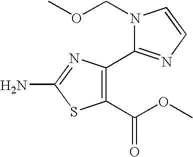 | NMR: 3.23 (s, 3 H), 3.71(s, 3 H), 5.51 (s, 2 H),7.87 (s, 1 H), 8.02 (s,1 H), 8.44 (s, 2 H). | |
| 19 | Methyl 2-amino-4-[1-(2-methoxyethyl)-1H- | MS (ES) (M + H)⁺: 283 | Intermediate 47 |
| | imidazol-2-yl]-1,3-thiazole-5-carboxylate | for C₁₁H₁₄N₄O₃S | |
| | 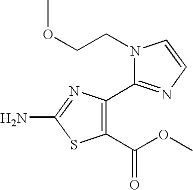 | NMR: 3.22 (s, 3 H), 3.61(m, 2 H), 3.69 (s, 3 H),4.32 (m, 2 H), 7.91 (s,2 H), 8.41 (s, 2 H). | |
| 20 | Methyl 2-amino-4-(1-methyl-1H-1,2,4- | MS (ES) (M + H)⁺: 240 | Intermediate 44 |
| | triazol-5-yl)-1,3-thiazole-5-carboxylate | for C₈H₉N₅O₂S | |
| | 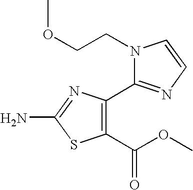 | NMR: 3.61 (s, 3 H), 3.71(s, 3 H), 7.96 (s, 1 H),8.10 (s, 2 H). | |
| 21 | Methyl 2-amino-4-(1,4,5-trimethyl-1H- | MS (ES) (M + H)⁺: 267 | Intermediate 49 |
| | imidazol-2-yl)-1,3-thiazole-5-carboxylate | for C₉H₁₀N₄O₂S | |
| | 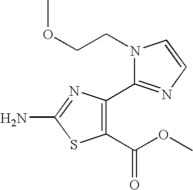 | NMR: 2.01 (s, 3 H), 2.14(s, 3 H), 3.32 (s, 3 H),3.61 (s, 3 H), 7.89 (s,2 H). | |
| 22 | Ethyl 2-amino-4-(1-methyl-1H-imidazol-4- | MS (ES) (M + H)⁺: 253 | Intermediate 43 |
| | yl)-1,3-thiazole-5-carboxylate | for C₁₀H₁₂N₄O₂S | |
| | 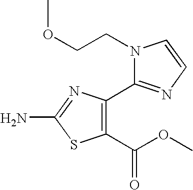 | NMR: 1.15 (t, 3 H), 3.62(s, 3 H), 4.15 (q, 2 H),7.65 (s, 1 H), 7.95 (s,1 H). | |

### Intermediate 23

### Ethyl 2-amino-4-pyrimidin-2-yl-1,3-thiazole-5-carboxylate

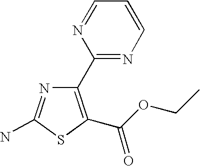

A suspension of ethyl 2-iodo-3-oxo-3-pyrimidin-2-ylpropanoate (Intermediate 30; 1.73 g, 5.4 mmol) and thiourea (0.62 g, 8.1 mmol) in EtOH was heated at reflux for 1 hour. After cooling to room temperature, the reaction was concentrated. The residue was suspended in water and basified with saturated aqueous Na₂CO₃. The resulting precipitate was filtered off and the filtrate was extracted with EtOAc (×3). The organic extracts were dried with MgSO₄ and concentrated to an orange oil (0.55 g, 41%). MS (ES) (M+H)^{|}: 251 for C₁₀H₁₀N₄O₂S; NMR: 0.97 (t, 3H), 3.95 (q, 2H), 7.55 (t, 1H), 7.94 (s, 1H), 8.85 (d, 1H), 9.05 (d, 1H)

### Intermediates 24-29

The following Intermediates were prepared by the procedure described in Intermediate 23 from the starting materials (SM) indicated.

| Int | Compound | Data | SM |
|---|---|---|---|
| 24 | Ethyl 2-amino-4-pyrimidin-4- | MS (ES) (M + H)⁺: 251 for | Intermediate 31 |
| | yl-1,3-thiazole-5-carboxylate | C₁₀H₁₀N₄O₂S | |
| | 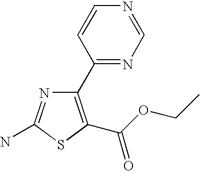 | NMR: 1.08 (t, 3 H), 4.05 (q, 2 H),7.68 (d, 1 H), 7.98 (s, 2 H), 8.86 (d,1 H), 9.21 (s, 1 H) | |
| 25 | Methyl 2′-amino-2,4′-bi-1,3- | MS (ES) (M + H)⁺: 242 for | Intermediate 32 |
| | thiazole-5′-carboxylate | C₈H₇N₃O₂S₂ | |
| | 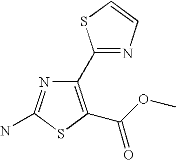 | NMR: 3.67 (s, 3 H), 7.86 (d, 1 H),7.91 (d, 1 H), 7.92 (s, 2 H) | |
| 26 | Ethyl 2-amino-4-[2,6- | MS (ES) (M + H)⁺: 337 for | Intermediate 33 |
| | bis(dimethylamino)pyrimidin-4- | C₁₄H₂₀N₆O₂S | |
| | yl]-1,3-thiazole-5-carboxylate | NMR: 1.22 (t, 3 H), 3.19 (s, 12 H), | |
| | 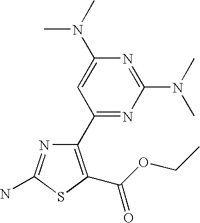 | 4.21 (q, 2 H), 6.75 (s, 1 H), 8.21 (s,2 H) | |
| 27 | Ethyl 2-amino-4-(4,6- | MS (ES) (M + H)⁺: 311 for | Intermediate 34 |
| | dimethoxypyrimidin-2-yl)-1,3- | C₁₂H₁₄N₄O₄S | |
| | thiazole-5-carboxylate | NMR: 1.02 (t, 3 H), 3.86 (s, 6 H), | |
| | 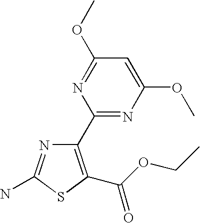 | 4.03 (q, 2 H), 6.27 (s, 1 H), 7.94 (s,2 H) | |
| 28 | Ethyl 2-amino-4-pyrazin-2-yl- | MS (ES) (M + H)⁺: 251 for | Intermediate 35 |
| | 1,3-thiazole-5-carboxylate | C₁₀H₁₀N₄O₂S | |
| | 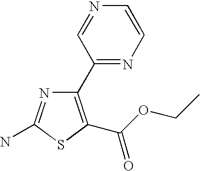 | NMR: 1.07 (t, 3 H), 4.04 (q, 2 H),7.99 (s, 2 H), 8.65 (d, 1 H), 8.69 (d,1 H), 8.81 (s, 1 H) | |
| 29 | Ethyl 2-amino-4-(1,3- | MS (ES) (M + H)⁺: 306 for | Intermediate 36 |
| | benzothiazol-2-yl)-1,3-thiazole- | C₁₃H₁₁N₃O₂S₂ | |
| | 5-carboxylate | NMR: 1.16 (t, 3 H), 4.15 (q, 2 H), | |
| | 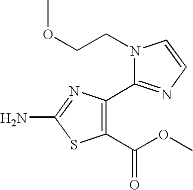 | 7.51 (m, 2 H), 8.06 (m, 3 H), 8.12(d, 1 H) | |

### Intermediate 30

### Ethyl 2-iodo-3-oxo-3-pyrimidin-2-ylpropanoate

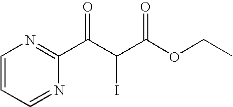

To a suspension of ethyl 3-oxo-3-pyrimidin-2-ylpropanoate (Intermediate 37; 1.19 g, 6.1 mmol) in EtOAc was added N-iodosuccinamide (1.38 g, 6.1 mmol) and Amberlyst-15 resin (1.19 g). After stirring at room temperature for 30 mins, LCMS shows a mixture of desired product and bis-iodinated product. (Note: workup reaction as soon as it is finished. Excessive reaction times results in decomposition). The reaction mixture was filtered to remove the Amberlyst-15 resin. The filtrate was concentrated to an orange oil which was then suspended in diethyl ether. The resulting precipitate was filtered and washed with ether. The filtrate was concentrated to an orange oil (1.73 g, 89%). MS (ES) (M+H)^{|}: 321 for C₉H₉IN₂O₃

### Intermediates 31-36

The following Intermediates were prepared by the procedure described in Intermediate 30 from the starting materials (SM) indicated.

| Int | Compound | Data | SM |
|---|---|---|---|
| 31 | Ethyl 2-iodo-3-oxo-3-pyrimidin- | MS (ES) (M + H)⁺: 321 for | Intermediate 38 |
| | 4-ylpropanoate | C₉H₉IN₂O₃ | |
| | 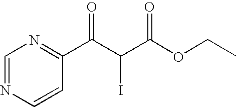 | NMR: 1.11 (t, 3H), 4.12 (q, 2H),6.41 (s, 1H), 8.03 (d, 1H), 9.16 (d,1H), 9.42 (s, 1H) | |
| 32 | Methyl 2-iodo-3-oxo-3-(1,3- | MS (ES) (M + H)⁺: 312 for | Intermediate 50 |
| | thiazol-2-yl)propanoate | C₇H₈INO₃S | |
| | 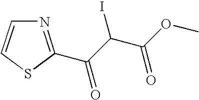 | | |
| 33 | Ethyl 3-[2,6- | MS (ES) (M + H)⁺: 407 for | Intermediate 39 |
| | bis(dimethylamino)pyrimidin-4- | C₁₃H₁₉IN₄O₃ | |
| | yl]-2-iodo-3-oxopropanoate | NMR: 1.19 (t, 3H), 3.06 (s, 6H), | |
| | 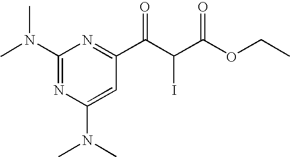 | 3.17 (s, 6H), 4.09 (q, 2H), 6.41 (s,1H) | |
| 34 | Ethyl 3-(4,6-dimethoxypyrimidin- | MS (ES) (M + H)⁺: 381 for | Intermediate 40 |
| | 2-yl)-2-iodo-3-oxopropanoate | C₁₁H₁₃IN₂O₅ | |
| | 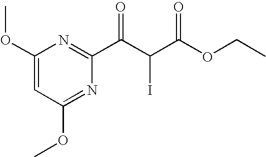 | | |
| 35 | Ethyl 2-iodo-3-oxo-3-pyrazin-2- | MS (ES) (M + H)⁺: 321 for | Intermediate 41 |
| | ylpropanoate | C₉H₉IN₂O₃ | |
| | 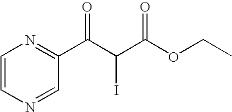 | | |
| 36 | Ethyl 3-(1,3-benzothiazol-2-yl)-2- | MS (ES) (M + H)⁺: 376 for | Intermediate 42 |
| | iodo-3-oxopropanoate | C₁₂H₁₀INO₃S | |
| | 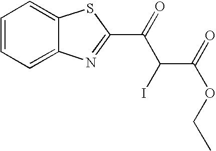 | | |

### Intermediate 37

### Ethyl 3-oxo-3-pyrimidin-2-ylpropanoate

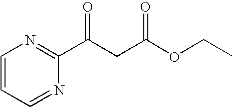

To a solution of pyrimidine-2-carboxylic acid (0.99 g, 7.98 mmol) in anhydrous THF (20 ml) was added carbonyl diimidazole (1.55 g, 9.57 mmol) and the suspension was heated at reflux for 2 hours. The mixture was then cooled to room temperature and used without workup or purification. In a separate flask, mono-ethyl malonate (0.94 ml, 7.98 mmol) was suspended in anhydrous THF (20 ml) and cooled to 0° C. Methyl magnesium bromide (5.32 ml, 15.96 mmol, 3.0 M in diethyl ether) was added dropwise. After stirring at 0° C. for 20 mins, the crude imidazolide solution prepared earlier was added slowly. The reaction was then heated at reflux overnight. After cooling to room temperature, the reaction mixture was diluted with water and acidified with concentrated HCl to pH 5. The solution was extracted with EtOAc (×3), dried with MgSO₄ and concentrated to a yellow oil (1.19 g, 77%). NMR showed a 2:1 mixture of the keto:enol forms. MS (ES) (M+H)⁺: 195 for C₉H₁₀N₂O₃; NMR: 1.13-1.29 (t, 3H), 4.05-4.28 (q, 2H), 4.18 (s, 2H), 7.62-7.76 (t, 1H), 8.95-9.06 (d, 2H), 11.79 (s, 4H)

### Intermediates 38-43

The following Intermediates were prepared by the procedure described in Intermediate 37 from the starting materials (SM) indicated.

| Int | Compound | Data | SM |
|---|---|---|---|
| 38 | Ethyl 3-oxo-3-pyrimidin-4- | MS (ES) (M + H)⁺: 195 for | pyrimidine-4- |
| | ylpropanoate | C₉H₁₀N₂O₃ | carboxylic acid |
| | 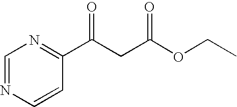 | NMR: 1.14 (t, 3H, 2/3) 1.28 (t,3H, 1/3) 4.17 (q, 2H, 2/3) 4.19 (s,2H), 4.25 (q, 2H, 1/3) 6.34 (s, 1H,1/3) 7.91 (d, 1H 1/3) 7.95 (d, 1H,2/3) 9.04 (d, 1H, 1/3) 9.15 (d, 1H,2/3) 9.30 (s, 1H, 1/3) 9.43 (s, 1H,2/3) 12.03 (s, 1H, 2/3) | |
| 39 | Ethyl 3-[2,6- | MS (ES) (M + H)⁺: 281 for | 2,6- |
| | bis(dimethylamino)pyrimidin-4- | C₁₃H₂₀N₄O₃ | bis(dimethylamino) |
| | yl]-3-oxopropanoate | NMR: 1.19 (t, 3H), 3.06 (s, 6H), | pyrimidine-4- |
| | 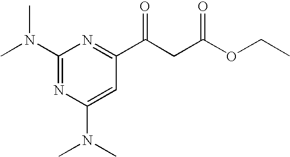 | 3.08 (s, 6H), 3.92 (s, 2H), 4.09 (q,2H), 6.35 (s, 1H) | carboxylic acid |
| 40 | Ethyl 3-(4,6- | MS (ES) (M + H)⁺: 255 for | 4,6- |
| | dimethoxypyrimidin-2-yl)-3- | C11H₁₄N₂O₅ | dimethoxypyrimidine- |
| | oxopropanoate | | 2- |
| | 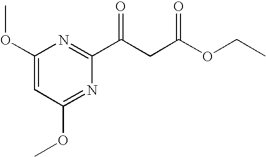 | | carboxylic acid |
| 41 | Ethyl 3-oxo-3-pyrazin-2- | MS (ES) (M + H)⁺: 195 for | pyrazine-2- |
| | ylpropanoate | C₉H₁₀N₂O₃ | carboxylic acid |
| | 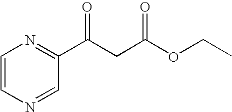 | NMR: 1.15 (t, 3H), 4.11 (q, 2H),4.18 (s, 2H), 8.82 (s, 1H), 8.94 (s,1H), 9.17 (s, 1H) | |
| 42 | Ethyl 3-(1,3-benzothiazol-2-yl)- | MS (ES) (M + H)⁺: 250 for | 1,3- |
| | 3-oxopropanoate | C₁₂H₁₁NO₃S | benzothiazole-2- |
| | 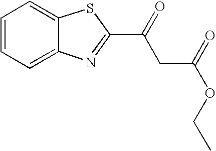 | NMR: 1.17 (t, 3H), 4.15 (q, 2H),4.32 (s, 2H), 7.68 (m, 2H), 8.27(m, 2H) | carboxylic acid |
| 43 | Ethyl 3-(1-methyl-1H-imidazol- | NMR: 1.25 (t, 3H), 3.72 (s, 3H), | 1-methyl-1H- |
| | 4-yl)-3-oxopropanoate | 4.01 (s, 2H), 4.18 (q, 2H), 7.43 (s, | imidazole-4- |
| | 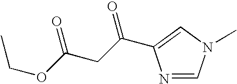 | 1H), 7.57 (s, 1H). | carboxylic acid |

### Intermediate 44

### Methyl 3-(1-methyl-1H-1,2,4-triazol-5-yl)-3-oxopropanoate

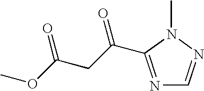

NaH (7.84 g, 196 mmol of a 60% dispersion in oil) was added portionwise to a solution of 6.18 g (34.5 mmol) of 1-(1-methyl-1H-1,2,4-triazol-5-yl)ethanone (Ohta, S.; Kawasaki, I.; Fukuno, A.; Yamashita, M.; Tada, T.; Kawabata, T. Chem. Pharm. Bull. (1993), 41(7), 1226-31) in 100 ml dimethylcarbonate. The mixture was heated to 90° C. for 2 hour forming a thick slurry. After cooling to room temperature, the mixture was slowly transferred to 1N HCl over ice. The pH of the mixture was brought to about 7 with NaHCO₃ before being saturated with NaCl and extracted 4 times with EtOAc. The EtOAc was dried (MgSO₄) and concentrated to give an oil that was chromatographed on silica gel (100% DCM followed by gradient elution to 50% EtOAc in DCM). Product (5.3 g) was obtained as an oil. NMR: 3.78 (s, 3H), 4.11 (s, 2H), 4.22 (s, 3H), 7.94 (s, 1H).

### Intermediates 45-50

The following Intermediates were synthesized by an analogous method to Intermediate 44 from the starting materials (SM) given in the table below.

| Int | Compound | Data | SM |
|---|---|---|---|
| 45 | Methyl 3-oxo-3-(1-{[2- | MS (ES) (M − H)⁻: 297 | Intermediate 51 |
| | (trimethylsilyl)ethoxy]methyl}-1H- | for C₁₃H₂₂NO₂Si | |
| | imidazol-2-yl)propanoate | NMR: −0.14 (s, 9H), | |
| | 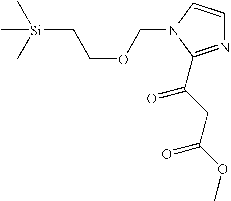 | 0.82 (m, 2H), 3.52 (t,2H), 3.77 (s, 3H), 4.10(s, 2H), 5.71 (s, 2H),7.21 (s, 1H), 7.71 (s,1 H). | |
| 46 | Methyl 3-[1-(methoxymethyl)-1H- | MS (ES) (M + H)⁺: 213 | Intermediate 52 |
| | imidazol-2-yl]-3-oxopropanoate | for C₉H₁₂N₂O₄ | |
| | 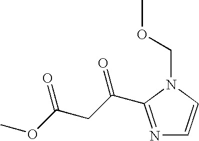 | NMR: 3.41 (s, 3H), 3.75(s, 3H), 4.22 (s, 2H),5.75 (s, 2H), 7.22 (s,1H), 7.31 (s, 1H). | |
| 47 | Methyl 3-[1-(2-methoxyethyl)-1H- | MS (ES) (M + H)⁺: 227 | Intermediate 53 |
| | imidazol-2-yl]-3-oxopropanoate | for C₁₀H₁₄N₂O₄ | |
| | 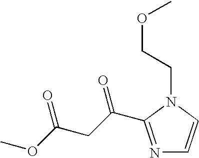 | NMR: 3.18 (s, 3H), 3.61(m, 5H), 4.07 (s, 2H),4.52 (m, 2H), 7.24 (s,1H), 7.61 (s, 1H). | |
| 48 | Methyl 3-(1-methyl-1H-imidazol-2- | MS (ES) (M + H)⁺: 183 | 1-(1-Methyl-1H- |
| | yl)-3-oxopropanoate | for C₈H₁₀N₂O_{3.} | imidazol-2- |
| | 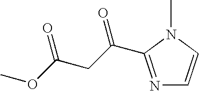 | | yl)ethanone (Abarca-Gonzalez, B.; Jones,R. A.; Medio-Simon,M.; Quilez-Pardo, J.;Sepulveda-Arques, J.;Zaballos-Garcia, E.Synth. Comm. (1990),20(3), 321-31). |
| 49 | Methyl 3-oxo-3-(1,4,5-trimethyl-1H- | NMR: 2.21 (s, 6H), 3.72 | Intermediate 54 |
| | imidazol-2-yl)propanoate | (s, 3H), 3.86 (s, 3H), | |
| | 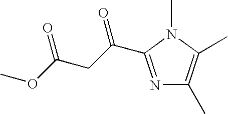 | 4.10 (s, 2H). | |
| 50 | Methyl 3-oxo-3-(1,3-thiazol-2- | MS (ES) (M + H)⁺: 186 | 1-(1,3-thiazol-2- |
| | yl)propanoate | for C₇H₇NO₃S | yl)ethanone |
| | 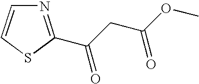 | NMR: 3.65 (s, 3H), 4.22(s, 2H), 8.18 (d, 1H),8.29 (d, 1H) | |

### Intermediate 51

### 1-(1-{[2-(Trimethylsilyl)ethoxy]methyl}-1H-imidazol-5-yl)ethanone

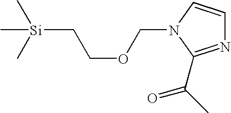

A solution of 30 ml (75 mmol) of 2.5 M n-butyllithium in hexanes was added slowly to a solution of 8.48 g (61.3 mmol) 1-{[2-(trimethylsilyl)ethoxy]methyl}-1H-imidazole (Lipshutz, B. H.; Huff, B.; Hagen, W. Tetrahedron Lett. (1988), 29(28), 3411-14) in 200 ml THF cooled in a dry ice-acetone bath. After stirring 1 hour, 8 ml (75 mmol) of N-methoxy-N-methylacetamide was added quickly, and the solution was allowed to warm to room temperature over 30 min. After quenching with aqueous NH₄Cl, the mixture was diluted with water and extracted twice with EtOAc, which was washed with brine, dried (MgSO₄) and concentrated to give an oil that was chromatographed on silica gel (100% DCM followed by gradient elution to 50% EtOAc in DCM). Product (8.5 g) was obtained as a mobile oil. NMR: −0.13 (s, 9H), 0.82 (m, 2H), 2.55 (s, 3H), 3.48 (m, 2H), 5.74 (s, 2H), 7.21 (s, 1H), 7.72 (s, 1H).

### Intermediates 52-54

The following Intermediates were synthesized by an analogous method to Intermediate 51 from the starting materials (SM) given in the table below.

| Int | Compound | Data | SM |
|---|---|---|---|
| 52 | 1-[1-(Methoxymethyl)-1H- | NMR: 2.72 (s, 3H), 3.35 | 1-(methoxymethyl)-1H- |
| | imidazol-2-yl]ethanone | (s, 3H), 5.75 (s, 2H), 7.22 | imidazole (Manoharn, T. |
| | 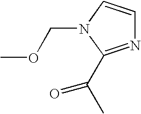 | (s, 1H), 7.34 (s, 1H). | S.; Brown, R. S. J. Org.Chem. (1989), 54(6),1439-42). |
| 53 | 1-[1-(2-Methoxyethyl)-1H- | MS (ES) (M + H)⁺: 169 for | 1-(2-Methoxyethyl)-1H- |
| | imidazol-2-yl]ethanone | C₈H₁₂CN₂O₂ | imidazole (WO |
| | 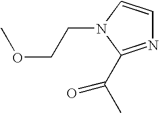 | NMR: 2.69 (s, 3H), 3.34(s, 3H), 3.71 (m, 2H), 4.61(m, 2H), 7.12 (s, 1H), 7.26(s, 1H). | 2003055876 A1) |
| 54 | 1-(1,4,5-Trimethyl-1H-imidazol- | NMR: 2.15 (s, 3H), 2.22 | 1,4,5-trimethyl-1H- |
| | 2-yl)ethanone | (s, 3H), 2.57 (s, 3H), 3.88 | imidazole (U.S. Pat. No. 6177575 |
| | 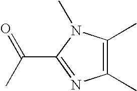 | (s, 3H), 3.34 (s, 3H), 3.71(m, 2H). | B1) |

### Intermediate 55

### Methyl 2′-amino-2-chloro-4,4′-bi-1,3-thiazole-5-carboxylate

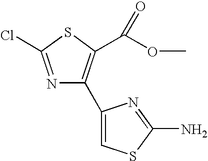

A solution of 770 mg (3.6 mmol) of methyl 2-chloro-4-(chloroacetyl)-1,3-thiazole-5-carboxylate (Intermediate 61) and 270 mg (3.6 mmol) thiourea in 2 ml MeOH was heated at reflux for 90 min. Solvent was removed, and the residue was taken up in water and treated with aqueous Na₂CO₃ precipitating solids that were collected, washed with water and dried in vacuo to give 670 mg of product: MS (ES) (M+H)⁺: 276 for C₈H₆ClN₃O₂S₂; NMR (CDCl₃): 3.91 (s, 3H), 5.08 (s, 2H), 7.28 (s, 1H).

### Intermediates 56-57

The following Intermediates were synthesized by an analogous method to Intermediate 55 from the starting materials (SM) given in the table below.

| Int | Compound | Data | SM |
|---|---|---|---|
| 56 | Methyl 2-chloro-2′-(methylamino)-4,4′- | MS (ES) (M + H)⁺: 290 | Intermediate 61 and |
| | bi-1,3-thiazole-5-carboxylate | for C₉H₈CN₃O₂S₂ | methylthiourea |
| | 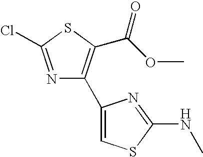 | NMR (CDCl₃): 3.01 (d,3H), 3.35 (s, 3H), 5.32(s, 1H), 3.87 (s, 3H),7.71 (s, 1H). | |
| 57 | Methyl 2-chloro-2′-(dimethylamino)- | MS (ES) (M + H)⁺: 304 | Intermediate 61 and |
| | 4,4′-bi-1,3-thiazole-5-carboxylate | for C₁₀H₁₀CN₃O₂S₂ | N,N- |
| | 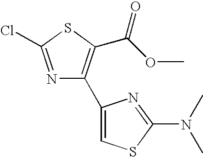 | NMR (CDCl₃): 3.11 (s,6H), 3.91 (s, 3H), 7.43(s, 1H). | dimethylthiourea |

### Intermediate 58

### Methyl 2-chloro-4-[(2E)-3-(dimethylamino)prop-2-enoyl]-1,3-thiazole-5-carboxylate

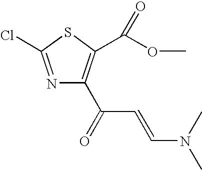

A solution of 1 g (4.55 mmol) methyl 4-acetyl-2-chloro-1,3-thiazole-5-carboxylate (WO 2006087543 A1) and 0.61 ml (4.6 mmol) dimethylformamide dimethylacetal in 4 ml toluene was heated at 100° C. for 3 hours in a microwave reactor. Solvent was removed and the residue was diluted with water, which was then saturated with NaCl. The solution was extracted 3 times with EtOAc, which was dried (MgSO₄) and concentrated. The residue was chromatographed on silica gel (100% DCM followed by gradient elution to 50% EtOAc in DCM) to give 480 mg of product as a viscous oil. NMR (CDCl₃): 2.88 (s, 3H), 3.11 (s, 3H), 3.87 (s, 3H), 5.61 (d, 1H, 7.82 (s, 1H).

### Intermediate 59

### Methyl 2-chloro-4-isoxazol-5-yl-1,3-thiazole-5-carboxylate

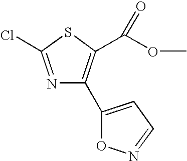

A solution of 1.18 mg (4.3 mmol) methyl 2-chloro-4-[(2E)-3-(dimethylamino)prop-2-enoyl]-1,3-thiazole-5-carboxylate (Intermediate 58) and 285 mg (4.1 mmol) hydroxylamine hydrochloride in 4 ml AcOH was heated at 120° C. for 90 min. Solvent was removed and the residue was partitioned between aqueous NaHCO₃ and EtOAc. The EtOAc was separated, washed with brine, dried (MgSO₄) and concentrated. The residue was chromatographed on silica gel (1:1 hexanes/DCM followed by gradient elution to 100% DCM) to give 450 mg of product as solid: MS (ES) (M+H)⁺: 245 for C₈H₅ClN₂O₃S; NMR: 3.91 (s, 3H), 7.17(s, 1H), 8.26 (s, 1H).

### Intermediate 60

### Methyl 2-chloro-4-(1H-pyrazol-5-yl)-1,3-thiazole-5-carboxylate

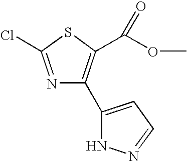

A solution of 700 mg (2.6 mmol) methyl 2-chloro-4-[(2E)-3-(dimethylamino)prop-2-enoyl]-1,3-thiazole-5-carboxylate (Intermediate 58) and 193 mg (2.8 mmol) hydrazine hydrochloride in 4 ml AcOH was heated at 120° C. for 1 hour. Solvent was removed and the residue was partitioned between aqueous NaHCO₃ and EtOAc. The EtOAc was separated, washed with brine, dried (MgSO₄) and concentrated. The residue was chromatographed on silica gel (100% DCM followed by gradient elution to 100% EtOAc) to give 200 mg of product as a white solid. NMR: 4.00 (s, 3H), 7.12 (s, 1H), 7.73 (s, 1H), 13.04 (s, 1H).

### Intermediate 61

### Methyl 2-chloro-4-(chloroacetyl)-1,3-thiazole-5-carboxylate

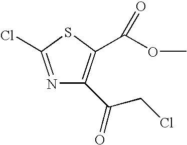

A solution of 2.0 g (9.1 mmol) of methyl 4-acetyl-2-chloro-1,3-thiazole-5-carboxylate (WO 2006087543 A1) and 6.3 g (18.2 mmol) trimethylbenzylammonium dichloroiodide in 50 ml 1,2-dichloroethane and 20 ml MeOH was heated at reflux for 3 hours. Solvent was removed, and the residue was taken up in EtOAc and washed with aqueous NaHSO₃, water and brine. Drying (MgSO₄) and removal of solvent gave 2.3 g of an oil: MS (ES) (M+H)⁺: 222 for C₇H₅Cl₂NO₃S; NMR (CDCl₃): 3.91 (s, 3H), 4.72 (s, 2H).

### Intermediate 62

### Ethyl (3S,4R)-4-{[(3-chloro-4-cyano-5-methyl-1H-pyrrol-2-yl)carbonyl]amino}-3-methoxypiperidine-1-carboxylate

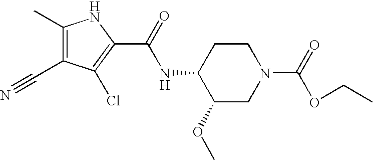

A solution of 503 mg (2.7 mmol) 3-chloro-4-cyano-5-methyl-1H-pyrrole-2-carboxylic acid (Intermediate 68), ethyl (3S,4R)-4-amino-3-methoxypiperidine-1-carboxylate (551 mg, 2.7 mmol) (WO 2006087543 A1), hydroxybenzotriazole (0.358 mg, 2.7 mmol) and N-methylmorpholine (0.58 ml, 3 mmol) in DCM (100 ml) was stirred at room temperature before adding 1-(3-dimethylaminopropyl)-3-ethylcarbodiimide hydrochloride (0.588 mg, 3 mmol). After stirring at room temperature for 4 hours, the crude reaction mixture was diluted with EtOAc and washed with saturated aqueous sodium carbonate, water, 1N HCl, water, and brine. The organic portion was then dried with MgSO₄ and concentrated. The residue was chromatographed on silica gel (100% DCM followed by gradient elution to 100% EtOAc) affording a solid that was triturated with MeOH to give 515 mg of product as a solid: MS (ES) (M+H)⁺: 367 for C₁₆H₂₁ClN₄O₄; NMR: 1.23 (t, 3H), 1.57 (m, 2H), 2.33 (s, 3H), 2.67-3.04 (m, 2H), 3.34 (m, 4H), 3.41 (m, 1H), 3.76-4.33 (m, 5H), 7.25 (d, 1H), 12.73 (s, 1H).

### Intermediate 63

The following Intermediate were synthesized by an analogous method to Intermediate 62 from the starting materials (SM) given in the table below.

| Int | Compound | Data | SM |
|---|---|---|---|
| 63 | Ethyl (3S,4R)-4-{[(4-cyano-5- | MS (ES) (M − H)⁻: 333 for | Intermediate 69 and |
| | methyl-1H-pyrrol-2- | C₁₆H₂₂N₄O₄ | ethyl (3S,4R)-4- |
| | yl)carbonyl]amino}-3- | NMR: 1.22 (t, 3H), 1.52 | amino-3- |
| | methoxypiperidine-1-carboxylate | (m, 1H), 1.79 (m, 1H), | methoxypiperidine- |
| | 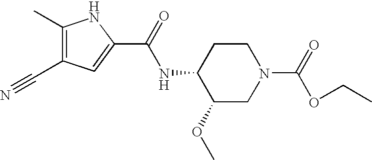 | 2.32 (s, 3H), 2.77-3.14(m, 2H), 3.32-3.43 (m,4H), 3.79-4.33 (m, 5H),7.21 (s, 1H), 7.89 (d,1H), 12.21 (s, 1H). | 1-carboxylate (WO2006087543 A1) |

### Intermediate 64

### 3-Chloro-4-cyano-N-[(3S,4R)-3-methoxypiperidin-4-yl]-5-methyl-1H-pyrrole-2-carboxamide

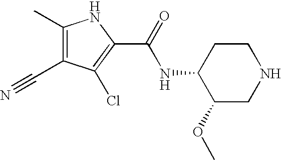

A solution of ethyl (3S,4R)-4-{[(3-chloro-4-cyano-5-methyl-1H-pyrrol-2-yl)carbonyl]amino}-3-methoxypiperidine-1-carboxylate (Intermediate 62; 578 mg, 1.57 mmol) and 0.83 ml (15.6 mmol) 50% NaOH in 7 ml MeOH was heated at 120° C. for 2 hours in a microwave reactor. The mixture was diluted with water and saturated with NaCl before being extracted 5 times with THF. The THF extracts were dried (MgSO₄) and concentrated to give material that was triturated with MeOH to give 215 mg of a white solid: MS (ES) (M+H)⁺: 297 for C₁₃H₁₇ClN₄O₄; NMR: 1.62 (m, 2H), 2.34 (s, 3H), 2.61 (dm, 2H), 2.91 (dm, 1H), 3.14 (dm, 1H), 3.37 (m, 4H), 7.23 (d, 1H), 7.71 (s, 1H).

### Intermediate 65

### (3S,4R)-4-{[(4-Cyano-5-methyl-1H-pyrrol-2-yl)carbonyl]amino}-3-methoxypiperidinium trifluoroacetate

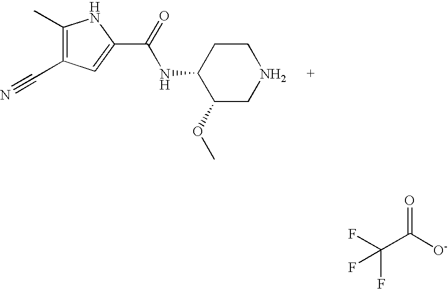

A solution of ethyl (3S,4R)-4-{[(4-cyano-5-methyl-1H-pyrrol-2-yl)carbonyl]amino}-3-methoxypiperidine-1-carboxylate (Intermediate 63; 340 mg, 1.0 mmol) and 0.54 ml (10 mmol) 50% NaOH in 5 ml MeOH was heated at 120° C. for 2 hours in a microwave reactor. The mixture was diluted with water and saturated with NaCl before being extracted 5 times with THF. The THF extracts were dried (MgSO₄) and concentrated to give material that was purified by reverse phase HPLC (10-20% gradient acetonitrile in water with 0. 1% TFA) to give 200 mg of product as the TFA salt: MS (ES) (M−H)⁻: 261 for C₁₆H₂₂N₄O₄; NMR: 1.71 (m, 1H), 2.04 (m, 1H), 2.34 (s, 3H), 3.02-3.34 (m, 3H), 3.41 (m, 4H), 3.52 (m, 1H), 3.61 (m, 1H), 4.18 (m, 2H), 7.21 (s, 1H), 8.05 (d, 1H), 8.25 (d, 1H), 8.78 (m, 1H), 12.21 (s, 1H).

### Intermediate 66

### Ethyl 4-cyano-5-methyl-1H-pyrrole-2-carboxylate

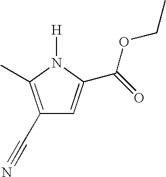

A solution of 3.13 g (20 mmol) ethyl 5-methyl-1H-pyrrole-2-carboxylate (Curran, T. P.; Keaney, M. T. J. Org. Chem. (1996), 61(25), 9068-9069) and 4 ml DMF in 60 ml acetonitrile was cooled in an ice water bath. Chlorosulfonyl isocyanate (2 ml, 23 mmol) was added, and the mixture was stirred overnight with warming to room temperature. Additional chlorosulfonyl isocyanate (0.4 ml, 4.6 mmol) was added, and the mixture was stirred 3 hours. Saturated aqueous Na₂CO₃ (40 ml) was added and solvent was removed. The residue was taken up in water and extracted 2 times with 1:1 EtOAc-diethyl ether with each extract being washed twice more with water and once with brine. The combined organic layers were dried (MgSO₄), and solvent was removed to give 3.5 g of a white solid: MS (ES) (M+H)⁺: 179 for C₉H₁₀N₂O₂; NMR: 1.32 (t, 3H), 2.35 (s, 3H), 4.17 (q, 1H), 7.16 (s, 1H), 12.59 (s, 1H).

### Intermediate 67

### Ethyl 3-chloro-4-cyano-5-methyl-1H-pyrrole-2-carboxylate

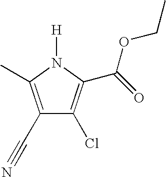

A solution of 1.72 g (11.5 mmol) of ethyl 4-cyano-5-methyl-1H-pyrrole-2-carboxylate (Intermediate 66) and 1.53 g (11.5 mmol) N-chlorosuccinimide in 20 ml DMF was heated to 70° C. in a microwave reactor for 1 hour. Solvent was removed and the residue was diluted with water and extracted twice with diethyl ether, each extract being washed 2 times more with water and once with brine. The combined organic layers were dried (MgSO₄), and solvent was removed. The residue was chromatographed on silica gel (100% DCM followed by gradient elution to 100% EtOAc) to afford 790 mg of product as a solid: MS (ES) (M−H)⁻: 211 for C₉H₉ClN₂O₂; NMR: 1.32 (t, 3H), 2.35 (s, 3H), 4.32 (q, 1H), 12.85 (s, 1H).

### Intermediate 68

### 3-Chloro-4-cyano-5-methyl-1H-pyrrole-2-carboxylic acid

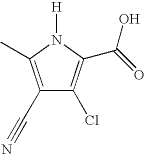

A solution of 760 mg (3.6 mmol) of ethyl 3-chloro-4-cyano-5-methyl-1H-pyrrole-2-carboxylate (Intermediate 67) and 3.6 ml (7.2 mmol) of 2N LiOH in 15 ml MeOH was heated at 100° C. for 3 hours in a microwave reactor. Additional 2N LiOH (1.0 ml) was added, and the mixture was heated at 100° C. for 1 hour. The mixture was diluted with water and acidified with 1N HCl before being extracted 2 times with EtOAc. The EtOAc extracts were washed with brine, dried (MgSO₄) and concentrated to give 610 mg of a solid: MS (ES) (M−H)⁻: 183 for C₇H₅ClN₂O₂; NMR: 2.31 (s, 3H), 12.72 (s, 1H), 13.31 (s, 1H).

### Intermediate 69

### 4-Cyano-5-methyl-1H-pyrrole-2-carboxylic acid

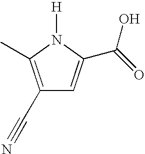

A solution of 390 mg (2.6 mmol) of ethyl 4-cyano-5-methyl-1H-pyrrole-2-carboxylate (Intermediate 66) and 2.6 ml (7.2 mmol) of 2N LiOH in 15 ml MeOH was heated at 100° C. for 3 hours in a microwave reactor. Additional 2N LiOH (0.5 ml) was added, and the mixture was heated at 100° C. for 1 hour. The mixture was diluted with water and acidified with 1N HCl before being extracted 2 times with EtOAc. The EtOAc extracts were washed with brine, dried (MgSO₄) and concentrated to give 317 mg of a solid: MS (ES) (M+H)⁺: 151 for C₇H₆N₂O₂; NMR: 2.31 (s, 3H), 7.01 (s, 1H), 12.47 (s, 1H), 12.75 (s, 1H).

### Intermediate 70

### Methyl 2-chloro-4-(4-methoxypyrimidin-2-yl)thiazole-5-carboxylate

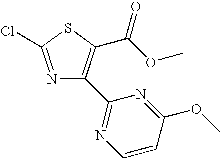

Methyl 2-amino-4-(4-methoxypyrimidin-2-yl)thiazole-5-carboxylate (Intermediate 71; 100 mg, 0.38 mmol) was suspended in 4 ml of glacial acetic acid and 6 ml of hydrochloric acid, The solution was cooled down to 0° C. and sodium nitrite (78 mg, 1.13 mmol) in 3 ml of H₂O was added dropwise. After stirring at 0° C. for 10 min, it was warmed up to room temperature and stirred for 30 min until LC-MS showed no starting material remaining. A solution of urea (50 mg) in 2 ml of H₂O was added dropwise, stirred for 20 min. Volume of the solution was then reduced under reduced pressure to 7 ml, Na₂CO₃ saturated aqueous solution was added slowly while stirring to neutralize the solution. The resulting liquid was extracted with EtOAc (10 ml×3), The combined EtOAc layer was washed with saturated NaHCO₃ solution and brine, dried over MgSO₄ and concentrated to an oil. This was then purified by column chromatography eluted with Hexanes/EtOAc to give the desired product as an oil (75 mg). MS (ES) (M+H)⁺: 286 for C₁₀H₈N₃O₃S; NMR (CDCl₃): 3.81 (s, 3H), 3.97 (s, 3H), 6.74 (d, 1H), 8.53 (d, 1H).

### Intermediate 71

### Methyl 2-amino-4-(4-methoxypyrimidin-2-yl)thiazole-5-carboxylate

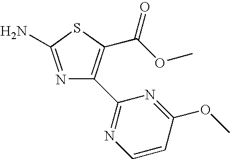

Methyl 3-(4-methoxypyrimidin-2-yl)-3-oxopropanoate (Intermediate 72; 250 mg, 1.19 mmol) was dissolved in EtOAc (10 ml), Amberlyst 15 ion-exchange resin (230 mg) and n-iodo succinimide (282 mg, 1.19 mmol) were added, and the reaction mixture was stirred at room temperature for 1 hr. The mixture was filtered and the filter cake was rinsed with MeOH, the combined filtrate was concentrated to dryness. Either was added and the resulting precipitate was filtered off, the filtrate was concentrated to an oil and dried under high vacuum. To this crude thiourea (136 mg, 1.78 mmol) and MeOH (10.00 ml) was added and the mixture was refluxed for 1.5 hr, cooled down to rt and yellowish precipitate was filtered and washed with MeOH, the filter cake was washed with saturated Na₂CO₃ aqueous solution and kept as the desired product (off-white solid), the filtrate was then concentrated to dryness and was suspended into saturated Na₂CO₃ solution (10 ml), the resulting precipitate was collected by filtration. LC-MS showed both solids were the desired product. (97 mg). MS (ES) (M+H)⁺: 267 for C₁₀H₁₀N₄O₃S; NMR (CDCl₃): 3.55 (s, 3H), 3.89 (s, 3H), 6.95 (d, 1H), 7.95 (br, 2H), 8.62 (d, 1H).

### Intermediate 72

### Methyl 3-(4-methoxypyrimidin-2-yl)-3-oxopropanoate

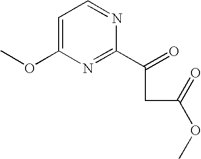

1-(4-Methoxypyrimidin-2-yl)ethanone (Intermediate 73; 462 mg, 3.04 mmol) was dissolved in dimethyl carbonate (20 ml, 237.35 mmol) and the reaction mixture was cooled down to 0° C., sodium hydride (291 mg, 12.15 mmol) was added and the mixture was stirred at 0° C. for 10 min, raised the temperature to 100° C., refluxed for 1 hr. The mixture was cooled down to RT and was poured slowed into the ice cold 1N HCl solution (20 ml), stirred for 5 min, pH was brought back to ˜7 and extracted with EtOAc. Organic layer was washed with brine and dried over MgSO₄, concentrated to oil. Chromatography gave a mixture of 2 compounds. Purified by column eluted with DCM/EtOAc (100˜30%) gave the desired product as an oil (250 mg), it was carried over to the next step without further purification. MS (ES) (M+H)⁺: 211 for C₉H₁₀N₂O₄S.

### Intermediate 73

### 1-(4-Methoxypyrimidin-2-yl)ethanone

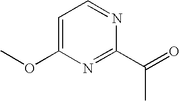

2-Iodo-4-methyoxypyrimidine (Intermediate 74; 1.62 g, 6.84 mmol) was dissolved in dry THF (20 ml), cooled down to −10° C., i-PrMgCl (2 M in ether, 3.42 ml, 6.84 mmol) was added and the reaction mixture was stirred at 0° C. for 1 hour. N-Methoxy-N-methyl acetamide (776 mg, 7.52 mmol) was added; the mixture was slowly warmed up to room temperature over night. Water (10 ml) was added and the reaction mixture was extracted with DCM. The organic layer was dried over MgSO₄, concentrated and purified by column chromatography (Hex/EtOAc, gradient) to give the desired product as a yellowish solid (470 mg). MS (ES) (M+H)⁺: 153 for C₇H₈N₂O₂; NMR (CDCl₃): 2.77 (s, 3H), 4.10 (s, 3H), 6.89 (d, 1H), 8.62 (d, 2H).

### Intermediate 74

### 2-Iodo-4-methoxypyrimidine

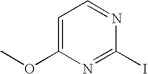

2-Chloro-4-methoxypyrimidine (1.05 g, 7.26 mmol) was dissolved in DCM (20 ml), cooled down to −5° C., hydrogen iodide (55%, 36 mmol) was added drop wise, the mixture was stirred at 0° C. for 4 hours then slowly warmed up to room temperature over night. The reaction mixture was cooled down to 0° C. and K₂CO₃ was added to neutralize the solution to pH=7, followed by the addition of 30% sodium meta bisulfite. This mixture was extracted with DCM (20 ml×3). Organic layer was dried over anhydrous Na₂SO₄, concentrated to give a white solid as the desired product (1.62 g). MS (ES) (M+H)⁺: 237 for C₅H₅IN₂O; NMR (CDCl₃): 4.00 (s, 3H), 6.73 (d, 1H), 8.13 (d, 1H).

### Intermediate 75

### Ethyl 2-chloro-4-(1,3,4-oxadiazol-2-yl)thiazole-5-carboxylate

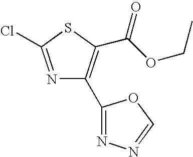

Ethyl 2-chloro-4-(2-formylhydrazinecarbonyl)thiazole-5-carboxylate (Intermediate 76; 220 mg, 0.79 mmol), phosphorus triphenyl (416 mg, 1.58 mmol), carbon tetrachloride (0.154 ml, 1.58 mmol) and DIEA (0.415 ml, 2.38 mmol) were mixed in anhydrous acetonitrile (10 ml), stirred at room temperature over night, LC-MS indicated the SM was disappeared and the desired product mass and a side product. Solvent was evaporated and the residue was purified by column chromatography (DCM/EtOAc) afforded the desired product (78 mg). MS (ES) (M+H)⁺: 260 for C₈H₆ClN₃O₃S; NMR (CDCl₃): 1.36 (t, 3H), 4.40 (q, 2H), 8.59 (s, 1H).

### Intermediate76

### Ethyl 2-chloro-4-(2-formylhydrazinecarbonyl)thiazole-5-carboxylate

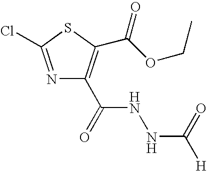

Ethyl 2-chloro-4-(chlorocarbonyl)thiazole-5-carboxylate (Intermediate 77; 300 mg, 1.18 mmol)and 2,6-lutidine (0.206 ml, 1.77 mmol) were mixed in 5 ml of dry DCM, cooled down to 0° C., formohydrazide (73.8 mg, 1.20 mmol) was added, the reaction mixture was stirred at 0° C. for 5 min and gradually warmed up to room temperature overnight. Solvent was removed and the residual was purified by column eluted with DCM/MeOH gradient. Product was obtained as yellowish solid. (230 mg). MS (ES) (M+H)⁺: 278 for C₈H₈ClN₃O₄S; NMR (CDCl₃): 1.38 (t, 3H), 4.44 (q, 2H), 8.24 (s, 1H), 9.41 (br, 1H), 11.68 (br, 1H).

### Intermediate 77

### Ethyl 2-chloro-4-(chlorocarbonyl)thiazole-5-carboxylate

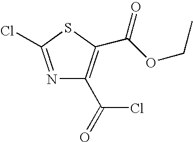

2-Chloro-5-(ethoxycarbonyl)thiazole-4-carboxylic acid (2 g, 8.49 mmol) was dissolved in dry DCM (15 ml), oxalyl dichloride (1.131 g, 8.91 mmol) was added and the mixture was cooled to 0° C., 2 drops of DMF was added and the reaction mixture was stirred at room temperature until the bubbling stopped. Solvent was removed and the residual was dried under high vacuum over night afforded the desired product as a crystal (2.15 g). NMR (CDCl₃): 1.35 (t, 3H), 4.37 (q, 2H).

### Intermediate 78

### Ethyl 2-chloro-4-(3-methyl-1,2,4-oxadiazol-5-yl)thiazole-5-carboxylate

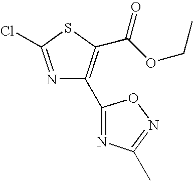

Ethyl 2-chloro-4-(chlorocarbonyl)thiazole-5-carboxylate (Intermediate 77; 300 mg, 1.18 mmol) and 2,6-lutidine (0.206 ml, 1.77 mmol) were mixed in 5 ml of dry DCM, cooled down to 0° C., (Z)-N′-hydroxyacetimidamide (WO200032565, 91 mg, 1.18 mmol) was added, the reaction mixture was stirred at 0° C. for 5 min, DMF (2 ml) was added to assist the solvation of the SM and the reaction mixture was gradually warmed up to room temperature overnight. DCM was removed and the remaining solution was heated to 55° C. and stirred for 4 days. LC-MS showed the reaction was completed. The reaction mixture was diluted with EtOAc (20 ml), washed with water (10 ml×3), the organic phase was washed with brine and dried over MgSO₄, concentrated and purified by column chromatography (Hex/EtOAc) to give the desired product (yellowish crystal, 140 mg). MS (ES) (M+H)⁺: 274 for C₉H₈ClN₃O₃S; NMR (CDCl₃): 1.36 (t, 3H), 2.55 (s, 3H), 4.40 (q, 2H).

### Intermediate 79

### Ethyl 2-chloro-4-(5-methyl-1,3,4-oxadiazol-2-yl)thiazole-5-carboxulate

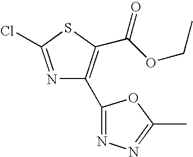

Ethyl 4-(2-acetylhydrazinecarbonyl)-2-chlorothiazole-5-carboxylate (Intermediate 80; 264 mg, 0.91 mmol), phosphorus triphenyl (475 mg, 1.81 mmol), carbon tetrachloride (0.176 ml, 1.81 mmol) and diisopropyl ethylamine (0.484 ml, 2.72 mmol) were mixed in anhydrous acetonitrile (10 ml). The mixture was stirred at room temperature over night. solvent was then evaporated. The residue was purified by column chromatography (DCM/EtOAc) to give the desired product as a solid (211 mg). MS (ES) (M+H)⁺: 274 for C₉H₈ClN₃O₃S; NMR (CDCl₃): 1.37 (t, 3H), 2.67 (s, 3H), 4.40 (q, 2H).

### Intermediate 80

### Ethyl 4-(2-acetylhydrazinecarbonyl)-2-chlorothiazole-5-carboxylate

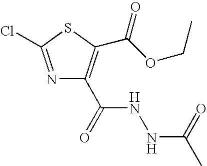

Ethyl 2-chloro-4-(chlorocarbonyl)thiazole-5-carboxylate (Intermediate 77; 300 mg, 1.18 mmol) and 2,6-lutidine (0.206 ml, 1.77 mmol) were mixed in 5 ml of dry DCM, cooled down to 0° C., acetohydrazide (97 mg, 1.18 mmol) was added, the reaction mixture was stirred at 0° C. for 5 min and gradually warmed up to room temperature overnight. Solvent was removed and the residual was purified by column eluted with DCM/MeOH gradient. Product was obtained as yellowish solid. (264 mg). MS (ES) (M+H)⁺: 274 for C₉H₁₀ClN₃O₄S; NMR (CDCl₃): 1.39 (t, 3H), 2.12 (s, 3H), 4.45 (q, 2H), 8.55 (d, 1H), 11.66 (d, 1H).

### Intermediate 81-86

The following Intermediates were synthesized by an analogous method to Intermediate 1 from the starting materials (SM) given in the table below.

| Int | Compound | Data | SM |
|---|---|---|---|
| 81 | methyl 2-chloro-4-(1-methyl-1H- | MS (ES): 259 (MH⁺) for | Intermediate 87 |
| | 1,2,3-triazol-4-yl)-1,3-thiazole-5- | C₈H₇ClN₄O₂S | |
| | carboxylate | ¹H-NMR (CDCl₃) δ: | |
| | 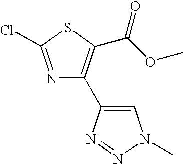 | 3.9 (s, 3H); 4.18 (s, 3H);8.71 (s, 1H). | |
| 82 | methyl 2-chloro-4-(3,5- | MS (ES): 284 (MH⁺) for | Intermediate 88 |
| | dimethylpyrazin-2-yl)-1,3-thiazole- | C₁₁H₁₀ClN₃O₂S | |
| | 5-carboxylate | ¹H-NMR (CDCl₃): 2.43 | |
| | 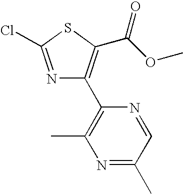 | (s, 3H); 2.57 (s, 3H);3.76 (s, 3H); 8.42 (s, 1H). | |
| 83 | methyl 2-chloro-4-(6- | MS (ES): 285 (MH⁺) for | Intermediate 89 |
| | methoxypyridin-2-yl)-1,3-thiazole- | C₁₁H₉ClN₂O₃S | |
| | 5-carboxylate | ¹H-NMR (CDCl₃): 3.85 | |
| | 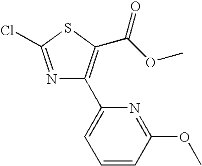 | (s, 3H); 3.93 (s, 3H);6.85 (d, 1H); 7.41 (d,1H); 7.73 (t, 1H). | |
| 84 | ethyl 2-chloro-4-(4-methoxypyridin- | MS (ES) 299 (MH⁺) for | Intermediate 90 |
| | 2-yl)-1,3-thiazole-5-carboxylate | C₁₂H₁₁ClN₂O₃S | |
| | 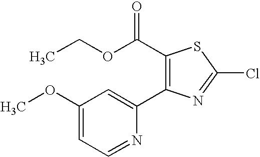 | ¹H-NMR (CDCl₃): 1.32(t, 3H); 3.77 (s, 3H); 4.21(q, 2H); 6.77 (dd, 1H);7.25 (d, 1H); 8.40 (d,1H). | |
| 85 | ethyl 2-chloro-4-(5-methylpyrazin- | MS (ES): 284 (MH⁺) for | Intermediate 91 |
| | 2-yl)-1,3-thiazole-5-carboxylate | C₁₁H₁₀ClN₃O₂S | |
| | 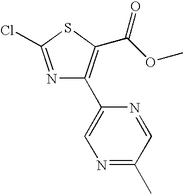 | ¹H-NMR (CDCl₃): 2.43(s, 3H); 2.57 (s, 3H);3.76 (s, 3H); 8.42 (s, 1H). | |
| 86 | ethyl 2-chloro-4-(3-fluoropyridin-2- | MS (ES): 287 (MH⁺) for | Intermediate 92 |
| | yl)-1,3-thiazole-5-carboxylate | C₁₁H₈ClFN₂O₂₅S | |
| | 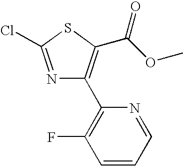 | ¹H-NMR: 1.25 (t, 3H);4.28 (q, 2H); 7.55 (dd,1H); 7.62 (t, 1H); 8.62(d, 1H). | |

### Intermediate 87-92

The following Intermediates were synthesized by an analogous method to Intermediate 16 from the starting materials (SM) given in the table below.

| Int | Compound | Data | SM |
|---|---|---|---|
| 87 | 2-methyl 2-amino-4-(1-methyl-1H- | MS (ES) 240 (MH⁺) for | Intermediate 93 |
| | 1,2,3-triazol-4-yl)-1,3-thiazole-5- | C₈H₉N₅O₂S | |
| | carboxylate | ¹H-NMR (CDCl₃): 3.80 | |
| | 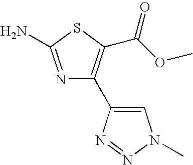 | (s, 3H); 4.18 (s, 3H);8.16 (s, 1H). | |
| 88 | methyl 2-amino-4-(3,5- | MS (ES): 278 (MH⁺) for | Intermediate 94 |
| | dimethylpyrazin-2-yl)-1,3-thiazole- | C₁₁H₁₂N₄O₂S | |
| | 5-carboxylate | ¹H-NMR (CDCl₃): 2.49 | |
| | 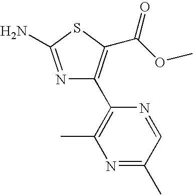 | (s, 3H); 2.59 (s, 3H);3.71 (s, 3H); 6.90 (br,2H); 8.44 (s, 1H). | |
| 89 | methyl 2-amino-4-(6- | MS (ES): 266 (MH⁺) for | Intermediate 95 |
| | methoxypyridin-2-yl)-1,3-thiazole- | C₁₁H₁₁N₃O₃S | |
| | 5-carboxylate | ¹H-NMR (CDCl₃): 3.75 | |
| | 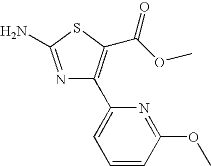 | (s, 3H); 3.98 (s, 3H);6.27 (br, 2H); 6.77 (d,1H); 7.42 (d, 1H); 7.63(t, 1H). | |
| 90 | ethyl 2-amino-4-(4-methoxypyridin- | MS (ES): 280 (MH⁺) for | Intermediate 96 |
| | 2-yl)-1,3-thiazole-5-carboxylate | C₁₂H₁₃N₃O₃S | |
| | 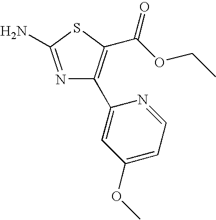 | ¹H-NMR (CDCl₃): 1.20(t, 3H); 3.87 (s, 3H); 4.15(q, 2H); 6.27 (br, 2H);6.83 (dd, 1H); 7.33 (d,1H); 8.47 (d, 1H). | |
| 91 | ethyl 2-amino-4-(5-methylpyrazin- | MS (ES): 278 (MH⁺) for | Intermediate 97 |
| | 2-yl)-1,3-thiazole-5-carboxylate | C₁₁H₁₂N₄O₂S | |
| | 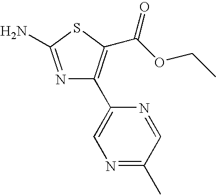 | ¹H-NMR (CDCl₃): 1.30(t, 3H); 1.57 (s, 2H); 2.65(s, 3H); 4.30 (q, 2H);8.55 (s, 1H); 8.91 (s, 1H). | |
| 92 | ethyl 2-amino-4-(3-fluoropyridin-2- | MS (ES): 268 (MH⁺) for | Intermediate 98 |
| | yl)-1,3-thiazole-5-carboxylate | C₁₁H₁₀FN₃O₂S | |
| | 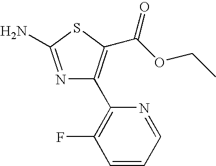 | ¹H-NMR: 1.01 (t, 3H);3.99 (q, 2H); 7.53 (dd,1H); 7.77 (t, 1H); 7.95 (s,2H); 8.44 (d, 1H). | |

### Intermediate 93-98

The following Intermediates were synthesized by an analogous method to Intermediate 44 from the starting materials (SM) given in the table below.

| Int | Compound | Data | SM |
|---|---|---|---|
| 93 | methyl 3-(1-methyl-1H-1,2,3- | MS (ES): 184 (MH⁺) for | 1-(1-methyl-1H- |
| | triazol-4-yl)-3-oxopropanoate | C₇H₉N₃O₃ | 1,2,3-triazol-4- |
| | 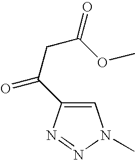 | ¹H-NMR (CDCl₃): 3.74(s, 3H); 4.14 (s, 2H);4.16 (s, 3H); 8.10 (s, 1H). | yl)ethanone BullSoc Chim Belg[BSCBAG]1991,100(4), 289-290 |
| 94 | methyl 3-(3,5-dimethylpyrazin-2- | MS (ES): 209 (MH⁺) for | 1-(3,5- |
| | yl)-3-oxopropanoate | C₁₀H₁₂N₂O₃ | dimethylpyrazin-2- |
| | 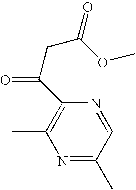 | ¹H-NMR (CDCl₃): 2.55(s, 3H); 2.81 (s, 3H);3.72 (s, 3H); 4.15 (s,2H); 8.48 (s, 1H). | yl)ethanone |
| 95 | methyl 3-(6-methoxypyridin-2-yl)- | MS (ES): 210 (MH⁺) for | 1-(6- |
| | 3-oxopropanoate | C₁₀H₁₁NO₄ | methoxypyridin-2- |
| | 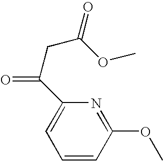 | ¹H-NMR (CDCl₃): 3.71(s, 3H); 3.94 (s, 3H);4.09 (s, 2H); 6.95 (d,1H); 7.68 (d, 1H); 7.72(t, 1H). | yl)ethanone |
| 96 | ethyl 3-(4-methoxypyridin-2-yl)-3- | MS (ES): 224 (MH⁺) for | 3-(4- |
| | oxopropanoate | C₁₁H₁₃NO₄ | methoxypyridin-2- |
| | 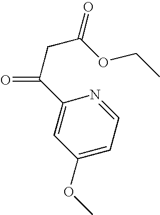 | | yl)ethanone |
| 97 | ethyl 3-(5-methylpyrazin-2-yl)-3- | MS (ES): 209 (MH⁺) for | 1-(5- |
| | oxopropanoate | C₁₀H₁₂N₂O₃ | methylpyrazin-2- |
| | 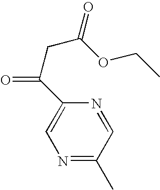 | ¹H-NMR (CDCl₃: 1.23(t, 3H); 2.66 (s, 3H); 4.12(s, 2H); 4.20 (q, 2H);8.48 (s, 1H); 9.13 (s, 1H). | yl)ethanone |
| 98 | ethyl 3-(3-fluoropyridin-2-yl)-3- | MS (ES): 212 (MH⁺) for | 1-(3-fluoropyridin- |
| | oxopropanoate | C₁₀H₁₀FNO₃ | 2-yl)ethanone |
| | 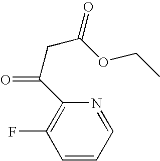 | ¹H-NMR: 1.15 (t, 3H);3.99 (q, 2H); 4.10 (s,2H); 7.45 (dd, 1H); 8.36(t, 1H); 8.56 (d, 1H). | |

### Intermediate 99

### Ethyl (3S,4R)-4-amino-3-(benzyloxy)piperidine-1-carboxylate

Racemic Ethyl-4-amino-3-(benzyloxy)piperidine-1-carboxylate (WO2006087543) was separated by chiral HPLC (Chiralpak AD 5×50 cm, 20 u, 85% Hexane, 15% ethanol:methanol (1:1), 0.1% diethylamine) to give the desired product.

### Intermediate 100-102

The following Intermediates were synthesized by an analogous method to Intermediate 62 from the starting materials (SM) given in the table below.

| Int | Compound | Data | SM |
|---|---|---|---|
| 100 | Ethyl(3S,4R)-3-(benzyloxy)-4-{[(3,4- | MS (APCI): 454 (M + H) | Intermediate 99 and |
| | dichloro-5-methyl-1H-pyrrol-2- | ¹H NMR: δ 1.22 (br s, | 3,4-dichloro-5- |
| | yl)carbonyl]amino}piperidine-1- | 3H), 1.65 (m, 2H), 1.93 | methyl-1H-pyrrole- |
| | carboxylate: | (s, 3H), 3.30 (m, 2H), | 2-carboxylic acid |
| | 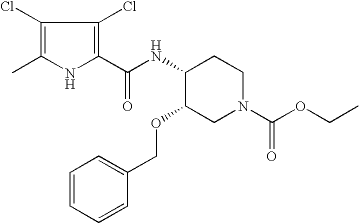 | 3.65 (br s, 1H), 4.09 (brs, 2H), 4.22 (m, 1H),4.45 (m, 2H), 4.75 (d,1H), 7.12 (br s, 1H),7.35 (m, 5H), 12.16 (s,1H). | (WO2006087543) |
| 101 | Ethyl (3S,4R)-3-(benzyloxy)-4-{[(4- | ¹H NMR: 1.19 (t, 3H), | Intermediate 99 and |
| | chloro-5-methyl-1H-pyrrol-2- | 1.53 (m, 1H), 1.86 (m, | 4-chloro-5-methyl- |
| | yl)carbonyl]amino}piperidine-1- | 1H), 2.18 (s, 1H), 3.08 | 1H-pyrrole-2- |
| | carboxylate | (m, 2H), 3.65 (s, 1H), | carboxylic acid |
| | 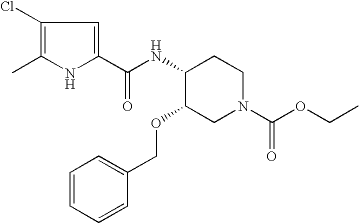 | 4.09 (q, 4H), 4.38 (m,3H), 4.63 (d, 2H), 6.89(s, 1H), 7.27 (d, 5H),7.64 (m, 1H), 11.56 (s,1H). | (WO2006087543) |
| 102 | Ethyl (3S,4R)-3-(benzyloxy)-4- | ¹H NMR: 1.21 (t, 3H), | Intermediate 99 and |
| | {[(4-chloro-3-cyano-5-methyl-1H- | 1.61 (m, 1H), 2.21 (s, | 4-chloro-3-cyano- |
| | pyrrol-2-yl)carbonyl]amino}piperidine- | 3H), 3.04 (m, 2H), 3.68 | 5-methyl-1H- |
| | 1-carboxylate | (s, 1H), 4.06 (m, 3H), | pyrrole-2- |
| | 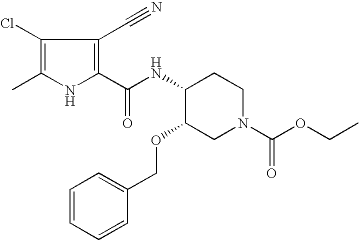 | 4.24 (q, 2H), 4.48 (d,1H), 4.64 (d, 1H), 7.29(d, 3H), 7.38 (s, 2H),7.62 (s, 1H), 12.61 (s,1H). | carboxylic acid |

### Intermediate 103-105

The following Intermediates were synthesized by an analogous method to Intermediate 64 from the starting materials (SM) given in the table below.

| Int | Compound | Data | SM |
|---|---|---|---|
| 103 | N-[(3S,4R)-3-(benzyloxy)piperidin-4- | MS (APCI): 382 (M + H) | Intermediate 100 |
| | yl]-3,4-dichloro-5-methyl-1H-pyrrole-2- | ¹H NMR: 2.17 (s, 3H), | |
| | carboxamide | 2.58 (m, 2H), 2.88 (d, | |
| | 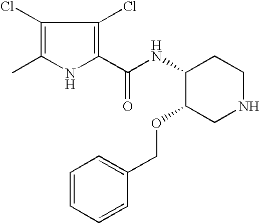 | 1H), 3.18 (d, 1H), 3.58 (s,1H), 4.16 (br s, 1H), 4.43(d, 1H), 4.69 (d, 1H), 7.36(m, 5H), 7.09 (br s, 1H),12.09 (m, 1H).HPLC: 96.39% | |
| 104 | N-[(3S,4R)-3-(benzyloxy)piperidin- | ¹H NMR: 1.43 (m, 1H), | Intermediate 101 |
| | 4-yl]-4-chloro-5-methyl-1H-pyrrole- | 1.84 (m, 1H), 2.20 (s, | |
| | 2-carboxamide | 3H), 2.45 (m, 1H), 2.94 | |
| | 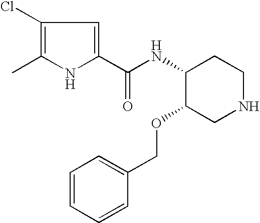 | (d, 2H), 3.16 (m, 1H),3.56 (s, 1H), 4.06 (br s,1H), 4.45 (d, 1H), 4.64(d, 1H), 6.87 (s, 1H),7.32 (dd, 5H), 7.59 (d,1H), 11.62 (s, 1H). | |
| 105 | N-[(3S,4R)-3-(benzyloxy)piperidin- | ¹H NMR: 1.64 (m, 1H), | Intermediate 102 |
| | 4-yl]-4-chloro-3-cyano-5-methyl-1H- | 1.78 (m, 1H), 2.19 (s, | |
| | pyrrole-2-carboxamide | 3H), 2.78 (m, 2H), 2.98 | |
| | 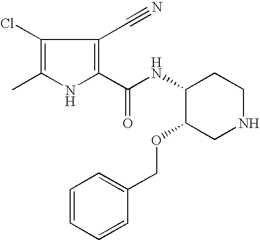 | (d, 1H), 3.61 (s, 1H),3.27 (d, 1H), 4.19 (br s,1H), 4.51 (d, 1H), 4.67(d, 1H), 7.27 (m, 3H),7.43 (d, 2H), 7.54 (d,1H). | |

### Intermediate 106

### Ethyl 4-(benzyloxy)-3-oxobutanoate

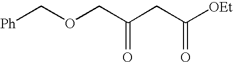

Sodium hydride (145 g, 6.06 mol) was suspended in tetrahydrofuran (3.0 L). Benzyl alcohol (328 g, 3.03 mol) and ethyl 4-chloro-3-oxobutanoate (500 g, 3.03 mol) were added to the suspension and the reaction mixture was stirred at 40° C. for 2 h. The reaction mixture was cooled to room temperature, quenched with ice water and extracted with ethyl acetate. The combined organic layers were dried over anhydrous sodium sulphate and concentrated under vacuum to afford 500 g (69%) of Ethyl 4-(benzyloxy)-3-oxobutanoate.

¹H NMR (400 MHz, CDCl₃): δ1.29 (t, 3H), 3.53 (s, 2H), 4.22 (q, 2H), 4.22 (s, 2H), 4.60 (s, 2H), 7.38 (m, 5H).

### Intermediate 107

### Ethyl 2-amino-4-[(benzyloxy)methyl]-1,3-thiazole-5-carboxylate

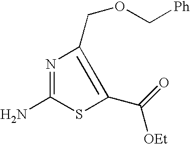

Ethyl 4-(benzyloxy)-3-oxobutanoate (Intermediate 106, 100 g, 0.42 mol) was dissolved in ethyl acetate (800 mL), amberlyst 15 ion exchange resin (100 g) and N-iodo-succinimide (104.4 g, 0.47 mol) were added, and the reaction mixture was stirred at room temperature for 1 h. The reaction mixture was filtered and rinsed with ethyl acetate and the combined filtrate was concentrated under reduced pressure. The resulting residue was partitioned between ethyl acetate and water (1:1) and the resulting organic layer was dried over sodium sulphate and concentrated to dryness to yield a solid, which was dried under high vacuum. To this residue, thiourea (48.0 g, 0.63 mol) and methanol (500 mL) were added and the reaction mixture was heated at reflux for 50 min, cooled to room temperature and the solvent was removed under reduced pressure. The resulting residue was suspended in saturated aqueous sodium bicarbonate solution (500 mL) and extracted with ethyl acetate (5×300 mL). The resulting organic layer was dried over sodium sulphate and concentrated under reduced pressure, diethyl ether was added, and the resulting mixture was stirred for 2 h. The solid that formed was collected by filtration and washed with diethyl ether to yield ethyl 2-amino-4-[(benzyloxy)methyl]-1,3-thiazole-5-carboxylate as a white solid 25 g (20%)

¹H NMR (400 MHz, DMSO-d₆): δ1.23 (t, 3H), 4.19 (q, 2H), 4.51 (s, 2H), 4.72 (s, 2H), 7.35 (m, 5H), 7.80 (s, 2H).

### Intermediate 108

### Ethyl 2-amino-4-(hydroxymethyl)-1,3-thiazole-5-carboxylate

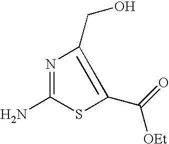

To an anisole solution of aluminium chloride (9.1 g, 68.4 mmol) was added ethyl 2-amino-4-[(benzyloxy)methyl]-1,3-thiazole-5-carboxylate (Intermediate 107, 2.0 g, 6.84 mmol) at room temperature and stirred for 3 h. The reaction mixture was cooled to 0° C. and methanol was added to the reaction mixture followed by removal of the solvent under vacuum. Ice-cold water was added to the resulting residue and stirred for ½ h. The solid that formed was collected by filteration and washed with ethyl acetate to obtain Ethyl 2-amino-4-(hydroxymethyl)-1,3-thiazole-5-carboxylate (0.6 g, 47%) as solid.

¹H NMR (400 MHz, DMSO-d₆): δ1.22 (t, 3H), 4.14 (q, 2H), 4.56 (s, 2H), 4.84 (s, 1H), 7.74 (s, 2H).

### Intermediate 109

### Ethyl 2-amino-4-formyl-1,3-thiazole-5-carboxylate

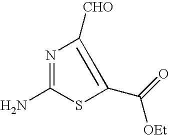

To a solution of ethyl 2-amino-4-(hydroxymethyl)-1,3-thiazole-5-carboxylate (Intermediate 108, 1.0 g, 4.9 mmol) in tetrahydrofuran (50 mL) was added manganese dioxide (6.5 g, 74.2 mmol) and stirred for 8 h at room temperature. The reaction mixture was filtered through celite bed and washed with ethyl acetate. The filtrate was concentrated under reduced pressure to give ethyl 2-amino-4-formyl-1,3-thiazole-5-carboxylate (0.7 g, 70%) as yellow solid. ¹H NMR (400 MHz, DMSO-d₆): δ1.27 (t, 3H), 4.28 (q, 2H), 8.01 (s, 2H), 10.28 (s, 1H).

### Intermediate 110

### Ethyl 2-amino-4-cyano-1,3-thiazole-5-carboxylate

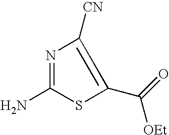

To a solution of ethyl 2-amino-4-formyl-1,3-thiazole-5-carboxylate (Intermediate 109, 7.0 g, 35.1 mmol) in tetrahydrofuran (35 mL) was added aqueous ammonia solution (50 mL) and iodine (8.89 g, 35.1 mMmol) at room temperature. The reaction mixture was stirred for 5 h and the solid that formed was collected by filtration and washed with water to afford ethyl 2-amino-4-cyano-1,3-thiazole-5-carboxylate (3.0 g; 48%) as solid

¹H NMR (400 MHz, DMSO-dd₆): δ1.27 (t, 3H), 4.26 (q, 2H), 8.31 (s, 2H).

### Intermediate 111

### Ethyl 2-chloro-4-cyano-1,3-thiazole-5-carboxylate

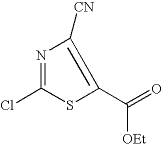

To a 0° C. suspension of ethyl 2-amino-4-cyano-1,3-thiazole-5-carboxylate (Intermediate 110, 1.0 g, 5.5 mmol) in acetic acid (3 mL) and HCl (10 mL), sodium nitrite (1.05 g, 15.1 mM) in water (10 mL) was added drop wise. After stirring at 0° C. for 10 min the reaction mixture was warmed up to room temperature and stirred for 30 min. A solution of urea (0.6 g, 10.1 mmol) in water (5 mL) was added dropwise and the reaction mixture was stirred for 10 min. The reaction mixture was neutralized with solid sodium carbonate and saturated aqueous sodium bicarbonate solution. The resulting liquid was extracted with ethyl acetate (5×30 mL) and the combined ethyl acetate layer was washed with saturated sodium bicarbonate solution and brine, dried over sodium sulphate and concentrated under reduced pressure to an oil, which was purified by column chromatography over silica gel to afford Ethyl 2-chloro-4-cyano-1,3-thiazole-5-carboxylate (0.6 g, 60%).

¹H NMR (400 MHz, CDCl₃): δ1.41 (t, 3H), 4.47 (q, 2H).

### Intermediate 112

### Ethyl 2-chloro-4-(1H-tetrazol-5-yl)-1,3-thiazole-5-carboxylate

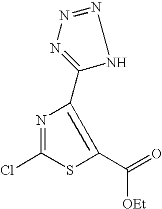

To a solution of ethyl 2-chloro-4-cyano-1,3-thiazole-5-carboxylate (Intermediate 111, 8.0 g, 37.1 mmol) in dioxane (300 mL) was added azido-trimethylsilane (21.2 g, 185.1 mmol) and dimethyl tin oxide (0.6 g, 3.7 mmol). The reaction mixture was heated to reflux for 12 h and concentrated under reduced pressure. Purification by preparative HPLC afforded ethyl 2-chloro-4-(1H-tetrazol-5-yl)-1,3-thiazole-5-carboxylate 3.0 g (31%).

¹H NMR (400 MHz, DMSO-d₆): δ1.19 (t, 3H), 4.27 (q, 2H)

### Intermediate 113

### ethyl (3S,4R)-4-amino-3-propoxypiperidine-1-carboxylate

Racemic ethyl 4-amino-3-propoxypiperidine-1-carboxylate (WO2006087543) was separated by chiral HPLC (ChiralpakAD 5×50 cm, 20 u, 85% Hexane, 15% ethanol:methanol (1:1), 0.1% diethylamine) to give the desired product.

### Intermediate 114

The following Intermediate was synthesized by an analogous method to Intermediate 62 from the starting materials (SM) given in the table below.

| Int | Compound | Data | SM |
|---|---|---|---|
| 114 | Ethyl (3S,4R)-4-{[(3,4-dichloro-5- | ¹H NMR (400 MHz, | Intermediate 113 |
| | methyl-1H-pyrrol-2- | DMSO-d₆): 0.86 (t, 3H), | and ethyl (3S,4R)- |
| | yl)carbonyl]amino}-3- | 1.18 (t, 3H), 1.50-1.52 | 3,4-dichloro-5- |
| | propoxypiperidine-1-carboxylate | (m, 4H), 2.18 (s, 3H), | methyl-1H-pyrrole- |
| | 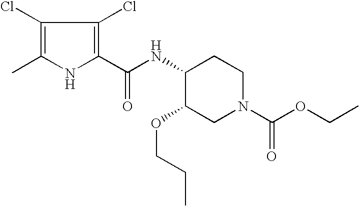 | 2.95 (m, 3H), 3.29 (br s,1H), 4.01-4.02 (q, 2H),4.12-4.23 (m, 4H). | 2-carboxylic acid(WO2006087543) |

### Intermediate 115

The following Intermediate was synthesized by an analogous method to Intermediate 64 from the starting materials (SM) given in the table below.

| Int | Compound | Data | SM |
|---|---|---|---|
| 115 | 3,4-dichloro-5-methyl-N-[(3S,4R)-3- | ¹H NMR (400 MHz, | Intermediate 114 |
| | propoxypiperidin-4-yl]-1H-pyrrole- | DMSO-d₆): 0.88 (t, | |
| | 2-carboxamide | 3H), 1.51-1.52 (q, 4H), | |
| | 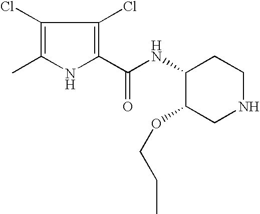 | 2.17 (s, 3H), 2.81-2.84(m, 1H), 3.02-3.03 (d,1H), 3.05-3.06 (m, 1H),3.26 (m, 2H), 3.34 (br s,1H), 3.49 (q, 2H), 4.06(br s, 1H), 7.05 (d, 1H). | |

### Intermediate 116

### tert-Butyl (3S,4R)-4-{[(4-chloro-3,5-dimethyl-1H-pyrrol-2-yl)carbonyl]amino}-3-fluoropiperidine-1-carboxylate

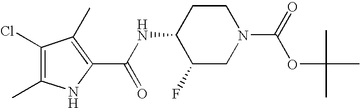

In a 100 mL round bottom flask was taken 4-chloro-3,5-dimethyl-1H-pyrrole-2-carboxylic acid (WO 2006087543, 1.5 g, 8.64 mmol), HATU (3.94 g, 10.37 mmol) and DIEA (3.32 ml, 19.01 mmol) in DCM (43.2 ml) to give a brown solution. To this was added (3S,4R)-tert-butyl 4-amino-3-fluoropiperidine-1-carboxylate (WO 2006087543, 1.886 g, 8.64 mmol) and the mixture was allowed to stir at RT overnight. The solvent was then removed under vacuum and the residue was taken in MeOH (2 mlL) and water was added to precipitate the product. The solid thus obtained was filtered, washed with water then hexane and dried under vacuum to afford the title compound (3.00 g).

MS (ES+-55): 318 for C₁₃Hl₇CIFN₃O₃ (M-55)

### Intermediate 117

### 4-chloro-N-[(3S,4R)-3-fluoropiperidin-4-yl]-3,5-dimethyl-1H-pyrrole-2-carboxamide

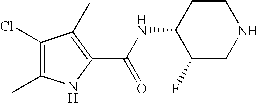

In a 50 mL round-bottomed flask was taken tert-Butyl (3S,4R)-4-{[(4-chloro-3,5-dimethyl-1H-pyrrol-2-yl)carbonyl]amino}-3-fluoropiperidine-1-carboxylate (Intermediate 116, 3.0 g, 8.02 mmol) in 4N HCl in dioxane (20.06 ml, 80.25 mmol) to give a yellow solution which was heated at 55° C. for 6.5 h. TLC and LCMS indicated completion of reaction. The reaction mixture was evaporated to dryness and methanol (15 ml) was added to the residue then the mixture was evaporated to dryness. Water (15 ml) was added to the residue and the mixture was neutralized with 14% NaOH to pH˜7.0. The solid that precipitated was dried under vacuum to afford 4-chloro-N-[(3S,4R)-3-fluoropiperidin-4-yl]-3,5-dimethyl-1H-pyrrole-2-carboxamide (1.212 g).

MS (ES⁺): 274 for C₁₂H₁₇ClFN₃O

### Intermediate 118

### Ethyl (3S,4R)-4-{[(4-chloro-3-cyano-5-methyl-1H-pyrrol-2-yl)carbonvylamino1-3-methoxypiperidine-1-carboxylate

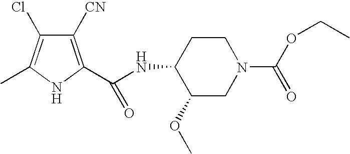

In a 50 ml round bottom flask 4-chloro-3-cyano-5-methyl-1H-pyrrole-2-carboxylic acid (WO 2006087543,540 mg, 2.93 mmol) was dissolved in CH₂Cl₂ (25 ml) and DIEA (2.55 ml, 14.63 mmol), HATU (1335 mg, 3.51 mmol) was added and stirred for 5 minutes and then (3S,4R)-ethyl 4-amino-3-methoxypiperidine-1-carboxylate ((1R)-7,7-dimethyl-2-oxobicyclo[2.2.1]heptan-1-yl) methanesulfonate (WO2006087543, 1271 mg, 2.93 mmol) was added portion wise and the resulting mixture was stirred for overnight at RT. The progress of the reaction was monitored through LCMS which showed completion of the reaction after stirring the reaction mixture for overnight. The reaction mixture was diluted with DCM and washed with water. The organic layer was dried over sodium sulphate and concentrated under vacuum to give ethyl (3S,4R)-4-{[(4-chloro-3-cyano-5-methyl-1H-pyrrol-2-yl)carbonyl]amino}-3-methoxypiperidine-1-carboxylate (1000 mg).

MS (ES⁺): 369 for C₁₆H₂₁ClN₄O₄

### Intermediate 119

### 4-Chloro-3-cyano-N-[(3S,4R)-3-methoxypiperidin-4-yl]-5-methyl-1H-pyrrole-2-carboxamide

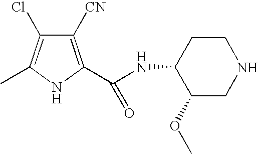

In a 250 mL round-bottomed flask (3S,4R)-ethyl 4-(4-chloro-3-cyano-5-methyl-1H-pyrrole-2-carboxamido)-3-methoxypiperidine-1-carboxylate (Intermediate 118, 1 g, 2.71 mmol) was dissolved in EtOH (10 mL). NaOH (10M solution) (1.084 g, 27.11 mmol) was then added and the reaction mixture was heated to 80° C. for 2 days. The progress of the reaction was monitored through LCMS. The reaction mixture was evaporated in vacuo, ice cold water (25 ml) was added and the mixture was neutralized with 6N HCl (pH 7), sonicated and the solid precipitate was filtered and dried under high vacuum afforded the product as pale brown solid (0.650 g).

MS (ES⁺): 297 for C₁₃H₁₇ClN₄O₂

1. A compound of formula (Ia): 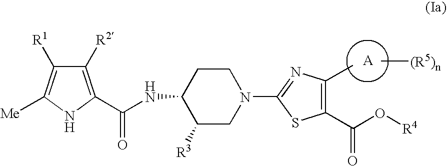
R¹ is chloro or cyano;
R^{2′} is hydrogen, chloro, cyano or methyl;
R³ is fluoro, methyl, methoxy, ethoxy, propoxy, allyloxy and benzyloxy;
R⁴ is hydrogen or C₁₋₄alkyl;
Ring A is carbocyclyl or heterocyclyl; wherein if said heterocyclyl contains an —NH— moiety that nitrogen may be optionally substituted by a group selected from R⁶; and wherein if said heterocyclyl contains an —N═ moiety that nitrogen may form a quaternary compound with a methyl group;
R⁵ is a substituent on carbon and is selected from halo, nitro, cyano, hydroxy, amino, carboxy, carbamoyl, mercapto, sulphamoyl, sulfo, formyl, ureido, hydroxyiminomethyl, N-hydroxyformamido, hydrazinocarbonyl, N-hydroxyethanimidoyl, amino(hydroxyimino)methyl, C₁₋₄alkyl, C₂₋₄alkenyl, C₂₋₄alkynyl, C₁₋₄alkoxy, C₁₋₄alkanoyl, C₁₋₄alkanoyloxy, N—(C₁₋₄alkyl)amino, N,N—(C₁₋₄alkyl)₂amino, C₁₋₄alkanoylamino, N—(C₁₋₄alkyl)carbamoyl, N,N—(C₁₋₄alkyl)₂carbamoyl, N—(C₁₋₄alkoxy)carbamoyl, N′—(C₁₋₄alkyl)ureido, N′,N′—(C₁₋₄alkyl)₂ureido, N—(C₁₋₄alkyl)-N—(C₁₋₄alkoxy)carbamoyl, C₁₋₄alkylS(O)ₐ wherein a is 0 to 2, C₁₋₄alkoxycarbonyl, C₁₋₄alkoxycarbonylamino, N—(C₁₋₄alkyl)sulphamoyl, N,N—(C₁₋₄alkyl)₂sulphamoyl, C₁₋₄alkylsulphonylamino, C₁₋₄alkylsulphonylaminocarbonyl, N′—(C₁₋₄alkyl)hydrazinocarbonyl, N′,N′—(C₁₋₄alkyl)₂hydrazinocarbonyl, carbocyclyl-R⁷— or heterocyclyl-R⁸—; wherein R⁵ may be optionally substituted on carbon by one or more R⁹; and wherein if said heterocyclyl contains an —NH— moiety that nitrogen may be optionally substituted by a group selected from R¹⁰;
n is 0, 1, 2, or 3;
R⁹ is selected from halo, nitro, cyano, hydroxy, amino, carboxy, carbamoyl, mercapto, sulphamoyl, C₁₋₄alkyl, C₂₋₄alkenyl, C₂₋₄alkynyl, C₁₋₄alkoxy, C₁₋₄alkanoyl, C₁₋₄alkanoyloxy, N—(C₁₋₄alkyl)amino, N,N—(C₁₋₄alkyl)₂amino, C₁₋₄alkanoylamino, N—(C₁₋₄alkyl)carbamoyl, N,N—(C₁₋₄alkyl)₂carbamoyl, C₁₋₄alkylS(O)₃ wherein a is 0 to 2, C₁₋₄alkoxycarbonyl, N—(C₁₋₄alkyl)sulphamoyl, N,N—(C₁₋₄alkyl)₂sulphamoyl, C₁₋₄alkylsulphonylamino, C₁₋₄alkoxycarbonylamino, carbocyclyl-R¹¹— or heterocyclyl-R¹²—; wherein R⁹ may be optionally substituted on carbon by one or more R¹³; and wherein if said heterocyclyl contains an —NH— moiety that nitrogen may be optionally substituted by a group selected from R¹⁴;
R⁶, R¹⁰ and R¹⁴ are independently selected from C₁₋₄alkyl, C₁₋₄alkanoyl, C₁₋₄alkylsulphonyl, C₁₋₄alkoxycarbonyl, carbamoyl, N—(C₁₋₄alkyl)carbamoyl, N,N—(C₁₋₄alkyl)carbamoyl, benzyl, benzyloxycarbonyl, benzoyl and phenylsulphonyl; wherein R⁶, R¹⁰ and R¹⁴ may be independently optionally substituted on carbon by a group selected from R²⁰;
R⁷, R⁸, R¹¹ and R¹² are independently selected from a direct bond,—O—, —N(R¹⁵)—, —C(O)—, —N(R¹⁶)C(O)—, —C(O)N(R¹⁷)—, —S(O)ₚ—, —SO₂N(R¹⁸)— or —N(R¹⁹)SO₂—; wherein R¹⁵, R¹⁶, R¹⁷, R¹⁸ and R¹⁹ are independently selected from hydrogen or C₁₋₄alkyl and p is 0-2;
R¹³ and R²⁰ are independently selected from halo, nitro, cyano, hydroxy, trifluoromethoxy, trifluoromethyl, amino, carboxy, carbamoyl, mercapto, sulphamoyl, methyl, ethyl, ethenyl, ethynyl, methoxy, ethoxy, 2-trimethylsilylethoxy, acetyl, acetoxy, methylamino, ethylamino, dimethylamino, diethylamino, N-methyl-N-ethylamino, acetylamino, N-methylcarbamoyl, N-ethylcarbamoyl, N,N-dimethylcarbamoyl, N,N-diethylcarbamoyl, N-methyl-N-ethylcarbamoyl, methylthio, ethylthio, methylsulphinyl, ethylsulphinyl, mesyl, ethylsulphonyl, methoxycarbonyl, ethoxycarbonyl, N-methylsulphamoyl, N-ethylsulphamoyl, N,N-dimethylsulphamoyl, N,N-diethylsulphamoyl or N-methyl-N-ethylsulphamoyl;
or a pharmaceutically acceptable salt thereof.

2. The compound of claim 1, wherein ring A is a heterocyclyl; wherein if said heterocyclyl contains an —NH— moiety that nitrogen may be optionally substituted by a group selected from R⁶; and wherein if said heterocyclyl contains an —N═ moiety that nitrogen may form a quaternary compound with a methyl group; wherein:
R⁶ is C₁₋₄alkyl; wherein R⁶ may be optionally substituted on carbon by a group selected from R²⁰; and
R²⁰ is selected from methoxy or ethoxy.

3. The compound of claim 2, wherein Ring A is pyridyl, 2H-pyrazolyl, isoxazolyl, imidazolyl, pyrazinyl, thiazolyl, pyrimidinyl, 1,2,4-oxadiazolyl, benzothiazolyl, 1,2,4-triazolyl or 1,3,4-oxadiazolyl wherein said imidazolyl or 1,2,4-triazolyl may be optionally substituted on nitrogen by a group selected from R⁶; and wherein if said imidazolyl may form a quaternary compound on an —N═ moiety with a methyl group; wherein
R⁶ is methyl or ethyl; wherein R⁶ may be optionally substituted on carbon by a group selected from R²⁰; and
R²⁰ is selected from methoxy or ethoxy.

4. The compound of claim 3 Ring A is 1-(2-methoxyethyl)imidazol-2-yl, 1-(2-trimethylsilylethoxymethyl)imidazol-2-yl, 1-(methoxymethyl)imidazol-2-yl, 1,2,4-oxadiazol-5-yl, 1,3,4-oxadiazol-2-yl, 1,3-dimethylimidazol-2-yl, 1H-imidazol-2-yl, 1-methylimidazol-4-yl, 2H-pyrazol-3-yl, 2-methyl-1,2,4-triazol-3-yl, 2-pyridyl, benzothiazol-2-yl, isoxazol-5-yl, pyrazin-2-yl, pyrimidin-2-yl, pyrimidin-4-yl, thiazol-2-yl or thiazol-4-yl.

5. The compound of claim 4, wherein Ring A is 2-methyl-1,2,4-triazol-3-yl.

6. The compound of claim 4, wherein Ring A is pyrimidin-2-yl.

7. The compound of claim 1, wherein Ring A is a carbocyclyl.

8. The compound of claim 2 wherein n is 0.

9. The compound of claim 2, wherein n is 1, 2, or 3 and R⁵ is selected from the group consisting of amino, N-methylamino, N,N-dimethylamino, methyl, and methoxy.

10. The compound of claim 2, wherein Ring A, R⁵ and n together form 1H-imidazol-2-yl, 2,6-bis(dimethylamino)pyrimidin-4-yl, 2-pyridyl, 2H-pyrazol-3-yl, pyrimidin-4-yl, isoxazol-5-yl, 1-methylimidazol-4-yl, pyrazin-2-yl, 2-aminothiazol-4-yl, 2-dimethylaminothiazol-4-yl, 2-methylaminothiazol-4-yl, 1,3-dimethylimidazol-2-yl, 1,4,5-trimethylimidazol-2-yl, 4,6-dimethoxypyrimidin-2-yl, 3-methyl-1,2,4-oxadiazol-5-yl, benzothiazol-2-yl, 4-methoxypyrimidin-2-yl, pyrimidin-2-yl, 1-methylimidazol-2-yl, 1-(2-methoxyethyl)imidazol-2-yl, 1-(methoxymethyl)imidazol-2-yl, 1-(2-trimethylsilylethoxymethyl)imidazol-2-yl, thiazol-2-yl, 2-methyl-1,2,4-triazol-3-yl, 5-methyl-1,3,4-oxadiazol-2-yl or 1,3,4-oxadiazol-2-yl.

11. The compound of claim 2, wherein R¹ is chloro.

12. The compound of claim 2, wherein R¹ is cyano.

13. The compound of claim 2, wherein R^{2′} is hydrogen.

14. The compound of claim 2, wherein R^{2′} is chloro.

15. The compound of claim 2, wherein R³ is selected from fluoro, methyl, methoxy, ethoxy, propoxy, allyloxy, and benzyloxy.

16. The compound of claim 15, wherein R³ is methoxy.

17. The compound of claim 2, wherein R⁴ is hydrogen or a C₁₋₄alkyl.

18. The compound of claim 17, wherein R⁴ is hydrogen.

19. The compound of claim 1, wherein:
R¹ is chloro or cyano;
R^{2′} is hydrogen or chloro;
R³ is fluoro or methoxy;
R⁴ is hydrogen or C₁₋₄alkyl;
Ring A is pyridyl, 2H-pyrazolyl, isoxazolyl, imidazolyl, pyrazinyl, thiazolyl, pyrimidinyl, 1,2,4-oxadiazolyl, benzothiazolyl, 1,2,4-triazolyl or 1,3,4-oxadiazolyl wherein said imidazolyl or 1,2,4-triazolyl may be optionally substituted on nitrogen by a group selected from R⁶; and wherein if said imidazolyl may form a quaternary compound on an —N═ moiety with a methyl group;
R⁶ is methyl or ethyl; wherein R⁶ may be optionally substituted on carbon by a group selected from R²⁰;
R²⁰ is selected from methoxy or ethoxy;
R⁵ is a substituent on carbon and is selected from amino, C₁₋₄alkyl, C₁₋₄alkoxy, N—(C₁₋₄alkyl)amino or N,N—(C₁₋₄alkyl)₂amino; and
n is 0-2;
or a pharmaceutically acceptable salt thereof.

20. The compound of claim 1, wherein:
R¹ is chloro or cyano;
R^{2′} is hydrogen or chloro;
R³ is fluoro or methoxy;
R⁴ is hydrogen, methyl or ethyl;
Ring A is 1-(2-methoxyethyl)imidazol-2-yl, 1-(2-trimethylsilylethoxymethyl)imidazol-2-yl, 1-(methoxymethyl)imidazol-2-yl, 1,2,4-oxadiazol-5-yl, 1,3,4-oxadiazol-2-yl, 1,3-dimethylimidazol-2-yl, 1H-imidazol-2-yl, 1-methylimidazol-4-yl, 2H-pyrazol-3-yl, 2-methyl-1,2,4-triazol-3-yl, 2-pyridyl, benzothiazol-2-yl, isoxazol-5-yl, pyrazin-2-yl, pyrimidin-2-yl, pyrimidin-4-yl, thiazol-2-yl or thiazol-4-yl;
R⁵ is a substituent on carbon and is selected from amino, methyl, methoxy, methylamio or dimethylamino;
n is 0-2;
or a pharmaceutically acceptable salt thereof.

21. The compound of claim 1, wherein:
R¹ and R^{2′} are chloro;
R³ is methoxy;
R⁴ is hydrogen;
Ring A is 2-methyl-1,2,4-triazol-3-yl or pyrimidin-2-yl;
n is 0;
or a pharmaceutically acceptable salt thereof.

22. A compound selected from the group consisting of:
2-((3S,4R)-4-{[(3,4-Dichloro-5-methyl-1H-pyrrol-2-yl)carbonyl]amino}-3-methoxypiperidin-1-yl)-4-pyrimidin-2-yl-1,3-thiazole-5-carboxylic acid;
2-((3S,4R)-4-{[(3,4-Dichloro-5-methyl-1H-pyrrol-2-yl)carbonyl]amino}-3-methoxypiperidin-1-yl)-4-pyrimidin-4-yl-1,3-thiazole-5-carboxylic acid;
2′-((3S,4R)-4-{[(3,4-Dichloro-5-methyl-1H-pyrrol-2-yl)carbonyl]amino}-3-methoxypiperidin-1-yl)-2,4′-bi-1,3-thiazole-5′-carboxylic acid;
2-((3S,4R)-4-{[(3,4-Dichloro-5-methyl-1H-pyrrol-2-yl)carbonyl]amino}-3-fluoropiperidin-1-yl)-4-pyrimidin-2-yl-1,3-thiazole-5-carboxylic acid;
2-((3S,4R)-4-{[(3,4-Dichloro-5-methyl-1H-pyrrol-2-yl)carbonyl]amino}-3-fluoropiperidin-1-yl)-4-pyrimidin-4-yl-1,3-thiazole-5-carboxylic acid;
2′-((3S,4R)-4-{[(3,4-Dichloro-5-methyl-1H-pyrrol-2-yl)carbonyl]amino}-3- fluoropiperidin-1-yl)-2,4′-bi-1,3-thiazole-5′-carboxylic acid;
2-((3S,4R)-4-{[(3,4-Dichloro-5-methyl-1H-pyrrol-2-yl)carbonyl]amino}-3- methoxypiperidin-1-yl)-4-pyridin-2-yl-1,3-thiazole-5-carboxylic acid;
2-((3S,4R)-4-{[(3,4-Dichloro-5-methyl-1H-pyrrol-2-yl)carbonyl]amino}-3-fluoropiperidin-1-yl)-4-pyridin-2-yl-1,3-thiazole-5-carboxylic acid;
Bis(dimethylamino)pyrimidin-4-yl]-2-((3S,4R)-4-{[(3,4-dichloro-5-methyl-1H-pyrrol-2-yl)carbonyl]amino}-3-methoxypiperidin-1-yl)-1,3-thiazole-5-carboxylic acid;
Bis(dimethylamino)pyrimidin-4-yl]-2-((3S,4R)-4-{[(3,4-dichloro-5-methyl-1-pyrrol-2-yl)carbonyl]amino}-3-fluoropiperidin-1-yl)-1,3-thiazole-5-carboxylic acid;
2-((3S,4R)-4-{[(3,4-Dichloro-5-methyl-1H-pyrrol-2-yl)carbonyl]amino}-3-methoxypiperidin-1-yl)-4-(4,6-dimethoxypyrimidin-2-yl)-1,3-thiazole-5-carboxylic acid;
2-((3S,4R)-4-{[(3,4-Dichloro-5-methyl-1H-pyrrol-2-yl)carbonyl]amino}-3-methoxypiperidin-1-yl)-4-pyrazin-2-yl-1,3-thiazole-5-carboxylic acid;
4-(1,3-Benzothiazol-2-yl)-2-((3S,4R)-4-{[(3,4-dichloro-5-methyl-1H-pyrro-2-yl)carbonyl]amino}-3-methoxypiperidin-1-yl)-1,3-thiazole-5-carboxylic acid;
2-((3S,4R)-4-{[(3,4-Dichloro-5-methyl-1H-pyrrol-2-yl)carbonyl]amino}-3-methoxypiperidin-1-yl)-4-pyrazin-2-yl-1,3-thiazole-5-carboxylic acid;
4-(1,3-Benzothiazol-2-yl)-2-((3S,4R)-4-{[(3,4-dichloro-5-methyl-1H-pyrro-2-yl)carbonyl]amino}-3-fluoropiperidin-1-yl)-1,3-thiazole-5-carboxylic acid;
2-((3S,4R)-4-{[(3,4-Dichloro-5-methyl-1H-pyrrol-2-yl)carbonyl]amino}-3-methoxypiperidin-1-yl)-4-(1-{[2-(trimethylsilyl)ethoxy]methyl}-1H-imidazol-2-yl)-1,3-thiazole-5-carboxylic acid;
2-((3S,4R)-4-{[(3,4-Dichloro-5-methyl-1H-pyrrol-2-yl)carbonyl]amino}-3-methoxypiperidin-1-yl)-4-[1-(methoxymethyl)-1H-imidazol-2-yl]-1,3-thiazole-5-carboxylic acid;
2-((3S,4R)-4-{[(3,4-Dichloro-5-methyl-1H-pyrrol-2-yl)carbonyl]amino}-3-methoxypiperidin-1-yl)-4-(1H-imidazol-2-yl)-1,3-thiazole-5-carboxylic acid;
2-((3S,4R)-4-{[(3,4-Dichloro-5-methyl-1H-pyrrol-2-yl)carbonyl]amino}-3-fluoropiperidin-1-yl)-4-[1-(methoxymethyl)-1H-imidazol-2-yl]-1,3-thiazole-5-carboxylic acid;
2-((3S,4R)-4-{[(3,4-Dichloro-5-methyl-1H-pyrrol-2-yl)carbonyl]amino}-3-fluoropiperidin-1-yl)-4-(1H-imidazol-2-yl)-1,3-thiazole-5-carboxylic acid;
2-((3S,4R)-4-{[(3,4-Dichloro-5-methyl-1H-pyrrol-2-yl)carbonyl]amino}-3-methoxypiperidin-1-yl)-4-(1-methyl-1H-imidazol-2-yl)-1,3-thiazole-5-carboxylic acid;
2-[5-Carboxy-2-((3S,4R)-4-{[(3,4-dichloro-5-methyl-1H-pyrrol-2-yl)carbonyl]amino}-3-methoxypiperidin-1-yl)-1,3-thiazol-4-yl]-1,3-dimethyl-1H-imidazol-3-ium trifluoroacetate;
2-((3S,4R)-4-{[(3,4-Dichloro-5-methyl-1H-pyrrol-2-yl)carbonyl]amino}-3-methoxypiperidin-1-yl)-4-(1H-pyrazol-5-yl)-1,3-thiazole-5-carboxylic acid;
2-((3S,4R)-4-{[(3,4-Dichloro-5-methyl-1H-pyrrol-2-yl)carbonyl]amino}-3-fluoropiperidin-1-yl)-4-(1H-pyrazol-5-yl)-1,3-thiazole-5-carboxylic acid;
2-((3S,4R)-4-{[(3,4-Dichloro-5-methyl-1H-pyrrol-2-yl)carbonyl]amino}-3-methoxypiperidin-1-yl)-4- [1-(2-methoxyethyl)-1H-imidazol-2-yl]-1,3-thiazole-5-carboxylic acid;
2-((3S,4R)-4-{[(3,4-Dichloro-5-methyl-1H-pyrrol-2-yl)carbonyl]amino}-3-fluoropiperidin-1-yl)-4-[1-(2-methoxyethyl)-1H-imidazol-2-yl]-1,3-thiazole-5-carboxylic acid;
2-((3S,4R)-4-{[(3,4-Dichloro-5-methyl-1H-pyrrol-2-yl)carbonyl]amino}-3-methoxypiperidin-1-yl)-4-(1-methyl-1H-1,2,4-triazol-5-yl)-1,3-thiazole-5-carboxylic acid;
2-((3S,4R)-4-{[(3,4-Dichloro-5-methyl-1H-pyrrol-2-yl)carbonyl]amino}-3-fluoropiperidin-1-yl)-4-(1-methyl-1H-1,2,4-triazol-5-yl)-1,3-thiazole-5-carboxylic acid;
2-((3S,4R)-4-{[(3-Chloro-4-cyano-5-methyl-1H-pyrrol-2-yl)carbonyl]amino}-3-methoxypiperidin-1-yl)-4-(1-methyl-1H-1,2,4-triazol-5-yl)-1,3-thiazole-5-carboxylic acid;
2-((3S,4R)-4-{[(3-Chloro-4-cyano-5-methyl-1H-pyrrol-2-yl)carbonyl]amino}-3-methoxypiperidin-1-yl)-4- [1-(2-methoxyethyl)-1H-imidazol-2-yl]-1,3-thiazole-5-carboxylic acid;
2-((3S,4R)-4-{[(4-Cyano-5-methyl-1H-pyrrol-2-yl)carbonyl]amino}-3-methoxypiperidin-1-yl)-4-(1-methyl-1H-1,2,4-triazol-5-yl)-1,3-thiazole-5-carboxylic acid;
2-((3S,4R)-4-{[(3,4-Dichloro-5-methyl-1H-pyrrol-2-yl)carbonyl]amino}-3- methoxypiperidin-1-yl)-4-(1,4 ,5-trimethyl-1H-imidazol-2-yl)-1,3-thiazole-5-carboxylic acid;
2′-Amino-2-((3S,4R)-4-{[(3,4-dichloro-5-methyl-1H-pyrrol-2-yl)carbonyl]amin0}-3- methoxypiperidin-1-yl)-4,4′-bi-1,3-thiazole-5-carboxylic acid;
2-((3S,4R)-4-{[(3,4-Dichloro-5-methyl-1H-pyrrol-2-yl)carbonyl]amino}-3-methoxypiperidin-1-yl)-2′-(methylamino)-4,4′-bi-1,3-thiazole-5-carboxylic acid;
2-((3S,4R)-4-{[(3,4-Dichloro-5-methyl-1H-pyrrol-2-yl)carbonyl]amino}-3-methoxypiperidin-1-yl)-2′-(dimethylamino)-4,4′-bi-1,3-thiazole-5-carboxylic acid;
2′-Amino-2-((3S,4R)-4-{[(3,4-dichloro-5-methyl-1H-pyrrol-2-yl)carbonyl]amino}-3-fluoropiperidin-1-yl)-4,4′-bi-1,3-thiazole-5-carboxylic acid;
2-((3S,4R)-4-{[(3,4-Dichloro-5-methyl-1H-pyrrol-2-yl)carbonyl]amino}-3-fluoropiperidin-1-yl)-2′-(methylamino)-4,4′-bi-1,3-thiazole-5-carboxylic acid;
2-((3S,4R)-4-{[(3,4-Dichloro-5-methyl-1H-pyrrol-2-yl)carbonyl]amino}-3-fluoropiperidin-1-yl)-2′-(dimethylamino)-4,4′-bi-1,3-thiazole-5-carboxylic acid;
2-((3S,4R)-4-{[(3,4-Dichloro-5-methyl-1H-pyrrol-2-yl)carbonyl]amino}-3-fluoropiperidin-1-yl)-4-(1-methyl-1H-imidazol-2-yl)-1,3-thiazole-5-carboxylic acid;
2-((3S,4R)-4-{[(3,4-Dichloro-5-methyl-1H-pyrrol-2-yl)carbonyl]amino}-3-methoxypiperidin-1-yl)-4-(1-methyl-1H-imidazol-4-yl)-1,3-thiazole-5-carboxylic acid;
2-((3S,4R)-4-{[(3,4-Dichloro-5-methyl-1H-pyrrol-2-yl)carbonyl]amino}-3-fluoropiperidin-1-yl)-4-(1-methyl-1H-imidazol-4-yl)-1,3-thiazole-5-carboxylic acid;
2-((3S,4R)-4-{[(3,4-Dichloro-5-methyl-1H-pyrrol-2-yl)carbonyl]amino}-3-methoxypiperidin-1-yl)-4-(4-methoxypyrimidin-2-yl)-1,3-thiazole-5-carboxylic acid;
Ethyl 2-((3S,4R)-4-{[(3,4-dichloro-5-methyl-1H-pyrrol-2-yl)carbonyl]amino}-3-methoxypiperidin-1-yl)-4-pyrimidin-2-yl-1,3-thiazole-5-carboxylate;
Methyl 2′-((3S,4R)-4-{[(3,4-dichloro-5-methyl-1H-pyrrol-2-yl)carbonyl]amino}-3-methoxypiperidin-1-yl)-2,4′-bi-1,3-thiazole-5′-carboxylate;
Ethyl 2-((3S,4R)-4-{[(3,4-dichloro-5-methyl-1H-pyrrol-2-yl)carbonyl]amino}-3-fluoropiperidin-1-yl)-4-pyrimidin-2-yl-1,3-thiazole-5-carboxylate;
Ethyl 2-((3S,4R)-4-{[(3,4-dichloro-5-methyl-1H-pyrrol-2-yl)carbonyl]amino}-3-fluoropiperidin-1-yl)-4-pyrimidin-4-yl-1,3-thiazole-5-carboxylate;
Methyl 2′-((3S,4R)-4-{[(3,4-dichloro-5-methyl-1H-pyrrol-2-yl)carbonyl]amino}-3-fluoropiperidin-1-yl)-2,4′-bi-1,3-thiazole-5′-carboxylate;
Ethyl 2-((3S,4R)-4-{[(3,4-dichloro-5-methyl-1H-pyrrol-2-yl)carbonyl]amino}-3-methoxypiperidin-1-yl)-4-pyridin-2-yl-1,3-thiazole-5-carboxylate;
Ethyl 2-((3S,4R)-4-{[(3,4-dichloro-5-methyl-1H-pyrrol-2-yl)carbonyl]amino}-3-fluoropiperidin-1-yl)-4-pyridin-2-yl-1,3-thiazole-5-carboxylate;
Ethyl 4- [2,6-bis(dimethylamino)pyrimidin-4-yl]-2-((3S,4R)-4-{[(3,4-dichloro-5-methyl-1H-pyrrol-2-yl)carbonyl]amino}-3-methoxypiperidin-1-yl)-1,3-thiazole-5-carboxylate;
Ethyl 4- [2,6-bis(dimethylamino)pyrimidin-4-yl]-2-((3S,4R)-4-{[(3,4-dichloro-5-methyl-1H-pyrrol-2-yl)carbonyl]amino}-3-fluoropiperidin-1-yl)-1,3-thiazole-5-carboxylate;
Ethyl 2-((3S,4R)-4-{[(3,4-dichloro-5-methyl-1H-pyrrol-2-yl)carbonyl]amino}-5-methoxypiperidin-1-yl)-4-(4,6-dimethoxypyrimidin-2-yl)-1,3-thiazole-5-carboxylate;
Ethyl 2-((3S,4R)-4-{[(3,4-dichloro-5-methyl-1H-pyrrol-2-yl)carbonyl]amino}-3-methoxypiperidin-1-yl)-4-pyrazin-2-yl-1,3-thiazole-5-carboxylate;
Ethyl 4-(1,3-benzothiazol-2-yl)-2-((3S,4R)-4-{[(3,4-dichloro-5-methyl-1H-pyrrol-2-yl)carbonyl]amino}-3-methoxypiperidin-1-yl)-1,3-thiazole-5-carboxylate;
Ethyl 2-((3S,4R)-4-{[(3,4-dichloro-5-methyl-1H-pyrrol-2-yl)carbonyl]amino}-3-fluoropiperidin-1-yl)-4-pyrazin-2-yl-1,3-thiazole-5-carboxylate;
Ethyl 4-(1,3-benzothiazol-2-yl)-2-((3S,4R)-4-{[(3,4-dichloro-5-methyl-1H-pyrrol-2-yl)carbonyl]amino}-3-fluoropiperidin-1-yl)-1,3-thiazole-5-carboxylate;
Methyl 2-((3S,4R)-4-{[(3,4-dichloro-5-methyl-1H-pyrrol-2-yl)carbonyl]amino}-3-methoxypiperidin-1-yl)-4-(1-{[2-(trimethylsilyl)ethoxy]methyl}-1H-imidazol-2-yl)-1,3-thiazole-5-carboxylate;
Methyl 2-((3S,4R)-4-{[(3,4-dichloro-5-methyl-1H-pyrrol-2-yl)carbonyl]amino}-3-methoxypiperidin-1-yl)-4- [1-(methoxymethyl)-1H-imidazol-2-yl]-1,3-thiazole-5-carboxylate;
Methyl 2-((3S,4R)-4-{[(3,4-dichloro-5-methyl-1H-pyrrol-2-yl)carbonyl]amino}-3-fluoropiperidin-1-yl)-4-[1-(methoxymethyl)-1H-imidazol-2-yl]-1,3-thiazole-5-carboxylate;
Methyl 2-((3S,4R)-4-{[(3,4-dichloro-5-methyl-1H-pyrrol-2-yl)carbonyl]amino}-3-methoxypiperidin-1-yl)-4-isoxazol-5-yl-1,3-thiazole-5-carboxylate;
Methyl 2-((3S,4R)-4-{[(3,4-dichloro-5-methyl-1H-pyrrol-2-yl)carbonyl]amino}-3-fluoropiperidin-1-yl)-4-isoxazol-5-yl-1,3-thiazole-5-carboxylate;
Methyl 2-((3S,4R)-4-{[(3,4-dichloro-5-methyl-1H-pyrrol-2-yl)carbonyl]amino}-3-methoxypiperidin-1-yl)-4-(1H-pyrazol-5-yl)-1,3-thiazole-5-carboxylate;
Methyl 2-((3S,4R)-4-{[(3,4-dichloro-5-methyl-1H-pyrrol-2-yl)carbonyl]amino}-3-fluoropiperidin-1-yl)-4-(1H-pyrazol-5-yl)-1,3-thiazole-5-carboxylate;
Methyl 2-((3S,4R)-4-{[(3,4-dichloro-5-methyl-1H-pyrrol-2-yl)carbonyl]amino}-3-methoxypiperidin-1-yl)-4-[1-(2-methoxyethyl)-1H-imidazol-2-yl]-1,3-thiazole-5-carboxylate;
Methyl 2-((3S,4R)-4-{[(3,4-dichloro-5-methyl-1H-pyrrol-2-yl)carbonyl]amino}-3-fluoropiperidin-1-yl)-4-[1-(2-methoxyethyl)-1H-imidazol-2-yl]-1,3-thiazole-5-carboxylate;
Methyl 2-((3S,4R)-4-{[(3,4-dichloro-5-methyl-1H-pyrrol-2-yl)carbonyl]amino}-3-methoxypiperidin-1-yl)-4-(1-methyl-1H-1,2,4-triazol-5-yl)-1,3-thiazole-5-carboxylate;
Methyl 2-((3S,4R)-4-{[(3,4-dichloro-5-methyl-1H-pyrrol-2-yl)carbonyl]amino}-3-fluoropiperidin-1-yl)-4-(1-methyl-1H-1,2,4-triazol-5-yl)-1,3-thiazole-5-carboxylate;
Methyl 2-((3S,4R)-4-{[(4-cyano-5-methyl-1H-pyrrol-2-yl)carbonyl]amino}-3-methoxypiperidin-1-yl)-4-(1-methyl-1H-1,2,4-triazol-5-yl)-1,3-thiazole-5-carboxylate;
Methyl 2-((3S,4R)-4-{[(3-chloro-4-cyano-5-methyl-1H-pyrrol-2-yl)carbonyl]amino}-3-methoxypiperidin-1-yl)-4-[1-(2-methoxyethyl)-1H-imidazol-2-yl]-1,3-thiazole-5-carboxylate;
Methyl 2-((3S,4R)-4-{[(3,4-dichloro-5-methyl-1H-pyrrol-2-yl)carbonyl]amino}-3-methoxypiperidin-1-yl)-4-(1,4,5-trimethyl-1H-imidazol-2-yl)-1,3-thiazole-5-carboxylate;
Methyl 2′-amino-2-((3S,4R)-4-{[(3,4-dichloro-5-methyl-1H-pyrrol-2-yl)carbonyl]amino}-3-methoxypiperidin-1-yl)-4,4′-bi-1,3-thiazole-5-carboxylate;
Methyl 2-((3S,4R)-4-{[(3,4-dichloro-5-methyl-1H-pyrrol-2-yl)carbonyl]amino}-3-methoxypiperidin-1-yl)-2′-(methylamino)-4,4′-bi-1,3-thiazole-5-carboxylate;
Methyl 2-((3S,4R)-4-{[(3,4-dichloro-5-methyl-1H-pyrrol-2-yl)carbonyl]amino}-3-methoxypiperidin-1-yl)-2′-(dimethylamino)-4,4′-bi-1,3-thiazole-5-carboxylate;
Methyl 2′-amino-2-((3S,4R)-4-{[(3,4-dichloro-5-methyl-1H-pyrrol-2-yl)carbonyl]amino}-3-fluoropiperidin-1-yl)-4,4′-bi-1,3-thiazole-5-carboxylate;
Methyl 2-((3S,4R)-4-{[(3,4-dichloro-5-methyl-1H-pyrrol-2-yl)carbonyl]amino}-3-fluoropiperidin-1-yl)-2′-(methylamino)-4,4′-bi-1,3-thiazole-5-carboxylate;
Methyl 2-((3S,4R)-4-{[(3,4-dichloro-5-methyl-1H-pyrrol-2-yl)carbonyl]amino}-3-fluoropiperidin-1-yl)-2′-(dimethylamino)-4,4′-bi-1,3-thiazole-5-carboxylate;
Ethyl 2-((3S,4R)-4-{[(3,4-dichloro-5-methyl-1H-pyrrol-2-yl)carbonyl]amino}-3-methoxypiperidin-1-yl)-4-(1-methyl-1H-imidazol-4-yl)-1,3-thiazole-5-carboxylate;
Ethyl 2-((3S,4R)-4-{[(3,4-dichloro-5-methyl-1H-pyrrol-2-yl)carbonyl]amino}-3-fluoropiperidin-1-yl)-4-(1-methyl-1H-imidazol-4-yl)-1,3-thiazole-5-carboxylate;
Methyl 2-((3S,4R)-4-{[(3,4-dichloro-5-methyl-1H-pyrrol-2-yl)carbonyl]amino}-3-fluoropiperidin-1-yl)-4-(1-methyl-1H-imidazol-2-yl)-1,3-thiazole-5-carboxylate;
Methyl 2-((3S,4R)-4-{[(3-chloro-4-cyano-5-methyl-1H-pyrrol-2-yl)carbonyl]amino}-3-methoxypiperidin-1-yl)-4-(1-methyl-1H-1,2,4-triazol-5-yl)-1,3-thiazole-5-carboxylate;
Ethyl 2-((3S,4R)-4-{[(3,4-dichloro-5-methyl-1H-pyrrol-2-yl)carbonyl]amino}-3-methoxypiperidin-1-yl)-4-pyrimidin-4-yl-1,3-thiazole-5-carboxylate;
Methyl 2-((3S,4R)-4-{[(3,4-dichloro-5-methyl- 1H-pyrrol-2-yl)carbonyl]amino}-3-methoxypiperidin-1-yl)-4-(4-methoxypyrimidin-2-yl)-1,3-thiazole-5-carboxylate;
Ethyl 2-((3S,4R)-4-{[(3,4-dichloro-5-methyl-1H-pyrrol-2-yl)carbonyl]amino}-3-methoxypiperidin-1-yl)-4-(1,3,4-oxadiazol-2-yl)-1,3-thiazole-5-carboxylate;
Ethyl 2-((3S,4R)-4-{[(3,4-dichloro-5-methyl-1H-pyrrol-2-yl)carbonyl]amino}-3-methoxypiperidin-1-yl)-4-(3-methyl-1,2,4-oxadiazol-5-yl)-1,3-thiazole-5-carboxylate;
Ethyl 2-((3S,4R)-4-{[(3,4-dichloro-5-methyl-1H-pyrrol-2-yl)carbonyl]amino}-3-methoxypiperidin-1-yl)-4-(5-methyl-1,3 ,4-oxadiazol-2-yl)-1,3-thiazole-5-carboxylate;
Methyl 2-((3S,4R)-4-{[(3,4-dichloro-5-methyl-1H-pyrrol-2-yl)carbonyl]amino}-3-methoxypiperidin-1-yl)-4-(1H-imidazol-2-yl)-1,3-thiazole-5-carboxylate;
Methyl 2-((3S,4R)-4-{[(3,4-dichloro-5-methyl-1H-pyrrol-2-yl)carbonyl]amino}-3-fluoropiperidin-1-yl)-4-(1H-imidazol-2-yl)-1,3-thiazole-5-carboxylate;
Methyl 2-((3S,4R)-4-{[(3,4-dichloro-5-methyl-1H-pyrrol-2-yl)carbonyl]amino}-3-methoxypiperidin-1-yl)-4-(1-methyl-1H-imidazol-2-yl)-1,3-thiazole-5-carboxylate;
2-[2-((3S,4R)-4-{[(3,4-Dichloro-5-methyl-1H-pyrrol-2-yl)carbonyl]amino}-3-methoxypiperidin-1-yl)-5-(methoxycarbonyl)-1,3-thiazol-4-yl]-1,3-dimethyl-1H-imidazol-3-ium iodide;
2-((3S,4R)-4-{[(3,4-Dichloro-5-methyl-1H-pyrrol-2-yl)carbonyl]amino}-3-methoxypiperidin-1-yl)-4-(5-methyl-1,3,4-oxadiazol-2-yl)-1,3-thiazole-5-carboxylic acid;
2-((3S,4R)-4-{[(3,4-Dichloro-5-methyl-1H-pyrrol-2-yl)carbonyl]amino}-3-methoxypiperidin-1-yl)-4-(5-methyl-1,3,4-oxadiazol-2-yl)-1,3-thiazole-5-carboxylic acid;
Methyl 2-((3S,4R)-4-(3,4-dichloro-5-methyl-1H-pyrrole-2-carboxamido)-3-methoxypiperidin-1-yl)-4-(1-methyl-1H-1,2,3-triazol-4-yl)thiazole-5-carboxylate;
Methyl 2-((3S,4R)-4-(3,4-dichloro-5-methyl-1H-pyrrole-2-carboxamido)-3-methoxypiperidin-1-yl)-4-(3 ,5-dimethylpyrazin-2-yl)thiazole-5-carboxylate;
Methyl 2-((3S,4R)-4-(3,4-dichloro-5-methyl-1H-pyrrole-2-carboxamido)-3-methoxypiperidin-1-yl)-4-(6-methoxypyridin-2-yl)thiazole-5-carboxylate;
Ethyl 2-((3S,4R)-4-(3,4-dichloro-5-methyl-1H-pyrrole-2-carboxamido)-3-methoxypiperidin-1-yl)-4-(4-methoxypyridin-2-yl)thiazole-5-carboxylate;
Ethyl 2-((3S,4R)-4-(3,4-dichloro-5-methyl-1H-pyrrole-2-carboxamido)-3-methoxypiperidin-1-yl)-4-(5-methylpyrazin-2-yl)thiazole-5-carboxylate;
Ethyl 2-((3S,4R)-4-(3,4-dichloro-5-methyl-1H-pyrrole-2-carboxamido)-3-methoxypiperidin-1-yl)-4-(3-fluoropyridin-2-yl)thiazole-5-carboxylate;
Ethyl 2-((3S,4R)-4-(3,4-dichloro-5-methyl-1H-pyrrole-2-carboxamido)-3-fluoropiperidin-1-yl)-4-(4-methoxypyridin-2-yl)thiazole-5-carboxylate;
Methyl 2-((3S,4R)-4-(3,4-dichloro-5-methyl-1H-pyrrole-2-carboxamido)-3-fluoropiperidin-1-yl)-4-(1-methyl-1H-1,2,3-triazol-4-yl)thiazole-5-carboxylate;
Methyl 2-((3S,4R)-4-(3,4-dichloro-5-methyl-1H-pyrrole-2-carboxamido)-3-fluoropiperidin-1-yl)-4-(6-methoxypyridin-2-yl)thiazole-5-carboxylate;
Methyl 2-((3S,4R)-4-(3,4-dichloro-5-methyl-1H-pyrrole-2-carboxamido)-3-fluoropiperidin-1-yl)-4-(6-methoxypyridin-2-yl)thiazole-5-carboxylate;
Ethyl 2-((3S,4R)-4-(3,4-dichloro-5-methyl-1H-pyrrole-2-carboxamido)-3-fluoropiperidin-1-yl)-4-(3-fluoropyridin-2-yl)thiazole-5-carboxylate;
Ethyl 2-((3S,4R)-4-(3,4-dichloro-5-methyl-1H-pyrrole-2-carboxamido)-3-fluoropiperidin-1-yl)-4-(4-methoxypyridin-2-yl)thiazole-5-carboxylate;
Methyl 2-((3S,4R)-4-(4-chloro-3-cyano-5-methyl-1H-pyrrole-2-carboxamido)-3-methoxypiperidin-1-yl)-4-(1-methyl-1H-1,2,3-triazol-4-yl)thiazole-5-carboxylate;
Methyl 2-((3S,4R)-4-(4-chloro-3-cyano-5-methyl-1H-pyrrole-2-carboxamido)-3-methoxypiperidin-1-yl)-4-(1-methyl-1H-1,2,4-triazol-5-yl)thiazole-5-carboxylate;
Ethyl 2-((3S,4R)-4-(4-chloro-3-cyano-5-methyl-1H-pyrrole-2-carboxamido)-3-methoxypiperidin-1-yl)-4-(pyrimidin-2-yl)thiazole-5-carboxylate;
Ethyl 2-((3S,4R)-4-(4-chloro-3-cyano-5-methyl-1H-pyrrole-2-carboxamido)-3-methoxypiperidin-1-yl)-4-(3-fluoropyridin-2-yl)thiazole-5-carboxylate;
Ethyl 2-((3S,4R)-4-(4-chloro-3-cyano-5-methyl-1H-pyrrole-2-carboxamido)-3-methoxypiperidin-1-yl)-4-(5-methylpyrazin-2-yl)thiazole-5-carboxylate;
Ethyl 2-((3S,4R)-4-(4-chloro-3-cyano-5-methyl-1H-pyrrole-2-carboxamido)-3-methoxypiperidin-1-yl)-4-(1-methyl-1H-imidazol-4-yl)thiazole-5-carboxylate;
2-((3S,4R)-4-(3,4-Dichloro-5-methyl-1H-pyrrole-2-carboxamido)-3-methoxypiperidin-1-yl)-4-(1-methyl-1H-1,2,3-triazol-4-yl)thiazole-5-carboxylic acid;
2-((3S,4R)-4-(3,4-Dichloro-5-methyl-1H-pyrrole-2-carboxamido)-3-methoxypiperidin-1-yl)-4-(3,5-dimethylpyrazin-2-yl)thiazole-5-carboxylic acid;
2-((3S,4R)-4-(3,4-Dichloro-5-methyl-1H-pyrrole-2-carboxamido)-3-methoxypiperidin-1-yl)-4-(6-methoxypyridin-2-yl)thiazole-5-carboxylic acid;
2-((3S,4R)-4-(3,4-Dichloro-5-methyl-1H-pyrrole-2-carboxamido)-3-methoxypiperidin-1-yl)-4-(4-methoxypyridin-2-yl)thiazole-5-carboxylic acid;
2-((3S,4R)-4-(3,4-Dichloro-5-methyl-1H-pyrrole-2-carboxamido)-3-methoxypiperidin-1-yl)-4-(5-methylpyrazin-2-yl)thiazole-5-carboxylic acid;
2-((3S,4R)-4-(3,4-Dichloro-5-methyl-1H-pyrrole-2-carboxamido)-3-methoxypiperidin-1-yl)-4-(3-fluoropyridin-2-yl)thiazole-5-carboxylic acid;
2-((3S,4R)-4-(3,4-Dichloro-5-methyl-1H-pyrrole-2-carboxamido)-3-fluoropiperidin-1-yl)-4-(3-fluoropyridin-2-yl)thiazole-5-carboxylic acid;
2-((3S,4R)-4-(3,4-Dichloro-5-methyl-1H-pyrrole-2-carboxamido)-3-fluoropiperidin-1-yl)-4-(1-methyl-1H-1,2,3-triazol-4-yl)thiazole-5-carboxylic acid;
2-((3S,4R)-4-(3,4-Dichloro-5-methyl-1H-pyrrole-2-carboxamido)-3-fluoropiperidin-1-yl)-4-(6-methoxypyridin-2-yl)thiazole-5-carboxylic acid;
2-((3S,4R)-4-(3,4-Dichloro-5-methyl-1H-pyrrole-2-carboxamido)-3-fluoropiperidin-1-yl)-4-(5-methylpyrazin-2-yl)thiazole-5-carboxylic acid;
2-((3S,4R)-4-(3,4-Dichloro-5-methyl-1H-pyrrole-2-carboxamido)-3-fluoropiperidin-1-yl)-4-(3-fluoropyridin-2-yl)thiazole-5-carboxylic acid;
2-((3S,4R)-4-(3,4-Dichloro-5-methyl-1H-pyrrole-2-carboxamido)-3-fluoropiperidin-1-yl)-4-(4-methoxypyridin-2-yl)thiazole-5-carboxylic acid;
2-((3S,4R)-4-(4-Chloro-3-cyano-5-methyl-1H-pyrrole-2-carboxamido)-3-methoxypiperidin-1-yl)-4-(1-methyl-1H-1,2,3-triazol-4-yl)thiazole-5-carboxylic acid;
2-((3S,4R)-4-(4-Chloro-3-cyano-5-methyl-1H-pyrrole-2-carboxamido)-3-methoxypiperidin-1-yl)-4-(1-methyl-1H-1,2,4-triazol-5-yl)thiazole-5-carboxylic acid;
2-((3S,4R)-4-(4-Chloro-3-cyano-5-methyl-1H-pyrrole-2-carboxamido)-3-methoxypiperidin-1-yl)-4-(pyrimidin-2-yl)thiazole-5-carboxylic acid;
2-((3S,4R)-4-(4-Chloro-3-cyano-5-methyl-1H-pyrrole-2-carboxamido)-3-methoxypiperidin-1-yl)-4-(3-fluoropyridin-2-yl)thiazole-5-carboxylic acid;
2-((3S,4R)-4-(4-Chloro-3-cyano-5-methyl-1H-pyrrole-2-carboxamido)-3-methoxypiperidin-1-yl)-4-(5-methylpyrazin-2-yl)thiazole-5-carboxylic acid;
2-((3S,4R)-4-(4-Chloro-3-cyano-5-methyl-1H-pyrrole-2-carboxamido)-3-methoxypiperidin-1-yl)-4-(1-methyl-1H-imidazol-4-yl)thiazole-5-carboxylic acid;
2-{(3S,4R)-4-[(4-Chloro-3-cyano-5-methyl-1H-pyrrole-2-carbonyl)-amino]-3-piperidin-1-yl}-4-pyrazin-2-yl-thiazole-5-carboxylic acid ethyl ester;
2-{(3S,4R)-4-[(4-Chloro-3, 5-dimethyl-1H-pyrrole-2-carbonyl)-amino]-3-fluoropiperidin-1-yl}-4-pyrazin-2-yl-thiazole-5-carboxylic acid ethyl ester;
2-{(3S,4R)-4-[(4-chloro-3, 5-dimethyl-1H-pyrrole-2carbonyl)-amino]-3-fluoropiperidin-1-yl}-4-pyrimidin-4-yl-thiazole-5-carboxylic acid ethyl ester;
2-{(3S,4R)-4-[(4-Chloro-3-cyano-5-methyl-1H-pyrrole-2-carbonyl)-amino]-3-methoxypiperidin-1-yl}-4-pyrimidin-4-yl-thiazole-5-carboxylic acid ethyl ester;
2-{(3S,4R)-4-[(4-Chloro-3-cyano-5-methyl-1H-pyrrole-2-carbonyl) amino]-3-methoxypiperidin-1-yl}-4-(1-methyl-1H-pyrazol-3-yl)-thiazole-5-carboxylic acid ethyl ester;
2-{(3S,4R)-4-[(3,4-Dichloro-5-methyl-1H-pyrrole-2-carbonyl)-amino]-3-methoxypiperidin-1-yl}-4-(1-methyl-1H-pyrazol-3-yl)-thiazole-5-carboxylic acid ethyl ester;
2-{(3S,4R)-4-[(4-Chloro-3-cyano-5-methyl-1H-pyrrole-2-carbonyl)-amino]-3-piperidin-1-yl}-4-pyrazin-2-yl-thiazole-5-carboxylic acid;
2-[(3S,4R)-4-{[(4-chloro-3 ,5-dimethyl-1H-pyrrol-2-yl)carbonyl]amino}-3-fluoropiperidin-1-yl]-4-pyrazin-2-yl-1,3-thiazole-5-carboxylic acid;
2-[(3S,4R)-4-{[(4-chloro-3 ,5-dimethyl-1H-pyrrol-2-yl)carbonyl]amino}-3-fluoropiperidin-1-yl]-4-pyrimidin-4-yl-1,3-thiazole-5-carboxylic acid;
2-[(3S,4R)-4-{[(4-chloro-3-cyano-5-methyl-1H-pyrrol-2-yl)carbonyl]amino}-3-methoxypiperidin-1-yl]-4-pyrimidin-4-yl-1,3-thiazole-5-carboxylic acid;
2-[(3S,4R)-4-{[(4-chloro-3-cyano-5-methyl-1H-pyrrol-2-yl)carbonyl]amino}-3-methoxypiperidin-1-yl]-4-(1-methyl-1H-pyrazol-3-yl)-1,3-thiazole-5-carboxylic acid;
2-[(3S,4R)-4-{[(3,4-dichloro-5-methyl-1H-pyrrol-2-yl)carbonyl]amino}-3-methoxypiperidin-1-yl]-4-(1-methyl-1H-pyrazol-3-yl)-1,3-thiazole-5-carboxylic acid;
or a pharmaceutically acceptable salt thereof.

23. A pharmaceutical composition comprising a compound of claim 1, or a pharmaceutically acceptable salt thereof, and a pharmaceutically acceptable excipient or carrier.

24. A method of inhibiting bacterial DNA gyrase and/or bacterial topoisomerase IV in a warm-blooded animal in need of such treatment, comprising administering to the animal an effective amount of a compound of claim 1, or a pharmaceutically acceptable salt thereof.

25. A method of producing an antibacterial effect in a warm-blooded animal in need of such treatment, comprising administering to the animal an effective amount of a compound of claim 1, or a pharmaceutically acceptable salt thereof.

26. A method of treating a bacterial infection in a warm-blooded animal in need thereof, comprising administering to the animal an effective amount of a compound of claim 1, or a pharmaceutically acceptable salt thereof.

27. The method of claim 26, wherein the bacterial infection is selected from the group consisting of community-acquired pneumoniae, hospital-acquired pneumoniae, skin and skin structure infections, acute exacerbation of chronic bronchitis, acute sinusitis, acute otitis media, catheter-related sepsis, febrile neutropenia, osteomyelitis, endocarditis, urinary tract infections and infections caused by drug resistant bacteria such as Penicillin-resistant Streptococcus pneumoniae, methicillin-resistant Staphylococcus aureus, methicillin-resistant Staphylococcus epidermidis and Vancomycin-Resistant Enterococci.

28. The method of any one of claim 26, wherein the warm-blooded animal is a human.

29. A process for preparing a compound of claim 1, or a pharmaceutically-acceptable salts thereof, comprising:
Process a) reacting a compound of formula (II′): 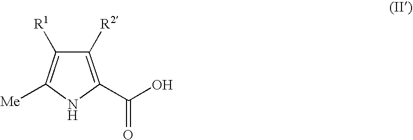
or an activated acid derivative thereof, with a compound of formula (III): 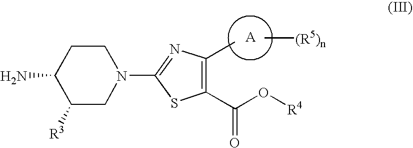
or
Process b) reacting a compound of formula (IV′): 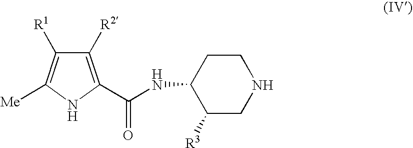
with a compound of formula (V): 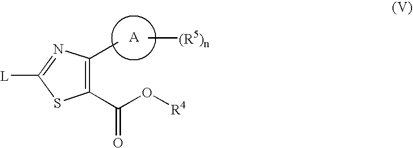
wherein L is a displaceable group; or
Process c) preparing a compound of formula (Ia), wherein R⁴ is C₁₋₄alkyl, comprising reacting a compound of formula (Ia), which is a compound of formula (VI′): 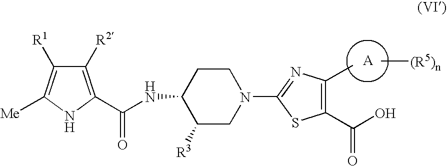
with a compound of formula (VII):
R^{4a}—OH (VI)
wherein R⁴a is C₁₋₄alkyl;
or
Process d) for preparing compounds of formula (Ia) wherein R⁴ is hydrogen; comprising deprotecting a compound of formula (VlIII′): 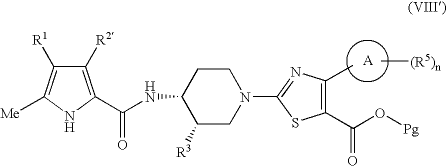
wherein Pg is a carboxylic acid protecting group;
and thereafter if necessary:
i) converting a compound of the formula (Ia) into another compound of the formula (Ia);
ii) removing any protecting groups;
iii) forming a pharmaceutically acceptable salt; and/or
iv) chirally purifying the compound of formula (Ia).
